SEQUENCE LISTING (1278 sequences)

SEQ ID NO: 1 - DNA, 23 bp, synthetic construct
  misc_feature 1..23: note = Synthetic: Target sequence (including PAM)
  source 1..23: mol_type = other DNA
ttggcaagtggacattttactgg

SEQ ID NO: 2 - DNA, 23 bp, synthetic construct
  misc_feature 1..23: note = Synthetic: Target sequence (including PAM)
  source 1..23: mol_type = other DNA
tgtccacttgccaaagaagttgg

SEQ ID NO: 3 - DNA, 23 bp, synthetic construct
  misc_feature 1..23: note = Synthetic: Target sequence (including PAM)
  source 1..23: mol_type = other DNA
ggaccaacttctttggcaagtgg

SEQ ID NO: 4 - DNA, 23 bp, synthetic construct
  misc_feature 1..23: note = Synthetic: Target sequence (including PAM)
  source 1..23: mol_type = other DNA
gaaaaatggaccaacttctttgg

SEQ ID NO: 5 - DNA, 23 bp, synthetic construct
  misc_feature 1..23: note = Synthetic: Target sequence (including PAM)
  source 1..23: mol_type = other DNA
ccatttttcccactgctcacagg

SEQ ID NO: 6 - DNA, 23 bp, synthetic construct
  misc_feature 1..23: note = Synthetic: Target sequence (including PAM)
  source 1..23: mol_type = other DNA
cctgtgagcagtgggaaaaatgg

SEQ ID NO: 7 - DNA, 23 bp, synthetic construct
  misc_feature 1..23: note = Synthetic: Target sequence (including PAM)
  source 1..23: mol_type = other DNA
tttttcccactgctcacaggagg

SEQ ID NO: 8 - DNA, 23 bp, synthetic construct
  misc_feature 1..23: note = Synthetic: Target sequence (including PAM)
  source 1..23: mol_type = other DNA
tttcacctcctgtgagcagtggg

SEQ ID NO: 9 - DNA, 23 bp, synthetic construct
  misc_feature 1..23: note = Synthetic: Target sequence (including PAM)
  source 1..23: mol_type = other DNA
ttttcacctcctgtgagcagtgg

SEQ ID NO: 10 - DNA, 23 bp, synthetic construct
  misc_feature 1..23: note = Synthetic: Target sequence (including PAM)
  source 1..23: mol_type = other DNA
agatctttgaggagctctgaagg

SEQ ID NO: 11 - DNA, 23 bp, synthetic construct
  misc_feature 1..23: note = Synthetic: Target sequence (including PAM)
  source 1..23: mol_type = other DNA
aacagtatgaaagatctttgagg

SEQ ID NO: 12 - DNA, 23 bp, synthetic construct
  misc_feature 1..23: note = Synthetic: Target sequence (including PAM)
  source 1..23: mol_type = other DNA
agcataaactctaagctgtttgg

SEQ ID NO: 13 - DNA, 23 bp, synthetic construct
  misc_feature 1..23: note = Synthetic: Target sequence (including PAM)
  source 1..23: mol_type = other DNA
acagcttagagtttatgctaagg

SEQ ID NO: 14 - DNA, 23 bp, synthetic construct
  misc_feature 1..23: note = Synthetic: Target sequence (including PAM)
  source 1..23: mol_type = other DNA
cagcttagagtttatgctaaggg

SEQ ID NO: 15 - DNA, 23 bp, synthetic construct
  misc_feature 1..23: note = Synthetic: Target sequence (including PAM)
  source 1..23: mol_type = other DNA
tcttttagttttaagttggatgg

SEQ ID NO: 16 - DNA, 23 bp, synthetic construct
  misc_feature 1..23: note = Synthetic: Target sequence (including PAM)
  source 1..23: mol_type = other DNA
tttctcttttagttttaagttgg

SEQ ID NO: 17 - DNA, 23 bp, synthetic construct
  misc_feature 1..23: note = Synthetic: Target sequence (including PAM)
  source 1..23: mol_type = other DNA
gtgataatgggggctggggtggg

SEQ ID NO: 18 - DNA, 23 bp, synthetic construct
  misc_feature 1..23: note = Synthetic: Target sequence (including PAM)
  source 1..23: mol_type = other DNA
agtgataatgggggctggggtgg

SEQ ID NO: 19 - DNA, 23 bp, synthetic construct
  misc_feature 1..23: note = Synthetic: Target sequence (including PAM)
  source 1..23: mol_type = other DNA
cagagtgataatgggggctgggg

SEQ ID NO: 20 - DNA, 23 bp, synthetic construct
  misc_feature 1..23: note = Synthetic: Target sequence (including PAM)
  source 1..23: mol_type = other DNA
acagagtgataatgggggctggg

SEQ ID NO: 21 - DNA, 23 bp, synthetic construct
  misc_feature 1..23: note = Synthetic: Target sequence (including PAM)
  source 1..23: mol_type = other DNA
aacagagtgataatgggggctgg

SEQ ID NO: 22 - DNA, 23 bp, synthetic construct
  misc_feature 1..23: note = Synthetic: Target sequence (including PAM)
  source 1..23: mol_type = other DNA
aaagaacagagtgataatggggg

SEQ ID NO: 23 - DNA, 23 bp, synthetic construct
  misc_feature 1..23: note = Synthetic: Target sequence (including PAM)
  source 1..23: mol_type = other DNA
gaaagaacagagtgataatgggg

SEQ ID NO: 24 - DNA, 23 bp, synthetic construct
  misc_feature 1..23: note = Synthetic: Target sequence (including PAM)
  source 1..23: mol_type = other DNA
agaaagaacagagtgataatggg

SEQ ID NO: 25 - DNA, 23 bp, synthetic construct
  misc_feature 1..23: note = Synthetic: Target sequence (including PAM)
  source 1..23: mol_type = other DNA
aagaaagaacagagtgataatgg

SEQ ID NO: 26 - DNA, 23 bp, synthetic construct
  misc_feature 1..23: note = Synthetic: Target sequence (including PAM)
  source 1..23: mol_type = other DNA
tctgttctttctttttcctcagg

SEQ ID NO: 27 - DNA, 23 bp, synthetic construct
  misc_feature 1..23: note = Synthetic: Target sequence (including PAM)
  source 1..23: mol_type = other DNA
ttttcctcaggttcattagatgg

SEQ ID NO: 28 - DNA, 23 bp, synthetic construct
  misc_feature 1..23: note = Synthetic: Target sequence (including PAM)
  source 1..23: mol_type = other DNA
ttggccatctaatgaacctgagg

SEQ ID NO: 29 - DNA, 23 bp, synthetic construct
  misc_feature 1..23: note = Synthetic: Target sequence (including PAM)
  source 1..23: mol_type = other DNA
aatgtagcagtagtactgcttgg

SEQ ID NO: 30 - DNA, 23 bp, synthetic construct
  misc_feature 1..23: note = Synthetic: Target sequence (including PAM)
  source 1..23: mol_type = other DNA
agcagtactactgctacatttgg

SEQ ID NO: 31 - DNA, 23 bp, synthetic construct
  misc_feature 1..23: note = Synthetic: Target sequence (including PAM)
  source 1..23: mol_type = other DNA
tgaatcttgataacattatgggg

SEQ ID NO: 32 - DNA, 23 bp, synthetic construct
  misc_feature 1..23: note = Synthetic: Target sequence (including PAM)
  source 1..23: mol_type = other DNA
ctgaatcttgataacattatggg

SEQ ID NO: 33 - DNA, 23 bp, synthetic construct
  misc_feature 1..23: note = Synthetic: Target sequence (including PAM)
  source 1..23: mol_type = other DNA
ccataatgttatcaagattcagg

SEQ ID NO: 34 - DNA, 23 bp, synthetic construct
  misc_feature 1..23: note = Synthetic: Target sequence (including PAM)
  source 1..23: mol_type = other DNA
cctgaatcttgataacattatgg

SEQ ID NO: 35 - DNA, 23 bp, synthetic construct
  misc_feature 1..23: note = Synthetic: Target sequence (including PAM)
  source 1..23: mol_type = other DNA
aatgttatcaagattcaggttgg

SEQ ID NO: 36 - DNA, 23 bp, synthetic construct
  misc_feature 1..23: note = Synthetic: Target sequence (including PAM)
  source 1..23: mol_type = other DNA
gttatcaagattcaggttggagg

SEQ ID NO: 37 - DNA, 23 bp, synthetic construct
  misc_feature 1..23: note = Synthetic: Target sequence (including PAM)
  source 1..23: mol_type = other DNA
tgtttttctagagaggctgctgg

SEQ ID NO: 38 - DNA, 23 bp, synthetic construct
  misc_feature 1..23: note = Synthetic: Target sequence (including PAM)
  source 1..23: mol_type = other DNA
aaactagtgtttttctagagagg

SEQ ID NO: 39 - DNA, 23 bp, synthetic construct
  misc_feature 1..23: note = Synthetic: Target sequence (including PAM)
  source 1..23: mol_type = other DNA
gaaaaacactagtttcaccaagg

SEQ ID NO: 40 - DNA, 23 bp, synthetic construct
  misc_feature 1..23: note = Synthetic: Target sequence (including PAM)
  source 1..23: mol_type = other DNA
aacaacttttttcttctccttgg

SEQ ID NO: 41 - DNA, 23 bp, synthetic construct
  misc_feature 1..23: note = Synthetic: Target sequence (including PAM)
  source 1..23: mol_type = other DNA
ttgttttatattgaaaaccttgg

SEQ ID NO: 42 - DNA, 23 bp, synthetic construct
  misc_feature 1..23: note = Synthetic: Target sequence (including PAM)
  source 1..23: mol_type = other DNA
gaaaaccttggccataaacgtgg

SEQ ID NO: 43 - DNA, 23 bp, synthetic construct
  misc_feature 1..23: note = Synthetic: Target sequence (including PAM)
  source 1..23: mol_type = other DNA
cattgccacgtttatggccaagg

SEQ ID NO: 44 - DNA, 23 bp, synthetic construct
  misc_feature 1..23: note = Synthetic: Target sequence (including PAM)
  source 1..23: mol_type = other DNA
aatggacattgccacgtttatgg

SEQ ID NO: 45 - DNA, 23 bp, synthetic construct
  misc_feature 1..23: note = Synthetic: Target sequence (including PAM)
  source 1..23: mol_type = other DNA
tgtccatttccatctcgtatagg

SEQ ID NO: 46 - DNA, 23 bp, synthetic construct
  misc_feature 1..23: note = Synthetic: Target sequence (including PAM)
  source 1..23: mol_type = other DNA
aatcctatacgagatggaaatgg

SEQ ID NO: 47 - DNA, 23 bp, synthetic construct
  misc_feature 1..23: note = Synthetic: Target sequence (including PAM)
  source 1..23: mol_type = other DNA
caggcaaatcctatacgagatgg

SEQ ID NO: 48 - DNA, 23 bp, synthetic construct
  misc_feature 1..23: note = Synthetic: Target sequence (including PAM)
  source 1..23: mol_type = other DNA
tatttgggttcacatatgacagg

SEQ ID NO: 49 - DNA, 23 bp, synthetic construct
  misc_feature 1..23: note = Synthetic: Target sequence (including PAM)
  source 1..23: mol_type = other DNA
tggcacttttatttttatttggg

SEQ ID NO: 50 - DNA, 23 bp, synthetic construct
  misc_feature 1..23: note = Synthetic: Target sequence (including PAM)
  source 1..23: mol_type = other DNA
gtggcacttttatttttatttgg

SEQ ID NO: 51 - DNA, 23 bp, synthetic construct
  misc_feature 1..23: note = Synthetic: Target sequence (including PAM)
  source 1..23: mol_type = other DNA
aaatgagaatttagtgcaggtgg

SEQ ID NO: 52 - DNA, 23 bp, synthetic construct
  misc_feature 1..23: note = Synthetic: Target sequence (including PAM)
  source 1..23: mol_type = other DNA
acgaaatgagaatttagtgcagg

SEQ ID NO: 53 - DNA, 23 bp, synthetic construct
  misc_feature 1..23: note = Synthetic: Target sequence (including PAM)
  source 1..23: mol_type = other DNA
attctcatttcgtctctaacagg

SEQ ID NO: 54 - DNA, 23 bp, synthetic construct
  misc_feature 1..23: note = Synthetic: Target sequence (including PAM)
  source 1..23: mol_type = other DNA
aaataaatgctggagagagaggg

SEQ ID NO: 55 - DNA, 23 bp, synthetic construct
  misc_feature 1..23: note = Synthetic: Target sequence (including PAM)
  source 1..23: mol_type = other DNA
gaaataaatgctggagagagagg

SEQ ID NO: 56 - DNA, 23 bp, synthetic construct
  misc_feature 1..23: note = Synthetic: Target sequence (including PAM)
  source 1..23: mol_type = other DNA
attagggtcgaaataaatgctgg

SEQ ID NO: 57 - DNA, 23 bp, synthetic construct
  misc_feature 1..23: note = Synthetic: Target sequence (including PAM)
  source 1..23: mol_type = other DNA
gcatttatttcgaccctaattgg

SEQ ID NO: 58 - DNA, 23 bp, synthetic construct
  misc_feature 1..23: note = Synthetic: Target sequence (including PAM)
  source 1..23: mol_type = other DNA
aagaagagggaaaccaattaggg

SEQ ID NO: 59 - DNA, 23 bp, synthetic construct
  misc_feature 1..23: note = Synthetic: Target sequence (including PAM)
  source 1..23: mol_type = other DNA
gaagaagagggaaaccaattagg

SEQ ID NO: 60 - DNA, 23 bp, synthetic construct
  misc_feature 1..23: note = Synthetic: Target sequence (including PAM)
  source 1..23: mol_type = other DNA
actagatacgtcgaagaagaggg

SEQ ID NO: 61 - DNA, 23 bp, synthetic construct
  misc_feature 1..23: note = Synthetic: Target sequence (including PAM)
  source 1..23: mol_type = other DNA
cactagatacgtcgaagaagagg

SEQ ID NO: 62 - DNA, 23 bp, synthetic construct
  misc_feature 1..23: note = Synthetic: Target sequence (including PAM)
  source 1..23: mol_type = other DNA
ctcttcttcgacgtatctagtgg

SEQ ID NO: 63 - DNA, 23 bp, synthetic construct
  misc_feature 1..23: note = Synthetic: Target sequence (including PAM)
  source 1..23: mol_type = other DNA
tgcaggctctgactcagggaagg

SEQ ID NO: 64 - DNA, 23 bp, synthetic construct
  misc_feature 1..23: note = Synthetic: Target sequence (including PAM)
  source 1..23: mol_type = other DNA
tttttgcaggctctgactcaggg

SEQ ID NO: 65 - DNA, 23 bp, synthetic construct
  misc_feature 1..23: note = Synthetic: Target sequence (including PAM)
  source 1..23: mol_type = other DNA
ctttttgcaggctctgactcagg

SEQ ID NO: 66 - DNA, 23 bp, synthetic construct
  misc_feature 1..23: note = Synthetic: Target sequence (including PAM)
  source 1..23: mol_type = other DNA
tcagagcctgcaaaaagcaaagg

SEQ ID NO: 67 - DNA, 23 bp, synthetic construct
  misc_feature 1..23: note = Synthetic: Target sequence (including PAM)
  source 1..23: mol_type = other DNA
ttcgttcctttgctttttgcagg

SEQ ID NO: 68 - DNA, 23 bp, synthetic construct
  misc_feature 1..23: note = Synthetic: Target sequence (including PAM)
  source 1..23: mol_type = other DNA
gcaaaaagcaaaggaacgaatgg

SEQ ID NO: 69 - DNA, 23 bp, synthetic construct
  misc_feature 1..23: note = Synthetic: Target sequence (including PAM)
  source 1..23: mol_type = other DNA
agaaagtgcaacaagcagaaagg

SEQ ID NO: 70 - DNA, 23 bp, synthetic construct
  misc_feature 1..23: note = Synthetic: Target sequence (including PAM)
  source 1..23: mol_type = other DNA
gaaagtgcaacaagcagaaaggg

SEQ ID NO: 71 - DNA, 23 bp, synthetic construct
  misc_feature 1..23: note = Synthetic: Target sequence (including PAM)
  source 1..23: mol_type = other DNA
aaagtgcaacaagcagaaagggg

SEQ ID NO: 72 - DNA, 23 bp, synthetic construct
  misc_feature 1..23: note = Synthetic: Target sequence (including PAM)
  source 1..23: mol_type = other DNA
aagtgcaacaagcagaaaggggg

SEQ ID NO: 73 - DNA, 23 bp, synthetic construct
  misc_feature 1..23: note = Synthetic: Target sequence (including PAM)
  source 1..23: mol_type = other DNA
ggctgcaaagctgcctgcctagg

SEQ ID NO: 74 - DNA, 23 bp, synthetic construct
  misc_feature 1..23: note = Synthetic: Target sequence (including PAM)
  source 1..23: mol_type = other DNA
gctgcaaagctgcctgcctaggg

SEQ ID NO: 75 - DNA, 23 bp, synthetic construct
  misc_feature 1..23: note = Synthetic: Target sequence (including PAM)
  source 1..23: mol_type = other DNA
caggaaacgtagccctaggcagg

SEQ ID NO: 76 - DNA, 23 bp, synthetic construct
  misc_feature 1..23: note = Synthetic: Target sequence (including PAM)
  source 1..23: mol_type = other DNA
ctgcctagggctacgtttcctgg

SEQ ID NO: 77 - DNA, 23 bp, synthetic construct
  misc_feature 1..23: note = Synthetic: Target sequence (including PAM)
  source 1..23: mol_type = other DNA
ttgccaggaaacgtagccctagg

SEQ ID NO: 78 - DNA, 23 bp, synthetic construct
  misc_feature 1..23: note = Synthetic: Target sequence (including PAM)
  source 1..23: mol_type = other DNA
tggctttcggaagttttgccagg

SEQ ID NO: 79 - DNA, 23 bp, synthetic construct
  misc_feature 1..23: note = Synthetic: Target sequence (including PAM)
  source 1..23: mol_type = other DNA
tcttttggagaaatggctttcgg

SEQ ID NO: 80 - DNA, 23 bp, synthetic construct
  misc_feature 1..23: note = Synthetic: Target sequence (including PAM)
  source 1..23: mol_type = other DNA
aaagccatttctccaaaagaagg

SEQ ID NO: 81 - DNA, 23 bp, synthetic construct
  misc_feature 1..23: note = Synthetic: Target sequence (including PAM)
  source 1..23: mol_type = other DNA
tagaccttcttttggagaaatgg

SEQ ID NO: 82 - DNA, 23 bp, synthetic construct
  misc_feature 1..23: note = Synthetic: Target sequence (including PAM)
  source 1..23: mol_type = other DNA
tccaaaagaaggtctagaagagg

SEQ ID NO: 83 - DNA, 23 bp, synthetic construct
  misc_feature 1..23: note = Synthetic: Target sequence (including PAM)
  source 1..23: mol_type = other DNA
tcctcttctagaccttcttttgg

SEQ ID NO: 84 - DNA, 23 bp, synthetic construct
  misc_feature 1..23: note = Synthetic: Target sequence (including PAM)
  source 1..23: mol_type = other DNA
aaaagaaggtctagaagaggagg

SEQ ID NO: 85 - DNA, 23 bp, synthetic construct
  misc_feature 1..23: note = Synthetic: Target sequence (including PAM)
  source 1..23: mol_type = other DNA
agaaggtctagaagaggaggagg

SEQ ID NO: 86 - DNA, 23 bp, synthetic construct
  misc_feature 1..23: note = Synthetic: Target sequence (including PAM)
  source 1..23: mol_type = other DNA
aggtctagaagaggaggaggagg

SEQ ID NO: 87 - DNA, 23 bp, synthetic construct
  misc_feature 1..23: note = Synthetic: Target sequence (including PAM)
  source 1..23: mol_type = other DNA
tctagaagaggaggaggaggagg

SEQ ID NO: 88 - DNA, 23 bp, synthetic construct
  misc_feature 1..23: note = Synthetic: Target sequence (including PAM)
  source 1..23: mol_type = other DNA
agaggaggaggaggaggagaagg

SEQ ID NO: 89 - DNA, 23 bp, synthetic construct
  misc_feature 1..23: note = Synthetic: Target sequence (including PAM)
  source 1..23: mol_type = other DNA
ggaggaggaggaggagaaggagg

SEQ ID NO: 90 - DNA, 23 bp, synthetic construct
  misc_feature 1..23: note = Synthetic: Target sequence (including PAM)
  source 1..23: mol_type = other DNA
ggaggaggaggagaaggaggagg

SEQ ID NO: 91 - DNA, 23 bp, synthetic construct
  misc_feature 1..23: note = Synthetic: Target sequence (including PAM)
  source 1..23: mol_type = other DNA
ggaggaggagaaggaggaggagg

SEQ ID NO: 92 - DNA, 23 bp, synthetic construct
  misc_feature 1..23: note = Synthetic: Target sequence (including PAM)
  source 1..23: mol_type = other DNA
ggaggagaaggaggaggaggagg

SEQ ID NO: 93 - DNA, 23 bp, synthetic construct
  misc_feature 1..23: note = Synthetic: Target sequence (including PAM)
  source 1..23: mol_type = other DNA
ggagaaggaggaggaggaggagg

SEQ ID NO: 94 - DNA, 23 bp, synthetic construct
  misc_feature 1..23: note = Synthetic: Target sequence (including PAM)
  source 1..23: mol_type = other DNA
cagcatgaaagagccccacttgg

SEQ ID NO: 95 - DNA, 23 bp, synthetic construct
  misc_feature 1..23: note = Synthetic: Target sequence (including PAM)
  source 1..23: mol_type = other DNA
atgaaagagccccacttggaagg

SEQ ID NO: 96 - DNA, 23 bp, synthetic construct
  misc_feature 1..23: note = Synthetic: Target sequence (including PAM)
  source 1..23: mol_type = other DNA
aaagagccccacttggaaggcgg

SEQ ID NO: 97 - DNA, 23 bp, synthetic construct
  misc_feature 1..23: note = Synthetic: Target sequence (including PAM)
  source 1..23: mol_type = other DNA
gccccacttggaaggcggtttgg

SEQ ID NO: 98 - DNA, 23 bp, synthetic construct
  misc_feature 1..23: note = Synthetic: Target sequence (including PAM)
  source 1..23: mol_type = other DNA
tccaaaccgccttccaagtgggg

SEQ ID NO: 99 - DNA, 23 bp, synthetic construct
  misc_feature 1..23: note = Synthetic: Target sequence (including PAM)
  source 1..23: mol_type = other DNA
atccaaaccgccttccaagtggg

SEQ ID NO: 100 - DNA, 23 bp, synthetic construct
  misc_feature 1..23: note = Synthetic: Target sequence (including PAM)
  source 1..23: mol_type = other DNA
aatccaaaccgccttccaagtgg

SEQ ID NO: 101 - DNA, 23 bp, synthetic construct
  misc_feature 1..23: note = Synthetic: Target sequence (including PAM)
  source 1..23: mol_type = other DNA
gattttatttgtgtgttttgtgg

SEQ ID NO: 102 - DNA, 23 bp, synthetic construct
  misc_feature 1..23: note = Synthetic: Target sequence (including PAM)
  source 1..23: mol_type = other DNA
catcttacaccaaactcatctgg

SEQ ID NO: 103 - DNA, 23 bp, synthetic construct
  misc_feature 1..23: note = Synthetic: Target sequence (including PAM)
  source 1..23: mol_type = other DNA
tttttaatgccagatgagtttgg

SEQ ID NO: 104 - DNA, 23 bp, synthetic construct
  misc_feature 1..23: note = Synthetic: Target sequence (including PAM)
  source 1..23: mol_type = other DNA
attcattctcctgacatgtctgg

SEQ ID NO: 105 - DNA, 23 bp, synthetic construct
  misc_feature 1..23: note = Synthetic: Target sequence (including PAM)
  source 1..23: mol_type = other DNA
ttcattctcctgacatgtctggg

SEQ ID NO: 106 - DNA, 23 bp, synthetic construct
  misc_feature 1..23: note = Synthetic: Target sequence (including PAM)
  source 1..23: mol_type = other DNA
ctcctgacatgtctgggacttgg

SEQ ID NO: 107 - DNA, 23 bp, synthetic construct
  misc_feature 1..23: note = Synthetic: Target sequence (including PAM)
  source 1..23: mol_type = other DNA
aaccaagtcccagacatgtcagg

SEQ ID NO: 108 - DNA, 23 bp, synthetic construct
  misc_feature 1..23: note = Synthetic: Target sequence (including PAM)
  source 1..23: mol_type = other DNA
acatgtctgggacttggtttagg

SEQ ID NO: 109 - DNA, 23 bp, synthetic construct
  misc_feature 1..23: note = Synthetic: Target sequence (including PAM)
  source 1..23: mol_type = other DNA
ctgggacttggtttaggaaaagg

SEQ ID NO: 110 - DNA, 23 bp, synthetic construct
  misc_feature 1..23: note = Synthetic: Target sequence (including PAM)
  source 1..23: mol_type = other DNA
ggtttaggaaaaggaagcaaagg

SEQ ID NO: 111 - DNA, 23 bp, synthetic construct
  misc_feature 1..23: note = Synthetic: Target sequence (including PAM)
  source 1..23: mol_type = other DNA
gtttaggaaaaggaagcaaaggg

SEQ ID NO: 112 - DNA, 23 bp, synthetic construct
  misc_feature 1..23: note = Synthetic: Target sequence (including PAM)
  source 1..23: mol_type = other DNA
aggaaaaggaagcaaagggatgg

SEQ ID NO: 113 - DNA, 23 bp, synthetic construct
  misc_feature 1..23: note = Synthetic: Target sequence (including PAM)
  source 1..23: mol_type = other DNA
aggaagcaaagggatggagaagg

SEQ ID NO: 114 - DNA, 23 bp, synthetic construct
  misc_feature 1..23: note = Synthetic: Target sequence (including PAM)
  source 1..23: mol_type = other DNA
tggagttttacggctgtacttgg

SEQ ID NO: 115 - DNA, 23 bp, synthetic construct
  misc_feature 1..23: note = Synthetic: Target sequence (including PAM)
  source 1..23: mol_type = other DNA
gacacacttgtggagttttacgg

SEQ ID NO: 116 - DNA, 23 bp, synthetic construct
  misc_feature 1..23: note = Synthetic: Target sequence (including PAM)
  source 1..23: mol_type = other DNA
agcggaacttgacacacttgtgg

SEQ ID NO: 117 - DNA, 23 bp, synthetic construct
  misc_feature 1..23: note = Synthetic: Target sequence (including PAM)
  source 1..23: mol_type = other DNA
gtcgtaggatcagcacaaagcgg

SEQ ID NO: 118 - DNA, 23 bp, synthetic construct
  misc_feature 1..23: note = Synthetic: Target sequence (including PAM)
  source 1..23: mol_type = other DNA
ttggtaaatttcgtagtcgtagg

SEQ ID NO: 119 - DNA, 23 bp, synthetic construct
  misc_feature 1..23: note = Synthetic: Target sequence (including PAM)
  source 1..23: mol_type = other DNA
atttaccaaaacagtccaaaagg

SEQ ID NO: 120 - DNA, 23 bp, synthetic construct
  misc_feature 1..23: note = Synthetic: Target sequence (including PAM)
  source 1..23: mol_type = other DNA
tagaaccttttggactgttttgg

SEQ ID NO: 121 - DNA, 23 bp, synthetic construct
  misc_feature 1..23: note = Synthetic: Target sequence (including PAM)
  source 1..23: mol_type = other DNA
atacattctttagaaccttttgg

SEQ ID NO: 122 - DNA, 23 bp, synthetic construct
  misc_feature 1..23: note = Synthetic: Target sequence (including PAM)
  source 1..23: mol_type = other DNA
taggattcttaaaactagtatgg

SEQ ID NO: 123 - DNA, 23 bp, synthetic construct
  misc_feature 1..23: note = Synthetic: Target sequence (including PAM)
  source 1..23: mol_type = other DNA
atactagttttaagaatcctagg

SEQ ID NO: 124 - DNA, 23 bp, synthetic construct
  misc_feature 1..23: note = Synthetic: Target sequence (including PAM)
  source 1..23: mol_type = other DNA
tcctaggaaaagatgtaactagg

SEQ ID NO: 125 - DNA, 23 bp, synthetic construct
  misc_feature 1..23: note = Synthetic: Target sequence (including PAM)
  source 1..23: mol_type = other DNA
tcctagttacatcttttcctagg

SEQ ID NO: 126 - DNA, 23 bp, synthetic construct
  misc_feature 1..23: note = Synthetic: Target sequence (including PAM)
  source 1..23: mol_type = other DNA
taggaaaagatgtaactaggagg

SEQ ID NO: 127 - DNA, 23 bp, synthetic construct
  misc_feature 1..23: note = Synthetic: Target sequence (including PAM)
  source 1..23: mol_type = other DNA
taactaggaggtaagatgtaagg

SEQ ID NO: 128 - DNA, 23 bp, synthetic construct
  misc_feature 1..23: note = Synthetic: Target sequence (including PAM)
  source 1..23: mol_type = other DNA
ggaggtaagatgtaaggaacagg

SEQ ID NO: 129 - DNA, 23 bp, synthetic construct
  misc_feature 1..23: note = Synthetic: Target sequence (including PAM)
  source 1..23: mol_type = other DNA
taatgatgctttggattggtagg

SEQ ID NO: 130 - DNA, 23 bp, synthetic construct
  misc_feature 1..23: note = Synthetic: Target sequence (including PAM)
  source 1..23: mol_type = other DNA
aagctaatgatgctttggattgg

SEQ ID NO: 131 - DNA, 23 bp, synthetic construct
  misc_feature 1..23: note = Synthetic: Target sequence (including PAM)
  source 1..23: mol_type = other DNA
gttttaagctaatgatgctttgg

SEQ ID NO: 132 - DNA, 23 bp, synthetic construct
  misc_feature 1..23: note = Synthetic: Target sequence (including PAM)
  source 1..23: mol_type = other DNA
taaaactttaaagagacaactgg

SEQ ID NO: 133 - DNA, 23 bp, synthetic construct
  misc_feature 1..23: note = Synthetic: Target sequence (including PAM)
  source 1..23: mol_type = other DNA
aaaactttaaagagacaactggg

SEQ ID NO: 134 - DNA, 23 bp, synthetic construct
  misc_feature 1..23: note = Synthetic: Target sequence (including PAM)
  source 1..23: mol_type = other DNA
ggaaatggaaaatagaaaatagg

SEQ ID NO: 135 - DNA, 23 bp, synthetic construct
  misc_feature 1..23: note = Synthetic: Target sequence (including PAM)
  source 1..23: mol_type = other DNA
ttatttattgtttttggaaatgg

SEQ ID NO: 136 - DNA, 23 bp, synthetic construct
  misc_feature 1..23: note = Synthetic: Target sequence (including PAM)
  source 1..23: mol_type = other DNA
ttcgttttatttattgtttttgg

SEQ ID NO: 137 - DNA, 23 bp, synthetic construct
  misc_feature 1..23: note = Synthetic: Target sequence (including PAM)
  source 1..23: mol_type = other DNA
gtagtctcagtgttcagacatgg

SEQ ID NO: 138 - DNA, 23 bp, synthetic construct
  misc_feature 1..23: note = Synthetic: Target sequence (including PAM)
  source 1..23: mol_type = other DNA
ttcagacatggccaagttttagg

SEQ ID NO: 139 - DNA, 23 bp, synthetic construct
  misc_feature 1..23: note = Synthetic: Target sequence (including PAM)
  source 1..23: mol_type = other DNA
tcagacatggccaagttttaggg

SEQ ID NO: 140 - DNA, 23 bp, synthetic construct
  misc_feature 1..23: note = Synthetic: Target sequence (including PAM)
  source 1..23: mol_type = other DNA
cagacatggccaagttttagggg

SEQ ID NO: 141 - DNA, 23 bp, synthetic construct
  misc_feature 1..23: note = Synthetic: Target sequence (including PAM)
  source 1..23: mol_type = other DNA
acatggccaagttttaggggtgg

SEQ ID NO: 142 - DNA, 23 bp, synthetic construct
  misc_feature 1..23: note = Synthetic: Target sequence (including PAM)
  source 1..23: mol_type = other DNA
actaaaccacccctaaaacttgg

SEQ ID NO: 143 - DNA, 23 bp, synthetic construct
  misc_feature 1..23: note = Synthetic: Target sequence (including PAM)
  source 1..23: mol_type = other DNA
tttaggggtggtttagttttagg

SEQ ID NO: 144 - DNA, 23 bp, synthetic construct
  misc_feature 1..23: note = Synthetic: Target sequence (including PAM)
  source 1..23: mol_type = other DNA
ttaggggtggtttagttttaggg

SEQ ID NO: 145 - DNA, 23 bp, synthetic construct
  misc_feature 1..23: note = Synthetic: Target sequence (including PAM)
  source 1..23: mol_type = other DNA
taggggtggtttagttttagggg

SEQ ID NO: 146 - DNA, 23 bp, synthetic construct
  misc_feature 1..23: note = Synthetic: Target sequence (including PAM)
  source 1..23: mol_type = other DNA
tgtctatttttgctttccactgg

SEQ ID NO: 147 - DNA, 23 bp, synthetic construct
  misc_feature 1..23: note = Synthetic: Target sequence (including PAM)
  source 1..23: mol_type = other DNA
gtctatttttgctttccactggg

SEQ ID NO: 148 - DNA, 23 bp, synthetic construct
  misc_feature 1..23: note = Synthetic: Target sequence (including PAM)
  source 1..23: mol_type = other DNA
tctatttttgctttccactgggg

SEQ ID NO: 149 - DNA, 23 bp, synthetic construct
  misc_feature 1..23: note = Synthetic: Target sequence (including PAM)
  source 1..23: mol_type = other DNA
ataatggaatctcaccccagtgg

SEQ ID NO: 150 - DNA, 23 bp, synthetic construct
  misc_feature 1..23: note = Synthetic: Target sequence (including PAM)
  source 1..23: mol_type = other DNA
tggggtgagattccattatttgg

SEQ ID NO: 151 - DNA, 23 bp, synthetic construct
  misc_feature 1..23: note = Synthetic: Target sequence (including PAM)
  source 1..23: mol_type = other DNA
ggggtgagattccattatttggg

SEQ ID NO: 152 - DNA, 23 bp, synthetic construct
  misc_feature 1..23: note = Synthetic: Target sequence (including PAM)
  source 1..23: mol_type = other DNA
gggtgagattccattatttgggg

SEQ ID NO: 153 - DNA, 23 bp, synthetic construct
  misc_feature 1..23: note = Synthetic: Target sequence (including PAM)
  source 1..23: mol_type = other DNA
ccattatttggggtaatcagtgg

SEQ ID NO: 154 - DNA, 23 bp, synthetic construct
  misc_feature 1..23: note = Synthetic: Target sequence (including PAM)
  source 1..23: mol_type = other DNA
ccactgattaccccaaataatgg

SEQ ID NO: 155 - DNA, 23 bp, synthetic construct
  misc_feature 1..23: note = Synthetic: Target sequence (including PAM)
  source 1..23: mol_type = other DNA
cattatttggggtaatcagtggg

SEQ ID NO: 156 - DNA, 23 bp, synthetic construct
  misc_feature 1..23: note = Synthetic: Target sequence (including PAM)
  source 1..23: mol_type = other DNA
atttggggtaatcagtgggtagg

SEQ ID NO: 157 - DNA, 23 bp, synthetic construct
  misc_feature 1..23: note = Synthetic: Target sequence (including PAM)
  source 1..23: mol_type = other DNA
tttggggtaatcagtgggtaggg

SEQ ID NO: 158 - DNA, 23 bp, synthetic construct
  misc_feature 1..23: note = Synthetic: Target sequence (including PAM)
  source 1..23: mol_type = other DNA
atcagtgggtagggaattgaagg

SEQ ID NO: 159 - DNA, 23 bp, synthetic construct
  misc_feature 1..23: note = Synthetic: Target sequence (including PAM)
  source 1..23: mol_type = other DNA
ttttttttgagttttattactgg

SEQ ID NO: 160 - DNA, 23 bp, synthetic construct
  misc_feature 1..23: note = Synthetic: Target sequence (including PAM)
  source 1..23: mol_type = other DNA
tgtggtgtgatggaagattcagg

SEQ ID NO: 161 - DNA, 23 bp, synthetic construct
  misc_feature 1..23: note = Synthetic: Target sequence (including PAM)
  source 1..23: mol_type = other DNA
actataattttgtggtgtgatgg

SEQ ID NO: 162 - DNA, 23 bp, synthetic construct
  misc_feature 1..23: note = Synthetic: Target sequence (including PAM)
  source 1..23: mol_type = other DNA
agtttttaactataattttgtgg

SEQ ID NO: 163 - DNA, 23 bp, synthetic construct
  misc_feature 1..23: note = Synthetic: Target sequence (including PAM)
  source 1..23: mol_type = other DNA
aaagaccttcatatttaccaagg

SEQ ID NO: 164 - DNA, 23 bp, synthetic construct
  misc_feature 1..23: note = Synthetic: Target sequence (including PAM)
  source 1..23: mol_type = other DNA
tgaatccttggtaaatatgaagg

SEQ ID NO: 165 - DNA, 23 bp, synthetic construct
  misc_feature 1..23: note = Synthetic: Target sequence (including PAM)
  source 1..23: mol_type = other DNA
tttttaattggctgaatccttgg

SEQ ID NO: 166 - DNA, 23 bp, synthetic construct
  misc_feature 1..23: note = Synthetic: Target sequence (including PAM)
  source 1..23: mol_type = other DNA
ggacagtaataatttttaattgg

SEQ ID NO: 167 - DNA, 23 bp, synthetic construct
  misc_feature 1..23: note = Synthetic: Target sequence (including PAM)
  source 1..23: mol_type = other DNA
actgtcctttagattcctactgg

SEQ ID NO: 168 - DNA, 23 bp, synthetic construct
  misc_feature 1..23: note = Synthetic: Target sequence (including PAM)
  source 1..23: mol_type = other DNA
agaaaccagtaggaatctaaagg

SEQ ID NO: 169 - DNA, 23 bp, synthetic construct
  misc_feature 1..23: note = Synthetic: Target sequence (including PAM)
  source 1..23: mol_type = other DNA
cacttcagctagaaaccagtagg

SEQ ID NO: 170 - DNA, 23 bp, synthetic construct
  misc_feature 1..23: note = Synthetic: Target sequence (including PAM)
  source 1..23: mol_type = other DNA
tggtttctagctgaagtgtttgg

SEQ ID NO: 171 - DNA, 23 bp, synthetic construct
  misc_feature 1..23: note = Synthetic: Target sequence (including PAM)
  source 1..23: mol_type = other DNA
ggtttctagctgaagtgtttggg

SEQ ID NO: 172 - DNA, 23 bp, synthetic construct
  misc_feature 1..23: note = Synthetic: Target sequence (including PAM)
  source 1..23: mol_type = other DNA
agtgcggtaagaaagaacggtgg

SEQ ID NO: 173 - DNA, 23 bp, synthetic construct
  misc_feature 1..23: note = Synthetic: Target sequence (including PAM)
  source 1..23: mol_type = other DNA
ttcagtgcggtaagaaagaacgg

SEQ ID NO: 174 - DNA, 23 bp, synthetic construct
  misc_feature 1..23: note = Synthetic: Target sequence (including PAM)
  source 1..23: mol_type = other DNA
tgatttactggatttcagtgcgg

SEQ ID NO: 175 - DNA, 23 bp, synthetic construct
  misc_feature 1..23: note = Synthetic: Target sequence (including PAM)
  source 1..23: mol_type = other DNA
caaagagctgagtgatttactgg

SEQ ID NO: 176 - DNA, 23 bp, synthetic construct
  misc_feature 1..23: note = Synthetic: Target sequence (including PAM)
  source 1..23: mol_type = other DNA
cagctctttgtccgtccctaagg

SEQ ID NO: 177 - DNA, 23 bp, synthetic construct
  misc_feature 1..23: note = Synthetic: Target sequence (including PAM)
  source 1..23: mol_type = other DNA
gcgaatggctgccttagggacgg

SEQ ID NO: 178 - DNA, 23 bp, synthetic construct
  misc_feature 1..23: note = Synthetic: Target sequence (including PAM)
  source 1..23: mol_type = other DNA
aacagcgaatggctgccttaggg

SEQ ID NO: 179 - DNA, 23 bp, synthetic construct
  misc_feature 1..23: note = Synthetic: Target sequence (including PAM)
  source 1..23: mol_type = other DNA
caacagcgaatggctgccttagg

SEQ ID NO: 180 - DNA, 23 bp, synthetic construct
  misc_feature 1..23: note = Synthetic: Target sequence (including PAM)
  source 1..23: mol_type = other DNA
ctaaggcagccattcgctgttgg

SEQ ID NO: 181 - DNA, 23 bp, synthetic construct
  misc_feature 1..23: note = Synthetic: Target sequence (including PAM)
  source 1..23: mol_type = other DNA
aatgcatcaccaacagcgaatgg

SEQ ID NO: 182 - DNA, 23 bp, synthetic construct
  misc_feature 1..23: note = Synthetic: Target sequence (including PAM)
  source 1..23: mol_type = other DNA
atcacacaaacctagaaacatgg

SEQ ID NO: 183 - DNA, 23 bp, synthetic construct
  misc_feature 1..23: note = Synthetic: Target sequence (including PAM)
  source 1..23: mol_type = other DNA
gcggttatttccatgtttctagg

SEQ ID NO: 184 - DNA, 23 bp, synthetic construct
  misc_feature 1..23: note = Synthetic: Target sequence (including PAM)
  source 1..23: mol_type = other DNA
gggactggattttctgattgcgg

SEQ ID NO: 185 - DNA, 23 bp, synthetic construct
  misc_feature 1..23: note = Synthetic: Target sequence (including PAM)
  source 1..23: mol_type = other DNA
gaaaatccagtcccaatccttgg

SEQ ID NO: 186 - DNA, 23 bp, synthetic construct
  misc_feature 1..23: note = Synthetic: Target sequence (including PAM)
  source 1..23: mol_type = other DNA
ttttctccaaggattgggactgg

SEQ ID NO: 187 - DNA, 23 bp, synthetic construct
  misc_feature 1..23: note = Synthetic: Target sequence (including PAM)
  source 1..23: mol_type = other DNA
ttgtgttttctccaaggattggg

SEQ ID NO: 188 - DNA, 23 bp, synthetic construct
  misc_feature 1..23: note = Synthetic: Target sequence (including PAM)
  source 1..23: mol_type = other DNA
attgtgttttctccaaggattgg

SEQ ID NO: 189 - DNA, 23 bp, synthetic construct
  misc_feature 1..23: note = Synthetic: Target sequence (including PAM)
  source 1..23: mol_type = other DNA
atccttggagaaaacacaatcgg

SEQ ID NO: 190 - DNA, 23 bp, synthetic construct
  misc_feature 1..23: note = Synthetic: Target sequence (including PAM)
  source 1..23: mol_type = other DNA
atccgattgtgttttctccaagg

SEQ ID NO: 191 - DNA, 23 bp, synthetic construct
  misc_feature 1..23: note = Synthetic: Target sequences for wild type COL8A2
  source 1..23: mol_type = other DNA
ggggaggaggccagggcagcagg

SEQ ID NO: 192 - DNA, 23 bp, synthetic construct
  misc_feature 1..23: note = Synthetic: Target sequences for wild type COL8A2
  source 1..23: mol_type = other DNA
gggcagcaggaccccccccgcgg

SEQ ID NO: 193 - DNA, 23 bp, synthetic construct
  misc_feature 1..23: note = Synthetic: Target sequences for wild type COL8A2
  source 1..23: mol_type = other DNA
ggcagcaggaccccccccgcggg

SEQ ID NO: 194 - DNA, 23 bp, synthetic construct
  misc_feature 1..23: note = Synthetic: Target sequences for wild type COL8A2
  source 1..23: mol_type = other DNA
gaccccccccgcgggttatgtgg

SEQ ID NO: 195 - DNA, 23 bp, synthetic construct
  misc_feature 1..23: note = Synthetic: Target sequences for wild type COL8A2
  source 1..23: mol_type = other DNA
accccccccgcgggttatgtggg

SEQ ID NO: 196 - DNA, 23 bp, synthetic construct
  misc_feature 1..23: note = Synthetic: Target sequences for wild type COL8A2
  source 1..23: mol_type = other DNA
ccccccccgcgggttatgtgggg

SEQ ID NO: 197 - DNA, 23 bp, synthetic construct
  misc_feature 1..23: note = Synthetic: Target sequences for wild type COL8A2
  source 1..23: mol_type = other DNA
ccccacataacccgcgggggggg

SEQ ID NO: 198 - DNA, 23 bp, synthetic construct
  misc_feature 1..23: note = Synthetic: Target sequences for wild type COL8A2
  source 1..23: mol_type = other DNA
gccccacataacccgcggggggg

SEQ ID NO: 199 - DNA, 23 bp, synthetic construct
  misc_feature 1..23: note = Synthetic: Target sequences for wild type COL8A2
  source 1..23: mol_type = other DNA
tgccccacataacccgcgggggg

SEQ ID NO: 200 - DNA, 23 bp, synthetic construct
  misc_feature 1..23: note = Synthetic: Target sequences for wild type COL8A2
  source 1..23: mol_type = other DNA
ctgccccacataacccgcggggg

SEQ ID NO: 201 - DNA, 23 bp, synthetic construct
  misc_feature 1..23: note = Synthetic: Target sequences for wild type COL8A2
  source 1..23: mol_type = other DNA
tctgccccacataacccgcgggg

SEQ ID NO: 202 - DNA, 23 bp, synthetic construct
  misc_feature 1..23: note = Synthetic: Target sequences for wild type COL8A2
  source 1..23: mol_type = other DNA
ctctgccccacataacccgcggg

SEQ ID NO: 203 - DNA, 23 bp, synthetic construct
  misc_feature 1..23: note = Synthetic: Target sequences for wild type COL8A2
  source 1..23: mol_type = other DNA
gctctgccccacataacccgcgg

SEQ ID NO: 204 - DNA, 23 bp, synthetic construct
  misc_feature 1..23: note = Synthetic: Target sequences for wild type COL8A2
  source 1..23: mol_type = other DNA
gcagagcaagaatcctgaaaagg

SEQ ID NO: 205 - DNA, 23 bp, synthetic construct
  misc_feature 1..23: note = Synthetic: Target sequences for wild type COL8A2
  source 1..23: mol_type = other DNA
gagcaagaatcctgaaaaggagg

SEQ ID NO: 206 - DNA, 23 bp, synthetic construct
  misc_feature 1..23: note = Synthetic: Target sequences for wild type COL8A2
  source 1..23: mol_type = other DNA
agaatcctgaaaaggaggagtgg

SEQ ID NO: 207 - DNA, 23 bp, synthetic construct
  misc_feature 1..23: note = Synthetic: Target sequences for wild type COL8A2
  source 1..23: mol_type = other DNA
tacatccactcctccttttcagg

SEQ ID NO: 208 - DNA, 23 bp, synthetic construct
  misc_feature 1..23: note = Synthetic: Target sequences for wild type COL8A2
  source 1..23: mol_type = other DNA
ggaggagtggatgtactccgtgg

SEQ ID NO: 209 - DNA, 23 bp, synthetic construct
  misc_feature 1..23: note = Synthetic: Target sequences for wild type COL8A2
  source 1..23: mol_type = other DNA
ggatgtactccgtggagtagagg

SEQ ID NO: 210 - DNA, 23 bp, synthetic construct
  misc_feature 1..23: note = Synthetic: Target sequences for wild type COL8A2
  source 1..23: mol_type = other DNA
ctccgtggagtagaggccgttgg

SEQ ID NO: 211 - DNA, 23 bp, synthetic construct
  misc_feature 1..23: note = Synthetic: Target sequences for wild type COL8A2
  source 1..23: mol_type = other DNA
ggccaacggcctctactccacgg

SEQ ID NO: 212 - DNA, 23 bp, synthetic construct
  misc_feature 1..23: note = Synthetic: Target sequences for wild type COL8A2
  source 1..23: mol_type = other DNA
tggagtagaggccgttggcctgg

SEQ ID NO: 213 - DNA, 23 bp, synthetic construct
  misc_feature 1..23: note = Synthetic: Target sequences for wild type COL8A2
  source 1..23: mol_type = other DNA
aggccgttggcctggtccgacgg

SEQ ID NO: 214 - DNA, 23 bp, synthetic construct
  misc_feature 1..23: note = Synthetic: Target sequences for wild type COL8A2
  source 1..23: mol_type = other DNA
atgccgtcggaccaggccaacgg

SEQ ID NO: 215 - DNA, 23 bp, synthetic construct
  misc_feature 1..23: note = Synthetic: Target sequences for wild type COL8A2
  source 1..23: mol_type = other DNA
ggtgcagatgccgtcggaccagg

SEQ ID NO: 216 - DNA, 23 bp, synthetic construct
  misc_feature 1..23: note = Synthetic: Target sequences for wild type COL8A2
  source 1..23: mol_type = other DNA
ggtctgggtgcagatgccgtcgg

SEQ ID NO: 217 - DNA, 23 bp, synthetic construct
  misc_feature 1..23: note = Synthetic: Target sequences for wild type COL8A2
  source 1..23: mol_type = other DNA
acggcatctgcacccagacctgg

SEQ ID NO: 218 - DNA, 23 bp, synthetic construct
  misc_feature 1..23: note = Synthetic: Target sequences for wild type COL8A2
  source 1..23: mol_type = other DNA
ctgcacccagacctggtcgttgg

SEQ ID NO: 219 - DNA, 23 bp, synthetic construct
  misc_feature 1..23: note = Synthetic: Target sequences for wild type COL8A2
  source 1..23: mol_type = other DNA
tgcacccagacctggtcgttggg

SEQ ID NO: 220 - DNA, 23 bp, synthetic construct
  misc_feature 1..23: note = Synthetic: Target sequences for wild type COL8A2
  source 1..23: mol_type = other DNA
gcggcccaacgaccaggtctggg

SEQ ID NO: 221 - DNA, 23 bp, synthetic construct
  misc_feature 1..23: note = Synthetic: Target sequences for wild type COL8A2
  source 1..23: mol_type = other DNA
tgcggcccaacgaccaggtctgg

SEQ ID NO: 222 - DNA, 23 bp, synthetic construct
  misc_feature 1..23: note = Synthetic: Target sequences for wild type COL8A2
  source 1..23: mol_type = other DNA
cctggtcgttgggccgcagctgg

SEQ ID NO: 223 - DNA, 23 bp, synthetic construct
  misc_feature 1..23: note = Synthetic: Target sequences for wild type COL8A2
  source 1..23: mol_type = other DNA
ccagctgcggcccaacgaccagg

SEQ ID NO: 224 - DNA, 23 bp, synthetic construct
  misc_feature 1..23: note = Synthetic: Target sequences for wild type COL8A2
  source 1..23: mol_type = other DNA
gttgggccgcagctggagcacgg

SEQ ID NO: 225 - DNA, 23 bp, synthetic construct
  misc_feature 1..23: note = Synthetic: Target sequences for wild type COL8A2
  source 1..23: mol_type = other DNA
gtggggccgtgctccagctgcgg

SEQ ID NO: 226 - DNA, 23 bp, synthetic construct
  misc_feature 1..23: note = Synthetic: Target sequences for wild type COL8A2
  source 1..23: mol_type = other DNA
gcacggccccaccagatgcctgg

SEQ ID NO: 227 - DNA, 23 bp, synthetic construct
  misc_feature 1..23: note = Synthetic: Target sequences for wild type COL8A2
  source 1..23: mol_type = other DNA
ccccaccagatgcctggtccagg

SEQ ID NO: 228 - DNA, 23 bp, synthetic construct
  misc_feature 1..23: note = Synthetic: Target sequences for wild type COL8A2
  source 1..23: mol_type = other DNA
cctggaccaggcatctggtgggg

SEQ ID NO: 229 - DNA, 23 bp, synthetic construct
  misc_feature 1..23: note = Synthetic: Target sequences for wild type COL8A2
  source 1..23: mol_type = other DNA
acctggaccaggcatctggtggg

SEQ ID NO: 230 - DNA, 23 bp, synthetic construct
  misc_feature 1..23: note = Synthetic: Target sequences for wild type COL8A2
  source 1..23: mol_type = other DNA
tacctggaccaggcatctggtgg

SEQ ID NO: 231 - DNA, 23 bp, synthetic construct
  misc_feature 1..23: note = Synthetic: Target sequences for wild type COL8A2
  source 1..23: mol_type = other DNA
ggctacctggaccaggcatctgg

SEQ ID NO: 232 - DNA, 23 bp, synthetic construct
  misc_feature 1..23: note = Synthetic: Target sequences for wild type COL8A2
  source 1..23: mol_type = other DNA
caagaagggctacctggaccagg

SEQ ID NO: 233 - DNA, 23 bp, synthetic construct
  misc_feature 1..23: note = Synthetic: Target sequences for wild type COL8A2
  source 1..23: mol_type = other DNA
tgagtacaagaagggctacctgg

SEQ ID NO: 234 - DNA, 23 bp, synthetic construct
  misc_feature 1..23: note = Synthetic: Target sequences for wild type COL8A2
  source 1..23: mol_type = other DNA
gcccttcttgtactcatcgtagg

SEQ ID NO: 235 - DNA, 23 bp, synthetic construct
  misc_feature 1..23: note = Synthetic: Target sequences for wild type COL8A2
  source 1..23: mol_type = other DNA
acctacgatgagtacaagaaggg

SEQ ID NO: 236 - DNA, 23 bp, synthetic construct
  misc_feature 1..23: note = Synthetic: Target sequences for wild type COL8A2
  source 1..23: mol_type = other DNA
tacctacgatgagtacaagaagg

SEQ ID NO: 237 - DNA, 23 bp, synthetic construct
  misc_feature 1..23: note = Synthetic: Target sequences for wild type COL8A2
  source 1..23: mol_type = other DNA
cttgtactcatcgtaggtatagg

SEQ ID NO: 238 - DNA, 23 bp, synthetic construct
  misc_feature 1..23: note = Synthetic: Target sequences for wild type COL8A2
  source 1..23: mol_type = other DNA
gtactcatcgtaggtataggtgg

SEQ ID NO: 239 - DNA, 23 bp, synthetic construct
  misc_feature 1..23: note = Synthetic: Target sequences for wild type COL8A2
  source 1..23: mol_type = other DNA
tcatcgtaggtataggtggccgg

SEQ ID NO: 240 - DNA, 23 bp, synthetic construct
  misc_feature 1..23: note = Synthetic: Target sequences for wild type COL8A2
  source 1..23: mol_type = other DNA
ccggcacgttgttcttgtacagg

SEQ ID NO: 241 - DNA, 23 bp, synthetic construct
  misc_feature 1..23: note = Synthetic: Target sequences for wild type COL8A2
  source 1..23: mol_type = other DNA
cctgtacaagaacaacgtgccgg

SEQ ID NO: 242 - DNA, 23 bp, synthetic construct
  misc_feature 1..23: note = Synthetic: Target sequences for wild type COL8A2
  source 1..23: mol_type = other DNA
cggcacgttgttcttgtacaggg

SEQ ID NO: 243 - DNA, 23 bp, synthetic construct
  misc_feature 1..23: note = Synthetic: Target sequences for wild type COL8A2
  source 1..23: mol_type = other DNA
gtacagggccacccacacgttgg

SEQ ID NO: 244 - DNA, 23 bp, synthetic construct
  misc_feature 1..23: note = Synthetic: Target sequences for wild type COL8A2
  source 1..23: mol_type = other DNA
caagggcaccaacgtgtgggtgg

SEQ ID NO: 245 - DNA, 23 bp, synthetic construct
  misc_feature 1..23: note = Synthetic: Target sequences for wild type COL8A2
  source 1..23: mol_type = other DNA
cgtcaagggcaccaacgtgtggg

SEQ ID NO: 246 - DNA, 23 bp, synthetic construct
  misc_feature 1..23: note = Synthetic: Target sequences for wild type COL8A2
  source 1..23: mol_type = other DNA
acgtcaagggcaccaacgtgtgg

SEQ ID NO: 247 - DNA, 23 bp, synthetic construct
  misc_feature 1..23: note = Synthetic: Target sequences for wild type COL8A2
  source 1..23: mol_type = other DNA
tggtgcccttgacgtgcacatgg

SEQ ID NO: 248 - DNA, 23 bp, synthetic construct
  misc_feature 1..23: note = Synthetic: Target sequences for wild type COL8A2
  source 1..23: mol_type = other DNA
gcttaccatgtgcacgtcaaggg

SEQ ID NO: 249 - DNA, 23 bp, synthetic construct
  misc_feature 1..23: note = Synthetic: Target sequences for wild type COL8A2
  source 1..23: mol_type = other DNA
tgcttaccatgtgcacgtcaagg

SEQ ID NO: 250 - DNA, 23 bp, synthetic construct
  misc_feature 1..23: note = Synthetic: Target sequences for wild type COL8A2
  source 1..23: mol_type = other DNA
aagtagtagacgccgcccacagg

SEQ ID NO: 251 - DNA, 23 bp, synthetic construct
  misc_feature 1..23: note = Synthetic: Target sequences for wild type COL8A2
  source 1..23: mol_type = other DNA
agtagtagacgccgcccacaggg

SEQ ID NO: 252 - DNA, 23 bp, synthetic construct
  misc_feature 1..23: note = Synthetic: Target sequences for wild type COL8A2
  source 1..23: mol_type = other DNA
gtagacgccgcccacagggcagg

SEQ ID NO: 253 - DNA, 23 bp, synthetic construct
  misc_feature 1..23: note = Synthetic: Target sequences for wild type COL8A2
  source 1..23: mol_type = other DNA
atcttcacctgccctgtgggcgg

SEQ ID NO: 254 - DNA, 23 bp, synthetic construct
  misc_feature 1..23: note = Synthetic: Target sequences for wild type COL8A2
  source 1..23: mol_type = other DNA
ggcatcttcacctgccctgtggg

SEQ ID NO: 255 - DNA, 23 bp, synthetic construct
  misc_feature 1..23: note = Synthetic: Target sequences for wild type COL8A2
  source 1..23: mol_type = other DNA
tggcatcttcacctgccctgtgg

SEQ ID NO: 256 - DNA, 23 bp, synthetic construct
  misc_feature 1..23: note = Synthetic: Target sequences for wild type COL8A2
  source 1..23: mol_type = other DNA
agggcaggtgaagatgccagtgg

SEQ ID NO: 257 - DNA, 23 bp, synthetic construct
  misc_feature 1..23: note = Synthetic: Target sequences for wild type COL8A2
  source 1..23: mol_type = other DNA
caggtgaagatgccagtggctgg

SEQ ID NO: 258 - DNA, 23 bp, synthetic construct
  misc_feature 1..23: note = Synthetic: Target sequences for wild type COL8A2
  source 1..23: mol_type = other DNA
aggtgaagatgccagtggctggg

SEQ ID NO: 259 - DNA, 23 bp, synthetic construct
  misc_feature 1..23: note = Synthetic: Target sequences for wild type COL8A2
  source 1..23: mol_type = other DNA
agcggctacaacccagccactgg

SEQ ID NO: 260 - DNA, 23 bp, synthetic construct
  misc_feature 1..23: note = Synthetic: Target sequences for wild type COL8A2
  source 1..23: mol_type = other DNA
tggctgggttgtagccgctgtgg

SEQ ID NO: 261 - DNA, 23 bp, synthetic construct
  misc_feature 1..23: note = Synthetic: Target sequences for wild type COL8A2
  source 1..23: mol_type = other DNA
actctctacaatggccacagcgg

SEQ ID NO: 262 - DNA, 23 bp, synthetic construct
  misc_feature 1..23: note = Synthetic: Target sequences for wild type COL8A2
  source 1..23: mol_type = other DNA
tgtggccattgtagagagtccgg

SEQ ID NO: 263 - DNA, 23 bp, synthetic construct
  misc_feature 1..23: note = Synthetic: Target sequences for wild type COL8A2
  source 1..23: mol_type = other DNA
tttgaccggactctctacaatgg

SEQ ID NO: 264 - DNA, 23 bp, synthetic construct
  misc_feature 1..23: note = Synthetic: Target sequences for wild type COL8A2
  source 1..23: mol_type = other DNA
gagagtccggtcaaatttcacgg

SEQ ID NO: 265 - DNA, 23 bp, synthetic construct
  misc_feature 1..23: note = Synthetic: Target sequences for wild type COL8A2
  source 1..23: mol_type = other DNA
agagtccggtcaaatttcacggg

SEQ ID NO: 266 - DNA, 23 bp, synthetic construct
  misc_feature 1..23: note = Synthetic: Target sequences for wild type COL8A2
  source 1..23: mol_type = other DNA
gcatgcccgtgaaatttgaccgg

SEQ ID NO: 267 - DNA, 23 bp, synthetic construct
  misc_feature 1..23: note = Synthetic: Target sequences for wild type COL8A2
  source 1..23: mol_type = other DNA
aaatttcacgggcatgcccgagg

SEQ ID NO: 268 - DNA, 23 bp, synthetic construct
  misc_feature 1..23: note = Synthetic: Target sequences for wild type COL8A2
  source 1..23: mol_type = other DNA
tttcacgggcatgcccgaggcgg

SEQ ID NO: 269 - DNA, 23 bp, synthetic construct
  misc_feature 1..23: note = Synthetic: Target sequences for wild type COL8A2
  source 1..23: mol_type = other DNA
ttcacgggcatgcccgaggcggg

SEQ ID NO: 270 - DNA, 23 bp, synthetic construct
  misc_feature 1..23: note = Synthetic: Target sequences for wild type COL8A2
  source 1..23: mol_type = other DNA
tcacgggcatgcccgaggcgggg

SEQ ID NO: 271 - DNA, 23 bp, synthetic construct
  misc_feature 1..23: note = Synthetic: Target sequences for wild type COL8A2
  source 1..23: mol_type = other DNA
gggcatgcccgaggcggggaagg

SEQ ID NO: 272 - DNA, 23 bp, synthetic construct
  misc_feature 1..23: note = Synthetic: Target sequences for wild type COL8A2
  source 1..23: mol_type = other DNA
ggcatgcccgaggcggggaaggg

SEQ ID NO: 273 - DNA, 23 bp, synthetic construct
  misc_feature 1..23: note = Synthetic: Target sequences for wild type COL8A2
  source 1..23: mol_type = other DNA
gcccgaggcggggaagggcgagg

SEQ ID NO: 274 - DNA, 23 bp, synthetic construct
  misc_feature 1..23: note = Synthetic: Target sequences for wild type COL8A2
  source 1..23: mol_type = other DNA
acctcgcccttccccgcctcggg

SEQ ID NO: 275 - DNA, 23 bp, synthetic construct
  misc_feature 1..23: note = Synthetic: Target sequences for wild type COL8A2
  source 1..23: mol_type = other DNA
cacctcgcccttccccgcctcgg

SEQ ID NO: 276 - DNA, 23 bp, synthetic construct
  misc_feature 1..23: note = Synthetic: Target sequences for wild type COL8A2
  source 1..23: mol_type = other DNA
cgaggtgagcaccgcagtgaagg

SEQ ID NO: 277 - DNA, 23 bp, synthetic construct
  misc_feature 1..23: note = Synthetic: Target sequences for wild type COL8A2
  source 1..23: mol_type = other DNA
gtgagcaccgcagtgaaggccgg

SEQ ID NO: 278 - DNA, 23 bp, synthetic construct
  misc_feature 1..23: note = Synthetic: Target sequences for wild type COL8A2
  source 1..23: mol_type = other DNA
caccgcagtgaaggccggtgtgg

SEQ ID NO: 279 - DNA, 23 bp, synthetic construct
  misc_feature 1..23: note = Synthetic: Target sequences for wild type COL8A2
  source 1..23: mol_type = other DNA
tgccacaccggccttcactgcgg

SEQ ID NO: 280 - DNA, 23 bp, synthetic construct
  misc_feature 1..23: note = Synthetic: Target sequences for wild type COL8A2
  source 1..23: mol_type = other DNA
cagtgaaggccggtgtggcatgg

SEQ ID NO: 281 - DNA, 23 bp, synthetic construct
  misc_feature 1..23: note = Synthetic: Target sequences for wild type COL8A2
  source 1..23: mol_type = other DNA
agtgaaggccggtgtggcatggg

SEQ ID NO: 282 - DNA, 23 bp, synthetic construct
  misc_feature 1..23: note = Synthetic: Target sequences for wild type COL8A2
  source 1..23: mol_type = other DNA
gctgtctgcccatgccacaccgg

SEQ ID NO: 283 - DNA, 23 bp, synthetic construct
  misc_feature 1..23: note = Synthetic: Target sequences for wild type COL8A2
  source 1..23: mol_type = other DNA
agctcgcccagcccaaactgtgg

SEQ ID NO: 284 - DNA, 23 bp, synthetic construct
  misc_feature 1..23: note = Synthetic: Target sequences for wild type COL8A2
  source 1..23: mol_type = other DNA
ggcaagccacagtttgggctggg

SEQ ID NO: 285 - DNA, 23 bp, synthetic construct
  misc_feature 1..23: note = Synthetic: Target sequences for wild type COL8A2
  source 1..23: mol_type = other DNA
gggcaagccacagtttgggctgg

SEQ ID NO: 286 - DNA, 23 bp, synthetic construct
  misc_feature 1..23: note = Synthetic: Target sequences for wild type COL8A2
  source 1..23: mol_type = other DNA
aggggggcaagccacagtttggg

SEQ ID NO: 287 - DNA, 23 bp, synthetic construct
  misc_feature 1..23: note = Synthetic: Target sequences for wild type COL8A2
  source 1..23: mol_type = other DNA
aaggggggcaagccacagtttgg

SEQ ID NO: 288 - DNA, 23 bp, synthetic construct
  misc_feature 1..23: note = Synthetic: Target sequences for wild type COL8A2
  source 1..23: mol_type = other DNA
cttgccccccttgcccagcacgg

SEQ ID NO: 289 - DNA, 23 bp, synthetic construct
  misc_feature 1..23: note = Synthetic: Target sequences for wild type COL8A2
  source 1..23: mol_type = other DNA
ggtgccgtgctgggcaagggggg

SEQ ID NO: 290 - DNA, 23 bp, synthetic construct
  misc_feature 1..23: note = Synthetic: Target sequences for wild type COL8A2
  source 1..23: mol_type = other DNA
gggtgccgtgctgggcaaggggg

SEQ ID NO: 291 - DNA, 23 bp, synthetic construct
  misc_feature 1..23: note = Synthetic: Target sequences for wild type COL8A2
  source 1..23: mol_type = other DNA
agggtgccgtgctgggcaagggg

SEQ ID NO: 292 - DNA, 23 bp, synthetic construct
  misc_feature 1..23: note = Synthetic: Target sequences for wild type COL8A2
  source 1..23: mol_type = other DNA
gagggtgccgtgctgggcaaggg

SEQ ID NO: 293 - DNA, 23 bp, synthetic construct
  misc_feature 1..23: note = Synthetic: Target sequences for wild type COL8A2
  source 1..23: mol_type = other DNA
ggagggtgccgtgctgggcaagg

SEQ ID NO: 294 - DNA, 23 bp, synthetic construct
  misc_feature 1..23: note = Synthetic: Target sequences for wild type COL8A2
  source 1..23: mol_type = other DNA
ggtgtggagggtgccgtgctggg

SEQ ID NO: 295 - DNA, 23 bp, synthetic construct
  misc_feature 1..23: note = Synthetic: Target sequences for wild type COL8A2
  source 1..23: mol_type = other DNA
cggtgtggagggtgccgtgctgg

SEQ ID NO: 296 - DNA, 23 bp, synthetic construct
  misc_feature 1..23: note = Synthetic: Target sequences for wild type COL8A2
  source 1..23: mol_type = other DNA
ggcaccctccacaccgccgttgg

SEQ ID NO: 297 - DNA, 23 bp, synthetic construct
  misc_feature 1..23: note = Synthetic: Target sequences for wild type COL8A2
  source 1..23: mol_type = other DNA
gcaccctccacaccgccgttggg

SEQ ID NO: 298 - DNA, 23 bp, synthetic construct
  misc_feature 1..23: note = Synthetic: Target sequences for wild type COL8A2
  source 1..23: mol_type = other DNA
ctgcccaacggcggtgtggaggg

SEQ ID NO: 299 - DNA, 23 bp, synthetic construct
  misc_feature 1..23: note = Synthetic: Target sequences for wild type COL8A2
  source 1..23: mol_type = other DNA
cctccacaccgccgttgggcagg

SEQ ID NO: 300 - DNA, 23 bp, synthetic construct
  misc_feature 1..23: note = Synthetic: Target sequences for wild type COL8A2
  source 1..23: mol_type = other DNA
cctgcccaacggcggtgtggagg

SEQ ID NO: 301 - DNA, 23 bp, synthetic construct
  misc_feature 1..23: note = Synthetic: Target sequences for wild type COL8A2
  source 1..23: mol_type = other DNA
gcacctgcccaacggcggtgtgg

SEQ ID NO: 302 - DNA, 23 bp, synthetic construct
  misc_feature 1..23: note = Synthetic: Target sequences for wild type COL8A2
  source 1..23: mol_type = other DNA
ggcttgcacctgcccaacggcgg

SEQ ID NO: 303 - DNA, 23 bp, synthetic construct
  misc_feature 1..23: note = Synthetic: Target sequences for wild type COL8A2
  source 1..23: mol_type = other DNA
gcaggcttgcacctgcccaacgg

SEQ ID NO: 304 - DNA, 23 bp, synthetic construct
  misc_feature 1..23: note = Synthetic: Target sequences for wild type COL8A2
  source 1..23: mol_type = other DNA
ttcgatgagactggcatcgcagg

SEQ ID NO: 305 - DNA, 23 bp, synthetic construct
  misc_feature 1..23: note = Synthetic: Target sequences for wild type COL8A2
  source 1..23: mol_type = other DNA
tgcgatgccagtctcatcgaagg

SEQ ID NO: 306 - DNA, 23 bp, synthetic construct
  misc_feature 1..23: note = Synthetic: Target sequences for wild type COL8A2
  source 1..23: mol_type = other DNA
ccagtctcatcgaaggccccagg

SEQ ID NO: 307 - DNA, 23 bp, synthetic construct
  misc_feature 1..23: note = Synthetic: Target sequences for wild type COL8A2
  source 1..23: mol_type = other DNA
cctggggccttcgatgagactgg

SEQ ID NO: 308 - DNA, 23 bp, synthetic construct
  misc_feature 1..23: note = Synthetic: Target sequences for wild type COL8A2
  source 1..23: mol_type = other DNA
cagtctcatcgaaggccccaggg

SEQ ID NO: 309 - DNA, 23 bp, synthetic construct
  misc_feature 1..23: note = Synthetic: Target sequences for wild type COL8A2
  source 1..23: mol_type = other DNA
agtctcatcgaaggccccagggg

SEQ ID NO: 310 - DNA, 23 bp, synthetic construct
  misc_feature 1..23: note = Synthetic: Target sequences for wild type COL8A2
  source 1..23: mol_type = other DNA
tcgaaggccccaggggcaccagg

SEQ ID NO: 311 - DNA, 23 bp, synthetic construct
  misc_feature 1..23: note = Synthetic: Target sequences for wild type COL8A2
  source 1..23: mol_type = other DNA
cgaaggccccaggggcaccaggg

SEQ ID NO: 312 - DNA, 23 bp, synthetic construct
  misc_feature 1..23: note = Synthetic: Target sequences for wild type COL8A2
  source 1..23: mol_type = other DNA
gaaggccccaggggcaccagggg

SEQ ID NO: 313 - DNA, 23 bp, synthetic construct
  misc_feature 1..23: note = Synthetic: Target sequences for wild type COL8A2
  source 1..23: mol_type = other DNA
aaggccccaggggcaccaggggg

SEQ ID NO: 314 - DNA, 23 bp, synthetic construct
  misc_feature 1..23: note = Synthetic: Target sequences for wild type COL8A2
  source 1..23: mol_type = other DNA
gggaccccctggtgcccctgggg

SEQ ID NO: 315 - DNA, 23 bp, synthetic construct
  misc_feature 1..23: note = Synthetic: Target sequences for wild type COL8A2
  source 1..23: mol_type = other DNA
cgggaccccctggtgcccctggg

SEQ ID NO: 316 - DNA, 23 bp, synthetic construct
  misc_feature 1..23: note = Synthetic: Target sequences for wild type COL8A2
  source 1..23: mol_type = other DNA
ccaggggcaccagggggtcccgg

SEQ ID NO: 317 - DNA, 23 bp, synthetic construct
  misc_feature 1..23: note = Synthetic: Target sequences for wild type COL8A2
  source 1..23: mol_type = other DNA
ccgggaccccctggtgcccctgg

SEQ ID NO: 318 - DNA, 23 bp, synthetic construct
  misc_feature 1..23: note = Synthetic: Target sequences for wild type COL8A2
  source 1..23: mol_type = other DNA
caggggcaccagggggtcccggg

SEQ ID NO: 319 - DNA, 23 bp, synthetic construct
  misc_feature 1..23: note = Synthetic: Target sequences for wild type COL8A2
  source 1..23: mol_type = other DNA
aggggcaccagggggtcccgggg

SEQ ID NO: 320 - DNA, 23 bp, synthetic construct
  misc_feature 1..23: note = Synthetic: Target sequences for wild type COL8A2
  source 1..23: mol_type = other DNA
ggggcaccagggggtcccggggg

SEQ ID NO: 321 - DNA, 23 bp, synthetic construct
  misc_feature 1..23: note = Synthetic: Target sequences for wild type COL8A2
  source 1..23: mol_type = other DNA
accagggggtcccgggggcccgg

SEQ ID NO: 322 - DNA, 23 bp, synthetic construct
  misc_feature 1..23: note = Synthetic: Target sequences for wild type COL8A2
  source 1..23: mol_type = other DNA
ccagggggtcccgggggcccggg

SEQ ID NO: 323 - DNA, 23 bp, synthetic construct
  misc_feature 1..23: note = Synthetic: Target sequences for wild type COL8A2
  source 1..23: mol_type = other DNA
cccgggcccccgggaccccctgg

SEQ ID NO: 324 - DNA, 23 bp, synthetic construct
  misc_feature 1..23: note = Synthetic: Target sequences for wild type COL8A2
  source 1..23: mol_type = other DNA
gggggtcccgggggcccgggagg

SEQ ID NO: 325 - DNA, 23 bp, synthetic construct
  misc_feature 1..23: note = Synthetic: Target sequences for wild type COL8A2
  source 1..23: mol_type = other DNA
cccgggggcccgggaggccccgg

SEQ ID NO: 326 - DNA, 23 bp, synthetic construct
  misc_feature 1..23: note = Synthetic: Target sequences for wild type COL8A2
  source 1..23: mol_type = other DNA
ccggggcctcccgggcccccggg

SEQ ID NO: 327 - DNA, 23 bp, synthetic construct
  misc_feature 1..23: note = Synthetic: Target sequences for wild type COL8A2
  source 1..23: mol_type = other DNA
tccggggcctcccgggcccccgg

SEQ ID NO: 328 - DNA, 23 bp, synthetic construct
  misc_feature 1..23: note = Synthetic: Target sequences for wild type COL8A2
  source 1..23: mol_type = other DNA
gggggcccgggaggccccggagg

SEQ ID NO: 329 - DNA, 23 bp, synthetic construct
  misc_feature 1..23: note = Synthetic: Target sequences for wild type COL8A2
  source 1..23: mol_type = other DNA
ggggcccgggaggccccggaggg

SEQ ID NO: 330 - DNA, 23 bp, synthetic construct
  misc_feature 1..23: note = Synthetic: Target sequences for wild type COL8A2
  source 1..23: mol_type = other DNA
cgggccctccggggcctcccggg

SEQ ID NO: 331 - DNA, 23 bp, synthetic construct
  misc_feature 1..23: note = Synthetic: Target sequences for wild type COL8A2
  source 1..23: mol_type = other DNA
acgggccctccggggcctcccgg

SEQ ID NO: 332 - DNA, 23 bp, synthetic construct
  misc_feature 1..23: note = Synthetic: Target sequences for wild type COL8A2
  source 1..23: mol_type = other DNA
ctggaatcacgggccctccgggg

SEQ ID NO: 333 - DNA, 23 bp, synthetic construct
  misc_feature 1..23: note = Synthetic: Target sequences for wild type COL8A2
  source 1..23: mol_type = other DNA
cccggagggcccgtgattccagg

SEQ ID NO: 334 - DNA, 23 bp, synthetic construct
  misc_feature 1..23: note = Synthetic: Target sequences for wild type COL8A2
  source 1..23: mol_type = other DNA
cctggaatcacgggccctccggg

SEQ ID NO: 335 - DNA, 23 bp, synthetic construct
  misc_feature 1..23: note = Synthetic: Target sequences for wild type COL8A2
  source 1..23: mol_type = other DNA
ccggagggcccgtgattccaggg

SEQ ID NO: 336 - DNA, 23 bp, synthetic construct
  misc_feature 1..23: note = Synthetic: Target sequences for wild type COL8A2
  source 1..23: mol_type = other DNA
ccctggaatcacgggccctccgg

SEQ ID NO: 337 - DNA, 23 bp, synthetic construct
  misc_feature 1..23: note = Synthetic: Target sequences for wild type COL8A2
  source 1..23: mol_type = other DNA
cggagggcccgtgattccagggg

SEQ ID NO: 338 - DNA, 23 bp, synthetic construct
  misc_feature 1..23: note = Synthetic: Target sequences for wild type COL8A2
  source 1..23: mol_type = other DNA
cccgtgattccaggggagccagg

SEQ ID NO: 339 - DNA, 23 bp, synthetic construct
  misc_feature 1..23: note = Synthetic: Target sequences for wild type COL8A2
  source 1..23: mol_type = other DNA
cctggctcccctggaatcacggg

SEQ ID NO: 340 - DNA, 23 bp, synthetic construct
  misc_feature 1..23: note = Synthetic: Target sequences for wild type COL8A2
  source 1..23: mol_type = other DNA
ccgtgattccaggggagccaggg

SEQ ID NO: 341 - DNA, 23 bp, synthetic construct
  misc_feature 1..23: note = Synthetic: Target sequences for wild type COL8A2
  source 1..23: mol_type = other DNA
ccctggctcccctggaatcacgg

SEQ ID NO: 342 - DNA, 23 bp, synthetic construct
  misc_feature 1..23: note = Synthetic: Target sequences for wild type COL8A2
  source 1..23: mol_type = other DNA
ccaggggagccagggacccctgg

SEQ ID NO: 343 - DNA, 23 bp, synthetic construct
  misc_feature 1..23: note = Synthetic: Target sequences for wild type COL8A2
  source 1..23: mol_type = other DNA
ccaggggtccctggctcccctgg

SEQ ID NO: 344 - DNA, 23 bp, synthetic construct
  misc_feature 1..23: note = Synthetic: Target sequences for wild type COL8A2
  source 1..23: mol_type = other DNA
caggggagccagggacccctggg

SEQ ID NO: 345 - DNA, 23 bp, synthetic construct
  misc_feature 1..23: note = Synthetic: Target sequences for wild type COL8A2
  source 1..23: mol_type = other DNA
aggggagccagggacccctgggg

SEQ ID NO: 346 - DNA, 23 bp, synthetic construct
  misc_feature 1..23: note = Synthetic: Target sequences for wild type COL8A2
  source 1..23: mol_type = other DNA
ggggagccagggacccctggggg

SEQ ID NO: 347 - DNA, 23 bp, synthetic construct
  misc_feature 1..23: note = Synthetic: Target sequences for wild type COL8A2
  source 1..23: mol_type = other DNA
acggggcccccaggggtccctgg

SEQ ID NO: 348 - DNA, 23 bp, synthetic construct
  misc_feature 1..23: note = Synthetic: Target sequences for wild type COL8A2
  source 1..23: mol_type = other DNA
agggacccctgggggccccgtgg

SEQ ID NO: 349 - DNA, 23 bp, synthetic construct
  misc_feature 1..23: note = Synthetic: Target sequences for wild type COL8A2
  source 1..23: mol_type = other DNA
gggacccctgggggccccgtggg

SEQ ID NO: 350 - DNA, 23 bp, synthetic construct
  misc_feature 1..23: note = Synthetic: Target sequences for wild type COL8A2
  source 1..23: mol_type = other DNA
tgggcccacggggcccccagggg

SEQ ID NO: 351 - DNA, 23 bp, synthetic construct
  misc_feature 1..23: note = Synthetic: Target sequences for wild type COL8A2
  source 1..23: mol_type = other DNA
ctgggcccacggggcccccaggg

SEQ ID NO: 352 - DNA, 23 bp, synthetic construct
  misc_feature 1..23: note = Synthetic: Target sequences for wild type COL8A2
  source 1..23: mol_type = other DNA
gctgggcccacggggcccccagg

SEQ ID NO: 353 - DNA, 23 bp, synthetic construct
  misc_feature 1..23: note = Synthetic: Target sequences for wild type COL8A2
  source 1..23: mol_type = other DNA
ctggcacggctgggcccacgggg

SEQ ID NO: 354 - DNA, 23 bp, synthetic construct
  misc_feature 1..23: note = Synthetic: Target sequences for wild type COL8A2
  source 1..23: mol_type = other DNA
cccgtgggcccagccgtgccagg

SEQ ID NO: 355 - DNA, 23 bp, synthetic construct
  misc_feature 1..23: note = Synthetic: Target sequences for wild type COL8A2
  source 1..23: mol_type = other DNA
cctggcacggctgggcccacggg

SEQ ID NO: 356 - DNA, 23 bp, synthetic construct
  misc_feature 1..23: note = Synthetic: Target sequences for wild type COL8A2
  source 1..23: mol_type = other DNA
acctggcacggctgggcccacgg

SEQ ID NO: 357 - DNA, 23 bp, synthetic construct
  misc_feature 1..23: note = Synthetic: Target sequences for wild type COL8A2
  source 1..23: mol_type = other DNA
caggggaacctggcacggctggg

SEQ ID NO: 358 - DNA, 23 bp, synthetic construct
  misc_feature 1..23: note = Synthetic: Target sequences for wild type COL8A2
  source 1..23: mol_type = other DNA
gcaggggaacctggcacggctgg

SEQ ID NO: 359 - DNA, 23 bp, synthetic construct
  misc_feature 1..23: note = Synthetic: Target sequences for wild type COL8A2
  source 1..23: mol_type = other DNA
gagagcaggggaacctggcacgg

SEQ ID NO: 360 - DNA, 23 bp, synthetic construct
  misc_feature 1..23: note = Synthetic: Target sequences for wild type COL8A2
  source 1..23: mol_type = other DNA
gaggggagagcaggggaacctgg

SEQ ID NO: 361 - DNA, 23 bp, synthetic construct
  misc_feature 1..23: note = Synthetic: Target sequences for wild type COL8A2
  source 1..23: mol_type = other DNA
tcccctgctctcccctctccagg

SEQ ID NO: 362 - DNA, 23 bp, synthetic construct
  misc_feature 1..23: note = Synthetic: Target sequences for wild type COL8A2
  source 1..23: mol_type = other DNA
cccctgctctcccctctccaggg

SEQ ID NO: 363 - DNA, 23 bp, synthetic construct
  misc_feature 1..23: note = Synthetic: Target sequences for wild type COL8A2
  source 1..23: mol_type = other DNA
ccctggagaggggagagcagggg

SEQ ID NO: 364 - DNA, 23 bp, synthetic construct
  misc_feature 1..23: note = Synthetic: Target sequences for wild type COL8A2
  source 1..23: mol_type = other DNA
ccctgctctcccctctccagggg

SEQ ID NO: 365 - DNA, 23 bp, synthetic construct
  misc_feature 1..23: note = Synthetic: Target sequences for wild type COL8A2
  source 1..23: mol_type = other DNA
cccctggagaggggagagcaggg

SEQ ID NO: 366 - DNA, 23 bp, synthetic construct
  misc_feature 1..23: note = Synthetic: Target sequences for wild type COL8A2
  source 1..23: mol_type = other DNA
cctgctctcccctctccaggggg

SEQ ID NO: 367 - DNA, 23 bp, synthetic construct
  misc_feature 1..23: note = Synthetic: Target sequences for wild type COL8A2
  source 1..23: mol_type = other DNA
ccccctggagaggggagagcagg

SEQ ID NO: 368 - DNA, 23 bp, synthetic construct
  misc_feature 1..23: note = Synthetic: Target sequences for wild type COL8A2
  source 1..23: mol_type = other DNA
ctcccctctccagggggccctgg

SEQ ID NO: 369 - DNA, 23 bp, synthetic construct
  misc_feature 1..23: note = Synthetic: Target sequences for wild type COL8A2
  source 1..23: mol_type = other DNA
tgccagggccccctggagagggg

SEQ ID NO: 370 - DNA, 23 bp, synthetic construct
  misc_feature 1..23: note = Synthetic: Target sequences for wild type COL8A2
  source 1..23: mol_type = other DNA
ctgccagggccccctggagaggg

SEQ ID NO: 371 - DNA, 23 bp, synthetic construct
  misc_feature 1..23: note = Synthetic: Target sequences for wild type COL8A2
  source 1..23: mol_type = other DNA
cctctccagggggccctggcagg

SEQ ID NO: 372 - DNA, 23 bp, synthetic construct
  misc_feature 1..23: note = Synthetic: Target sequences for wild type COL8A2
  source 1..23: mol_type = other DNA
cctgccagggccccctggagagg

SEQ ID NO: 373 - DNA, 23 bp, synthetic construct
  misc_feature 1..23: note = Synthetic: Target sequences for wild type COL8A2
  source 1..23: mol_type = other DNA
ccagggggccctggcaggcctgg

SEQ ID NO: 374 - DNA, 23 bp, synthetic construct
  misc_feature 1..23: note = Synthetic: Target sequences for wild type COL8A2
  source 1..23: mol_type = other DNA
ccaggcctgccagggccccctgg

SEQ ID NO: 375 - DNA, 23 bp, synthetic construct
  misc_feature 1..23: note = Synthetic: Target sequences for wild type COL8A2
  source 1..23: mol_type = other DNA
agggggaaccaggcctgccaggg

SEQ ID NO: 376 - DNA, 23 bp, synthetic construct
  misc_feature 1..23: note = Synthetic: Target sequences for wild type COL8A2
  source 1..23: mol_type = other DNA
aagggggaaccaggcctgccagg

SEQ ID NO: 377 - DNA, 23 bp, synthetic construct
  misc_feature 1..23: note = Synthetic: Target sequences for wild type COL8A2
  source 1..23: mol_type = other DNA
gcaggcctggttcccccttcagg

SEQ ID NO: 378 - DNA, 23 bp, synthetic construct
  misc_feature 1..23: note = Synthetic: Target sequences for wild type COL8A2
  source 1..23: mol_type = other DNA
cctggttcccccttcaggcccgg

SEQ ID NO: 379 - DNA, 23 bp, synthetic construct
  misc_feature 1..23: note = Synthetic: Target sequences for wild type COL8A2
  source 1..23: mol_type = other DNA
ccgggcctgaagggggaaccagg

SEQ ID NO: 380 - DNA, 23 bp, synthetic construct
  misc_feature 1..23: note = Synthetic: Target sequences for wild type COL8A2
  source 1..23: mol_type = other DNA
gttcccccttcaggcccggcagg

SEQ ID NO: 381 - DNA, 23 bp, synthetic construct
  misc_feature 1..23: note = Synthetic: Target sequences for wild type COL8A2
  source 1..23: mol_type = other DNA
aggcctgccgggcctgaaggggg

SEQ ID NO: 382 - DNA, 23 bp, synthetic construct
  misc_feature 1..23: note = Synthetic: Target sequences for wild type COL8A2
  source 1..23: mol_type = other DNA
aaggcctgccgggcctgaagggg

SEQ ID NO: 383 - DNA, 23 bp, synthetic construct
  misc_feature 1..23: note = Synthetic: Target sequences for wild type COL8A2
  source 1..23: mol_type = other DNA
caaggcctgccgggcctgaaggg

SEQ ID NO: 384 - DNA, 23 bp, synthetic construct
  misc_feature 1..23: note = Synthetic: Target sequences for wild type COL8A2
  source 1..23: mol_type = other DNA
ccttcaggcccggcaggccttgg

SEQ ID NO: 385 - DNA, 23 bp, synthetic construct
  misc_feature 1..23: note = Synthetic: Target sequences for wild type COL8A2
  source 1..23: mol_type = other DNA
ccaaggcctgccgggcctgaagg

SEQ ID NO: 386 - DNA, 23 bp, synthetic construct
  misc_feature 1..23: note = Synthetic: Target sequences for wild type COL8A2
  source 1..23: mol_type = other DNA
cttcaggcccggcaggccttggg

SEQ ID NO: 387 - DNA, 23 bp, synthetic construct
  misc_feature 1..23: note = Synthetic: Target sequences for wild type COL8A2
  source 1..23: mol_type = other DNA
ttcaggcccggcaggccttgggg

SEQ ID NO: 388 - DNA, 23 bp, synthetic construct
  misc_feature 1..23: note = Synthetic: Target sequences for wild type COL8A2
  source 1..23: mol_type = other DNA
attgggccccaaggcctgccggg

SEQ ID NO: 389 - DNA, 23 bp, synthetic construct
  misc_feature 1..23: note = Synthetic: Target sequences for wild type COL8A2
  source 1..23: mol_type = other DNA
tattgggccccaaggcctgccgg

SEQ ID NO: 390 - DNA, 23 bp, synthetic construct
  misc_feature 1..23: note = Synthetic: Target sequences for wild type COL8A2
  source 1..23: mol_type = other DNA
ggcaggccttggggcccaatagg

SEQ ID NO: 391 - DNA, 23 bp, synthetic construct
  misc_feature 1..23: note = Synthetic: Target sequences for wild type COL8A2
  source 1..23: mol_type = other DNA
gcaggccttggggcccaataggg

SEQ ID NO: 392 - DNA, 23 bp, synthetic construct
  misc_feature 1..23: note = Synthetic: Target sequences for wild type COL8A2
  source 1..23: mol_type = other DNA
gctggccctattgggccccaagg

SEQ ID NO: 393 - DNA, 23 bp, synthetic construct
  misc_feature 1..23: note = Synthetic: Target sequences for wild type COL8A2
  source 1..23: mol_type = other DNA
tggggcccaatagggccagctgg

SEQ ID NO: 394 - DNA, 23 bp, synthetic construct
  misc_feature 1..23: note = Synthetic: Target sequences for wild type COL8A2
  source 1..23: mol_type = other DNA
agggtccagctggccctattggg

SEQ ID NO: 395 - DNA, 23 bp, synthetic construct
  misc_feature 1..23: note = Synthetic: Target sequences for wild type COL8A2
  source 1..23: mol_type = other DNA
cagggtccagctggccctattgg

SEQ ID NO: 396 - DNA, 23 bp, synthetic construct
  misc_feature 1..23: note = Synthetic: Target sequences for wild type COL8A2
  source 1..23: mol_type = other DNA
caatagggccagctggaccctgg

SEQ ID NO: 397 - DNA, 23 bp, synthetic construct
  misc_feature 1..23: note = Synthetic: Target sequences for wild type COL8A2
  source 1..23: mol_type = other DNA
ccagctggaccctggagtcctgg

SEQ ID NO: 398 - DNA, 23 bp, synthetic construct
  misc_feature 1..23: note = Synthetic: Target sequences for wild type COL8A2
  source 1..23: mol_type = other DNA
ccaggactccagggtccagctgg

SEQ ID NO: 399 - DNA, 23 bp, synthetic construct
  misc_feature 1..23: note = Synthetic: Target sequences for wild type COL8A2
  source 1..23: mol_type = other DNA
cagctggaccctggagtcctggg

SEQ ID NO: 400 - DNA, 23 bp, synthetic construct
  misc_feature 1..23: note = Synthetic: Target sequences for wild type COL8A2
  source 1..23: mol_type = other DNA
tcaggaatcccaggactccaggg

SEQ ID NO: 401 - DNA, 23 bp, synthetic construct
  misc_feature 1..23: note = Synthetic: Target sequences for wild type COL8A2
  source 1..23: mol_type = other DNA
ctcaggaatcccaggactccagg

SEQ ID NO: 402 - DNA, 23 bp, synthetic construct
  misc_feature 1..23: note = Synthetic: Target sequences for wild type COL8A2
  source 1..23: mol_type = other DNA
ctggagtcctgggattcctgagg

SEQ ID NO: 403 - DNA, 23 bp, synthetic construct
  misc_feature 1..23: note = Synthetic: Target sequences for wild type COL8A2
  source 1..23: mol_type = other DNA
tggagtcctgggattcctgaggg

SEQ ID NO: 404 - DNA, 23 bp, synthetic construct
  misc_feature 1..23: note = Synthetic: Target sequences for wild type COL8A2
  source 1..23: mol_type = other DNA
aggggtccctcaggaatcccagg

SEQ ID NO: 405 - DNA, 23 bp, synthetic construct
  misc_feature 1..23: note = Synthetic: Target sequences for wild type COL8A2
  source 1..23: mol_type = other DNA
ggattcctgagggacccctcagg

SEQ ID NO: 406 - DNA, 23 bp, synthetic construct
  misc_feature 1..23: note = Synthetic: Target sequences for wild type COL8A2
  source 1..23: mol_type = other DNA
cctgagggacccctcaggccagg

SEQ ID NO: 407 - DNA, 23 bp, synthetic construct
  misc_feature 1..23: note = Synthetic: Target sequences for wild type COL8A2
  source 1..23: mol_type = other DNA
cctggcctgaggggtccctcagg

SEQ ID NO: 408 - DNA, 23 bp, synthetic construct
  misc_feature 1..23: note = Synthetic: Target sequences for wild type COL8A2
  source 1..23: mol_type = other DNA
cccctcaggccaggctgcccagg

SEQ ID NO: 409 - DNA, 23 bp, synthetic construct
  misc_feature 1..23: note = Synthetic: Target sequences for wild type COL8A2
  source 1..23: mol_type = other DNA
cctgggcagcctggcctgagggg

SEQ ID NO: 410 - DNA, 23 bp, synthetic construct
  misc_feature 1..23: note = Synthetic: Target sequences for wild type COL8A2
  source 1..23: mol_type = other DNA
ccctcaggccaggctgcccaggg

SEQ ID NO: 411 - DNA, 23 bp, synthetic construct
  misc_feature 1..23: note = Synthetic: Target sequences for wild type COL8A2
  source 1..23: mol_type = other DNA
ccctgggcagcctggcctgaggg

SEQ ID NO: 412 - DNA, 23 bp, synthetic construct
  misc_feature 1..23: note = Synthetic: Target sequences for wild type COL8A2
  source 1..23: mol_type = other DNA
tccctgggcagcctggcctgagg

SEQ ID NO: 413 - DNA, 23 bp, synthetic construct
  misc_feature 1..23: note = Synthetic: Target sequences for wild type COL8A2
  source 1..23: mol_type = other DNA
ttggggctccctgggcagcctgg

SEQ ID NO: 414 - DNA, 23 bp, synthetic construct
  misc_feature 1..23: note = Synthetic: Target sequences for wild type COL8A2
  source 1..23: mol_type = other DNA
aaggtgacttggggctccctggg

SEQ ID NO: 415 - DNA, 23 bp, synthetic construct
  misc_feature 1..23: note = Synthetic: Target sequences for wild type COL8A2
  source 1..23: mol_type = other DNA
aaaggtgacttggggctccctgg

SEQ ID NO: 416 - DNA, 23 bp, synthetic construct
  misc_feature 1..23: note = Synthetic: Target sequences for wild type COL8A2
  source 1..23: mol_type = other DNA
tggggcagaaaggtgacttgggg

SEQ ID NO: 417 - DNA, 23 bp, synthetic construct
  misc_feature 1..23: note = Synthetic: Target sequences for wild type COL8A2
  source 1..23: mol_type = other DNA
ctggggcagaaaggtgacttggg

SEQ ID NO: 418 - DNA, 23 bp, synthetic construct
  misc_feature 1..23: note = Synthetic: Target sequences for wild type COL8A2
  source 1..23: mol_type = other DNA
ccaagtcacctttctgccccagg

SEQ ID NO: 419 - DNA, 23 bp, synthetic construct
  misc_feature 1..23: note = Synthetic: Target sequences for wild type COL8A2
  source 1..23: mol_type = other DNA
cctggggcagaaaggtgacttgg

SEQ ID NO: 420 - DNA, 23 bp, synthetic construct
  misc_feature 1..23: note = Synthetic: Target sequences for wild type COL8A2
  source 1..23: mol_type = other DNA
caagtcacctttctgccccaggg

SEQ ID NO: 421 - DNA, 23 bp, synthetic construct
  misc_feature 1..23: note = Synthetic: Target sequences for wild type COL8A2
  source 1..23: mol_type = other DNA
gcaggagccctggggcagaaagg

SEQ ID NO: 422 - DNA, 23 bp, synthetic construct
  misc_feature 1..23: note = Synthetic: Target sequences for wild type COL8A2
  source 1..23: mol_type = other DNA
caggggtggcaggagccctgggg

SEQ ID NO: 423 - DNA, 23 bp, synthetic construct
  misc_feature 1..23: note = Synthetic: Target sequences for wild type COL8A2
  source 1..23: mol_type = other DNA
cccagggctcctgccacccctgg

SEQ ID NO: 424 - DNA, 23 bp, synthetic construct
  misc_feature 1..23: note = Synthetic: Target sequences for wild type COL8A2
  source 1..23: mol_type = other DNA
ccaggggtggcaggagccctggg

SEQ ID NO: 425 - DNA, 23 bp, synthetic construct
  misc_feature 1..23: note = Synthetic: Target sequences for wild type COL8A2
  source 1..23: mol_type = other DNA
accaggggtggcaggagccctgg

SEQ ID NO: 426 - DNA, 23 bp, synthetic construct
  misc_feature 1..23: note = Synthetic: Target sequences for wild type COL8A2
  source 1..23: mol_type = other DNA
cctgccacccctggtcctccagg

SEQ ID NO: 427 - DNA, 23 bp, synthetic construct
  misc_feature 1..23: note = Synthetic: Target sequences for wild type COL8A2
  source 1..23: mol_type = other DNA
cctggaggaccaggggtggcagg

SEQ ID NO: 428 - DNA, 23 bp, synthetic construct
  misc_feature 1..23: note = Synthetic: Target sequences for wild type COL8A2
  source 1..23: mol_type = other DNA
ctgccacccctggtcctccaggg

SEQ ID NO: 429 - DNA, 23 bp, synthetic construct
  misc_feature 1..23: note = Synthetic: Target sequences for wild type COL8A2
  source 1..23: mol_type = other DNA
tcgccctggaggaccaggggtgg

SEQ ID NO: 430 - DNA, 23 bp, synthetic construct
  misc_feature 1..23: note = Synthetic: Target sequences for wild type COL8A2
  source 1..23: mol_type = other DNA
gggtcgccctggaggaccagggg

SEQ ID NO: 431 - DNA, 23 bp, synthetic construct
  misc_feature 1..23: note = Synthetic: Target sequences for wild type COL8A2
  source 1..23: mol_type = other DNA
cgggtcgccctggaggaccaggg

SEQ ID NO: 432 - DNA, 23 bp, synthetic construct
  misc_feature 1..23: note = Synthetic: Target sequences for wild type COL8A2
  source 1..23: mol_type = other DNA
acgggtcgccctggaggaccagg

SEQ ID NO: 433 - DNA, 23 bp, synthetic construct
  misc_feature 1..23: note = Synthetic: Target sequences for wild type COL8A2
  source 1..23: mol_type = other DNA
ggtttcacgggtcgccctggagg

SEQ ID NO: 434 - DNA, 23 bp, synthetic construct
  misc_feature 1..23: note = Synthetic: Target sequences for wild type COL8A2
  source 1..23: mol_type = other DNA
ccagggcgacccgtgaaacccgg

SEQ ID NO: 435 - DNA, 23 bp, synthetic construct
  misc_feature 1..23: note = Synthetic: Target sequences for wild type COL8A2
  source 1..23: mol_type = other DNA
ccgggtttcacgggtcgccctgg

SEQ ID NO: 436 - DNA, 23 bp, synthetic construct
  misc_feature 1..23: note = Synthetic: Target sequences for wild type COL8A2
  source 1..23: mol_type = other DNA
aagggtgagccgggtttcacggg

SEQ ID NO: 437 - DNA, 23 bp, synthetic construct
  misc_feature 1..23: note = Synthetic: Target sequences for wild type COL8A2
  source 1..23: mol_type = other DNA
caagggtgagccgggtttcacgg

SEQ ID NO: 438 - DNA, 23 bp, synthetic construct
  misc_feature 1..23: note = Synthetic: Target sequences for wild type COL8A2
  source 1..23: mol_type = other DNA
cgtgaaacccggctcacccttgg

SEQ ID NO: 439 - DNA, 23 bp, synthetic construct
  misc_feature 1..23: note = Synthetic: Target sequences for wild type COL8A2
  source 1..23: mol_type = other DNA
gtgaaacccggctcacccttggg

SEQ ID NO: 440 - DNA, 23 bp, synthetic construct
  misc_feature 1..23: note = Synthetic: Target sequences for wild type COL8A2
  source 1..23: mol_type = other DNA
actgggcccaagggtgagccggg

SEQ ID NO: 441 - DNA, 23 bp, synthetic construct
  misc_feature 1..23: note = Synthetic: Target sequences for wild type COL8A2
  source 1..23: mol_type = other DNA
aactgggcccaagggtgagccgg

SEQ ID NO: 442 - DNA, 23 bp, synthetic construct
  misc_feature 1..23: note = Synthetic: Target sequences for wild type COL8A2
  source 1..23: mol_type = other DNA
ggctcacccttgggcccagttgg

SEQ ID NO: 443 - DNA, 23 bp, synthetic construct
  misc_feature 1..23: note = Synthetic: Target sequences for wild type COL8A2
  source 1..23: mol_type = other DNA
cccttgggcccagttggtccagg

SEQ ID NO: 444 - DNA, 23 bp, synthetic construct
  misc_feature 1..23: note = Synthetic: Target sequences for wild type COL8A2
  source 1..23: mol_type = other DNA
cctggaccaactgggcccaaggg

SEQ ID NO: 445 - DNA, 23 bp, synthetic construct
  misc_feature 1..23: note = Synthetic: Target sequences for wild type COL8A2
  source 1..23: mol_type = other DNA
ccttgggcccagttggtccaggg

SEQ ID NO: 446 - DNA, 23 bp, synthetic construct
  misc_feature 1..23: note = Synthetic: Target sequences for wild type COL8A2
  source 1..23: mol_type = other DNA
ccctggaccaactgggcccaagg

SEQ ID NO: 447 - DNA, 23 bp, synthetic construct
  misc_feature 1..23: note = Synthetic: Target sequences for wild type COL8A2
  source 1..23: mol_type = other DNA
cttgggcccagttggtccagggg

SEQ ID NO: 448 - DNA, 23 bp, synthetic construct
  misc_feature 1..23: note = Synthetic: Target sequences for wild type COL8A2
  source 1..23: mol_type = other DNA
ttgggcccagttggtccaggggg

SEQ ID NO: 449 - DNA, 23 bp, synthetic construct
  misc_feature 1..23: note = Synthetic: Target sequences for wild type COL8A2
  source 1..23: mol_type = other DNA
atggaccccctggaccaactggg

SEQ ID NO: 450 - DNA, 23 bp, synthetic construct
  misc_feature 1..23: note = Synthetic: Target sequences for wild type COL8A2
  source 1..23: mol_type = other DNA
catggaccccctggaccaactgg

SEQ ID NO: 451 - DNA, 23 bp, synthetic construct
  misc_feature 1..23: note = Synthetic: Target sequences for wild type COL8A2
  source 1..23: mol_type = other DNA
cagttggtccagggggtccatgg

SEQ ID NO: 452 - DNA, 23 bp, synthetic construct
  misc_feature 1..23: note = Synthetic: Target sequences for wild type COL8A2
  source 1..23: mol_type = other DNA
agttggtccagggggtccatggg

SEQ ID NO: 453 - DNA, 23 bp, synthetic construct
  misc_feature 1..23: note = Synthetic: Target sequences for wild type COL8A2
  source 1..23: mol_type = other DNA
ccagggggtccatgggccccagg

SEQ ID NO: 454 - DNA, 23 bp, synthetic construct
  misc_feature 1..23: note = Synthetic: Target sequences for wild type COL8A2
  source 1..23: mol_type = other DNA
cctggggcccatggaccccctgg

SEQ ID NO: 455 - DNA, 23 bp, synthetic construct
  misc_feature 1..23: note = Synthetic: Target sequences for wild type COL8A2
  source 1..23: mol_type = other DNA
aggggacttcctggggcccatgg

SEQ ID NO: 456 - DNA, 23 bp, synthetic construct
  misc_feature 1..23: note = Synthetic: Target sequences for wild type COL8A2
  source 1..23: mol_type = other DNA
aggtgagaggggacttcctgggg

SEQ ID NO: 457 - DNA, 23 bp, synthetic construct
  misc_feature 1..23: note = Synthetic: Target sequences for wild type COL8A2
  source 1..23: mol_type = other DNA
caggtgagaggggacttcctggg

SEQ ID NO: 458 - DNA, 23 bp, synthetic construct
  misc_feature 1..23: note = Synthetic: Target sequences for wild type COL8A2
  source 1..23: mol_type = other DNA
ccaggaagtcccctctcacctgg

SEQ ID NO: 459 - DNA, 23 bp, synthetic construct
  misc_feature 1..23: note = Synthetic: Target sequences for wild type COL8A2
  source 1..23: mol_type = other DNA
ccaggtgagaggggacttcctgg

SEQ ID NO: 460 - DNA, 23 bp, synthetic construct
  misc_feature 1..23: note = Synthetic: Target sequences for wild type COL8A2
  source 1..23: mol_type = other DNA
caggaagtcccctctcacctggg

SEQ ID NO: 461 - DNA, 23 bp, synthetic construct
  misc_feature 1..23: note = Synthetic: Target sequences for wild type COL8A2
  source 1..23: mol_type = other DNA
cccctctcacctgggacccctgg

SEQ ID NO: 462 - DNA, 23 bp, synthetic construct
  misc_feature 1..23: note = Synthetic: Target sequences for wild type COL8A2
  source 1..23: mol_type = other DNA
ccaggggtcccaggtgagagggg

SEQ ID NO: 463 - DNA, 23 bp, synthetic construct
  misc_feature 1..23: note = Synthetic: Target sequences for wild type COL8A2
  source 1..23: mol_type = other DNA
accaggggtcccaggtgagaggg

SEQ ID NO: 464 - DNA, 23 bp, synthetic construct
  misc_feature 1..23: note = Synthetic: Target sequences for wild type COL8A2
  source 1..23: mol_type = other DNA
aaccaggggtcccaggtgagagg

SEQ ID NO: 465 - DNA, 23 bp, synthetic construct
  misc_feature 1..23: note = Synthetic: Target sequences for wild type COL8A2
  source 1..23: mol_type = other DNA
gctgggaaaccaggggtcccagg

SEQ ID NO: 466 - DNA, 23 bp, synthetic construct
  misc_feature 1..23: note = Synthetic: Target sequences for wild type COL8A2
  source 1..23: mol_type = other DNA
ggacccctggtttcccagccagg

SEQ ID NO: 467 - DNA, 23 bp, synthetic construct
  misc_feature 1..23: note = Synthetic: Target sequences for wild type COL8A2
  source 1..23: mol_type = other DNA
tggcctggctgggaaaccagggg

SEQ ID NO: 468 - DNA, 23 bp, synthetic construct
  misc_feature 1..23: note = Synthetic: Target sequences for wild type COL8A2
  source 1..23: mol_type = other DNA
gtggcctggctgggaaaccaggg

SEQ ID NO: 469 - DNA, 23 bp, synthetic construct
  misc_feature 1..23: note = Synthetic: Target sequences for wild type COL8A2
  source 1..23: mol_type = other DNA
agtggcctggctgggaaaccagg

SEQ ID NO: 470 - DNA, 23 bp, synthetic construct
  misc_feature 1..23: note = Synthetic: Target sequences for wild type COL8A2
  source 1..23: mol_type = other DNA
ggtttcccagccaggccactagg

SEQ ID NO: 471 - DNA, 23 bp, synthetic construct
  misc_feature 1..23: note = Synthetic: Target sequences for wild type COL8A2
  source 1..23: mol_type = other DNA
aggggcctagtggcctggctggg

SEQ ID NO: 472 - DNA, 23 bp, synthetic construct
  misc_feature 1..23: note = Synthetic: Target sequences for wild type COL8A2
  source 1..23: mol_type = other DNA
caggggcctagtggcctggctgg

SEQ ID NO: 473 - DNA, 23 bp, synthetic construct
  misc_feature 1..23: note = Synthetic: Target sequences for wild type COL8A2
  source 1..23: mol_type = other DNA
cagccaggccactaggcccctgg

SEQ ID NO: 474 - DNA, 23 bp, synthetic construct
  misc_feature 1..23: note = Synthetic: Target sequences for wild type COL8A2
  source 1..23: mol_type = other DNA
tgaccaggggcctagtggcctgg

SEQ ID NO: 475 - DNA, 23 bp, synthetic construct
  misc_feature 1..23: note = Synthetic: Target sequences for wild type COL8A2
  source 1..23: mol_type = other DNA
cgaggtgaccaggggcctagtgg

SEQ ID NO: 476 - DNA, 23 bp, synthetic construct
  misc_feature 1..23: note = Synthetic: Target sequences for wild type COL8A2
  source 1..23: mol_type = other DNA
ctggcattcgaggtgaccagggg

SEQ ID NO: 477 - DNA, 23 bp, synthetic construct
  misc_feature 1..23: note = Synthetic: Target sequences for wild type COL8A2
  source 1..23: mol_type = other DNA
ccctggtcacctcgaatgccagg

SEQ ID NO: 478 - DNA, 23 bp, synthetic construct
  misc_feature 1..23: note = Synthetic: Target sequences for wild type COL8A2
  source 1..23: mol_type = other DNA
cctggcattcgaggtgaccaggg

SEQ ID NO: 479 - DNA, 23 bp, synthetic construct
  misc_feature 1..23: note = Synthetic: Target sequences for wild type COL8A2
  source 1..23: mol_type = other DNA
gcctggcattcgaggtgaccagg

SEQ ID NO: 480 - DNA, 23 bp, synthetic construct
  misc_feature 1..23: note = Synthetic: Target sequences for wild type COL8A2
  source 1..23: mol_type = other DNA
cctcgaatgccaggcactcctgg

SEQ ID NO: 481 - DNA, 23 bp, synthetic construct
  misc_feature 1..23: note = Synthetic: Target sequences for wild type COL8A2
  source 1..23: mol_type = other DNA
ccaggagtgcctggcattcgagg

SEQ ID NO: 482 - DNA, 23 bp, synthetic construct
  misc_feature 1..23: note = Synthetic: Target sequences for wild type COL8A2
  source 1..23: mol_type = other DNA
ctcgaatgccaggcactcctggg

SEQ ID NO: 483 - DNA, 23 bp, synthetic construct
  misc_feature 1..23: note = Synthetic: Target sequences for wild type COL8A2
  source 1..23: mol_type = other DNA
tcgaatgccaggcactcctgggg

SEQ ID NO: 484 - DNA, 23 bp, synthetic construct
  misc_feature 1..23: note = Synthetic: Target sequences for wild type COL8A2
  source 1..23: mol_type = other DNA
cgaatgccaggcactcctggggg

SEQ ID NO: 485 - DNA, 23 bp, synthetic construct
  misc_feature 1..23: note = Synthetic: Target sequences for wild type COL8A2
  source 1..23: mol_type = other DNA
ggaggacccccaggagtgcctgg

SEQ ID NO: 486 - DNA, 23 bp, synthetic construct
  misc_feature 1..23: note = Synthetic: Target sequences for wild type COL8A2
  source 1..23: mol_type = other DNA
ggcactcctgggggtcctccagg

SEQ ID NO: 487 - DNA, 23 bp, synthetic construct
  misc_feature 1..23: note = Synthetic: Target sequences for wild type COL8A2
  source 1..23: mol_type = other DNA
gcagggcctggaggacccccagg

SEQ ID NO: 488 - DNA, 23 bp, synthetic construct
  misc_feature 1..23: note = Synthetic: Target sequences for wild type COL8A2
  source 1..23: mol_type = other DNA
aagggtgaggcagggcctggagg

SEQ ID NO: 489 - DNA, 23 bp, synthetic construct
  misc_feature 1..23: note = Synthetic: Target sequences for wild type COL8A2
  source 1..23: mol_type = other DNA
ccaggccctgcctcacccttagg

SEQ ID NO: 490 - DNA, 23 bp, synthetic construct
  misc_feature 1..23: note = Synthetic: Target sequences for wild type COL8A2
  source 1..23: mol_type = other DNA
cctaagggtgaggcagggcctgg

SEQ ID NO: 491 - DNA, 23 bp, synthetic construct
  misc_feature 1..23: note = Synthetic: Target sequences for wild type COL8A2
  source 1..23: mol_type = other DNA
ctgggcctaagggtgaggcaggg

SEQ ID NO: 492 - DNA, 23 bp, synthetic construct
  misc_feature 1..23: note = Synthetic: Target sequences for wild type COL8A2
  source 1..23: mol_type = other DNA
cctgcctcacccttaggcccagg

SEQ ID NO: 493 - DNA, 23 bp, synthetic construct
  misc_feature 1..23: note = Synthetic: Target sequences for wild type COL8A2
  source 1..23: mol_type = other DNA
cctgggcctaagggtgaggcagg

SEQ ID NO: 494 - DNA, 23 bp, synthetic construct
  misc_feature 1..23: note = Synthetic: Target sequences for wild type COL8A2
  source 1..23: mol_type = other DNA
ctgcctcacccttaggcccaggg

SEQ ID NO: 495 - DNA, 23 bp, synthetic construct
  misc_feature 1..23: note = Synthetic: Target sequences for wild type COL8A2
  source 1..23: mol_type = other DNA
tgcctcacccttaggcccagggg

SEQ ID NO: 496 - DNA, 23 bp, synthetic construct
  misc_feature 1..23: note = Synthetic: Target sequences for wild type COL8A2
  source 1..23: mol_type = other DNA
gcctcacccttaggcccaggggg

SEQ ID NO: 497 - DNA, 23 bp, synthetic construct
  misc_feature 1..23: note = Synthetic: Target sequences for wild type COL8A2
  source 1..23: mol_type = other DNA
gccccctgggcctaagggtgagg

SEQ ID NO: 498 - DNA, 23 bp, synthetic construct
  misc_feature 1..23: note = Synthetic: Target sequences for wild type COL8A2
  source 1..23: mol_type = other DNA
cgtgggccccctgggcctaaggg

SEQ ID NO: 499 - DNA, 23 bp, synthetic construct
  misc_feature 1..23: note = Synthetic: Target sequences for wild type COL8A2
  source 1..23: mol_type = other DNA
acgtgggccccctgggcctaagg

SEQ ID NO: 500 - DNA, 23 bp, synthetic construct
  misc_feature 1..23: note = Synthetic: Target sequences for wild type COL8A2
  source 1..23: mol_type = other DNA
ctggcagacgtgggccccctggg

SEQ ID NO: 501 - DNA, 23 bp, synthetic construct
  misc_feature 1..23: note = Synthetic: Target sequences for wild type COL8A2
  source 1..23: mol_type = other DNA
ccagggggcccacgtctgccagg

SEQ ID NO: 502 - DNA, 23 bp, synthetic construct
  misc_feature 1..23: note = Synthetic: Target sequences for wild type COL8A2
  source 1..23: mol_type = other DNA
cctggcagacgtgggccccctgg

SEQ ID NO: 503 - DNA, 23 bp, synthetic construct
  misc_feature 1..23: note = Synthetic: Target sequences for wild type COL8A2
  source 1..23: mol_type = other DNA
cagggcttcctggcagacgtggg

SEQ ID NO: 504 - DNA, 23 bp, synthetic construct
  misc_feature 1..23: note = Synthetic: Target sequences for wild type COL8A2
  source 1..23: mol_type = other DNA
gcagggcttcctggcagacgtgg

SEQ ID NO: 505 - DNA, 23 bp, synthetic construct
  misc_feature 1..23: note = Synthetic: Target sequences for wild type COL8A2
  source 1..23: mol_type = other DNA
ccaggaagccctgcagacccagg

SEQ ID NO: 506 - DNA, 23 bp, synthetic construct
  misc_feature 1..23: note = Synthetic: Target sequences for wild type COL8A2
  source 1..23: mol_type = other DNA
cctgggtctgcagggcttcctgg

SEQ ID NO: 507 - DNA, 23 bp, synthetic construct
  misc_feature 1..23: note = Synthetic: Target sequences for wild type COL8A2
  source 1..23: mol_type = other DNA
ctggacttcctgggtctgcaggg

SEQ ID NO: 508 - DNA, 23 bp, synthetic construct
  misc_feature 1..23: note = Synthetic: Target sequences for wild type COL8A2
  source 1..23: mol_type = other DNA
cctgcagacccaggaagtccagg

SEQ ID NO: 509 - DNA, 23 bp, synthetic construct
  misc_feature 1..23: note = Synthetic: Target sequences for wild type COL8A2
  source 1..23: mol_type = other DNA
cctggacttcctgggtctgcagg

SEQ ID NO: 510 - DNA, 23 bp, synthetic construct
  misc_feature 1..23: note = Synthetic: Target sequences for wild type COL8A2
  source 1..23: mol_type = other DNA
ctgcagacccaggaagtccaggg

SEQ ID NO: 511 - DNA, 23 bp, synthetic construct
  misc_feature 1..23: note = Synthetic: Target sequences for wild type COL8A2
  source 1..23: mol_type = other DNA
tgcagacccaggaagtccagggg

SEQ ID NO: 512 - DNA, 23 bp, synthetic construct
  misc_feature 1..23: note = Synthetic: Target sequences for wild type COL8A2
  source 1..23: mol_type = other DNA
gcagacccaggaagtccaggggg

SEQ ID NO: 513 - DNA, 23 bp, synthetic construct
  misc_feature 1..23: note = Synthetic: Target sequences for wild type COL8A2
  source 1..23: mol_type = other DNA
ggggtccccctggacttcctggg

SEQ ID NO: 514 - DNA, 23 bp, synthetic construct
  misc_feature 1..23: note = Synthetic: Target sequences for wild type COL8A2
  source 1..23: mol_type = other DNA
gggggtccccctggacttcctgg

SEQ ID NO: 515 - DNA, 23 bp, synthetic construct
  misc_feature 1..23: note = Synthetic: Target sequences for wild type COL8A2
  source 1..23: mol_type = other DNA
cagggtcttgggggtccccctgg

SEQ ID NO: 516 - DNA, 23 bp, synthetic construct
  misc_feature 1..23: note = Synthetic: Target sequences for wild type COL8A2
  source 1..23: mol_type = other DNA
gggggacccccaagaccctgtgg

SEQ ID NO: 517 - DNA, 23 bp, synthetic construct
  misc_feature 1..23: note = Synthetic: Target sequences for wild type COL8A2
  source 1..23: mol_type = other DNA
ggggacccccaagaccctgtggg

SEQ ID NO: 518 - DNA, 23 bp, synthetic construct
  misc_feature 1..23: note = Synthetic: Target sequences for wild type COL8A2
  source 1..23: mol_type = other DNA
cagggcccacagggtcttggggg

SEQ ID NO: 519 - DNA, 23 bp, synthetic construct
  misc_feature 1..23: note = Synthetic: Target sequences for wild type COL8A2
  source 1..23: mol_type = other DNA
gcagggcccacagggtcttgggg

SEQ ID NO: 520 - DNA, 23 bp, synthetic construct
  misc_feature 1..23: note = Synthetic: Target sequences for wild type COL8A2
  source 1..23: mol_type = other DNA
agcagggcccacagggtcttggg

SEQ ID NO: 521 - DNA, 23 bp, synthetic construct
  misc_feature 1..23: note = Synthetic: Target sequences for wild type COL8A2
  source 1..23: mol_type = other DNA
gagcagggcccacagggtcttgg

SEQ ID NO: 522 - DNA, 23 bp, synthetic construct
  misc_feature 1..23: note = Synthetic: Target sequences for wild type COL8A2
  source 1..23: mol_type = other DNA
ccctgtgggccctgctcccctgg

SEQ ID NO: 523 - DNA, 23 bp, synthetic construct
  misc_feature 1..23: note = Synthetic: Target sequences for wild type COL8A2
  source 1..23: mol_type = other DNA
ccaggggagcagggcccacaggg

SEQ ID NO: 524 - DNA, 23 bp, synthetic construct
  misc_feature 1..23: note = Synthetic: Target sequences for wild type COL8A2
  source 1..23: mol_type = other DNA
gccaggggagcagggcccacagg

SEQ ID NO: 525 - DNA, 23 bp, synthetic construct
  misc_feature 1..23: note = Synthetic: Target sequences for wild type COL8A2
  source 1..23: mol_type = other DNA
gatggggagccaggggagcaggg

SEQ ID NO: 526 - DNA, 23 bp, synthetic construct
  misc_feature 1..23: note = Synthetic: Target sequences for wild type COL8A2
  source 1..23: mol_type = other DNA
ggatggggagccaggggagcagg

SEQ ID NO: 527 - DNA, 23 bp, synthetic construct
  misc_feature 1..23: note = Synthetic: Target sequences for wild type COL8A2
  source 1..23: mol_type = other DNA
aggggaggatggggagccagggg

SEQ ID NO: 528 - DNA, 23 bp, synthetic construct
  misc_feature 1..23: note = Synthetic: Target sequences for wild type COL8A2
  source 1..23: mol_type = other DNA
caggggaggatggggagccaggg

SEQ ID NO: 529 - DNA, 23 bp, synthetic construct
  misc_feature 1..23: note = Synthetic: Target sequences for wild type COL8A2
  source 1..23: mol_type = other DNA
cctggctccccatcctcccctgg

SEQ ID NO: 530 - DNA, 23 bp, synthetic construct
  misc_feature 1..23: note = Synthetic: Target sequences for wild type COL8A2
  source 1..23: mol_type = other DNA
ccaggggaggatggggagccagg

SEQ ID NO: 531 - DNA, 23 bp, synthetic construct
  misc_feature 1..23: note = Synthetic: Target sequences for wild type COL8A2
  source 1..23: mol_type = other DNA
gggtgagccaggggaggatgggg

SEQ ID NO: 532 - DNA, 23 bp, synthetic construct
  misc_feature 1..23: note = Synthetic: Target sequences for wild type COL8A2
  source 1..23: mol_type = other DNA
ggggtgagccaggggaggatggg

SEQ ID NO: 533 - DNA, 23 bp, synthetic construct
  misc_feature 1..23: note = Synthetic: Target sequences for wild type COL8A2
  source 1..23: mol_type = other DNA
aggggtgagccaggggaggatgg

SEQ ID NO: 534 - DNA, 23 bp, synthetic construct
  misc_feature 1..23: note = Synthetic: Target sequences for wild type COL8A2
  source 1..23: mol_type = other DNA
ggacaggggtgagccaggggagg

SEQ ID NO: 535 - DNA, 23 bp, synthetic construct
  misc_feature 1..23: note = Synthetic: Target sequences for wild type COL8A2
  source 1..23: mol_type = other DNA
gggggacaggggtgagccagggg

SEQ ID NO: 536 - DNA, 23 bp, synthetic construct
  misc_feature 1..23: note = Synthetic: Target sequences for wild type COL8A2
  source 1..23: mol_type = other DNA
tgggggacaggggtgagccaggg

SEQ ID NO: 537 - DNA, 23 bp, synthetic construct
  misc_feature 1..23: note = Synthetic: Target sequences for wild type COL8A2
  source 1..23: mol_type = other DNA
ttgggggacaggggtgagccagg

SEQ ID NO: 538 - DNA, 23 bp, synthetic construct
  misc_feature 1..23: note = Synthetic: Target sequences for wild type COL8A2
  source 1..23: mol_type = other DNA
cccctgtcccccaagagtcctgg

SEQ ID NO: 539 - DNA, 23 bp, synthetic construct
  misc_feature 1..23: note = Synthetic: Target sequences for wild type COL8A2
  source 1..23: mol_type = other DNA
ccaggactcttgggggacagggg

SEQ ID NO: 540 - DNA, 23 bp, synthetic construct
  misc_feature 1..23: note = Synthetic: Target sequences for wild type COL8A2
  source 1..23: mol_type = other DNA
ccctgtcccccaagagtcctggg

SEQ ID NO: 541 - DNA, 23 bp, synthetic construct
  misc_feature 1..23: note = Synthetic: Target sequences for wild type COL8A2
  source 1..23: mol_type = other DNA
cccaggactcttgggggacaggg

SEQ ID NO: 542 - DNA, 23 bp, synthetic construct
  misc_feature 1..23: note = Synthetic: Target sequences for wild type COL8A2
  source 1..23: mol_type = other DNA
tcccaggactcttgggggacagg

SEQ ID NO: 543 - DNA, 23 bp, synthetic construct
  misc_feature 1..23: note = Synthetic: Target sequences for wild type COL8A2
  source 1..23: mol_type = other DNA
tggggtcccaggactcttggggg

SEQ ID NO: 544 - DNA, 23 bp, synthetic construct
  misc_feature 1..23: note = Synthetic: Target sequences for wild type COL8A2
  source 1..23: mol_type = other DNA
ctggggtcccaggactcttgggg

SEQ ID NO: 545 - DNA, 23 bp, synthetic construct
  misc_feature 1..23: note = Synthetic: Target sequences for wild type COL8A2
  source 1..23: mol_type = other DNA
gctggggtcccaggactcttggg

SEQ ID NO: 546 - DNA, 23 bp, synthetic construct
  misc_feature 1..23: note = Synthetic: Target sequences for wild type COL8A2
  source 1..23: mol_type = other DNA
agctggggtcccaggactcttgg

SEQ ID NO: 547 - DNA, 23 bp, synthetic construct
  misc_feature 1..23: note = Synthetic: Target sequences for wild type COL8A2
  source 1..23: mol_type = other DNA
aagagtcctgggaccccagctgg

SEQ ID NO: 548 - DNA, 23 bp, synthetic construct
  misc_feature 1..23: note = Synthetic: Target sequences for wild type COL8A2
  source 1..23: mol_type = other DNA
agagtcctgggaccccagctggg

SEQ ID NO: 549 - DNA, 23 bp, synthetic construct
  misc_feature 1..23: note = Synthetic: Target sequences for wild type COL8A2
  source 1..23: mol_type = other DNA
aggggcccagctggggtcccagg

SEQ ID NO: 550 - DNA, 23 bp, synthetic construct
  misc_feature 1..23: note = Synthetic: Target sequences for wild type COL8A2
  source 1..23: mol_type = other DNA
gggggacaggggcccagctgggg

SEQ ID NO: 551 - DNA, 23 bp, synthetic construct
  misc_feature 1..23: note = Synthetic: Target sequences for wild type COL8A2
  source 1..23: mol_type = other DNA
agggggacaggggcccagctggg

SEQ ID NO: 552 - DNA, 23 bp, synthetic construct
  misc_feature 1..23: note = Synthetic: Target sequences for wild type COL8A2
  source 1..23: mol_type = other DNA
aagggggacaggggcccagctgg

SEQ ID NO: 553 - DNA, 23 bp, synthetic construct
  misc_feature 1..23: note = Synthetic: Target sequences for wild type COL8A2
  source 1..23: mol_type = other DNA
agctgggcccctgtcccccttgg

SEQ ID NO: 554 - DNA, 23 bp, synthetic construct
  misc_feature 1..23: note = Synthetic: Target sequences for wild type COL8A2
  source 1..23: mol_type = other DNA
gctgggcccctgtcccccttggg

SEQ ID NO: 555 - DNA, 23 bp, synthetic construct
  misc_feature 1..23: note = Synthetic: Target sequences for wild type COL8A2
  source 1..23: mol_type = other DNA
ctgggcccctgtcccccttgggg

SEQ ID NO: 556 - DNA, 23 bp, synthetic construct
  misc_feature 1..23: note = Synthetic: Target sequences for wild type COL8A2
  source 1..23: mol_type = other DNA
cccctgtcccccttggggcctgg

SEQ ID NO: 557 - DNA, 23 bp, synthetic construct
  misc_feature 1..23: note = Synthetic: Target sequences for wild type COL8A2
  source 1..23: mol_type = other DNA
ccaggccccaagggggacagggg

SEQ ID NO: 558 - DNA, 23 bp, synthetic construct
  misc_feature 1..23: note = Synthetic: Target sequences for wild type COL8A2
  source 1..23: mol_type = other DNA
gccaggccccaagggggacaggg

SEQ ID NO: 559 - DNA, 23 bp, synthetic construct
  misc_feature 1..23: note = Synthetic: Target sequences for wild type COL8A2
  source 1..23: mol_type = other DNA
tgccaggccccaagggggacagg

SEQ ID NO: 560 - DNA, 23 bp, synthetic construct
  misc_feature 1..23: note = Synthetic: Target sequences for wild type COL8A2
  source 1..23: mol_type = other DNA
aggactgccaggccccaaggggg

SEQ ID NO: 561 - DNA, 23 bp, synthetic construct
  misc_feature 1..23: note = Synthetic: Target sequences for wild type COL8A2
  source 1..23: mol_type = other DNA
caggactgccaggccccaagggg

SEQ ID NO: 562 - DNA, 23 bp, synthetic construct
  misc_feature 1..23: note = Synthetic: Target sequences for wild type COL8A2
  source 1..23: mol_type = other DNA
cccttggggcctggcagtcctgg

SEQ ID NO: 563 - DNA, 23 bp, synthetic construct
  misc_feature 1..23: note = Synthetic: Target sequences for wild type COL8A2
  source 1..23: mol_type = other DNA
ccaggactgccaggccccaaggg

SEQ ID NO: 564 - DNA, 23 bp, synthetic construct
  misc_feature 1..23: note = Synthetic: Target sequences for wild type COL8A2
  source 1..23: mol_type = other DNA
gccaggactgccaggccccaagg

SEQ ID NO: 565 - DNA, 23 bp, synthetic construct
  misc_feature 1..23: note = Synthetic: Target sequences for wild type COL8A2
  source 1..23: mol_type = other DNA
tatgggatgccaggactgccagg

SEQ ID NO: 566 - DNA, 23 bp, synthetic construct
  misc_feature 1..23: note = Synthetic: Target sequences for wild type COL8A2
  source 1..23: mol_type = other DNA
tcctggcatcccatagccagtgg

SEQ ID NO: 567 - DNA, 23 bp, synthetic construct
  misc_feature 1..23: note = Synthetic: Target sequences for wild type COL8A2
  source 1..23: mol_type = other DNA
cctggcatcccatagccagtggg

SEQ ID NO: 568 - DNA, 23 bp, synthetic construct
  misc_feature 1..23: note = Synthetic: Target sequences for wild type COL8A2
  source 1..23: mol_type = other DNA
cccactggctatgggatgccagg

SEQ ID NO: 569 - DNA, 23 bp, synthetic construct
  misc_feature 1..23: note = Synthetic: Target sequences for wild type COL8A2
  source 1..23: mol_type = other DNA
ctggcatcccatagccagtgggg

SEQ ID NO: 570 - DNA, 23 bp, synthetic construct
  misc_feature 1..23: note = Synthetic: Target sequences for wild type COL8A2
  source 1..23: mol_type = other DNA
tgataggccccactggctatggg

SEQ ID NO: 571 - DNA, 23 bp, synthetic construct
  misc_feature 1..23: note = Synthetic: Target sequences for wild type COL8A2
  source 1..23: mol_type = other DNA
ctgataggccccactggctatgg

SEQ ID NO: 572 - DNA, 23 bp, synthetic construct
  misc_feature 1..23: note = Synthetic: Target sequences for wild type COL8A2
  source 1..23: mol_type = other DNA
ccagtggggcctatcagcccagg

SEQ ID NO: 573 - DNA, 23 bp, synthetic construct
  misc_feature 1..23: note = Synthetic: Target sequences for wild type COL8A2
  source 1..23: mol_type = other DNA
cctgggctgataggccccactgg

SEQ ID NO: 574 - DNA, 23 bp, synthetic construct
  misc_feature 1..23: note = Synthetic: Target sequences for wild type COL8A2
  source 1..23: mol_type = other DNA
cagtggggcctatcagcccaggg

SEQ ID NO: 575 - DNA, 23 bp, synthetic construct
  misc_feature 1..23: note = Synthetic: Target sequences for wild type COL8A2
  source 1..23: mol_type = other DNA
agtggggcctatcagcccagggg

SEQ ID NO: 576 - DNA, 23 bp, synthetic construct
  misc_feature 1..23: note = Synthetic: Target sequences for wild type COL8A2
  source 1..23: mol_type = other DNA
gtggggcctatcagcccaggggg

SEQ ID NO: 577 - DNA, 23 bp, synthetic construct
  misc_feature 1..23: note = Synthetic: Target sequences for wild type COL8A2
  source 1..23: mol_type = other DNA
tggggcctatcagcccagggggg

SEQ ID NO: 578 - DNA, 23 bp, synthetic construct
  misc_feature 1..23: note = Synthetic: Target sequences for wild type COL8A2
  source 1..23: mol_type = other DNA
cggggcccccctgggctgatagg

SEQ ID NO: 579 - DNA, 23 bp, synthetic construct
  misc_feature 1..23: note = Synthetic: Target sequences for wild type COL8A2
  source 1..23: mol_type = other DNA
ctatcagcccaggggggccccgg

SEQ ID NO: 580 - DNA, 23 bp, synthetic construct
  misc_feature 1..23: note = Synthetic: Target sequences for wild type COL8A2
  source 1..23: mol_type = other DNA
tatcagcccaggggggccccggg

SEQ ID NO: 581 - DNA, 23 bp, synthetic construct
  misc_feature 1..23: note = Synthetic: Target sequences for wild type COL8A2
  source 1..23: mol_type = other DNA
cagggacccggggcccccctggg

SEQ ID NO: 582 - DNA, 23 bp, synthetic construct
  misc_feature 1..23: note = Synthetic: Target sequences for wild type COL8A2
  source 1..23: mol_type = other DNA
ccaggggggccccgggtccctgg

SEQ ID NO: 583 - DNA, 23 bp, synthetic construct
  misc_feature 1..23: note = Synthetic: Target sequences for wild type COL8A2
  source 1..23: mol_type = other DNA
ccagggacccggggcccccctgg

SEQ ID NO: 584 - DNA, 23 bp, synthetic construct
  misc_feature 1..23: note = Synthetic: Target sequences for wild type COL8A2
  source 1..23: mol_type = other DNA
aaaggggagccagggacccgggg

SEQ ID NO: 585 - DNA, 23 bp, synthetic construct
  misc_feature 1..23: note = Synthetic: Target sequences for wild type COL8A2
  source 1..23: mol_type = other DNA
caaaggggagccagggacccggg

SEQ ID NO: 586 - DNA, 23 bp, synthetic construct
  misc_feature 1..23: note = Synthetic: Target sequences for wild type COL8A2
  source 1..23: mol_type = other DNA
ccgggtccctggctcccctttgg

SEQ ID NO: 587 - DNA, 23 bp, synthetic construct
  misc_feature 1..23: note = Synthetic: Target sequences for wild type COL8A2
  source 1..23: mol_type = other DNA
ccaaaggggagccagggacccgg

SEQ ID NO: 588 - DNA, 23 bp, synthetic construct
  misc_feature 1..23: note = Synthetic: Target sequences for wild type COL8A2
  source 1..23: mol_type = other DNA
caggggccaaaggggagccaggg

SEQ ID NO: 589 - DNA, 23 bp, synthetic construct
  misc_feature 1..23: note = Synthetic: Target sequences for wild type COL8A2
  source 1..23: mol_type = other DNA
tcaggggccaaaggggagccagg

SEQ ID NO: 590 - DNA, 23 bp, synthetic construct
  misc_feature 1..23: note = Synthetic: Target sequences for wild type COL8A2
  source 1..23: mol_type = other DNA
ggctcccctttggcccctgatgg

SEQ ID NO: 591 - DNA, 23 bp, synthetic construct
  misc_feature 1..23: note = Synthetic: Target sequences for wild type COL8A2
  source 1..23: mol_type = other DNA
gctcccctttggcccctgatggg

SEQ ID NO: 592 - DNA, 23 bp, synthetic construct
  misc_feature 1..23: note = Synthetic: Target sequences for wild type COL8A2
  source 1..23: mol_type = other DNA
gggcccatcaggggccaaagggg

SEQ ID NO: 593 - DNA, 23 bp, synthetic construct
  misc_feature 1..23: note = Synthetic: Target sequences for wild type COL8A2
  source 1..23: mol_type = other DNA
agggcccatcaggggccaaaggg

SEQ ID NO: 594 - DNA, 23 bp, synthetic construct
  misc_feature 1..23: note = Synthetic: Target sequences for wild type COL8A2
  source 1..23: mol_type = other DNA
cagggcccatcaggggccaaagg

SEQ ID NO: 595 - DNA, 23 bp, synthetic construct
  misc_feature 1..23: note = Synthetic: Target sequences for wild type COL8A2
  source 1..23: mol_type = other DNA
ttggcccctgatgggccctgtgg

SEQ ID NO: 596 - DNA, 23 bp, synthetic construct
  misc_feature 1..23: note = Synthetic: Target sequences for wild type COL8A2
  source 1..23: mol_type = other DNA
aggaccacagggcccatcagggg

SEQ ID NO: 597 - DNA, 23 bp, synthetic construct
  misc_feature 1..23: note = Synthetic: Target sequences for wild type COL8A2
  source 1..23: mol_type = other DNA
caggaccacagggcccatcaggg

SEQ ID NO: 598 - DNA, 23 bp, synthetic construct
  misc_feature 1..23: note = Synthetic: Target sequences for wild type COL8A2
  source 1..23: mol_type = other DNA
cctgatgggccctgtggtcctgg

SEQ ID NO: 599 - DNA, 23 bp, synthetic construct
  misc_feature 1..23: note = Synthetic: Target sequences for wild type COL8A2
  source 1..23: mol_type = other DNA
ccaggaccacagggcccatcagg

SEQ ID NO: 600 - DNA, 23 bp, synthetic construct
  misc_feature 1..23: note = Synthetic: Target sequences for wild type COL8A2
  source 1..23: mol_type = other DNA
gcagggttgccaggaccacaggg

SEQ ID NO: 601 - DNA, 23 bp, synthetic construct
  misc_feature 1..23: note = Synthetic: Target sequences for wild type COL8A2
  source 1..23: mol_type = other DNA
agcagggttgccaggaccacagg

SEQ ID NO: 602 - DNA, 23 bp, synthetic construct
  misc_feature 1..23: note = Synthetic: Target sequences for wild type COL8A2
  source 1..23: mol_type = other DNA
cctggcaaccctgctgcccctgg

SEQ ID NO: 603 - DNA, 23 bp, synthetic construct
  misc_feature 1..23: note = Synthetic: Target sequences for wild type COL8A2
  source 1..23: mol_type = other DNA
ccaggggcagcagggttgccagg

SEQ ID NO: 604 - DNA, 23 bp, synthetic construct
  misc_feature 1..23: note = Synthetic: Target sequences for wild type COL8A2
  source 1..23: mol_type = other DNA
ctggcaaccctgctgcccctggg

SEQ ID NO: 605 - DNA, 23 bp, synthetic construct
  misc_feature 1..23: note = Synthetic: Target sequences for wild type COL8A2
  source 1..23: mol_type = other DNA
tgggagtcccaggggcagcaggg

SEQ ID NO: 606 - DNA, 23 bp, synthetic construct
  misc_feature 1..23: note = Synthetic: Target sequences for wild type COL8A2
  source 1..23: mol_type = other DNA
gtgggagtcccaggggcagcagg

SEQ ID NO: 607 - DNA, 23 bp, synthetic construct
  misc_feature 1..23: note = Synthetic: Target sequences for wild type COL8A2
  source 1..23: mol_type = other DNA
agacggtgtgggagtcccagggg

SEQ ID NO: 608 - DNA, 23 bp, synthetic construct
  misc_feature 1..23: note = Synthetic: Target sequences for wild type COL8A2
  source 1..23: mol_type = other DNA
tagacggtgtgggagtcccaggg

SEQ ID NO: 609 - DNA, 23 bp, synthetic construct
  misc_feature 1..23: note = Synthetic: Target sequences for wild type COL8A2
  source 1..23: mol_type = other DNA
gtagacggtgtgggagtcccagg

SEQ ID NO: 610 - DNA, 23 bp, synthetic construct
  misc_feature 1..23: note = Synthetic: Target sequences for wild type COL8A2
  source 1..23: mol_type = other DNA
actcccacaccgtctactccagg

SEQ ID NO: 611 - DNA, 23 bp, synthetic construct
  misc_feature 1..23: note = Synthetic: Target sequences for wild type COL8A2
  source 1..23: mol_type = other DNA
cccacaccgtctactccaggagg

SEQ ID NO: 612 - DNA, 23 bp, synthetic construct
  misc_feature 1..23: note = Synthetic: Target sequences for wild type COL8A2
  source 1..23: mol_type = other DNA
cctcctggagtagacggtgtggg

SEQ ID NO: 613 - DNA, 23 bp, synthetic construct
  misc_feature 1..23: note = Synthetic: Target sequences for wild type COL8A2
  source 1..23: mol_type = other DNA
acctcctggagtagacggtgtgg

SEQ ID NO: 614 - DNA, 23 bp, synthetic construct
  misc_feature 1..23: note = Synthetic: Target sequences for wild type COL8A2
  source 1..23: mol_type = other DNA
aaaggacctcctggagtagacgg

SEQ ID NO: 615 - DNA, 23 bp, synthetic construct
  misc_feature 1..23: note = Synthetic: Target sequences for wild type COL8A2
  source 1..23: mol_type = other DNA
tctactccaggaggtccttttgg

SEQ ID NO: 616 - DNA, 23 bp, synthetic construct
  misc_feature 1..23: note = Synthetic: Target sequences for wild type COL8A2
  source 1..23: mol_type = other DNA
ctactccaggaggtccttttggg

SEQ ID NO: 617 - DNA, 23 bp, synthetic construct
  misc_feature 1..23: note = Synthetic: Target sequences for wild type COL8A2
  source 1..23: mol_type = other DNA
gtgggcccaaaaggacctcctgg

SEQ ID NO: 618 - DNA, 23 bp, synthetic construct
  misc_feature 1..23: note = Synthetic: Target sequences for wild type COL8A2
  source 1..23: mol_type = other DNA
ccttttgggcccacagctcctgg

SEQ ID NO: 619 - DNA, 23 bp, synthetic construct
  misc_feature 1..23: note = Synthetic: Target sequences for wild type COL8A2
  source 1..23: mol_type = other DNA
ccaggagctgtgggcccaaaagg

SEQ ID NO: 620 - DNA, 23 bp, synthetic construct
  misc_feature 1..23: note = Synthetic: Target sequences for wild type COL8A2
  source 1..23: mol_type = other DNA
aggggggagccaggagctgtggg

SEQ ID NO: 621 - DNA, 23 bp, synthetic construct
  misc_feature 1..23: note = Synthetic: Target sequences for wild type COL8A2
  source 1..23: mol_type = other DNA
caggggggagccaggagctgtgg

SEQ ID NO: 622 - DNA, 23 bp, synthetic construct
  misc_feature 1..23: note = Synthetic: Target sequences for wild type COL8A2
  source 1..23: mol_type = other DNA
cacagctcctggctcccccctgg

SEQ ID NO: 623 - DNA, 23 bp, synthetic construct
  misc_feature 1..23: note = Synthetic: Target sequences for wild type COL8A2
  source 1..23: mol_type = other DNA
acagctcctggctcccccctggg

SEQ ID NO: 624 - DNA, 23 bp, synthetic construct
  misc_feature 1..23: note = Synthetic: Target sequences for wild type COL8A2
  source 1..23: mol_type = other DNA
cagctcctggctcccccctgggg

SEQ ID NO: 625 - DNA, 23 bp, synthetic construct
  misc_feature 1..23: note = Synthetic: Target sequences for wild type COL8A2
  source 1..23: mol_type = other DNA
cctggctcccccctggggcctgg

SEQ ID NO: 626 - DNA, 23 bp, synthetic construct
  misc_feature 1..23: note = Synthetic: Target sequences for wild type COL8A2
  source 1..23: mol_type = other DNA
ccaggccccaggggggagccagg

SEQ ID NO: 627 - DNA, 23 bp, synthetic construct
  misc_feature 1..23: note = Synthetic: Target sequences for wild type COL8A2
  source 1..23: mol_type = other DNA
tggagttccaggccccagggggg

SEQ ID NO: 628 - DNA, 23 bp, synthetic construct
  misc_feature 1..23: note = Synthetic: Target sequences for wild type COL8A2
  source 1..23: mol_type = other DNA
ctggagttccaggccccaggggg

SEQ ID NO: 629 - DNA, 23 bp, synthetic construct
  misc_feature 1..23: note = Synthetic: Target sequences for wild type COL8A2
  source 1..23: mol_type = other DNA
cccctggggcctggaactccagg

SEQ ID NO: 630 - DNA, 23 bp, synthetic construct
  misc_feature 1..23: note = Synthetic: Target sequences for wild type COL8A2
  source 1..23: mol_type = other DNA
cctggagttccaggccccagggg

SEQ ID NO: 631 - DNA, 23 bp, synthetic construct
  misc_feature 1..23: note = Synthetic: Target sequences for wild type COL8A2
  source 1..23: mol_type = other DNA
tcctggagttccaggccccaggg

SEQ ID NO: 632 - DNA, 23 bp, synthetic construct
  misc_feature 1..23: note = Synthetic: Target sequences for wild type COL8A2
  source 1..23: mol_type = other DNA
ctcctggagttccaggccccagg

SEQ ID NO: 633 - DNA, 23 bp, synthetic construct
  misc_feature 1..23: note = Synthetic: Target sequences for wild type COL8A2
  source 1..23: mol_type = other DNA
ctggggcctggaactccaggagg

SEQ ID NO: 634 - DNA, 23 bp, synthetic construct
  misc_feature 1..23: note = Synthetic: Target sequences for wild type COL8A2
  source 1..23: mol_type = other DNA
tctgggcctcctggagttccagg

SEQ ID NO: 635 - DNA, 23 bp, synthetic construct
  misc_feature 1..23: note = Synthetic: Target sequences for wild type COL8A2
  source 1..23: mol_type = other DNA
aagggtgagtctgggcctcctgg

SEQ ID NO: 636 - DNA, 23 bp, synthetic construct
  misc_feature 1..23: note = Synthetic: Target sequences for wild type COL8A2
  source 1..23: mol_type = other DNA
caggagacaagggtgagtctggg

SEQ ID NO: 637 - DNA, 23 bp, synthetic construct
  misc_feature 1..23: note = Synthetic: Target sequences for wild type COL8A2
  source 1..23: mol_type = other DNA
ccagactcacccttgtctcctgg

SEQ ID NO: 638 - DNA, 23 bp, synthetic construct
  misc_feature 1..23: note = Synthetic: Target sequences for wild type COL8A2
  source 1..23: mol_type = other DNA
ccaggagacaagggtgagtctgg

SEQ ID NO: 639 - DNA, 23 bp, synthetic construct
  misc_feature 1..23: note = Synthetic: Target sequences for wild type COL8A2
  source 1..23: mol_type = other DNA
cagactcacccttgtctcctggg

SEQ ID NO: 640 - DNA, 23 bp, synthetic construct
  misc_feature 1..23: note = Synthetic: Target sequences for wild type COL8A2
  source 1..23: mol_type = other DNA
agactcacccttgtctcctgggg

SEQ ID NO: 641 - DNA, 23 bp, synthetic construct
  misc_feature 1..23: note = Synthetic: Target sequences for wild type COL8A2
  source 1..23: mol_type = other DNA
cccttgtctcctggggccccagg

SEQ ID NO: 642 - DNA, 23 bp, synthetic construct
  misc_feature 1..23: note = Synthetic: Target sequences for wild type COL8A2
  source 1..23: mol_type = other DNA
cctggggccccaggagacaaggg

SEQ ID NO: 643 - DNA, 23 bp, synthetic construct
  misc_feature 1..23: note = Synthetic: Target sequences for wild type COL8A2
  source 1..23: mol_type = other DNA
tcctggggccccaggagacaagg

SEQ ID NO: 644 - DNA, 23 bp, synthetic construct
  misc_feature 1..23: note = Synthetic: Target sequences for wild type COL8A2
  source 1..23: mol_type = other DNA
gatgggcttcctggggccccagg

SEQ ID NO: 645 - DNA, 23 bp, synthetic construct
  misc_feature 1..23: note = Synthetic: Target sequences for wild type COL8A2
  source 1..23: mol_type = other DNA
tggtttggatgggcttcctgggg

SEQ ID NO: 646 - DNA, 23 bp, synthetic construct
  misc_feature 1..23: note = Synthetic: Target sequences for wild type COL8A2
  source 1..23: mol_type = other DNA
ctggtttggatgggcttcctggg

SEQ ID NO: 647 - DNA, 23 bp, synthetic construct
  misc_feature 1..23: note = Synthetic: Target sequences for wild type COL8A2
  source 1..23: mol_type = other DNA
ccaggaagcccatccaaaccagg

SEQ ID NO: 648 - DNA, 23 bp, synthetic construct
  misc_feature 1..23: note = Synthetic: Target sequences for wild type COL8A2
  source 1..23: mol_type = other DNA
cctggtttggatgggcttcctgg

SEQ ID NO: 649 - DNA, 23 bp, synthetic construct
  misc_feature 1..23: note = Synthetic: Target sequences for wild type COL8A2
  source 1..23: mol_type = other DNA
taggcaaacctggtttggatggg

SEQ ID NO: 650 - DNA, 23 bp, synthetic construct
  misc_feature 1..23: note = Synthetic: Target sequences for wild type COL8A2
  source 1..23: mol_type = other DNA
ttaggcaaacctggtttggatgg

SEQ ID NO: 651 - DNA, 23 bp, synthetic construct
  misc_feature 1..23: note = Synthetic: Target sequences for wild type COL8A2
  source 1..23: mol_type = other DNA
tggcttaggcaaacctggtttgg

SEQ ID NO: 652 - DNA, 23 bp, synthetic construct
  misc_feature 1..23: note = Synthetic: Target sequences for wild type COL8A2
  source 1..23: mol_type = other DNA
ccaggtttgcctaagccagctgg

SEQ ID NO: 653 - DNA, 23 bp, synthetic construct
  misc_feature 1..23: note = Synthetic: Target sequences for wild type COL8A2
  source 1..23: mol_type = other DNA
ccagctggcttaggcaaacctgg

SEQ ID NO: 654 - DNA, 23 bp, synthetic construct
  misc_feature 1..23: note = Synthetic: Target sequences for wild type COL8A2
  source 1..23: mol_type = other DNA
ttgcctaagccagctggaccagg

SEQ ID NO: 655 - DNA, 23 bp, synthetic construct
  misc_feature 1..23: note = Synthetic: Target sequences for wild type COL8A2
  source 1..23: mol_type = other DNA
tgcctaagccagctggaccaggg

SEQ ID NO: 656 - DNA, 23 bp, synthetic construct
  misc_feature 1..23: note = Synthetic: Target sequences for wild type COL8A2
  source 1..23: mol_type = other DNA
ctccctggtccagctggcttagg

SEQ ID NO: 657 - DNA, 23 bp, synthetic construct
  misc_feature 1..23: note = Synthetic: Target sequences for wild type COL8A2
  source 1..23: mol_type = other DNA
ctaagccagctggaccagggagg

SEQ ID NO: 658 - DNA, 23 bp, synthetic construct
  misc_feature 1..23: note = Synthetic: Target sequences for wild type COL8A2
  source 1..23: mol_type = other DNA
gccagctggaccagggaggccgg

SEQ ID NO: 659 - DNA, 23 bp, synthetic construct
  misc_feature 1..23: note = Synthetic: Target sequences for wild type COL8A2
  source 1..23: mol_type = other DNA
ccagctggaccagggaggccggg

SEQ ID NO: 660 - DNA, 23 bp, synthetic construct
  misc_feature 1..23: note = Synthetic: Target sequences for wild type COL8A2
  source 1..23: mol_type = other DNA
cccggcctccctggtccagctgg

SEQ ID NO: 661 - DNA, 23 bp, synthetic construct
  misc_feature 1..23: note = Synthetic: Target sequences for wild type COL8A2
  source 1..23: mol_type = other DNA
cagctggaccagggaggccgggg

SEQ ID NO: 662 - DNA, 23 bp, synthetic construct
  misc_feature 1..23: note = Synthetic: Target sequences for wild type COL8A2
  source 1..23: mol_type = other DNA
agctggaccagggaggccggggg

SEQ ID NO: 663 - DNA, 23 bp, synthetic construct
  misc_feature 1..23: note = Synthetic: Target sequences for wild type COL8A2
  source 1..23: mol_type = other DNA
gctggaccagggaggccgggggg

SEQ ID NO: 664 - DNA, 23 bp, synthetic construct
  misc_feature 1..23: note = Synthetic: Target sequences for wild type COL8A2
  source 1..23: mol_type = other DNA
ctggaccagggaggccggggggg

SEQ ID NO: 665 - DNA, 23 bp, synthetic construct
  misc_feature 1..23: note = Synthetic: Target sequences for wild type COL8A2
  source 1..23: mol_type = other DNA
accagggaggccgggggggccgg

SEQ ID NO: 666 - DNA, 23 bp, synthetic construct
  misc_feature 1..23: note = Synthetic: Target sequences for wild type COL8A2
  source 1..23: mol_type = other DNA
ccagggaggccgggggggccggg

SEQ ID NO: 667 - DNA, 23 bp, synthetic construct
  misc_feature 1..23: note = Synthetic: Target sequences for wild type COL8A2
  source 1..23: mol_type = other DNA
cccggcccccccggcctccctgg

SEQ ID NO: 668 - DNA, 23 bp, synthetic construct
  misc_feature 1..23: note = Synthetic: Target sequences for wild type COL8A2
  source 1..23: mol_type = other DNA
cagggaggccgggggggccgggg

SEQ ID NO: 669 - DNA, 23 bp, synthetic construct
  misc_feature 1..23: note = Synthetic: Target sequences for wild type COL8A2
  source 1..23: mol_type = other DNA
agggaggccgggggggccggggg

SEQ ID NO: 670 - DNA, 23 bp, synthetic construct
  misc_feature 1..23: note = Synthetic: Target sequences for wild type COL8A2
  source 1..23: mol_type = other DNA
gggggtgcccccggcccccccgg

SEQ ID NO: 671 - DNA, 23 bp, synthetic construct
  misc_feature 1..23: note = Synthetic: Target sequences for wild type COL8A2
  source 1..23: mol_type = other DNA
ccgggggcacccccctgccctgg

SEQ ID NO: 672 - DNA, 23 bp, synthetic construct
  misc_feature 1..23: note = Synthetic: Target sequences for wild type COL8A2
  source 1..23: mol_type = other DNA
ccagggcaggggggtgcccccgg

SEQ ID NO: 673 - DNA, 23 bp, synthetic construct
  misc_feature 1..23: note = Synthetic: Target sequences for wild type COL8A2
  source 1..23: mol_type = other DNA
cgggggcacccccctgccctggg

SEQ ID NO: 674 - DNA, 23 bp, synthetic construct
  misc_feature 1..23: note = Synthetic: Target sequences for wild type COL8A2
  source 1..23: mol_type = other DNA
gggggcacccccctgccctgggg

SEQ ID NO: 675 - DNA, 23 bp, synthetic construct
  misc_feature 1..23: note = Synthetic: Target sequences for wild type COL8A2
  source 1..23: mol_type = other DNA
cccccctgccctggggccccagg

SEQ ID NO: 676 - DNA, 23 bp, synthetic construct
  misc_feature 1..23: note = Synthetic: Target sequences for wild type COL8A2
  source 1..23: mol_type = other DNA
cctggggccccagggcagggggg

SEQ ID NO: 677 - DNA, 23 bp, synthetic construct
  misc_feature 1..23: note = Synthetic: Target sequences for wild type COL8A2
  source 1..23: mol_type = other DNA
gcctggggccccagggcaggggg

SEQ ID NO: 678 - DNA, 23 bp, synthetic construct
  misc_feature 1..23: note = Synthetic: Target sequences for wild type COL8A2
  source 1..23: mol_type = other DNA
tgcctggggccccagggcagggg

SEQ ID NO: 679 - DNA, 23 bp, synthetic construct
  misc_feature 1..23: note = Synthetic: Target sequences for wild type COL8A2
  source 1..23: mol_type = other DNA
ctgcctggggccccagggcaggg

SEQ ID NO: 680 - DNA, 23 bp, synthetic construct
  misc_feature 1..23: note = Synthetic: Target sequences for wild type COL8A2
  source 1..23: mol_type = other DNA
gctgcctggggccccagggcagg

SEQ ID NO: 681 - DNA, 23 bp, synthetic construct
  misc_feature 1..23: note = Synthetic: Target sequences for wild type COL8A2
  source 1..23: mol_type = other DNA
ccctggggccccaggcagcccgg

SEQ ID NO: 682 - DNA, 23 bp, synthetic construct
  misc_feature 1..23: note = Synthetic: Target sequences for wild type COL8A2
  source 1..23: mol_type = other DNA
ccgggctgcctggggccccaggg

SEQ ID NO: 683 - DNA, 23 bp, synthetic construct
  misc_feature 1..23: note = Synthetic: Target sequences for wild type COL8A2
  source 1..23: mol_type = other DNA
cctggggccccaggcagcccggg

SEQ ID NO: 684 - DNA, 23 bp, synthetic construct
  misc_feature 1..23: note = Synthetic: Target sequences for wild type COL8A2
  source 1..23: mol_type = other DNA
cccgggctgcctggggccccagg

SEQ ID NO: 685 - DNA, 23 bp, synthetic construct
  misc_feature 1..23: note = Synthetic: Target sequences for wild type COL8A2
  source 1..23: mol_type = other DNA
gggccccaggcagcccgggctgg

SEQ ID NO: 686 - DNA, 23 bp, synthetic construct
  misc_feature 1..23: note = Synthetic: Target sequences for wild type COL8A2
  source 1..23: mol_type = other DNA
gggccagcccgggctgcctgggg

SEQ ID NO: 687 - DNA, 23 bp, synthetic construct
  misc_feature 1..23: note = Synthetic: Target sequences for wild type COL8A2
  source 1..23: mol_type = other DNA
tgggccagcccgggctgcctggg

SEQ ID NO: 688 - DNA, 23 bp, synthetic construct
  misc_feature 1..23: note = Synthetic: Target sequences for wild type COL8A2
  source 1..23: mol_type = other DNA
gtgggccagcccgggctgcctgg

SEQ ID NO: 689 - DNA, 23 bp, synthetic construct
  misc_feature 1..23: note = Synthetic: Target sequences for wild type COL8A2
  source 1..23: mol_type = other DNA
ataatggagtgggccagcccggg

SEQ ID NO: 690 - DNA, 23 bp, synthetic construct
  misc_feature 1..23: note = Synthetic: Target sequences for wild type COL8A2
  source 1..23: mol_type = other DNA
gataatggagtgggccagcccgg

SEQ ID NO: 691 - DNA, 23 bp, synthetic construct
  misc_feature 1..23: note = Synthetic: Target sequences for wild type COL8A2
  source 1..23: mol_type = other DNA
ctcaagggggataatggagtggg

SEQ ID NO: 692 - DNA, 23 bp, synthetic construct
  misc_feature 1..23: note = Synthetic: Target sequences for wild type COL8A2
  source 1..23: mol_type = other DNA
ccactccattatcccccttgagg

SEQ ID NO: 693 - DNA, 23 bp, synthetic construct
  misc_feature 1..23: note = Synthetic: Target sequences for wild type COL8A2
  source 1..23: mol_type = other DNA
cctcaagggggataatggagtgg

SEQ ID NO: 694 - DNA, 23 bp, synthetic construct
  misc_feature 1..23: note = Synthetic: Target sequences for wild type COL8A2
  source 1..23: mol_type = other DNA
cgaggcctcaagggggataatgg

SEQ ID NO: 695 - DNA, 23 bp, synthetic construct
  misc_feature 1..23: note = Synthetic: Target sequences for wild type COL8A2
  source 1..23: mol_type = other DNA
aggtgatcgaggcctcaaggggg

SEQ ID NO: 696 - DNA, 23 bp, synthetic construct
  misc_feature 1..23: note = Synthetic: Target sequences for wild type COL8A2
  source 1..23: mol_type = other DNA
caggtgatcgaggcctcaagggg

SEQ ID NO: 697 - DNA, 23 bp, synthetic construct
  misc_feature 1..23: note = Synthetic: Target sequences for wild type COL8A2
  source 1..23: mol_type = other DNA
cccttgaggcctcgatcacctgg

SEQ ID NO: 698 - DNA, 23 bp, synthetic construct
  misc_feature 1..23: note = Synthetic: Target sequences for wild type COL8A2
  source 1..23: mol_type = other DNA
ccaggtgatcgaggcctcaaggg

SEQ ID NO: 699 - DNA, 23 bp, synthetic construct
  misc_feature 1..23: note = Synthetic: Target sequences for wild type COL8A2
  source 1..23: mol_type = other DNA
ccttgaggcctcgatcacctggg

SEQ ID NO: 700 - DNA, 23 bp, synthetic construct
  misc_feature 1..23: note = Synthetic: Target sequences for wild type COL8A2
  source 1..23: mol_type = other DNA
cccaggtgatcgaggcctcaagg

SEQ ID NO: 701 - DNA, 23 bp, synthetic construct
  misc_feature 1..23: note = Synthetic: Target sequences for wild type COL8A2
  source 1..23: mol_type = other DNA
cttgaggcctcgatcacctgggg

SEQ ID NO: 702 - DNA, 23 bp, synthetic construct
  misc_feature 1..23: note = Synthetic: Target sequences for wild type COL8A2
  source 1..23: mol_type = other DNA
ttgaggcctcgatcacctggggg

SEQ ID NO: 703 - DNA, 23 bp, synthetic construct
  misc_feature 1..23: note = Synthetic: Target sequences for wild type COL8A2
  source 1..23: mol_type = other DNA
cctcgatcacctgggggcccagg

SEQ ID NO: 704 - DNA, 23 bp, synthetic construct
  misc_feature 1..23: note = Synthetic: Target sequences for wild type COL8A2
  source 1..23: mol_type = other DNA
cctgggcccccaggtgatcgagg

SEQ ID NO: 705 - DNA, 23 bp, synthetic construct
  misc_feature 1..23: note = Synthetic: Target sequences for wild type COL8A2
  source 1..23: mol_type = other DNA
cagggggagcctgggcccccagg

SEQ ID NO: 706 - DNA, 23 bp, synthetic construct
  misc_feature 1..23: note = Synthetic: Target sequences for wild type COL8A2
  source 1..23: mol_type = other DNA
ctgggggcccaggctccccctgg

SEQ ID NO: 707 - DNA, 23 bp, synthetic construct
  misc_feature 1..23: note = Synthetic: Target sequences for wild type COL8A2
  source 1..23: mol_type = other DNA
tgggggcccaggctccccctggg

SEQ ID NO: 708 - DNA, 23 bp, synthetic construct
  misc_feature 1..23: note = Synthetic: Target sequences for wild type COL8A2
  source 1..23: mol_type = other DNA
gggggcccaggctccccctgggg

SEQ ID NO: 709 - DNA, 23 bp, synthetic construct
  misc_feature 1..23: note = Synthetic: Target sequences for wild type COL8A2
  source 1..23: mol_type = other DNA
cagggccccagggggagcctggg

SEQ ID NO: 710 - DNA, 23 bp, synthetic construct
  misc_feature 1..23: note = Synthetic: Target sequences for wild type COL8A2
  source 1..23: mol_type = other DNA
ccaggctccccctggggccctgg

SEQ ID NO: 711 - DNA, 23 bp, synthetic construct
  misc_feature 1..23: note = Synthetic: Target sequences for wild type COL8A2
  source 1..23: mol_type = other DNA
ccagggccccagggggagcctgg

SEQ ID NO: 712 - DNA, 23 bp, synthetic construct
  misc_feature 1..23: note = Synthetic: Target sequences for wild type COL8A2
  source 1..23: mol_type = other DNA
gggggaaccagggccccaggggg

SEQ ID NO: 713 - DNA, 23 bp, synthetic construct
  misc_feature 1..23: note = Synthetic: Target sequences for wild type COL8A2
  source 1..23: mol_type = other DNA
agggggaaccagggccccagggg

SEQ ID NO: 714 - DNA, 23 bp, synthetic construct
  misc_feature 1..23: note = Synthetic: Target sequences for wild type COL8A2
  source 1..23: mol_type = other DNA
cagggggaaccagggccccaggg

SEQ ID NO: 715 - DNA, 23 bp, synthetic construct
  misc_feature 1..23: note = Synthetic: Target sequences for wild type COL8A2
  source 1..23: mol_type = other DNA
cctggggccctggttccccctgg

SEQ ID NO: 716 - DNA, 23 bp, synthetic construct
  misc_feature 1..23: note = Synthetic: Target sequences for wild type COL8A2
  source 1..23: mol_type = other DNA
ccagggggaaccagggccccagg

SEQ ID NO: 717 - DNA, 23 bp, synthetic construct
  misc_feature 1..23: note = Synthetic: Target sequences for wild type COL8A2
  source 1..23: mol_type = other DNA
caggattccagggggaaccaggg

SEQ ID NO: 718 - DNA, 23 bp, synthetic construct
  misc_feature 1..23: note = Synthetic: Target sequences for wild type COL8A2
  source 1..23: mol_type = other DNA
cctggttccccctggaatcctgg

SEQ ID NO: 719 - DNA, 23 bp, synthetic construct
  misc_feature 1..23: note = Synthetic: Target sequences for wild type COL8A2
  source 1..23: mol_type = other DNA
ccaggattccagggggaaccagg

SEQ ID NO: 720 - DNA, 23 bp, synthetic construct
  misc_feature 1..23: note = Synthetic: Target sequences for wild type COL8A2
  source 1..23: mol_type = other DNA
ctggttccccctggaatcctggg

SEQ ID NO: 721 - DNA, 23 bp, synthetic construct
  misc_feature 1..23: note = Synthetic: Target sequences for wild type COL8A2
  source 1..23: mol_type = other DNA
tggttccccctggaatcctgggg

SEQ ID NO: 722 - DNA, 23 bp, synthetic construct
  misc_feature 1..23: note = Synthetic: Target sequences for wild type COL8A2
  source 1..23: mol_type = other DNA
ggttccccctggaatcctggggg

SEQ ID NO: 723 - DNA, 23 bp, synthetic construct
  misc_feature 1..23: note = Synthetic: Target sequences for wild type COL8A2
  source 1..23: mol_type = other DNA
agggcccccaggattccaggggg

SEQ ID NO: 724 - DNA, 23 bp, synthetic construct
  misc_feature 1..23: note = Synthetic: Target sequences for wild type COL8A2
  source 1..23: mol_type = other DNA
cagggcccccaggattccagggg

SEQ ID NO: 725 - DNA, 23 bp, synthetic construct
  misc_feature 1..23: note = Synthetic: Target sequences for wild type COL8A2
  source 1..23: mol_type = other DNA
ccctggaatcctgggggccctgg

SEQ ID NO: 726 - DNA, 23 bp, synthetic construct
  misc_feature 1..23: note = Synthetic: Target sequences for wild type COL8A2
  source 1..23: mol_type = other DNA
ccagggcccccaggattccaggg

SEQ ID NO: 727 - DNA, 23 bp, synthetic construct
  misc_feature 1..23: note = Synthetic: Target sequences for wild type COL8A2
  source 1..23: mol_type = other DNA
gccagggcccccaggattccagg

SEQ ID NO: 728 - DNA, 23 bp, synthetic construct
  misc_feature 1..23: note = Synthetic: Target sequences for wild type COL8A2
  source 1..23: mol_type = other DNA
caaggggtgccagggcccccagg

SEQ ID NO: 729 - DNA, 23 bp, synthetic construct
  misc_feature 1..23: note = Synthetic: Target sequences for wild type COL8A2
  source 1..23: mol_type = other DNA
ctgggggccctggcaccccttgg

SEQ ID NO: 730 - DNA, 23 bp, synthetic construct
  misc_feature 1..23: note = Synthetic: Target sequences for wild type COL8A2
  source 1..23: mol_type = other DNA
tgggggccctggcaccccttggg

SEQ ID NO: 731 - DNA, 23 bp, synthetic construct
  misc_feature 1..23: note = Synthetic: Target sequences for wild type COL8A2
  source 1..23: mol_type = other DNA
caggtgcccaaggggtgccaggg

SEQ ID NO: 732 - DNA, 23 bp, synthetic construct
  misc_feature 1..23: note = Synthetic: Target sequences for wild type COL8A2
  source 1..23: mol_type = other DNA
cctggcaccccttgggcacctgg

SEQ ID NO: 733 - DNA, 23 bp, synthetic construct
  misc_feature 1..23: note = Synthetic: Target sequences for wild type COL8A2
  source 1..23: mol_type = other DNA
ccaggtgcccaaggggtgccagg

SEQ ID NO: 734 - DNA, 23 bp, synthetic construct
  misc_feature 1..23: note = Synthetic: Target sequences for wild type COL8A2
  source 1..23: mol_type = other DNA
tggaaaaccaggtgcccaagggg

SEQ ID NO: 735 - DNA, 23 bp, synthetic construct
  misc_feature 1..23: note = Synthetic: Target sequences for wild type COL8A2
  source 1..23: mol_type = other DNA
ctggaaaaccaggtgcccaaggg

SEQ ID NO: 736 - DNA, 23 bp, synthetic construct
  misc_feature 1..23: note = Synthetic: Target sequences for wild type COL8A2
  source 1..23: mol_type = other DNA
ccttgggcacctggttttccagg

SEQ ID NO: 737 - DNA, 23 bp, synthetic construct
  misc_feature 1..23: note = Synthetic: Target sequences for wild type COL8A2
  source 1..23: mol_type = other DNA
cctggaaaaccaggtgcccaagg

SEQ ID NO: 738 - DNA, 23 bp, synthetic construct
  misc_feature 1..23: note = Synthetic: Target sequences for wild type COL8A2
  source 1..23: mol_type = other DNA
cttgggcacctggttttccaggg

SEQ ID NO: 739 - DNA, 23 bp, synthetic construct
  misc_feature 1..23: note = Synthetic: Target sequences for wild type COL8A2
  source 1..23: mol_type = other DNA
attactatccctggaaaaccagg

SEQ ID NO: 740 - DNA, 23 bp, synthetic construct
  misc_feature 1..23: note = Synthetic: Target sequences for wild type COL8A2
  source 1..23: mol_type = other DNA
tccagggatagtaatgcctgagg

SEQ ID NO: 741 - DNA, 23 bp, synthetic construct
  misc_feature 1..23: note = Synthetic: Target sequences for wild type COL8A2
  source 1..23: mol_type = other DNA
ccagggatagtaatgcctgaggg

SEQ ID NO: 742 - DNA, 23 bp, synthetic construct
  misc_feature 1..23: note = Synthetic: Target sequences for wild type COL8A2
  source 1..23: mol_type = other DNA
ccctcaggcattactatccctgg

SEQ ID NO: 743 - DNA, 23 bp, synthetic construct
  misc_feature 1..23: note = Synthetic: Target sequences for wild type COL8A2
  source 1..23: mol_type = other DNA
cagggatagtaatgcctgagggg

SEQ ID NO: 744 - DNA, 23 bp, synthetic construct
  misc_feature 1..23: note = Synthetic: Target sequences for wild type COL8A2
  source 1..23: mol_type = other DNA
atagtaatgcctgaggggcccgg

SEQ ID NO: 745 - DNA, 23 bp, synthetic construct
  misc_feature 1..23: note = Synthetic: Target sequences for wild type COL8A2
  source 1..23: mol_type = other DNA
tagtaatgcctgaggggcccggg

SEQ ID NO: 746 - DNA, 23 bp, synthetic construct
  misc_feature 1..23: note = Synthetic: Target sequences for wild type COL8A2
  source 1..23: mol_type = other DNA
taatgcctgaggggcccgggagg

SEQ ID NO: 747 - DNA, 23 bp, synthetic construct
  misc_feature 1..23: note = Synthetic: Target sequences for wild type COL8A2
  source 1..23: mol_type = other DNA
cctgaggggcccgggaggccagg

SEQ ID NO: 748 - DNA, 23 bp, synthetic construct
  misc_feature 1..23: note = Synthetic: Target sequences for wild type COL8A2
  source 1..23: mol_type = other DNA
cctggcctcccgggcccctcagg

SEQ ID NO: 749 - DNA, 23 bp, synthetic construct
  misc_feature 1..23: note = Synthetic: Target sequences for wild type COL8A2
  source 1..23: mol_type = other DNA
ctgaggggcccgggaggccaggg

SEQ ID NO: 750 - DNA, 23 bp, synthetic construct
  misc_feature 1..23: note = Synthetic: Target sequences for wild type COL8A2
  source 1..23: mol_type = other DNA
tgaggggcccgggaggccagggg

SEQ ID NO: 751 - DNA, 23 bp, synthetic construct
  misc_feature 1..23: note = Synthetic: Target sequences for wild type COL8A2
  source 1..23: mol_type = other DNA
gaggggcccgggaggccaggggg

SEQ ID NO: 752 - DNA, 23 bp, synthetic construct
  misc_feature 1..23: note = Synthetic: Target sequences for wild type COL8A2
  source 1..23: mol_type = other DNA
cccgggaggccagggggtcctgg

SEQ ID NO: 753 - DNA, 23 bp, synthetic construct
  misc_feature 1..23: note = Synthetic: Target sequences for wild type COL8A2
  source 1..23: mol_type = other DNA
ccaggaccccctggcctcccggg

SEQ ID NO: 754 - DNA, 23 bp, synthetic construct
  misc_feature 1..23: note = Synthetic: Target sequences for wild type COL8A2
  source 1..23: mol_type = other DNA
ccgggaggccagggggtcctggg

SEQ ID NO: 755 - DNA, 23 bp, synthetic construct
  misc_feature 1..23: note = Synthetic: Target sequences for wild type COL8A2
  source 1..23: mol_type = other DNA
cccaggaccccctggcctcccgg

SEQ ID NO: 756 - DNA, 23 bp, synthetic construct
  misc_feature 1..23: note = Synthetic: Target sequences for wild type COL8A2
  source 1..23: mol_type = other DNA
cgggaggccagggggtcctgggg

SEQ ID NO: 757 - DNA, 23 bp, synthetic construct
  misc_feature 1..23: note = Synthetic: Target sequences for wild type COL8A2
  source 1..23: mol_type = other DNA
gggaggccagggggtcctggggg

SEQ ID NO: 758 - DNA, 23 bp, synthetic construct
  misc_feature 1..23: note = Synthetic: Target sequences for wild type COL8A2
  source 1..23: mol_type = other DNA
cggggacccccaggaccccctgg

SEQ ID NO: 759 - DNA, 23 bp, synthetic construct
  misc_feature 1..23: note = Synthetic: Target sequences for wild type COL8A2
  source 1..23: mol_type = other DNA
cagggggtcctgggggtccccgg

SEQ ID NO: 760 - DNA, 23 bp, synthetic construct
  misc_feature 1..23: note = Synthetic: Target sequences for wild type COL8A2
  source 1..23: mol_type = other DNA
ggggtcctgggggtccccggagg

SEQ ID NO: 761 - DNA, 23 bp, synthetic construct
  misc_feature 1..23: note = Synthetic: Target sequences for wild type COL8A2
  source 1..23: mol_type = other DNA
cagggcctccggggacccccagg

SEQ ID NO: 762 - DNA, 23 bp, synthetic construct
  misc_feature 1..23: note = Synthetic: Target sequences for wild type COL8A2
  source 1..23: mol_type = other DNA
ctgggggtccccggaggccctgg

SEQ ID NO: 763 - DNA, 23 bp, synthetic construct
  misc_feature 1..23: note = Synthetic: Target sequences for wild type COL8A2
  source 1..23: mol_type = other DNA
cgaggggaccagggcctccgggg

SEQ ID NO: 764 - DNA, 23 bp, synthetic construct
  misc_feature 1..23: note = Synthetic: Target sequences for wild type COL8A2
  source 1..23: mol_type = other DNA
acgaggggaccagggcctccggg

SEQ ID NO: 765 - DNA, 23 bp, synthetic construct
  misc_feature 1..23: note = Synthetic: Target sequences for wild type COL8A2
  source 1..23: mol_type = other DNA
tacgaggggaccagggcctccgg

SEQ ID NO: 766 - DNA, 23 bp, synthetic construct
  misc_feature 1..23: note = Synthetic: Target sequences for wild type COL8A2
  source 1..23: mol_type = other DNA
ccctggtcccctcgtattcctgg

SEQ ID NO: 767 - DNA, 23 bp, synthetic construct
  misc_feature 1..23: note = Synthetic: Target sequences for wild type COL8A2
  source 1..23: mol_type = other DNA
ccaggaatacgaggggaccaggg

SEQ ID NO: 768 - DNA, 23 bp, synthetic construct
  misc_feature 1..23: note = Synthetic: Target sequences for wild type COL8A2
  source 1..23: mol_type = other DNA
gccaggaatacgaggggaccagg

SEQ ID NO: 769 - DNA, 23 bp, synthetic construct
  misc_feature 1..23: note = Synthetic: Target sequences for wild type COL8A2
  source 1..23: mol_type = other DNA
gggggagccaggaatacgagggg

SEQ ID NO: 770 - DNA, 23 bp, synthetic construct
  misc_feature 1..23: note = Synthetic: Target sequences for wild type COL8A2
  source 1..23: mol_type = other DNA
ggggggagccaggaatacgaggg

SEQ ID NO: 771 - DNA, 23 bp, synthetic construct
  misc_feature 1..23: note = Synthetic: Target sequences for wild type COL8A2
  source 1..23: mol_type = other DNA
cggggggagccaggaatacgagg

SEQ ID NO: 772 - DNA, 23 bp, synthetic construct
  misc_feature 1..23: note = Synthetic: Target sequences for wild type COL8A2
  source 1..23: mol_type = other DNA
cctggctccccccgaagccccgg

SEQ ID NO: 773 - DNA, 23 bp, synthetic construct
  misc_feature 1..23: note = Synthetic: Target sequences for wild type COL8A2
  source 1..23: mol_type = other DNA
ccggggcttcggggggagccagg

SEQ ID NO: 774 - DNA, 23 bp, synthetic construct
  misc_feature 1..23: note = Synthetic: Target sequences for wild type COL8A2
  source 1..23: mol_type = other DNA
agggcagccggggcttcgggggg

SEQ ID NO: 775 - DNA, 23 bp, synthetic construct
  misc_feature 1..23: note = Synthetic: Target sequences for wild type COL8A2
  source 1..23: mol_type = other DNA
cagggcagccggggcttcggggg

SEQ ID NO: 776 - DNA, 23 bp, synthetic construct
  misc_feature 1..23: note = Synthetic: Target sequences for wild type COL8A2
  source 1..23: mol_type = other DNA
ccccgaagccccggctgccctgg

SEQ ID NO: 777 - DNA, 23 bp, synthetic construct
  misc_feature 1..23: note = Synthetic: Target sequences for wild type COL8A2
  source 1..23: mol_type = other DNA
ccagggcagccggggcttcgggg

SEQ ID NO: 778 - DNA, 23 bp, synthetic construct
  misc_feature 1..23: note = Synthetic: Target sequences for wild type COL8A2
  source 1..23: mol_type = other DNA
accagggcagccggggcttcggg

SEQ ID NO: 779 - DNA, 23 bp, synthetic construct
  misc_feature 1..23: note = Synthetic: Target sequences for wild type COL8A2
  source 1..23: mol_type = other DNA
caccagggcagccggggcttcgg

SEQ ID NO: 780 - DNA, 23 bp, synthetic construct
  misc_feature 1..23: note = Synthetic: Target sequences for wild type COL8A2
  source 1..23: mol_type = other DNA
cgaagccccggctgccctggtgg

SEQ ID NO: 781 - DNA, 23 bp, synthetic construct
  misc_feature 1..23: note = Synthetic: Target sequences for wild type COL8A2
  source 1..23: mol_type = other DNA
tcgggccaccagggcagccgggg

SEQ ID NO: 782 - DNA, 23 bp, synthetic construct
  misc_feature 1..23: note = Synthetic: Target sequences for wild type COL8A2
  source 1..23: mol_type = other DNA
gtcgggccaccagggcagccggg

SEQ ID NO: 783 - DNA, 23 bp, synthetic construct
  misc_feature 1..23: note = Synthetic: Target sequences for wild type COL8A2
  source 1..23: mol_type = other DNA
ggtcgggccaccagggcagccgg

SEQ ID NO: 784 - DNA, 23 bp, synthetic construct
  misc_feature 1..23: note = Synthetic: Target sequences for wild type COL8A2
  source 1..23: mol_type = other DNA
ctggcaaggtcgggccaccaggg

SEQ ID NO: 785 - DNA, 23 bp, synthetic construct
  misc_feature 1..23: note = Synthetic: Target sequences for wild type COL8A2
  source 1..23: mol_type = other DNA
cctggtggcccgaccttgccagg

SEQ ID NO: 786 - DNA, 23 bp, synthetic construct
  misc_feature 1..23: note = Synthetic: Target sequences for wild type COL8A2
  source 1..23: mol_type = other DNA
cctggcaaggtcgggccaccagg

SEQ ID NO: 787 - DNA, 23 bp, synthetic construct
  misc_feature 1..23: note = Synthetic: Target sequences for wild type COL8A2
  source 1..23: mol_type = other DNA
ctggtggcccgaccttgccaggg

SEQ ID NO: 788 - DNA, 23 bp, synthetic construct
  misc_feature 1..23: note = Synthetic: Target sequences for wild type COL8A2
  source 1..23: mol_type = other DNA
cagggctccctggcaaggtcggg

SEQ ID NO: 789 - DNA, 23 bp, synthetic construct
  misc_feature 1..23: note = Synthetic: Target sequences for wild type COL8A2
  source 1..23: mol_type = other DNA
ccgaccttgccagggagccctgg

SEQ ID NO: 790 - DNA, 23 bp, synthetic construct
  misc_feature 1..23: note = Synthetic: Target sequences for wild type COL8A2
  source 1..23: mol_type = other DNA
ccagggctccctggcaaggtcgg

SEQ ID NO: 791 - DNA, 23 bp, synthetic construct
  misc_feature 1..23: note = Synthetic: Target sequences for wild type COL8A2
  source 1..23: mol_type = other DNA
cgaccttgccagggagccctggg

SEQ ID NO: 792 - DNA, 23 bp, synthetic construct
  misc_feature 1..23: note = Synthetic: Target sequences for wild type COL8A2
  source 1..23: mol_type = other DNA
gaccttgccagggagccctgggg

SEQ ID NO: 793 - DNA, 23 bp, synthetic construct
  misc_feature 1..23: note = Synthetic: Target sequences for wild type COL8A2
  source 1..23: mol_type = other DNA
accttgccagggagccctggggg

SEQ ID NO: 794 - DNA, 23 bp, synthetic construct
  misc_feature 1..23: note = Synthetic: Target sequences for wild type COL8A2
  source 1..23: mol_type = other DNA
tcccccagggctccctggcaagg

SEQ ID NO: 795 - DNA, 23 bp, synthetic construct
  misc_feature 1..23: note = Synthetic: Target sequences for wild type COL8A2
  source 1..23: mol_type = other DNA
gctggtcccccagggctccctgg

SEQ ID NO: 796 - DNA, 23 bp, synthetic construct
  misc_feature 1..23: note = Synthetic: Target sequences for wild type COL8A2
  source 1..23: mol_type = other DNA
tgggcaaggctggtcccccaggg

SEQ ID NO: 797 - DNA, 23 bp, synthetic construct
  misc_feature 1..23: note = Synthetic: Target sequences for wild type COL8A2
  source 1..23: mol_type = other DNA
atgggcaaggctggtcccccagg

SEQ ID NO: 798 - DNA, 23 bp, synthetic construct
  misc_feature 1..23: note = Synthetic: Target sequences for wild type COL8A2
  source 1..23: mol_type = other DNA
ggggaccagccttgcccatccgg

SEQ ID NO: 799 - DNA, 23 bp, synthetic construct
  misc_feature 1..23: note = Synthetic: Target sequences for wild type COL8A2
  source 1..23: mol_type = other DNA
gggaccagccttgcccatccggg

SEQ ID NO: 800 - DNA, 23 bp, synthetic construct
  misc_feature 1..23: note = Synthetic: Target sequences for wild type COL8A2
  source 1..23: mol_type = other DNA
ttctcccggatgggcaaggctgg

SEQ ID NO: 801 - DNA, 23 bp, synthetic construct
  misc_feature 1..23: note = Synthetic: Target sequences for wild type COL8A2
  source 1..23: mol_type = other DNA
tggcttctcccggatgggcaagg

SEQ ID NO: 802 - DNA, 23 bp, synthetic construct
  misc_feature 1..23: note = Synthetic: Target sequences for wild type COL8A2
  source 1..23: mol_type = other DNA
ttgcccatccgggagaagccagg

SEQ ID NO: 803 - DNA, 23 bp, synthetic construct
  misc_feature 1..23: note = Synthetic: Target sequences for wild type COL8A2
  source 1..23: mol_type = other DNA
tgcccatccgggagaagccaggg

SEQ ID NO: 804 - DNA, 23 bp, synthetic construct
  misc_feature 1..23: note = Synthetic: Target sequences for wild type COL8A2
  source 1..23: mol_type = other DNA
gcccatccgggagaagccagggg

SEQ ID NO: 805 - DNA, 23 bp, synthetic construct
  misc_feature 1..23: note = Synthetic: Target sequences for wild type COL8A2
  source 1..23: mol_type = other DNA
cccatccgggagaagccaggggg

SEQ ID NO: 806 - DNA, 23 bp, synthetic construct
  misc_feature 1..23: note = Synthetic: Target sequences for wild type COL8A2
  source 1..23: mol_type = other DNA
ccccctggcttctcccggatggg

SEQ ID NO: 807 - DNA, 23 bp, synthetic construct
  misc_feature 1..23: note = Synthetic: Target sequences for wild type COL8A2
  source 1..23: mol_type = other DNA
gccccctggcttctcccggatgg

SEQ ID NO: 808 - DNA, 23 bp, synthetic construct
  misc_feature 1..23: note = Synthetic: Target sequences for wild type COL8A2
  source 1..23: mol_type = other DNA
ctgggccccctggcttctcccgg

SEQ ID NO: 809 - DNA, 23 bp, synthetic construct
  misc_feature 1..23: note = Synthetic: Target sequences for wild type COL8A2
  source 1..23: mol_type = other DNA
gagaagccagggggcccagcagg

SEQ ID NO: 810 - DNA, 23 bp, synthetic construct
  misc_feature 1..23: note = Synthetic: Target sequences for wild type COL8A2
  source 1..23: mol_type = other DNA
agaagccagggggcccagcaggg

SEQ ID NO: 811 - DNA, 23 bp, synthetic construct
  misc_feature 1..23: note = Synthetic: Target sequences for wild type COL8A2
  source 1..23: mol_type = other DNA
ccagggggcccagcagggccagg

SEQ ID NO: 812 - DNA, 23 bp, synthetic construct
  misc_feature 1..23: note = Synthetic: Target sequences for wild type COL8A2
  source 1..23: mol_type = other DNA
cctggccctgctgggccccctgg

SEQ ID NO: 813 - DNA, 23 bp, synthetic construct
  misc_feature 1..23: note = Synthetic: Target sequences for wild type COL8A2
  source 1..23: mol_type = other DNA
atgggcagcctggccctgctggg

SEQ ID NO: 814 - DNA, 23 bp, synthetic construct
  misc_feature 1..23: note = Synthetic: Target sequences for wild type COL8A2
  source 1..23: mol_type = other DNA
catgggcagcctggccctgctgg

SEQ ID NO: 815 - DNA, 23 bp, synthetic construct
  misc_feature 1..23: note = Synthetic: Target sequences for wild type COL8A2
  source 1..23: mol_type = other DNA
cagcagggccaggctgcccatgg

SEQ ID NO: 816 - DNA, 23 bp, synthetic construct
  misc_feature 1..23: note = Synthetic: Target sequences for wild type COL8A2
  source 1..23: mol_type = other DNA
ccaggctgcccatggagtcctgg

SEQ ID NO: 817 - DNA, 23 bp, synthetic construct
  misc_feature 1..23: note = Synthetic: Target sequences for wild type COL8A2
  source 1..23: mol_type = other DNA
ccaggactccatgggcagcctgg

SEQ ID NO: 818 - DNA, 23 bp, synthetic construct
  misc_feature 1..23: note = Synthetic: Target sequences for wild type COL8A2
  source 1..23: mol_type = other DNA
tgggaaagccaggactccatggg

SEQ ID NO: 819 - DNA, 23 bp, synthetic construct
  misc_feature 1..23: note = Synthetic: Target sequences for wild type COL8A2
  source 1..23: mol_type = other DNA
atgggaaagccaggactccatgg

SEQ ID NO: 820 - DNA, 23 bp, synthetic construct
  misc_feature 1..23: note = Synthetic: Target sequences for wild type COL8A2
  source 1..23: mol_type = other DNA
agtcctggctttcccatgcctgg

SEQ ID NO: 821 - DNA, 23 bp, synthetic construct
  misc_feature 1..23: note = Synthetic: Target sequences for wild type COL8A2
  source 1..23: mol_type = other DNA
aaaccaggcatgggaaagccagg

SEQ ID NO: 822 - DNA, 23 bp, synthetic construct
  misc_feature 1..23: note = Synthetic: Target sequences for wild type COL8A2
  source 1..23: mol_type = other DNA
tttcccatgcctggttttcctgg

SEQ ID NO: 823 - DNA, 23 bp, synthetic construct
  misc_feature 1..23: note = Synthetic: Target sequences for wild type COL8A2
  source 1..23: mol_type = other DNA
ttcccatgcctggttttcctggg

SEQ ID NO: 824 - DNA, 23 bp, synthetic construct
  misc_feature 1..23: note = Synthetic: Target sequences for wild type COL8A2
  source 1..23: mol_type = other DNA
ttcccaggaaaaccaggcatggg

SEQ ID NO: 825 - DNA, 23 bp, synthetic construct
  misc_feature 1..23: note = Synthetic: Target sequences for wild type COL8A2
  source 1..23: mol_type = other DNA
cttcccaggaaaaccaggcatgg

SEQ ID NO: 826 - DNA, 23 bp, synthetic construct
  misc_feature 1..23: note = Synthetic: Target sequences for wild type COL8A2
  source 1..23: mol_type = other DNA
cctggttttcctgggaagccagg

SEQ ID NO: 827 - DNA, 23 bp, synthetic construct
  misc_feature 1..23: note = Synthetic: Target sequences for wild type COL8A2
  source 1..23: mol_type = other DNA
cctggcttcccaggaaaaccagg

SEQ ID NO: 828 - DNA, 23 bp, synthetic construct
  misc_feature 1..23: note = Synthetic: Target sequences for wild type COL8A2
  source 1..23: mol_type = other DNA
ctggttttcctgggaagccaggg

SEQ ID NO: 829 - DNA, 23 bp, synthetic construct
  misc_feature 1..23: note = Synthetic: Target sequences for wild type COL8A2
  source 1..23: mol_type = other DNA
tggttttcctgggaagccagggg

SEQ ID NO: 830 - DNA, 23 bp, synthetic construct
  misc_feature 1..23: note = Synthetic: Target sequences for wild type COL8A2
  source 1..23: mol_type = other DNA
ggttttcctgggaagccaggggg

SEQ ID NO: 831 - DNA, 23 bp, synthetic construct
  misc_feature 1..23: note = Synthetic: Target sequences for wild type COL8A2
  source 1..23: mol_type = other DNA
gttttcctgggaagccagggggg

SEQ ID NO: 832 - DNA, 23 bp, synthetic construct
  misc_feature 1..23: note = Synthetic: Target sequences for wild type COL8A2
  source 1..23: mol_type = other DNA
cctgggaagccaggggggccagg

SEQ ID NO: 833 - DNA, 23 bp, synthetic construct
  misc_feature 1..23: note = Synthetic: Target sequences for wild type COL8A2
  source 1..23: mol_type = other DNA
cctggcccccctggcttcccagg

SEQ ID NO: 834 - DNA, 23 bp, synthetic construct
  misc_feature 1..23: note = Synthetic: Target sequences for wild type COL8A2
  source 1..23: mol_type = other DNA
ctgggaagccaggggggccaggg

SEQ ID NO: 835 - DNA, 23 bp, synthetic construct
  misc_feature 1..23: note = Synthetic: Target sequences for wild type COL8A2
  source 1..23: mol_type = other DNA
tgggaagccaggggggccagggg

SEQ ID NO: 836 - DNA, 23 bp, synthetic construct
  misc_feature 1..23: note = Synthetic: Target sequences for wild type COL8A2
  source 1..23: mol_type = other DNA
gggaagccaggggggccaggggg

SEQ ID NO: 837 - DNA, 23 bp, synthetic construct
  misc_feature 1..23: note = Synthetic: Target sequences for wild type COL8A2
  source 1..23: mol_type = other DNA
cggggtccccctggcccccctgg

SEQ ID NO: 838 - DNA, 23 bp, synthetic construct
  misc_feature 1..23: note = Synthetic: Target sequences for wild type COL8A2
  source 1..23: mol_type = other DNA
ggggggccagggggaccccgagg

SEQ ID NO: 839 - DNA, 23 bp, synthetic construct
  misc_feature 1..23: note = Synthetic: Target sequences for wild type COL8A2
  source 1..23: mol_type = other DNA
gccagggggaccccgaggcccgg

SEQ ID NO: 840 - DNA, 23 bp, synthetic construct
  misc_feature 1..23: note = Synthetic: Target sequences for wild type COL8A2
  source 1..23: mol_type = other DNA
ccagggggaccccgaggcccggg

SEQ ID NO: 841 - DNA, 23 bp, synthetic construct
  misc_feature 1..23: note = Synthetic: Target sequences for wild type COL8A2
  source 1..23: mol_type = other DNA
cccgggcctcggggtccccctgg

SEQ ID NO: 842 - DNA, 23 bp, synthetic construct
  misc_feature 1..23: note = Synthetic: Target sequences for wild type COL8A2
  source 1..23: mol_type = other DNA
ccccgaggcccgggcttcccagg

SEQ ID NO: 843 - DNA, 23 bp, synthetic construct
  misc_feature 1..23: note = Synthetic: Target sequences for wild type COL8A2
  source 1..23: mol_type = other DNA
cctgggaagcccgggcctcgggg

SEQ ID NO: 844 - DNA, 23 bp, synthetic construct
  misc_feature 1..23: note = Synthetic: Target sequences for wild type COL8A2
  source 1..23: mol_type = other DNA
cccgaggcccgggcttcccaggg

SEQ ID NO: 845 - DNA, 23 bp, synthetic construct
  misc_feature 1..23: note = Synthetic: Target sequences for wild type COL8A2
  source 1..23: mol_type = other DNA
ccctgggaagcccgggcctcggg

SEQ ID NO: 846 - DNA, 23 bp, synthetic construct
  misc_feature 1..23: note = Synthetic: Target sequences for wild type COL8A2
  source 1..23: mol_type = other DNA
ccgaggcccgggcttcccagggg

SEQ ID NO: 847 - DNA, 23 bp, synthetic construct
  misc_feature 1..23: note = Synthetic: Target sequences for wild type COL8A2
  source 1..23: mol_type = other DNA
cccctgggaagcccgggcctcgg

SEQ ID NO: 848 - DNA, 23 bp, synthetic construct
  misc_feature 1..23: note = Synthetic: Target sequences for wild type COL8A2
  source 1..23: mol_type = other DNA
cgaggcccgggcttcccaggggg

SEQ ID NO: 849 - DNA, 23 bp, synthetic construct
  misc_feature 1..23: note = Synthetic: Target sequences for wild type COL8A2
  source 1..23: mol_type = other DNA
gaggcccgggcttcccagggggg

SEQ ID NO: 850 - DNA, 23 bp, synthetic construct
  misc_feature 1..23: note = Synthetic: Target sequences for wild type COL8A2
  source 1..23: mol_type = other DNA
cccgggcttcccaggggggccgg

SEQ ID NO: 851 - DNA, 23 bp, synthetic construct
  misc_feature 1..23: note = Synthetic: Target sequences for wild type COL8A2
  source 1..23: mol_type = other DNA
ccggcccccctgggaagcccggg

SEQ ID NO: 852 - DNA, 23 bp, synthetic construct
  misc_feature 1..23: note = Synthetic: Target sequences for wild type COL8A2
  source 1..23: mol_type = other DNA
ccgggcttcccaggggggccggg

SEQ ID NO: 853 - DNA, 23 bp, synthetic construct
  misc_feature 1..23: note = Synthetic: Target sequences for wild type COL8A2
  source 1..23: mol_type = other DNA
cccggcccccctgggaagcccgg

SEQ ID NO: 854 - DNA, 23 bp, synthetic construct
  misc_feature 1..23: note = Synthetic: Target sequences for wild type COL8A2
  source 1..23: mol_type = other DNA
agggagagcccggcccccctggg

SEQ ID NO: 855 - DNA, 23 bp, synthetic construct
  misc_feature 1..23: note = Synthetic: Target sequences for wild type COL8A2
  source 1..23: mol_type = other DNA
aagggagagcccggcccccctgg

SEQ ID NO: 856 - DNA, 23 bp, synthetic construct
  misc_feature 1..23: note = Synthetic: Target sequences for wild type COL8A2
  source 1..23: mol_type = other DNA
gggggccgggctctcccttcagg

SEQ ID NO: 857 - DNA, 23 bp, synthetic construct
  misc_feature 1..23: note = Synthetic: Target sequences for wild type COL8A2
  source 1..23: mol_type = other DNA
atggacctgaagggagagcccgg

SEQ ID NO: 858 - DNA, 23 bp, synthetic construct
  misc_feature 1..23: note = Synthetic: Target sequences for wild type COL8A2
  source 1..23: mol_type = other DNA
ggctctcccttcaggtccatcgg

SEQ ID NO: 859 - DNA, 23 bp, synthetic construct
  misc_feature 1..23: note = Synthetic: Target sequences for wild type COL8A2
  source 1..23: mol_type = other DNA
ctgctgccgatggacctgaaggg

SEQ ID NO: 860 - DNA, 23 bp, synthetic construct
  misc_feature 1..23: note = Synthetic: Target sequences for wild type COL8A2
  source 1..23: mol_type = other DNA
gctgctgccgatggacctgaagg

SEQ ID NO: 861 - DNA, 23 bp, synthetic construct
  misc_feature 1..23: note = Synthetic: Target sequences for wild type COL8A2
  source 1..23: mol_type = other DNA
ttcaggtccatcggcagcagcgg

SEQ ID NO: 862 - DNA, 23 bp, synthetic construct
  misc_feature 1..23: note = Synthetic: Target sequences for wild type COL8A2
  source 1..23: mol_type = other DNA
tccatcggcagcagcggtagagg

SEQ ID NO: 863 - DNA, 23 bp, synthetic construct
  misc_feature 1..23: note = Synthetic: Target sequences for wild type COL8A2
  source 1..23: mol_type = other DNA
gcctctaccgctgctgccgatgg

SEQ ID NO: 864 - DNA, 23 bp, synthetic construct
  misc_feature 1..23: note = Synthetic: Target sequences for wild type COL8A2
  source 1..23: mol_type = other DNA
tttctgagaaagaaagagaaagg

SEQ ID NO: 865 - DNA, 23 bp, synthetic construct
  misc_feature 1..23: note = Synthetic: Target sequences for wild type COL8A2
  source 1..23: mol_type = other DNA
ttctgagaaagaaagagaaaggg

SEQ ID NO: 866 - DNA, 23 bp, synthetic construct
  misc_feature 1..23: note = Synthetic: Target sequences for wild type COL8A2
  source 1..23: mol_type = other DNA
tctgagaaagaaagagaaagggg

SEQ ID NO: 867 - DNA, 23 bp, synthetic construct
  misc_feature 1..23: note = Synthetic: Target sequences for wild type COL8A2
  source 1..23: mol_type = other DNA
agaaagagaaaggggcagtcagg

SEQ ID NO: 868 - DNA, 23 bp, synthetic construct
  misc_feature 1..23: note = Synthetic: Target sequences for wild type COL8A2
  source 1..23: mol_type = other DNA
gaaagagaaaggggcagtcaggg

SEQ ID NO: 869 - DNA, 23 bp, synthetic construct
  misc_feature 1..23: note = Synthetic: Target sequences for wild type COL8A2
  source 1..23: mol_type = other DNA
aaagagaaaggggcagtcagggg

SEQ ID NO: 870 - DNA, 23 bp, synthetic construct
  misc_feature 1..23: note = Synthetic: Target sequences for wild type COL8A2
  source 1..23: mol_type = other DNA
gcagtcaggggcctgaactgtgg

SEQ ID NO: 871 - DNA, 23 bp, synthetic construct
  misc_feature 1..23: note = Synthetic: Target sequences for wild type COL8A2
  source 1..23: mol_type = other DNA
cagtcaggggcctgaactgtggg

SEQ ID NO: 872 - DNA, 23 bp, synthetic construct
  misc_feature 1..23: note = Synthetic: Target sequences for wild type COL8A2
  source 1..23: mol_type = other DNA
agtcaggggcctgaactgtgggg

SEQ ID NO: 873 - DNA, 23 bp, synthetic construct
  misc_feature 1..23: note = Synthetic: Target sequences for wild type COL8A2
  source 1..23: mol_type = other DNA
ggggcctgaactgtggggacagg

SEQ ID NO: 874 - DNA, 23 bp, synthetic construct
  misc_feature 1..23: note = Synthetic: Target sequences for wild type COL8A2
  source 1..23: mol_type = other DNA
gggcctgaactgtggggacaggg

SEQ ID NO: 875 - DNA, 23 bp, synthetic construct
  misc_feature 1..23: note = Synthetic: Target sequences for wild type COL8A2
  source 1..23: mol_type = other DNA
ggcctgaactgtggggacagggg

SEQ ID NO: 876 - DNA, 23 bp, synthetic construct
  misc_feature 1..23: note = Synthetic: Target sequences for wild type COL8A2
  source 1..23: mol_type = other DNA
gtcccctgtccccacagttcagg

SEQ ID NO: 877 - DNA, 23 bp, synthetic construct
  misc_feature 1..23: note = Synthetic: Target sequences for wild type COL8A2
  source 1..23: mol_type = other DNA
aatgggggaatgggtagatgggg

SEQ ID NO: 878 - DNA, 23 bp, synthetic construct
  misc_feature 1..23: note = Synthetic: Target sequences for wild type COL8A2
  source 1..23: mol_type = other DNA
gaatgggggaatgggtagatggg

SEQ ID NO: 879 - DNA, 23 bp, synthetic construct
  misc_feature 1..23: note = Synthetic: Target sequences for wild type COL8A2
  source 1..23: mol_type = other DNA
ggaatgggggaatgggtagatgg

SEQ ID NO: 880 - DNA, 23 bp, synthetic construct
  misc_feature 1..23: note = Synthetic: Target sequences for wild type COL8A2
  source 1..23: mol_type = other DNA
tcatactggaatgggggaatggg

SEQ ID NO: 881 - DNA, 23 bp, synthetic construct
  misc_feature 1..23: note = Synthetic: Target sequences for wild type COL8A2
  source 1..23: mol_type = other DNA
ctcatactggaatgggggaatgg

SEQ ID NO: 882 - DNA, 23 bp, synthetic construct
  misc_feature 1..23: note = Synthetic: Target sequences for wild type COL8A2
  source 1..23: mol_type = other DNA
cattcccccattccagtatgagg

SEQ ID NO: 883 - DNA, 23 bp, synthetic construct
  misc_feature 1..23: note = Synthetic: Target sequences for wild type COL8A2
  source 1..23: mol_type = other DNA
tgtacctcatactggaatggggg

SEQ ID NO: 884 - DNA, 23 bp, synthetic construct
  misc_feature 1..23: note = Synthetic: Target sequences for wild type COL8A2
  source 1..23: mol_type = other DNA
gtgtacctcatactggaatgggg

SEQ ID NO: 885 - DNA, 23 bp, synthetic construct
  misc_feature 1..23: note = Synthetic: Target sequences for wild type COL8A2
  source 1..23: mol_type = other DNA
cgtgtacctcatactggaatggg

SEQ ID NO: 886 - DNA, 23 bp, synthetic construct
  misc_feature 1..23: note = Synthetic: Target sequences for wild type COL8A2
  source 1..23: mol_type = other DNA
ccattccagtatgaggtacacgg

SEQ ID NO: 887 - DNA, 23 bp, synthetic construct
  misc_feature 1..23: note = Synthetic: Target sequences for wild type COL8A2
  source 1..23: mol_type = other DNA
ccgtgtacctcatactggaatgg

SEQ ID NO: 888 - DNA, 23 bp, synthetic construct
  misc_feature 1..23: note = Synthetic: Target sequences for wild type COL8A2
  source 1..23: mol_type = other DNA
cattccagtatgaggtacacggg

SEQ ID NO: 889 - DNA, 23 bp, synthetic construct
  misc_feature 1..23: note = Synthetic: Target sequences for wild type COL8A2
  source 1..23: mol_type = other DNA
ctctcccgtgtacctcatactgg

SEQ ID NO: 890 - DNA, 23 bp, synthetic construct
  misc_feature 1..23: note = Synthetic: Target sequences for wild type COL8A2
  source 1..23: mol_type = other DNA
cagtatgaggtacacgggagagg

SEQ ID NO: 891 - DNA, 23 bp, synthetic construct
  misc_feature 1..23: note = Synthetic: Target sequences for wild type COL8A2
  source 1..23: mol_type = other DNA
ggtacacgggagaggaagaatgg

SEQ ID NO: 892 - DNA, 23 bp, synthetic construct
  misc_feature 1..23: note = Synthetic: Target sequences for wild type COL8A2
  source 1..23: mol_type = other DNA
gtacacgggagaggaagaatggg

SEQ ID NO: 893 - DNA, 23 bp, synthetic construct
  misc_feature 1..23: note = Synthetic: Target sequences for wild type COL8A2
  source 1..23: mol_type = other DNA
tacacgggagaggaagaatgggg

SEQ ID NO: 894 - DNA, 23 bp, synthetic construct
  misc_feature 1..23: note = Synthetic: Target sequences for wild type COL8A2
  source 1..23: mol_type = other DNA
gctgccccttcctgctctcatgg

SEQ ID NO: 895 - DNA, 23 bp, synthetic construct
  misc_feature 1..23: note = Synthetic: Target sequences for wild type COL8A2
  source 1..23: mol_type = other DNA
tcttccatgagagcaggaagggg

SEQ ID NO: 896 - DNA, 23 bp, synthetic construct
  misc_feature 1..23: note = Synthetic: Target sequences for wild type COL8A2
  source 1..23: mol_type = other DNA
atcttccatgagagcaggaaggg

SEQ ID NO: 897 - DNA, 23 bp, synthetic construct
  misc_feature 1..23: note = Synthetic: Target sequences for wild type COL8A2
  source 1..23: mol_type = other DNA
catcttccatgagagcaggaagg

SEQ ID NO: 898 - DNA, 23 bp, synthetic construct
  misc_feature 1..23: note = Synthetic: Target sequences for wild type COL8A2
  source 1..23: mol_type = other DNA
cttcctgctctcatggaagatgg

SEQ ID NO: 899 - DNA, 23 bp, synthetic construct
  misc_feature 1..23: note = Synthetic: Target sequences for wild type COL8A2
  source 1..23: mol_type = other DNA
ttcctgctctcatggaagatggg

SEQ ID NO: 900 - DNA, 23 bp, synthetic construct
  misc_feature 1..23: note = Synthetic: Target sequences for wild type COL8A2
  source 1..23: mol_type = other DNA
tcctgctctcatggaagatgggg

SEQ ID NO: 901 - DNA, 23 bp, synthetic construct
  misc_feature 1..23: note = Synthetic: Target sequences for wild type COL8A2
  source 1..23: mol_type = other DNA
accccatcttccatgagagcagg

SEQ ID NO: 902 - DNA, 23 bp, synthetic construct
  misc_feature 1..23: note = Synthetic: Target sequences for wild type COL8A2
  source 1..23: mol_type = other DNA
ctctcatggaagatggggtttgg

SEQ ID NO: 903 - DNA, 23 bp, synthetic construct
  misc_feature 1..23: note = Synthetic: Target sequences for wild type COL8A2
  source 1..23: mol_type = other DNA
tctcatggaagatggggtttggg

SEQ ID NO: 904 - DNA, 23 bp, synthetic construct
  misc_feature 1..23: note = Synthetic: Target sequences for wild type COL8A2
  source 1..23: mol_type = other DNA
ctcatggaagatggggtttgggg

SEQ ID NO: 905 - DNA, 23 bp, synthetic construct
  misc_feature 1..23: note = Synthetic: Target sequences for wild type COL8A2
  source 1..23: mol_type = other DNA
tcatggaagatggggtttggggg

SEQ ID NO: 906 - DNA, 23 bp, synthetic construct
  misc_feature 1..23: note = Synthetic: Target sequences for wild type COL8A2
  source 1..23: mol_type = other DNA
tggaagatggggtttgggggtgg

SEQ ID NO: 907 - DNA, 23 bp, synthetic construct
  misc_feature 1..23: note = Synthetic: Target sequences for wild type COL8A2
  source 1..23: mol_type = other DNA
atggggtttgggggtggcccagg

SEQ ID NO: 908 - DNA, 23 bp, synthetic construct
  misc_feature 1..23: note = Synthetic: Target sequences for wild type COL8A2
  source 1..23: mol_type = other DNA
tggggtttgggggtggcccaggg

SEQ ID NO: 909 - DNA, 23 bp, synthetic construct
  misc_feature 1..23: note = Synthetic: Target sequences for wild type COL8A2
  source 1..23: mol_type = other DNA
ggggtttgggggtggcccagggg

SEQ ID NO: 910 - DNA, 23 bp, synthetic construct
  misc_feature 1..23: note = Synthetic: Target sequences for wild type COL8A2
  source 1..23: mol_type = other DNA
gggtggcccaggggacatcttgg

SEQ ID NO: 911 - DNA, 23 bp, synthetic construct
  misc_feature 1..23: note = Synthetic: Target sequences for wild type COL8A2
  source 1..23: mol_type = other DNA
ggtggcccaggggacatcttggg

SEQ ID NO: 912 - DNA, 23 bp, synthetic construct
  misc_feature 1..23: note = Synthetic: Target sequences for wild type COL8A2
  source 1..23: mol_type = other DNA
gtggcccaggggacatcttgggg

SEQ ID NO: 913 - DNA, 23 bp, synthetic construct
  misc_feature 1..23: note = Synthetic: Target sequences for wild type COL8A2
  source 1..23: mol_type = other DNA
tggcccaggggacatcttggggg

SEQ ID NO: 914 - DNA, 23 bp, synthetic construct
  misc_feature 1..23: note = Synthetic: Target sequences for wild type COL8A2
  source 1..23: mol_type = other DNA
ttgcccccaagatgtcccctggg

SEQ ID NO: 915 - DNA, 23 bp, synthetic construct
  misc_feature 1..23: note = Synthetic: Target sequences for wild type COL8A2
  source 1..23: mol_type = other DNA
gttgcccccaagatgtcccctgg

SEQ ID NO: 916 - DNA, 23 bp, synthetic construct
  misc_feature 1..23: note = Synthetic: Target sequences for wild type COL8A2
  source 1..23: mol_type = other DNA
ggggacatcttgggggcaacagg

SEQ ID NO: 917 - DNA, 23 bp, synthetic construct
  misc_feature 1..23: note = Synthetic: Target sequences for wild type COL8A2
  source 1..23: mol_type = other DNA
gggacatcttgggggcaacaggg

SEQ ID NO: 918 - DNA, 23 bp, synthetic construct
  misc_feature 1..23: note = Synthetic: Target sequences for wild type COL8A2
  source 1..23: mol_type = other DNA
gcaacagggtgtcctccttaagg

SEQ ID NO: 919 - DNA, 23 bp, synthetic construct
  misc_feature 1..23: note = Synthetic: Target sequences for wild type COL8A2
  source 1..23: mol_type = other DNA
caacagggtgtcctccttaaggg

SEQ ID NO: 920 - DNA, 23 bp, synthetic construct
  misc_feature 1..23: note = Synthetic: Target sequences for wild type COL8A2
  source 1..23: mol_type = other DNA
ggtgttaggagcccttaaggagg

SEQ ID NO: 921 - DNA, 23 bp, synthetic construct
  misc_feature 1..23: note = Synthetic: Target sequences for wild type COL8A2
  source 1..23: mol_type = other DNA
ttgggtgttaggagcccttaagg

SEQ ID NO: 922 - DNA, 23 bp, synthetic construct
  misc_feature 1..23: note = Synthetic: Target sequences for wild type COL8A2
  source 1..23: mol_type = other DNA
tcctaacacccaacctacctagg

SEQ ID NO: 923 - DNA, 23 bp, synthetic construct
  misc_feature 1..23: note = Synthetic: Target sequences for wild type COL8A2
  source 1..23: mol_type = other DNA
gcctaggtaggttgggtgttagg

SEQ ID NO: 924 - DNA, 23 bp, synthetic construct
  misc_feature 1..23: note = Synthetic: Target sequences for wild type COL8A2
  source 1..23: mol_type = other DNA
aacacccaacctacctaggctgg

SEQ ID NO: 925 - DNA, 23 bp, synthetic construct
  misc_feature 1..23: note = Synthetic: Target sequences for wild type COL8A2
  source 1..23: mol_type = other DNA
acacccaacctacctaggctggg

SEQ ID NO: 926 - DNA, 23 bp, synthetic construct
  misc_feature 1..23: note = Synthetic: Target sequences for wild type COL8A2
  source 1..23: mol_type = other DNA
aggcccagcctaggtaggttggg

SEQ ID NO: 927 - DNA, 23 bp, synthetic construct
  misc_feature 1..23: note = Synthetic: Target sequences for wild type COL8A2
  source 1..23: mol_type = other DNA
gaggcccagcctaggtaggttgg

SEQ ID NO: 928 - DNA, 23 bp, synthetic construct
  misc_feature 1..23: note = Synthetic: Target sequences for wild type COL8A2
  source 1..23: mol_type = other DNA
ggaggaggcccagcctaggtagg

SEQ ID NO: 929 - DNA, 23 bp, synthetic construct
  misc_feature 1..23: note = Synthetic: Target sequences for wild type COL8A2
  source 1..23: mol_type = other DNA
tcatggaggaggcccagcctagg

SEQ ID NO: 930 - DNA, 23 bp, synthetic construct
  misc_feature 1..23: note = Synthetic: Target sequences for wild type COL8A2
  source 1..23: mol_type = other DNA
ctgggcctcctccatgagcctgg

SEQ ID NO: 931 - DNA, 23 bp, synthetic construct
  misc_feature 1..23: note = Synthetic: Target sequences for wild type COL8A2
  source 1..23: mol_type = other DNA
atcagccaggctcatggaggagg

SEQ ID NO: 932 - DNA, 23 bp, synthetic construct
  misc_feature 1..23: note = Synthetic: Target sequences for wild type COL8A2
  source 1..23: mol_type = other DNA
agaatcagccaggctcatggagg

SEQ ID NO: 933 - DNA, 23 bp, synthetic construct
  misc_feature 1..23: note = Synthetic: Target sequences for wild type COL8A2
  source 1..23: mol_type = other DNA
gtgagaatcagccaggctcatgg

SEQ ID NO: 934 - DNA, 23 bp, synthetic construct
  misc_feature 1..23: note = Synthetic: Target sequences for wild type COL8A2
  source 1..23: mol_type = other DNA
atgagaggtgagaatcagccagg

SEQ ID NO: 935 - DNA, 23 bp, synthetic construct
  misc_feature 1..23: note = Synthetic: Target sequences for wild type COL8A2
  source 1..23: mol_type = other DNA
tcaggtcatgcagggatgagagg

SEQ ID NO: 936 - DNA, 23 bp, synthetic construct
  misc_feature 1..23: note = Synthetic: Target sequences for wild type COL8A2
  source 1..23: mol_type = other DNA
ctcatccctgcatgacctgaagg

SEQ ID NO: 937 - DNA, 23 bp, synthetic construct
  misc_feature 1..23: note = Synthetic: Target sequences for wild type COL8A2
  source 1..23: mol_type = other DNA
atccctgcatgacctgaaggtgg

SEQ ID NO: 938 - DNA, 23 bp, synthetic construct
  misc_feature 1..23: note = Synthetic: Target sequences for wild type COL8A2
  source 1..23: mol_type = other DNA
ctccaccttcaggtcatgcaggg

SEQ ID NO: 939 - DNA, 23 bp, synthetic construct
  misc_feature 1..23: note = Synthetic: Target sequences for wild type COL8A2
  source 1..23: mol_type = other DNA
actccaccttcaggtcatgcagg

SEQ ID NO: 940 - DNA, 23 bp, synthetic construct
  misc_feature 1..23: note = Synthetic: Target sequences for wild type COL8A2
  source 1..23: mol_type = other DNA
tgcatgacctgaaggtggagtgg

SEQ ID NO: 941 - DNA, 23 bp, synthetic construct
  misc_feature 1..23: note = Synthetic: Target sequences for wild type COL8A2
  source 1..23: mol_type = other DNA
ctggtggccactccaccttcagg

SEQ ID NO: 942 - DNA, 23 bp, synthetic construct
  misc_feature 1..23: note = Synthetic: Target sequences for wild type COL8A2
  source 1..23: mol_type = other DNA
ctgaaggtggagtggccaccagg

SEQ ID NO: 943 - DNA, 23 bp, synthetic construct
  misc_feature 1..23: note = Synthetic: Target sequences for wild type COL8A2
  source 1..23: mol_type = other DNA
aaggtggagtggccaccaggtgg

SEQ ID NO: 944 - DNA, 23 bp, synthetic construct
  misc_feature 1..23: note = Synthetic: Target sequences for wild type COL8A2
  source 1..23: mol_type = other DNA
gggctgctggtgccacctggtgg

SEQ ID NO: 945 - DNA, 23 bp, synthetic construct
  misc_feature 1..23: note = Synthetic: Target sequences for wild type COL8A2
  source 1..23: mol_type = other DNA
ggtgggctgctggtgccacctgg

SEQ ID NO: 946 - DNA, 23 bp, synthetic construct
  misc_feature 1..23: note = Synthetic: Target sequences for wild type COL8A2
  source 1..23: mol_type = other DNA
cgggctctaaggtgggctgctgg

SEQ ID NO: 947 - DNA, 23 bp, synthetic construct
  misc_feature 1..23: note = Synthetic: Target sequences for wild type COL8A2
  source 1..23: mol_type = other DNA
gcagcccaccttagagcccgtgg

SEQ ID NO: 948 - DNA, 23 bp, synthetic construct
  misc_feature 1..23: note = Synthetic: Target sequences for wild type COL8A2
  source 1..23: mol_type = other DNA
cagcccaccttagagcccgtggg

SEQ ID NO: 949 - DNA, 23 bp, synthetic construct
  misc_feature 1..23: note = Synthetic: Target sequences for wild type COL8A2
  source 1..23: mol_type = other DNA
gctcccacgggctctaaggtggg

SEQ ID NO: 950 - DNA, 23 bp, synthetic construct
  misc_feature 1..23: note = Synthetic: Target sequences for wild type COL8A2
  source 1..23: mol_type = other DNA
tgctcccacgggctctaaggtgg

SEQ ID NO: 951 - DNA, 23 bp, synthetic construct
  misc_feature 1..23: note = Synthetic: Target sequences for wild type COL8A2
  source 1..23: mol_type = other DNA
ctctgctcccacgggctctaagg

SEQ ID NO: 952 - DNA, 23 bp, synthetic construct
  misc_feature 1..23: note = Synthetic: Target sequences for wild type COL8A2
  source 1..23: mol_type = other DNA
aggtggggctctgctcccacggg

SEQ ID NO: 953 - DNA, 23 bp, synthetic construct
  misc_feature 1..23: note = Synthetic: Target sequences for wild type COL8A2
  source 1..23: mol_type = other DNA
gaggtggggctctgctcccacgg

SEQ ID NO: 954 - DNA, 23 bp, synthetic construct
  misc_feature 1..23: note = Synthetic: Target sequences for wild type COL8A2
  source 1..23: mol_type = other DNA
aactgggaagttgggaggtgggg

SEQ ID NO: 955 - DNA, 23 bp, synthetic construct
  misc_feature 1..23: note = Synthetic: Target sequences for wild type COL8A2
  source 1..23: mol_type = other DNA
gaactgggaagttgggaggtggg

SEQ ID NO: 956 - DNA, 23 bp, synthetic construct
  misc_feature 1..23: note = Synthetic: Target sequences for wild type COL8A2
  source 1..23: mol_type = other DNA
tgaactgggaagttgggaggtgg

SEQ ID NO: 957 - DNA, 23 bp, synthetic construct
  misc_feature 1..23: note = Synthetic: Target sequences for wild type COL8A2
  source 1..23: mol_type = other DNA
agatgaactgggaagttgggagg

SEQ ID NO: 958 - DNA, 23 bp, synthetic construct
  misc_feature 1..23: note = Synthetic: Target sequences for wild type COL8A2
  source 1..23: mol_type = other DNA
gggagatgaactgggaagttggg

SEQ ID NO: 959 - DNA, 23 bp, synthetic construct
  misc_feature 1..23: note = Synthetic: Target sequences for wild type COL8A2
  source 1..23: mol_type = other DNA
ggggagatgaactgggaagttgg

SEQ ID NO: 960 - DNA, 23 bp, synthetic construct
  misc_feature 1..23: note = Synthetic: Target sequences for wild type COL8A2
  source 1..23: mol_type = other DNA
ttcccagttcatctcccccttgg

SEQ ID NO: 961 - DNA, 23 bp, synthetic construct
  misc_feature 1..23: note = Synthetic: Target sequences for wild type COL8A2
  source 1..23: mol_type = other DNA
ttccaagggggagatgaactggg

SEQ ID NO: 962 - DNA, 23 bp, synthetic construct
  misc_feature 1..23: note = Synthetic: Target sequences for wild type COL8A2
  source 1..23: mol_type = other DNA
cttccaagggggagatgaactgg

SEQ ID NO: 963 - DNA, 23 bp, synthetic construct
  misc_feature 1..23: note = Synthetic: Target sequences for wild type COL8A2
  source 1..23: mol_type = other DNA
gcacaggtggtcttccaaggggg

SEQ ID NO: 964 - DNA, 23 bp, synthetic construct
  misc_feature 1..23: note = Synthetic: Target sequences for wild type COL8A2
  source 1..23: mol_type = other DNA
ggcacaggtggtcttccaagggg

SEQ ID NO: 965 - DNA, 23 bp, synthetic construct
  misc_feature 1..23: note = Synthetic: Target sequences for wild type COL8A2
  source 1..23: mol_type = other DNA
tggcacaggtggtcttccaaggg

SEQ ID NO: 966 - DNA, 23 bp, synthetic construct
  misc_feature 1..23: note = Synthetic: Target sequences for wild type COL8A2
  source 1..23: mol_type = other DNA
ctggcacaggtggtcttccaagg

SEQ ID NO: 967 - DNA, 23 bp, synthetic construct
  misc_feature 1..23: note = Synthetic: Target sequences for wild type COL8A2
  source 1..23: mol_type = other DNA
gtgcagttagctggcacaggtgg

SEQ ID NO: 968 - DNA, 23 bp, synthetic construct
  misc_feature 1..23: note = Synthetic: Target sequences for wild type COL8A2
  source 1..23: mol_type = other DNA
acggtgcagttagctggcacagg

SEQ ID NO: 969 - DNA, 23 bp, synthetic construct
  misc_feature 1..23: note = Synthetic: Target sequences for wild type COL8A2
  source 1..23: mol_type = other DNA
ctggaaacggtgcagttagctgg

SEQ ID NO: 970 - DNA, 23 bp, synthetic construct
  misc_feature 1..23: note = Synthetic: Target sequences for wild type COL8A2
  source 1..23: mol_type = other DNA
cagctaactgcaccgtttccagg

SEQ ID NO: 971 - DNA, 23 bp, synthetic construct
  misc_feature 1..23: note = Synthetic: Target sequences for wild type COL8A2
  source 1..23: mol_type = other DNA
tgcaccgtttccaggccctctgg

SEQ ID NO: 972 - DNA, 23 bp, synthetic construct
  misc_feature 1..23: note = Synthetic: Target sequences for wild type COL8A2
  source 1..23: mol_type = other DNA
gcaccgtttccaggccctctggg

SEQ ID NO: 973 - DNA, 23 bp, synthetic construct
  misc_feature 1..23: note = Synthetic: Target sequences for wild type COL8A2
  source 1..23: mol_type = other DNA
caccgtttccaggccctctgggg

SEQ ID NO: 974 - DNA, 23 bp, synthetic construct
  misc_feature 1..23: note = Synthetic: Target sequences for wild type COL8A2
  source 1..23: mol_type = other DNA
taccccagagggcctggaaacgg

SEQ ID NO: 975 - DNA, 23 bp, synthetic construct
  misc_feature 1..23: note = Synthetic: Target sequences for wild type COL8A2
  source 1..23: mol_type = other DNA
tccaggccctctggggtattagg

SEQ ID NO: 976 - DNA, 23 bp, synthetic construct
  misc_feature 1..23: note = Synthetic: Target sequences for wild type COL8A2
  source 1..23: mol_type = other DNA
tcctaataccccagagggcctgg

SEQ ID NO: 977 - DNA, 23 bp, synthetic construct
  misc_feature 1..23: note = Synthetic: Target sequences for wild type COL8A2
  source 1..23: mol_type = other DNA
gtttttcctaataccccagaggg

SEQ ID NO: 978 - DNA, 23 bp, synthetic construct
  misc_feature 1..23: note = Synthetic: Target sequences for wild type COL8A2
  source 1..23: mol_type = other DNA
tgtttttcctaataccccagagg

SEQ ID NO: 979 - DNA, 23 bp, synthetic construct
  misc_feature 1..23: note = Synthetic: Target sequences for wild type COL8A2
  source 1..23: mol_type = other DNA
ggtattaggaaaaacactgaagg

SEQ ID NO: 980 - DNA, 23 bp, synthetic construct
  misc_feature 1..23: note = Synthetic: Target sequences for wild type COL8A2
  source 1..23: mol_type = other DNA
ttaggaaaaacactgaaggtagg

SEQ ID NO: 981 - DNA, 23 bp, synthetic construct
  misc_feature 1..23: note = Synthetic: Target sequences for wild type COL8A2
  source 1..23: mol_type = other DNA
aacactgaaggtaggaaaattgg

SEQ ID NO: 982 - DNA, 23 bp, synthetic construct
  misc_feature 1..23: note = Synthetic: Target sequences for wild type COL8A2
  source 1..23: mol_type = other DNA
actgaaggtaggaaaattggtgg

SEQ ID NO: 983 - DNA, 23 bp, synthetic construct
  misc_feature 1..23: note = Synthetic: Target sequences for wild type COL8A2
  source 1..23: mol_type = other DNA
ctgaaggtaggaaaattggtggg

SEQ ID NO: 984 - DNA, 23 bp, synthetic construct
  misc_feature 1..23: note = Synthetic: Target sequences for wild type COL8A2
  source 1..23: mol_type = other DNA
tgaaggtaggaaaattggtgggg

SEQ ID NO: 985 - DNA, 23 bp, synthetic construct
  misc_feature 1..23: note = Synthetic: Target sequences for wild type COL8A2
  source 1..23: mol_type = other DNA
aggaaaattggtggggaatgagg

SEQ ID NO: 986 - DNA, 23 bp, synthetic construct
  misc_feature 1..23: note = Synthetic: Target sequences for wild type COL8A2
  source 1..23: mol_type = other DNA
tggtggggaatgaggagctgtgg

SEQ ID NO: 987 - DNA, 23 bp, synthetic construct
  misc_feature 1..23: note = Synthetic: Target sequences for wild type COL8A2
  source 1..23: mol_type = other DNA
tggggaatgaggagctgtggagg

SEQ ID NO: 988 - DNA, 23 bp, synthetic construct
  misc_feature 1..23: note = Synthetic: Target sequences for wild type COL8A2
  source 1..23: mol_type = other DNA
ggggaatgaggagctgtggaggg

SEQ ID NO: 989 - DNA, 23 bp, synthetic construct
  misc_feature 1..23: note = Synthetic: Target sequences for wild type COL8A2
  source 1..23: mol_type = other DNA
ggagctgtggagggcgcctgagg

SEQ ID NO: 990 - DNA, 23 bp, synthetic construct
  misc_feature 1..23: note = Synthetic: Target sequences for wild type COL8A2
  source 1..23: mol_type = other DNA
gagggcgcctgaggatctgatgg

SEQ ID NO: 991 - DNA, 23 bp, synthetic construct
  misc_feature 1..23: note = Synthetic: Target sequences for wild type COL8A2
  source 1..23: mol_type = other DNA
ctgagagccatcagatcctcagg

SEQ ID NO: 992 - DNA, 23 bp, synthetic construct
  misc_feature 1..23: note = Synthetic: Target sequences for wild type COL8A2
  source 1..23: mol_type = other DNA
ctgaggatctgatggctctcagg

SEQ ID NO: 993 - DNA, 23 bp, synthetic construct
  misc_feature 1..23: note = Synthetic: Target sequences for wild type COL8A2
  source 1..23: mol_type = other DNA
tgaggatctgatggctctcaggg

SEQ ID NO: 994 - DNA, 23 bp, synthetic construct
  misc_feature 1..23: note = Synthetic: Target sequences for wild type COL8A2
  source 1..23: mol_type = other DNA
ggatctgatggctctcagggagg

SEQ ID NO: 995 - DNA, 23 bp, synthetic construct
  misc_feature 1..23: note = Synthetic: Target sequences for wild type COL8A2
  source 1..23: mol_type = other DNA
ctgatggctctcagggaggcagg

SEQ ID NO: 996 - DNA, 23 bp, synthetic construct
  misc_feature 1..23: note = Synthetic: Target sequences for wild type COL8A2
  source 1..23: mol_type = other DNA
tgatggctctcagggaggcaggg

SEQ ID NO: 997 - DNA, 23 bp, synthetic construct
  misc_feature 1..23: note = Synthetic: Target sequences for wild type COL8A2
  source 1..23: mol_type = other DNA
gatggctctcagggaggcagggg

SEQ ID NO: 998 - DNA, 23 bp, synthetic construct
  misc_feature 1..23: note = Synthetic: Target sequences for wild type COL8A2
  source 1..23: mol_type = other DNA
tctcagggaggcaggggatttgg

SEQ ID NO: 999 - DNA, 23 bp, synthetic construct
  misc_feature 1..23: note = Synthetic: Target sequences for wild type COL8A2
  source 1..23: mol_type = other DNA
ctcagggaggcaggggatttggg

SEQ ID NO: 1000 - DNA, 23 bp, synthetic construct
  misc_feature 1..23: note = Synthetic: Target sequences for wild type COL8A2
  source 1..23: mol_type = other DNA
tcagggaggcaggggatttgggg

SEQ ID NO: 1001 - DNA, 23 bp, synthetic construct
  misc_feature 1..23: note = Synthetic: Target sequences for wild type COL8A2
  source 1..23: mol_type = other DNA
cagggaggcaggggatttggggg

SEQ ID NO: 1002 - DNA, 23 bp, synthetic construct
  misc_feature 1..23: note = Synthetic: Target sequences for wild type COL8A2
  source 1..23: mol_type = other DNA
gaggcaggggatttgggggctgg

SEQ ID NO: 1003 - DNA, 23 bp, synthetic construct
  misc_feature 1..23: note = Synthetic: Target sequences for wild type COL8A2
  source 1..23: mol_type = other DNA
aggcaggggatttgggggctggg

SEQ ID NO: 1004 - DNA, 23 bp, synthetic construct
  misc_feature 1..23: note = Synthetic: Target sequences for wild type COL8A2
  source 1..23: mol_type = other DNA
tgggggctgggagcgatttgagg

SEQ ID NO: 1005 - DNA, 23 bp, synthetic construct
  misc_feature 1..23: note = Synthetic: Target sequences for wild type COL8A2
  source 1..23: mol_type = other DNA
gggagcgatttgaggcactgtgg

SEQ ID NO: 1006 - DNA, 23 bp, synthetic construct
  misc_feature 1..23: note = Synthetic: Target sequences for wild type COL8A2
  source 1..23: mol_type = other DNA
ggagcgatttgaggcactgtggg

SEQ ID NO: 1007 - DNA, 23 bp, synthetic construct
  misc_feature 1..23: note = Synthetic: Target sequences for wild type COL8A2
  source 1..23: mol_type = other DNA
gagcgatttgaggcactgtgggg

SEQ ID NO: 1008 - DNA, 23 bp, synthetic construct
  misc_feature 1..23: note = Synthetic: Target sequences for wild type COL8A2
  source 1..23: mol_type = other DNA
atttgaggcactgtggggtgagg

SEQ ID NO: 1009 - DNA, 23 bp, synthetic construct
  misc_feature 1..23: note = Synthetic: Target sequences for wild type COL8A2
  source 1..23: mol_type = other DNA
tgaggcactgtggggtgaggagg

SEQ ID NO: 1010 - DNA, 23 bp, synthetic construct
  misc_feature 1..23: note = Synthetic: Target sequences for wild type COL8A2
  source 1..23: mol_type = other DNA
gggtgaggaggctctcacccagg

SEQ ID NO: 1011 - DNA, 23 bp, synthetic construct
  misc_feature 1..23: note = Synthetic: Target sequences for wild type COL8A2
  source 1..23: mol_type = other DNA
ggaggctctcacccaggtactgg

SEQ ID NO: 1012 - DNA, 23 bp, synthetic construct
  misc_feature 1..23: note = Synthetic: Target sequences for wild type COL8A2
  source 1..23: mol_type = other DNA
gagggcaaaggccagtacctggg

SEQ ID NO: 1013 - DNA, 23 bp, synthetic construct
  misc_feature 1..23: note = Synthetic: Target sequences for wild type COL8A2
  source 1..23: mol_type = other DNA
tgagggcaaaggccagtacctgg

SEQ ID NO: 1014 - DNA, 23 bp, synthetic construct
  misc_feature 1..23: note = Synthetic: Target sequences for wild type COL8A2
  source 1..23: mol_type = other DNA
ggtactggcctttgccctcacgg

SEQ ID NO: 1015 - DNA, 23 bp, synthetic construct
  misc_feature 1..23: note = Synthetic: Target sequences for wild type COL8A2
  source 1..23: mol_type = other DNA
ctggcctttgccctcacggaagg

SEQ ID NO: 1016 - DNA, 23 bp, synthetic construct
  misc_feature 1..23: note = Synthetic: Target sequences for wild type COL8A2
  source 1..23: mol_type = other DNA
tggcctttgccctcacggaaggg

SEQ ID NO: 1017 - DNA, 23 bp, synthetic construct
  misc_feature 1..23: note = Synthetic: Target sequences for wild type COL8A2
  source 1..23: mol_type = other DNA
cctttgccctcacggaagggcgg

SEQ ID NO: 1018 - DNA, 23 bp, synthetic construct
  misc_feature 1..23: note = Synthetic: Target sequences for wild type COL8A2
  source 1..23: mol_type = other DNA
ccgcccttccgtgagggcaaagg

SEQ ID NO: 1019 - DNA, 23 bp, synthetic construct
  misc_feature 1..23: note = Synthetic: Target sequences for wild type COL8A2
  source 1..23: mol_type = other DNA
gtgggaccgcccttccgtgaggg

SEQ ID NO: 1020 - DNA, 23 bp, synthetic construct
  misc_feature 1..23: note = Synthetic: Target sequences for wild type COL8A2
  source 1..23: mol_type = other DNA
tgtgggaccgcccttccgtgagg

SEQ ID NO: 1021 - DNA, 23 bp, synthetic construct
  misc_feature 1..23: note = Synthetic: Target sequences for wild type COL8A2
  source 1..23: mol_type = other DNA
tcacggaagggcggtcccacagg

SEQ ID NO: 1022 - DNA, 23 bp, synthetic construct
  misc_feature 1..23: note = Synthetic: Target sequences for wild type COL8A2
  source 1..23: mol_type = other DNA
tcccacaggtcctttctgcatgg

SEQ ID NO: 1023 - DNA, 23 bp, synthetic construct
  misc_feature 1..23: note = Synthetic: Target sequences for wild type COL8A2
  source 1..23: mol_type = other DNA
cccacaggtcctttctgcatggg

SEQ ID NO: 1024 - DNA, 23 bp, synthetic construct
  misc_feature 1..23: note = Synthetic: Target sequences for wild type COL8A2
  source 1..23: mol_type = other DNA
cccatgcagaaaggacctgtggg

SEQ ID NO: 1025 - DNA, 23 bp, synthetic construct
  misc_feature 1..23: note = Synthetic: Target sequences for wild type COL8A2
  source 1..23: mol_type = other DNA
gcccatgcagaaaggacctgtgg

SEQ ID NO: 1026 - DNA, 23 bp, synthetic construct
  misc_feature 1..23: note = Synthetic: Target sequences for wild type COL8A2
  source 1..23: mol_type = other DNA
caggtcctttctgcatgggctgg

SEQ ID NO: 1027 - DNA, 23 bp, synthetic construct
  misc_feature 1..23: note = Synthetic: Target sequences for wild type COL8A2
  source 1..23: mol_type = other DNA
tacatccagcccatgcagaaagg

SEQ ID NO: 1028 - DNA, 23 bp, synthetic construct
  misc_feature 1..23: note = Synthetic: Target sequences for wild type COL8A2
  source 1..23: mol_type = other DNA
atgggctggatgtacttcactgg

SEQ ID NO: 1029 - DNA, 23 bp, synthetic construct
  misc_feature 1..23: note = Synthetic: Target sequences for wild type COL8A2
  source 1..23: mol_type = other DNA
tgggctggatgtacttcactggg

SEQ ID NO: 1030 - DNA, 23 bp, synthetic construct
  misc_feature 1..23: note = Synthetic: Target sequences for wild type COL8A2
  source 1..23: mol_type = other DNA
gggctggatgtacttcactgggg

SEQ ID NO: 1031 - DNA, 23 bp, synthetic construct
  misc_feature 1..23: note = Synthetic: Target sequences for wild type COL8A2
  source 1..23: mol_type = other DNA
ggcatagcccgccgccccaccgg

SEQ ID NO: 1032 - DNA, 23 bp, synthetic construct
  misc_feature 1..23: note = Synthetic: Target sequences for wild type COL8A2
  source 1..23: mol_type = other DNA
ggcggggccggtggggcggcggg

SEQ ID NO: 1033 - DNA, 23 bp, synthetic construct
  misc_feature 1..23: note = Synthetic: Target sequences for wild type COL8A2
  source 1..23: mol_type = other DNA
tggcggggccggtggggcggcgg

SEQ ID NO: 1034 - DNA, 23 bp, synthetic construct
  misc_feature 1..23: note = Synthetic: Target sequences for wild type COL8A2
  source 1..23: mol_type = other DNA
tggtggcggggccggtggggcgg

SEQ ID NO: 1035 - DNA, 23 bp, synthetic construct
  misc_feature 1..23: note = Synthetic: Target sequences for wild type COL8A2
  source 1..23: mol_type = other DNA
ctctggtggcggggccggtgggg

SEQ ID NO: 1036 - DNA, 23 bp, synthetic construct
  misc_feature 1..23: note = Synthetic: Target sequences for wild type COL8A2
  source 1..23: mol_type = other DNA
cccaccggccccgccaccagagg

SEQ ID NO: 1037 - DNA, 23 bp, synthetic construct
  misc_feature 1..23: note = Synthetic: Target sequences for wild type COL8A2
  source 1..23: mol_type = other DNA
cctctggtggcggggccggtggg

SEQ ID NO: 1038 - DNA, 23 bp, synthetic construct
  misc_feature 1..23: note = Synthetic: Target sequences for wild type COL8A2
  source 1..23: mol_type = other DNA
tcctctggtggcggggccggtgg

SEQ ID NO: 1039 - DNA, 23 bp, synthetic construct
  misc_feature 1..23: note = Synthetic: Target sequences for wild type COL8A2
  source 1..23: mol_type = other DNA
gcgtcctctggtggcggggccgg

SEQ ID NO: 1040 - DNA, 23 bp, synthetic construct
  misc_feature 1..23: note = Synthetic: Target sequences for wild type COL8A2
  source 1..23: mol_type = other DNA
gcgggcgtcctctggtggcgggg

SEQ ID NO: 1041 - DNA, 23 bp, synthetic construct
  misc_feature 1..23: note = Synthetic: Target sequences for wild type COL8A2
  source 1..23: mol_type = other DNA
cgcgggcgtcctctggtggcggg

SEQ ID NO: 1042 - DNA, 23 bp, synthetic construct
  misc_feature 1..23: note = Synthetic: Target sequences for wild type COL8A2
  source 1..23: mol_type = other DNA
ccgccaccagaggacgcccgcgg

SEQ ID NO: 1043 - DNA, 23 bp, synthetic construct
  misc_feature 1..23: note = Synthetic: Target sequences for wild type COL8A2
  source 1..23: mol_type = other DNA
ccgcgggcgtcctctggtggcgg

SEQ ID NO: 1044 - DNA, 23 bp, synthetic construct
  misc_feature 1..23: note = Synthetic: Target sequences for wild type COL8A2
  source 1..23: mol_type = other DNA
gggccgcgggcgtcctctggtgg

SEQ ID NO: 1045 - DNA, 23 bp, synthetic construct
  misc_feature 1..23: note = Synthetic: Target sequences for wild type COL8A2
  source 1..23: mol_type = other DNA
tgtgggccgcgggcgtcctctgg

SEQ ID NO: 1046 - DNA, 23 bp, synthetic construct
  misc_feature 1..23: note = Synthetic: Target sequences for wild type COL8A2
  source 1..23: mol_type = other DNA
ggtgctggggtgtgggccgcggg

SEQ ID NO: 1047 - DNA, 23 bp, synthetic construct
  misc_feature 1..23: note = Synthetic: Target sequences for wild type COL8A2
  source 1..23: mol_type = other DNA
tggtgctggggtgtgggccgcgg

SEQ ID NO: 1048 - DNA, 23 bp, synthetic construct
  misc_feature 1..23: note = Synthetic: Target sequences for wild type COL8A2
  source 1..23: mol_type = other DNA
tggtgctggtgctggggtgtggg

SEQ ID NO: 1049 - DNA, 23 bp, synthetic construct
  misc_feature 1..23: note = Synthetic: Target sequences for wild type COL8A2
  source 1..23: mol_type = other DNA
ctggtgctggtgctggggtgtgg

SEQ ID NO: 1050 - DNA, 23 bp, synthetic construct
  misc_feature 1..23: note = Synthetic: Target sequences for wild type COL8A2
  source 1..23: mol_type = other DNA
tgctactggtgctggtgctgggg

SEQ ID NO: 1051 - DNA, 23 bp, synthetic construct
  misc_feature 1..23: note = Synthetic: Target sequences for wild type COL8A2
  source 1..23: mol_type = other DNA
ctgctactggtgctggtgctggg

SEQ ID NO: 1052 - DNA, 23 bp, synthetic construct
  misc_feature 1..23: note = Synthetic: Target sequences for wild type COL8A2
  source 1..23: mol_type = other DNA
gctgctactggtgctggtgctgg

SEQ ID NO: 1053 - DNA, 23 bp, synthetic construct
  misc_feature 1..23: note = Synthetic: Target sequences for wild type COL8A2
  source 1..23: mol_type = other DNA
gctgctgctgctactggtgctgg

SEQ ID NO: 1054 - DNA, 23 bp, synthetic construct
  misc_feature 1..23: note = Synthetic: Target sequences for wild type COL8A2
  source 1..23: mol_type = other DNA
ttcgctgctgctgctgctactgg

SEQ ID NO: 1055 - DNA, 23 bp, synthetic construct
  misc_feature 1..23: note = Synthetic: Target sequences for wild type COL8A2
  source 1..23: mol_type = other DNA
gcagcagcagcagcgaagacagg

SEQ ID NO: 1056 - DNA, 23 bp, synthetic construct
  misc_feature 1..23: note = Synthetic: Target sequences for wild type COL8A2
  source 1..23: mol_type = other DNA
cagcagcagcagcgaagacaggg

SEQ ID NO: 1057 - DNA, 23 bp, synthetic construct
  misc_feature 1..23: note = Synthetic: Target sequences for wild type COL8A2
  source 1..23: mol_type = other DNA
agcagcagcagcgaagacagggg

SEQ ID NO: 1058 - DNA, 23 bp, synthetic construct
  misc_feature 1..23: note = Synthetic: Target sequences for wild type COL8A2
  source 1..23: mol_type = other DNA
gggtgtcagagtccccagcatgg

SEQ ID NO: 1059 - DNA, 23 bp, synthetic construct
  misc_feature 1..23: note = Synthetic: Target sequences for wild type COL8A2
  source 1..23: mol_type = other DNA
agtccccagcatggcgtccgtgg

SEQ ID NO: 1060 - DNA, 23 bp, synthetic construct
  misc_feature 1..23: note = Synthetic: Target sequences for wild type COL8A2
  source 1..23: mol_type = other DNA
cgtccacggacgccatgctgggg

SEQ ID NO: 1061 - DNA, 23 bp, synthetic construct
  misc_feature 1..23: note = Synthetic: Target sequences for wild type COL8A2
  source 1..23: mol_type = other DNA
acgtccacggacgccatgctggg

SEQ ID NO: 1062 - DNA, 23 bp, synthetic construct
  misc_feature 1..23: note = Synthetic: Target sequences for wild type COL8A2
  source 1..23: mol_type = other DNA
cacgtccacggacgccatgctgg

SEQ ID NO: 1063 - DNA, 23 bp, synthetic construct
  misc_feature 1..23: note = Synthetic: Target sequences for wild type COL8A2
  source 1..23: mol_type = other DNA
tcttctttgcagcacgtccacgg

SEQ ID NO: 1064 - DNA, 23 bp, synthetic construct
  misc_feature 1..23: note = Synthetic: Target sequence for COL8A2 Gln455Lys Mutation
  source 1..23: mol_type = other DNA
cccctcaggccaggcttcccagg

SEQ ID NO: 1065 - DNA, 23 bp, synthetic construct
  misc_feature 1..23: note = Synthetic: Target sequence for COL8A2 Gln455Lys Mutation
  source 1..23: mol_type = other DNA
cctgggaagcctggcctgagggg

SEQ ID NO: 1066 - DNA, 23 bp, synthetic construct
  misc_feature 1..23: note = Synthetic: Target sequence for COL8A2 Gln455Lys Mutation
  source 1..23: mol_type = other DNA
ccctcaggccaggttgcccaggg

SEQ ID NO: 1067 - DNA, 23 bp, synthetic construct
  misc_feature 1..23: note = Synthetic: Target sequence for COL8A2 Gln455Lys Mutation
  source 1..23: mol_type = other DNA
ccctgggaagcctggcctgaggg

SEQ ID NO: 1068 - DNA, 23 bp, synthetic construct
  misc_feature 1..23: note = Synthetic: Target sequence for COL8A2 Gln455Lys Mutation
  source 1..23: mol_type = other DNA
tccctgggaagcctggcctgagg

SEQ ID NO: 1069 - DNA, 23 bp, synthetic construct
  misc_feature 1..23: note = Synthetic: Target sequence for COL8A2 Gln455Lys Mutation
  source 1..23: mol_type = other DNA
ttggggctccctgggaagcctgg

SEQ ID NO: 1070 - DNA, 23 bp, synthetic construct
  misc_feature 1..23: note = Synthetic: Target sequence for COL8A2 Gln455Val Mutation
  source 1..23: mol_type = other DNA
cccctcaggccaggcaccccagg

SEQ ID NO: 1071 - DNA, 23 bp, synthetic construct
  misc_feature 1..23: note = Synthetic: Target sequence for COL8A2 Gln455Val Mutation
  source 1..23: mol_type = other DNA
cctggggtgcctggcctgagggg

SEQ ID NO: 1072 - DNA, 23 bp, synthetic construct
  misc_feature 1..23: note = Synthetic: Target sequence for COL8A2 Gln455Val Mutation
  source 1..23: mol_type = other DNA
ccctcaggccaggcaccccaggg

SEQ ID NO: 1073 - DNA, 23 bp, synthetic construct
  misc_feature 1..23: note = Synthetic: Target sequence for COL8A2 Gln455Val Mutation
  source 1..23: mol_type = other DNA
ccctggggtgcctggcctgaggg

SEQ ID NO: 1074 - DNA, 23 bp, synthetic construct
  misc_feature 1..23: note = Synthetic: Target sequence for COL8A2 Gln455Val Mutation
  source 1..23: mol_type = other DNA
tccctggggtgcctggcctgagg

SEQ ID NO: 1075 - DNA, 23 bp, synthetic construct
  misc_feature 1..23: note = Synthetic: Target sequence for COL8A2 Gln455Val Mutation
  source 1..23: mol_type = other DNA
ttggggctccctggggtgcctgg

SEQ ID NO: 1076 - DNA, 23 bp, synthetic construct
  misc_feature 1..23: note = Synthetic: Target sequences for COL8A2 Leu450Trp Mutation
  source 1..23: mol_type = other DNA
tgggggctccctgggcagcctgg

SEQ ID NO: 1077 - DNA, 23 bp, synthetic construct
  misc_feature 1..23: note = Synthetic: Target sequences for COL8A2 Leu450Trp Mutation
  source 1..23: mol_type = other DNA
aaggtgactgggggctccctggg

SEQ ID NO: 1078 - DNA, 23 bp, synthetic construct
  misc_feature 1..23: note = Synthetic: Target sequences for COL8A2 Leu450Trp Mutation
  source 1..23: mol_type = other DNA
aaaggtgactgggggctccctgg

SEQ ID NO: 1079 - DNA, 23 bp, synthetic construct
  misc_feature 1..23: note = Synthetic: Target sequences for COL8A2 Leu450Trp Mutation
  source 1..23: mol_type = other DNA
tggggcagaaaggtgactggggg

SEQ ID NO: 1080 - DNA, 23 bp, synthetic construct
  misc_feature 1..23: note = Synthetic: Target sequences for COL8A2 Leu450Trp Mutation
  source 1..23: mol_type = other DNA
ctggggcagaaaggtgactgggg

SEQ ID NO: 1081 - DNA, 23 bp, synthetic construct
  misc_feature 1..23: note = Synthetic: Target sequences for COL8A2 Leu450Trp Mutation
  source 1..23: mol_type = other DNA
cccagtcacctttctgccccagg

SEQ ID NO: 1082 - DNA, 23 bp, synthetic construct
  misc_feature 1..23: note = Synthetic: Target sequences for COL8A2 Leu450Trp Mutation
  source 1..23: mol_type = other DNA
cctggggcagaaaggtgactggg

SEQ ID NO: 1083 - DNA, 23 bp, synthetic construct
  misc_feature 1..23: note = Synthetic: Target sequences for COL8A2 Leu450Trp Mutation
  source 1..23: mol_type = other DNA
ccagtcacctttctgccccaggg

SEQ ID NO: 1084 - DNA, 23 bp, synthetic construct
  misc_feature 1..23: note = Synthetic: Target sequences for COL8A2 Leu450Trp Mutation
  source 1..23: mol_type = other DNA
ccctggggcagaaaggtgactgg

SEQ ID NO: 1085 - DNA, 1857 bp, synthetic construct
  misc_feature 1..1857: note = Synthetic: TCF4 intron 3 sequence with flanking exons, reverse strand (GRCh37/hg19)
  source 1..1857: mol_type = other DNA
gtttgtgtgattttgctaaaatgcatcaccaacagcgaatggctgccttagggacggacaaagagctgagtgatttactggatttcagtgcggtaagaaagaacggtggaaactaacaacagctgtgaaaaaaacaaaacaaaaacccaaacacttcagctagaaaccagtaggaatctaaaggacagtaataatttttaattggctgaatccttggtaaatatgaaggtctttttgacaagtttttaactataattttgtggtgtgatggaagattcaggcttttttttttttttgagttttattactggccttcaattccctacccactgattaccccaaataatggaatctcaccccagtggaaagcaaaaatagacacccctaaaactaaaccacccctaaaacttggccatgtctgaacactgagactactaatactttgcacactactcttcgttttatttattgtttttggaaatggaaaatagaaaataggagacccagttgtctctttaaagttttaagctaatgatgctttggattggtaggacctgttccttacatcttacctcctagttacatcttttcctaggattcttaaaactagtatggatatgctgagcatacattctttagaaccttttggactgttttggtaaatttcgtagtcgtaggatcagcacaaagcggaacttgacacacttgtggagttttacggctgtacttggtccttctccatccctttgcttccttttcctaaaccaagtcccagacatgtcaggagaatgaattcatttttaatgccagatgagtttggtgtaagatgcatttgtaaagcaaaataaaaagaatccacaaaacacacaaataaaatccaaaccgccttccaagtggggctctttcatgctgctgctgctgctgctgctgctgctgctgctgctgctgctgctgctgctgctgctgctgctgctgctgctgctcctcctcctcctcctccttctcctcctcctcctcctcttctagaccttcttttggagaaatggctttcggaagttttgccaggaaacgtagccctaggcaggcagctttgcagccccctttctgcttgttgcactttctccattcgttcctttgctttttgcaggctctgactcagggaaggtgtgcattatccactagatacgtcgaagaagagggaaaccaattagggtcgaaataaatgctggagagagagggagtgaaagagagagtgagagtgagagagagagagagtcttgcttcaaattgctctcctgttagagacgaaatgagaatttagtgcaggtggcacttttatttttatttgggttcacatatgacaggcaaatcctatacgagatggaaatggacattgccacgtttatggccaaggttttcaatataaaacaaaacaacttttttcttctccttggtgaaactagtgtttttctagagaggctgctggcctccaacctgaatcttgataacattatggggactgtgtttgttccaaatgtagcagtagtactgcttggccatctaatgaacctgaggaaaaagaaagaacagagtgataatgggggctggggtgggatctgtaatgttgtttctcttttagttttaagttggatggtgatgtattttactaaataaacccttagcataaactctaagctgtttggtaacagtatgaaagatctttgaggagctctgaaggcacaagtgtcttcttttcaactgtaatatttctttgtttcttttagatgttttcacctcctgtgagcagtgggaaaaatggaccaacttctttggcaagtggacattttactggctcaa

SEQ ID NO: 1086 - RNA, 100 nt, synthetic construct
  misc_feature 1..100: note = Synthetic: sgRNA modified sequence
  modified_base 1..3: mod_base = OTHER; note = 2'-O-methyl nucleotide, phosphorothioate linkage
  variation 1..20: note = n may be any natural or non-natural nucleotide
  modified_base 29..40: mod_base = OTHER; note = 2'-O-methyl nucleotide
  modified_base 69..100: mod_base = OTHER; note = 2'-O-methyl nucleotide
  modified_base 97..99: mod_base = OTHER; note = phosphorothioate linkage
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 1087 - RNA, 42 nt, synthetic construct
  misc_feature 1..42: note = Synthetic: crRNA sequence
  variation 1..20: note = n may be any natural or non-natural nucleotide
  source 1..42: mol_type = other RNA
nnnnnnnnnnnnnnnnnnnngttttagagctatgctgttttg

SEQ ID NO: 1088 - RNA, 74 nt, synthetic construct
  misc_feature 1..74: note = Synthetic: trRNA sequence
  source 1..74: mol_type = other RNA
aacagcatagcaagttaaaataaggctagtccgttatcaacttgaaaaagtggcaccgagtcggtgcttttttt

SEQ ID NO: 1089 - RNA, 20 nt, synthetic construct
  misc_feature 1..20: note = Synthetic: Guide Sequence
  source 1..20: mol_type = other RNA
ttggcaagtggacattttac

SEQ ID NO: 1090 - RNA, 20 nt, synthetic construct
  misc_feature 1..20: note = Synthetic: Guide Sequence
  source 1..20: mol_type = other RNA
tgtccacttgccaaagaagt

SEQ ID NO: 1091 - RNA, 20 nt, synthetic construct
  misc_feature 1..20: note = Synthetic: Guide Sequence
  source 1..20: mol_type = other RNA
ggaccaacttctttggcaag

SEQ ID NO: 1092 - RNA, 20 nt, synthetic construct
  misc_feature 1..20: note = Synthetic: Guide Sequence
  source 1..20: mol_type = other RNA
gaaaaatggaccaacttctt

SEQ ID NO: 1093 - RNA, 20 nt, synthetic construct
  misc_feature 1..20: note = Synthetic: Guide Sequence
  source 1..20: mol_type = other RNA
ccatttttcccactgctcac

SEQ ID NO: 1094 - RNA, 20 nt, synthetic construct
  misc_feature 1..20: note = Synthetic: Guide Sequence
  source 1..20: mol_type = other RNA
cctgtgagcagtgggaaaaa

SEQ ID NO: 1095 - RNA, 20 nt, synthetic construct
  misc_feature 1..20: note = Synthetic: Guide Sequence
  source 1..20: mol_type = other RNA
tttttcccactgctcacagg

SEQ ID NO: 1096 - RNA, 20 nt, synthetic construct
  misc_feature 1..20: note = Synthetic: Guide Sequence
  source 1..20: mol_type = other RNA
tttcacctcctgtgagcagt

SEQ ID NO: 1097 - RNA, 20 nt, synthetic construct
  misc_feature 1..20: note = Synthetic: Guide Sequence
  source 1..20: mol_type = other RNA
ttttcacctcctgtgagcag

SEQ ID NO: 1098 - RNA, 20 nt, synthetic construct
  misc_feature 1..20: note = Synthetic: Guide Sequence
  source 1..20: mol_type = other RNA
agatctttgaggagctctga

SEQ ID NO: 1099 - RNA, 20 nt, synthetic construct
  misc_feature 1..20: note = Synthetic: Guide Sequence
  source 1..20: mol_type = other RNA
aacagtatgaaagatctttg

SEQ ID NO: 1100 - RNA, 20 nt, synthetic construct
  misc_feature 1..20: note = Synthetic: Guide Sequence
  source 1..20: mol_type = other RNA
agcataaactctaagctgtt

SEQ ID NO: 1101 - RNA, 20 nt, synthetic construct
  misc_feature 1..20: note = Synthetic: Guide Sequence
  source 1..20: mol_type = other RNA
acagcttagagtttatgcta

SEQ ID NO: 1102 - RNA, 20 nt, synthetic construct
  misc_feature 1..20: note = Synthetic: Guide Sequence
  source 1..20: mol_type = other RNA
cagcttagagtttatgctaa

SEQ ID NO: 1103 - RNA, 20 nt, synthetic construct
  misc_feature 1..20: note = Synthetic: Guide Sequence
  source 1..20: mol_type = other RNA
tcttttagttttaagttgga

SEQ ID NO: 1104 - RNA, 20 nt, synthetic construct
  misc_feature 1..20: note = Synthetic: Guide Sequence
  source 1..20: mol_type = other RNA
tttctcttttagttttaagt

SEQ ID NO: 1105 - RNA, 20 nt, synthetic construct
  misc_feature 1..20: note = Synthetic: Guide Sequence
  source 1..20: mol_type = other RNA
gtgataatgggggctggggt

SEQ ID NO: 1106 - RNA, 20 nt, synthetic construct
  misc_feature 1..20: note = Synthetic: Guide Sequence
  source 1..20: mol_type = other RNA
agtgataatgggggctgggg

SEQ ID NO: 1107 - RNA, 20 nt, synthetic construct
  misc_feature 1..20: note = Synthetic: Guide Sequence
  source 1..20: mol_type = other RNA
cagagtgataatgggggctg

SEQ ID NO: 1108 - RNA, 20 nt, synthetic construct
  misc_feature 1..20: note = Synthetic: Guide Sequence
  source 1..20: mol_type = other RNA
acagagtgataatgggggct

SEQ ID NO: 1109 - RNA, 20 nt, synthetic construct
  misc_feature 1..20: note = Synthetic: Guide Sequence
  source 1..20: mol_type = other RNA
aacagagtgataatgggggc

SEQ ID NO: 1110 - RNA, 20 nt, synthetic construct
  misc_feature 1..20: note = Synthetic: Guide Sequence
  source 1..20: mol_type = other RNA
aaagaacagagtgataatgg

SEQ ID NO: 1111 - RNA, 20 nt, synthetic construct
  misc_feature 1..20: note = Synthetic: Guide Sequence
  source 1..20: mol_type = other RNA
gaaagaacagagtgataatg

SEQ ID NO: 1112 - RNA, 20 nt, synthetic construct
  misc_feature 1..20: note = Synthetic: Guide Sequence
  source 1..20: mol_type = other RNA
agaaagaacagagtgataat

SEQ ID NO: 1113 - RNA, 20 nt, synthetic construct
  misc_feature 1..20: note = Synthetic: Guide Sequence
  source 1..20: mol_type = other RNA
aagaaagaacagagtgataa

SEQ ID NO: 1114 - RNA, 20 nt, synthetic construct
  misc_feature 1..20: note = Synthetic: Guide Sequence
  source 1..20: mol_type = other RNA
tctgttctttctttttcctc

SEQ ID NO: 1115 - RNA, 20 nt, synthetic construct
  misc_feature 1..20: note = Synthetic: Guide Sequence
  source 1..20: mol_type = other RNA
ttttcctcaggttcattaga

SEQ ID NO: 1116 - RNA, 20 nt, synthetic construct
  misc_feature 1..20: note = Synthetic: Guide Sequence
  source 1..20: mol_type = other RNA
ttggccatctaatgaacctg

SEQ ID NO: 1117 - RNA, 20 nt, synthetic construct
  misc_feature 1..20: note = Synthetic: Guide Sequence
  source 1..20: mol_type = other RNA
aatgtagcagtagtactgct

SEQ ID NO: 1118 - RNA, 20 nt, synthetic construct
  misc_feature 1..20: note = Synthetic: Guide Sequence
  source 1..20: mol_type = other RNA
agcagtactactgctacatt

SEQ ID NO: 1119 - RNA, 20 nt, synthetic construct
  misc_feature 1..20: note = Synthetic: Guide Sequence
  source 1..20: mol_type = other RNA
tgaatcttgataacattatg

SEQ ID NO: 1120 - RNA, 20 nt, synthetic construct
  misc_feature 1..20: note = Synthetic: Guide Sequence
  source 1..20: mol_type = other RNA
ctgaatcttgataacattat

SEQ ID NO: 1121 - RNA, 20 nt, synthetic construct
  misc_feature 1..20: note = Synthetic: Guide Sequence
  source 1..20: mol_type = other RNA
ccataatgttatcaagattc

SEQ ID NO: 1122 - RNA, 20 nt, synthetic construct
  misc_feature 1..20: note = Synthetic: Guide Sequence
  source 1..20: mol_type = other RNA
cctgaatcttgataacatta

SEQ ID NO: 1123 - RNA, 20 nt, synthetic construct
  misc_feature 1..20: note = Synthetic: Guide Sequence
  source 1..20: mol_type = other RNA
aatgttatcaagattcaggt

SEQ ID NO: 1124 - RNA, 20 nt, synthetic construct
  misc_feature 1..20: note = Synthetic: Guide Sequence
  source 1..20: mol_type = other RNA
gttatcaagattcaggttgg

SEQ ID NO: 1125 - RNA, 20 nt, synthetic construct
  misc_feature 1..20: note = Synthetic: Guide Sequence
  source 1..20: mol_type = other RNA
tgtttttctagagaggctgc

SEQ ID NO: 1126 - RNA, 20 nt, synthetic construct
  misc_feature 1..20: note = Synthetic: Guide Sequence
  source 1..20: mol_type = other RNA
aaactagtgtttttctagag

SEQ ID NO: 1127 - RNA, 20 nt, synthetic construct
  misc_feature 1..20: note = Synthetic: Guide Sequence
  source 1..20: mol_type = other RNA
gaaaaacactagtttcacca

SEQ ID NO: 1128 - RNA, 20 nt, synthetic construct
  misc_feature 1..20: note = Synthetic: Guide Sequence
  source 1..20: mol_type = other RNA
aacaacttttttcttctcct

SEQ ID NO: 1129 - RNA, 20 nt, synthetic construct
  misc_feature 1..20: note = Synthetic: Guide Sequence
  source 1..20: mol_type = other RNA
ttgttttatattgaaaacct

SEQ ID NO: 1130 - RNA, 20 nt, synthetic construct
  misc_feature 1..20: note = Synthetic: Guide Sequence
  source 1..20: mol_type = other RNA
gaaaaccttggccataaacg

SEQ ID NO: 1131 - RNA, 20 nt, synthetic construct
  misc_feature 1..20: note = Synthetic: Guide Sequence
  source 1..20: mol_type = other RNA
cattgccacgtttatggcca

SEQ ID NO: 1132 - RNA, 20 nt, synthetic construct
  misc_feature 1..20: note = Synthetic: Guide Sequence
  source 1..20: mol_type = other RNA
aatggacattgccacgttta

SEQ ID NO: 1133 - RNA, 20 nt, synthetic construct
  misc_feature 1..20: note = Synthetic: Guide Sequence
  source 1..20: mol_type = other RNA
tgtccatttccatctcgtat

SEQ ID NO: 1134 - RNA, 20 nt, synthetic construct
  misc_feature 1..20: note = Synthetic: Guide Sequence
  source 1..20: mol_type = other RNA
aatcctatacgagatggaaa

SEQ ID NO: 1135 - RNA, 20 nt, synthetic construct
  misc_feature 1..20: note = Synthetic: Guide Sequence
  source 1..20: mol_type = other RNA
caggcaaatcctatacgaga

SEQ ID NO: 1136 - RNA, 20 nt, synthetic construct
  misc_feature 1..20: note = Synthetic: Guide Sequence
  source 1..20: mol_type = other RNA
tatttgggttcacatatgac

SEQ ID NO: 1137 - RNA, 20 nt, synthetic construct
  misc_feature 1..20: note = Synthetic: Guide Sequence
  source 1..20: mol_type = other RNA
tggcacttttatttttattt

SEQ ID NO: 1138 - RNA, 20 nt, synthetic construct
  misc_feature 1..20: note = Synthetic: Guide Sequence
  source 1..20: mol_type = other RNA
gtggcacttttatttttatt

SEQ ID NO: 1139 - RNA, 20 nt, synthetic construct
  misc_feature 1..20: note = Synthetic: Guide Sequence
  source 1..20: mol_type = other RNA
aaatgagaatttagtgcagg

SEQ ID NO: 1140 - RNA, 20 nt, synthetic construct
  misc_feature 1..20: note = Synthetic: Guide Sequence
  source 1..20: mol_type = other RNA
acgaaatgagaatttagtgc

SEQ ID NO: 1141 - RNA, 20 nt, synthetic construct
  misc_feature 1..20: note = Synthetic: Guide Sequence
  source 1..20: mol_type = other RNA
attctcatttcgtctctaac

SEQ ID NO: 1142 - RNA, 20 nt, synthetic construct
  misc_feature 1..20: note = Synthetic: Guide Sequence
  source 1..20: mol_type = other RNA
aaataaatgctggagagaga

SEQ ID NO: 1143 - RNA, 20 nt, synthetic construct
  misc_feature 1..20: note = Synthetic: Guide Sequence
  source 1..20: mol_type = other RNA
gaaataaatgctggagagag

SEQ ID NO: 1144 - RNA, 20 nt, synthetic construct
  misc_feature 1..20: note = Synthetic: Guide Sequence
  source 1..20: mol_type = other RNA
attagggtcgaaataaatgc

SEQ ID NO: 1145 - RNA, 20 nt, synthetic construct
  misc_feature 1..20: note = Synthetic: Guide Sequence
  source 1..20: mol_type = other RNA
gcatttatttcgaccctaat

SEQ ID NO: 1146 - RNA, 20 nt, synthetic construct
  misc_feature 1..20: note = Synthetic: Guide Sequence
  source 1..20: mol_type = other RNA
aagaagagggaaaccaatta

SEQ ID NO: 1147 - RNA, 20 nt, synthetic construct
  misc_feature 1..20: note = Synthetic: Guide Sequence
  source 1..20: mol_type = other RNA
gaagaagagggaaaccaatt

SEQ ID NO: 1148 - RNA, 20 nt, synthetic construct
  misc_feature 1..20: note = Synthetic: Guide Sequence
  source 1..20: mol_type = other RNA
actagatacgtcgaagaaga

SEQ ID NO: 1149 - RNA, 20 nt, synthetic construct
  misc_feature 1..20: note = Synthetic: Guide Sequence
  source 1..20: mol_type = other RNA
cactagatacgtcgaagaag

SEQ ID NO: 1150 - RNA, 20 nt, synthetic construct
  misc_feature 1..20: note = Synthetic: Guide Sequence
  source 1..20: mol_type = other RNA
ctcttcttcgacgtatctag

SEQ ID NO: 1151 - RNA, 20 nt, synthetic construct
  misc_feature 1..20: note = Synthetic: Guide Sequence
  source 1..20: mol_type = other RNA
tgcaggctctgactcaggga

SEQ ID NO: 1152 - RNA, 20 nt, synthetic construct
  misc_feature 1..20: note = Synthetic: Guide Sequence
  source 1..20: mol_type = other RNA
tttttgcaggctctgactca

SEQ ID NO: 1153 - RNA, 20 nt, synthetic construct
  misc_feature 1..20: note = Synthetic: Guide Sequence
  source 1..20: mol_type = other RNA
ctttttgcaggctctgactc

SEQ ID NO: 1154 - RNA, 20 nt, synthetic construct
  misc_feature 1..20: note = Synthetic: Guide Sequence
  source 1..20: mol_type = other RNA
tcagagcctgcaaaaagcaa

SEQ ID NO: 1155 - RNA, 20 nt, synthetic construct
  misc_feature 1..20: note = Synthetic: Guide Sequence
  source 1..20: mol_type = other RNA
ttcgttcctttgctttttgc

SEQ ID NO: 1156 - DNA, 20 bp, synthetic construct
  misc_feature 1..20: note = Synthetic: Guide Sequence
  source 1..20: mol_type = other DNA
gcaaaaagcaaaggaacgaa

SEQ ID NO: 1157 - RNA, 20 nt, synthetic construct
  misc_feature 1..20: note = Synthetic: Guide Sequence
  source 1..20: mol_type = other RNA
agaaagtgcaacaagcagaa

SEQ ID NO: 1158 - RNA, 20 nt, synthetic construct
  misc_feature 1..20: note = Synthetic: Guide Sequence
  source 1..20: mol_type = other RNA
gaaagtgcaacaagcagaaa

SEQ ID NO: 1159 - RNA, 20 nt, synthetic construct
  misc_feature 1..20: note = Synthetic: Guide Sequence
  source 1..20: mol_type = other RNA
aaagtgcaacaagcagaaag

SEQ ID NO: 1160 - RNA, 20 nt, synthetic construct
  misc_feature 1..20: note = Synthetic: Guide Sequence
  source 1..20: mol_type = other RNA
aagtgcaacaagcagaaagg

SEQ ID NO: 1161 - RNA, 20 nt, synthetic construct
  misc_feature 1..20: note = Synthetic: Guide Sequence
  source 1..20: mol_type = other RNA
ggctgcaaagctgcctgcct

SEQ ID NO: 1162 - RNA, 20 nt, synthetic construct
  misc_feature 1..20: note = Synthetic: Guide Sequence
  source 1..20: mol_type = other RNA
gctgcaaagctgcctgccta

SEQ ID NO: 1163 - RNA, 20 nt, synthetic construct
  misc_feature 1..20: note = Synthetic: Guide Sequence
  source 1..20: mol_type = other RNA
caggaaacgtagccctaggc

SEQ ID NO: 1164 - RNA, 20 nt, synthetic construct
  misc_feature 1..20: note = Synthetic: Guide Sequence
  source 1..20: mol_type = other RNA
ctgcctagggctacgtttcc

SEQ ID NO: 1165 - RNA, 20 nt, synthetic construct
  misc_feature 1..20: note = Synthetic: Guide Sequence
  source 1..20: mol_type = other RNA
ttgccaggaaacgtagccct

SEQ ID NO: 1166 - RNA, 20 nt, synthetic construct
  misc_feature 1..20: note = Synthetic: Guide Sequence
  source 1..20: mol_type = other RNA
tggctttcggaagttttgcc

SEQ ID NO: 1167 - RNA, 20 nt, synthetic construct
  misc_feature 1..20: note = Synthetic: Guide Sequence
  source 1..20: mol_type = other RNA
tcttttggagaaatggcttt

SEQ ID NO: 1168 - RNA, 20 nt, synthetic construct
  misc_feature 1..20: note = Synthetic: Guide Sequence
  source 1..20: mol_type = other RNA
aaagccatttctccaaaaga

SEQ ID NO: 1169 - RNA, 20 nt, synthetic construct
  misc_feature 1..20: note = Synthetic: Guide Sequence
  source 1..20: mol_type = other RNA
tagaccttcttttggagaaa

SEQ ID NO: 1170 - RNA, 20 nt, synthetic construct
  misc_feature 1..20: note = Synthetic: Guide Sequence
  source 1..20: mol_type = other RNA
tccaaaagaaggtctagaag

SEQ ID NO: 1171 - RNA, 20 nt, synthetic construct
  misc_feature 1..20: note = Synthetic: Guide Sequence
  source 1..20: mol_type = other RNA
tcctcttctagaccttcttt

SEQ ID NO: 1172 - RNA, 20 nt, synthetic construct
  misc_feature 1..20: note = Synthetic: Guide Sequence
  source 1..20: mol_type = other RNA
aaaagaaggtctagaagagg

SEQ ID NO: 1173 - RNA, 20 nt, synthetic construct
  misc_feature 1..20: note = Synthetic: Guide Sequence
  source 1..20: mol_type = other RNA
agaaggtctagaagaggagg

SEQ ID NO: 1174 - RNA, 20 nt, synthetic construct
  misc_feature 1..20: note = Synthetic: Guide Sequence
  source 1..20: mol_type = other RNA
aggtctagaagaggaggagg

SEQ ID NO: 1175 - RNA, 20 nt, synthetic construct
  misc_feature 1..20: note = Synthetic: Guide Sequence
  source 1..20: mol_type = other RNA
tctagaagaggaggaggagg

SEQ ID NO: 1176 - DNA, 20 bp, synthetic construct
  misc_feature 1..20: note = Synthetic: Guide Sequence
  source 1..20: mol_type = other DNA
agaggaggaggaggaggaga

SEQ ID NO: 1177 - DNA, 20 bp, synthetic construct
  misc_feature 1..20: note = Synthetic: Guide Sequence
  source 1..20: mol_type = other DNA
ggaggaggaggaggagaagg

SEQ ID NO: 1178 - DNA, 20 bp, synthetic construct
  misc_feature 1..20: note = Synthetic: Guide Sequence
  source 1..20: mol_type = other DNA
ggaggaggaggagaaggagg

SEQ ID NO: 1179 - DNA, 20 bp, synthetic construct
  misc_feature 1..20: note = Synthetic: Guide Sequence
  source 1..20: mol_type = other DNA
ggaggaggagaaggaggagg

SEQ ID NO: 1180 - DNA, 20 bp, synthetic construct
  misc_feature 1..20: note = Synthetic: Guide Sequence
  source 1..20: mol_type = other DNA
ggaggagaaggaggaggagg

SEQ ID NO: 1181 - DNA, 20 bp, synthetic construct
  misc_feature 1..20: note = Synthetic: Guide Sequence
  source 1..20: mol_type = other DNA
ggagaaggaggaggaggagg

SEQ ID NO: 1182 - RNA, 20 nt, synthetic construct
  misc_feature 1..20: note = Synthetic: Guide Sequence
  source 1..20: mol_type = other RNA
cagcatgaaagagccccact

SEQ ID NO: 1183 - RNA, 20 nt, synthetic construct
  misc_feature 1..20: note = Synthetic: Guide Sequence
  source 1..20: mol_type = other RNA
atgaaagagccccacttgga

SEQ ID NO: 1184 - RNA, 20 nt, synthetic construct
  misc_feature 1..20: note = Synthetic: Guide Sequence
  source 1..20: mol_type = other RNA
aaagagccccacttggaagg

SEQ ID NO: 1185 - RNA, 20 nt, synthetic construct
  misc_feature 1..20: note = Synthetic: Guide Sequence
  source 1..20: mol_type = other RNA
gccccacttggaaggcggtt

SEQ ID NO: 1186 - RNA, 20 nt, synthetic construct
  misc_feature 1..20: note = Synthetic: Guide Sequence
  source 1..20: mol_type = other RNA
tccaaaccgccttccaagtg

SEQ ID NO: 1187 - RNA, 20 nt, synthetic construct
  misc_feature 1..20: note = Synthetic: Guide Sequence
  source 1..20: mol_type = other RNA
atccaaaccgccttccaagt

SEQ ID NO: 1188 - RNA, 20 nt, synthetic construct
  misc_feature 1..20: note = Synthetic: Guide Sequence
  source 1..20: mol_type = other RNA
aatccaaaccgccttccaag

SEQ ID NO: 1189 - RNA, 20 nt, synthetic construct
  misc_feature 1..20: note = Synthetic: Guide Sequence
  source 1..20: mol_type = other RNA
gattttatttgtgtgttttg

SEQ ID NO: 1190 - RNA, 20 nt, synthetic construct
  misc_feature 1..20: note = Synthetic: Guide Sequence
  source 1..20: mol_type = other RNA
catcttacaccaaactcatc

SEQ ID NO: 1191 - RNA, 20 nt, synthetic construct
  misc_feature 1..20: note = Synthetic: Guide Sequence
  source 1..20: mol_type = other RNA
tttttaatgccagatgagtt

SEQ ID NO: 1192 - RNA, 20 nt, synthetic construct
  misc_feature 1..20: note = Synthetic: Guide Sequence
  source 1..20: mol_type = other RNA
attcattctcctgacatgtc

SEQ ID NO: 1193 - RNA, 20 nt, synthetic construct
  misc_feature 1..20: note = Synthetic: Guide Sequence
  source 1..20: mol_type = other RNA
ttcattctcctgacatgtct

SEQ ID NO: 1194 - RNA, 20 nt, synthetic construct
  misc_feature 1..20: note = Synthetic: Guide Sequence
  source 1..20: mol_type = other RNA
ctcctgacatgtctgggact

SEQ ID NO: 1195 - RNA, 20 nt, synthetic construct
  misc_feature 1..20: note = Synthetic: Guide Sequence
  source 1..20: mol_type = other RNA
aaccaagtcccagacatgtc

SEQ ID NO: 1196 - RNA, 20 nt, synthetic construct
  misc_feature 1..20: note = Synthetic: Guide Sequence
  source 1..20: mol_type = other RNA
acatgtctgggacttggttt

SEQ ID NO: 1197 - RNA, 20 nt, synthetic construct
  misc_feature 1..20: note = Synthetic: Guide Sequence
  source 1..20: mol_type = other RNA
ctgggacttggtttaggaaa

SEQ ID NO: 1198 - RNA, 20 nt, synthetic construct
  misc_feature 1..20: note = Synthetic: Guide Sequence
  source 1..20: mol_type = other RNA
ggtttaggaaaaggaagcaa

SEQ ID NO: 1199 - RNA, 20 nt, synthetic construct
  misc_feature 1..20: note = Synthetic: Guide Sequence
  source 1..20: mol_type = other RNA
gtttaggaaaaggaagcaaa

SEQ ID NO: 1200 - DNA, 20 bp, synthetic construct
  misc_feature 1..20: note = Synthetic: Guide Sequence
  source 1..20: mol_type = other DNA
aggaaaaggaagcaaaggga

SEQ ID NO: 1201 - RNA, 20 nt, synthetic construct
  misc_feature 1..20: note = Synthetic: Guide Sequence
  source 1..20: mol_type = other RNA
aggaagcaaagggatggaga

SEQ ID NO: 1202 - RNA, 20 nt, synthetic construct
  misc_feature 1..20: note = Synthetic: Guide Sequence
  source 1..20: mol_type = other RNA
tggagttttacggctgtact

SEQ ID NO: 1203 - RNA, 20 nt, synthetic construct
  misc_feature 1..20: note = Synthetic: Guide Sequence
  source 1..20: mol_type = other RNA
gacacacttgtggagtttta

SEQ ID NO: 1204 - RNA, 20 nt, synthetic construct
  misc_feature 1..20: note = Synthetic: Guide Sequence
  source 1..20: mol_type = other RNA
agcggaacttgacacacttg

SEQ ID NO: 1205 - RNA, 20 nt, synthetic construct
  misc_feature 1..20: note = Synthetic: Guide Sequence
  source 1..20: mol_type = other RNA
gtcgtaggatcagcacaaag

SEQ ID NO: 1206 - RNA, 20 nt, synthetic construct
  misc_feature 1..20: note = Synthetic: Guide Sequence
  source 1..20: mol_type = other RNA
ttggtaaatttcgtagtcgt

SEQ ID NO: 1207 - RNA, 20 nt, synthetic construct
  misc_feature 1..20: note = Synthetic: Guide Sequence
  source 1..20: mol_type = other RNA
atttaccaaaacagtccaaa

SEQ ID NO: 1208 - RNA, 20 nt, synthetic construct
  misc_feature 1..20: note = Synthetic: Guide Sequence
  source 1..20: mol_type = other RNA
tagaaccttttggactgttt

SEQ ID NO: 1209 - RNA, 20 nt, synthetic construct
  misc_feature 1..20: note = Synthetic: Guide Sequence
  source 1..20: mol_type = other RNA
atacattctttagaaccttt

SEQ ID NO: 1210 - RNA, 20 nt, synthetic construct
  misc_feature 1..20: note = Synthetic: Guide Sequence
  source 1..20: mol_type = other RNA
taggattcttaaaactagta

SEQ ID NO: 1211 - RNA, 20 nt, synthetic construct
  misc_feature 1..20: note = Synthetic: Guide Sequence
  source 1..20: mol_type = other RNA
atactagttttaagaatcct

SEQ ID NO: 1212 - RNA, 20 nt, synthetic construct
  misc_feature 1..20: note = Synthetic: Guide Sequence
  source 1..20: mol_type = other RNA
tcctaggaaaagatgtaact

SEQ ID NO: 1213 - RNA, 20 nt, synthetic construct
  misc_feature 1..20: note = Synthetic: Guide Sequence
  source 1..20: mol_type = other RNA
tcctagttacatcttttcct

SEQ ID NO: 1214 - RNA, 20 nt, synthetic construct
  misc_feature 1..20: note = Synthetic: Guide Sequence
  source 1..20: mol_type = other RNA
taggaaaagatgtaactagg

SEQ ID NO: 1215 - RNA, 20 nt, synthetic construct
  misc_feature 1..20: note = Synthetic: Guide Sequence
  source 1..20: mol_type = other RNA
taactaggaggtaagatgta

SEQ ID NO: 1216 - RNA, 20 nt, synthetic construct
  misc_feature 1..20: note = Synthetic: Guide Sequence
  source 1..20: mol_type = other RNA
ggaggtaagatgtaaggaac

SEQ ID NO: 1217 - RNA, 20 nt, synthetic construct
  misc_feature 1..20: note = Synthetic: Guide Sequence
  source 1..20: mol_type = other RNA
taatgatgctttggattggt

SEQ ID NO: 1218 - RNA, 20 nt, synthetic construct
  misc_feature 1..20: note = Synthetic: Guide Sequence
  source 1..20: mol_type = other RNA
aagctaatgatgctttggat

SEQ ID NO: 1219 - RNA, 20 nt, synthetic construct
  misc_feature 1..20: note = Synthetic: Guide Sequence
  source 1..20: mol_type = other RNA
gttttaagctaatgatgctt

SEQ ID NO: 1220 - RNA, 20 nt, synthetic construct
  misc_feature 1..20: note = Synthetic: Guide Sequence
  source 1..20: mol_type = other RNA
taaaactttaaagagacaac

SEQ ID NO: 1221 - RNA, 20 nt, synthetic construct
  misc_feature 1..20: note = Synthetic: Guide Sequence
  source 1..20: mol_type = other RNA
aaaactttaaagagacaact

SEQ ID NO: 1222 - RNA, 20 nt, synthetic construct
  misc_feature 1..20: note = Synthetic: Guide Sequence
  source 1..20: mol_type = other RNA
ggaaatggaaaatagaaaat

SEQ ID NO: 1223 - RNA, 20 nt, synthetic construct
  misc_feature 1..20: note = Synthetic: Guide Sequence
  source 1..20: mol_type = other RNA
ttatttattgtttttggaaa

SEQ ID NO: 1224 - RNA, 20 nt, synthetic construct
  misc_feature 1..20: note = Synthetic: Guide Sequence
  source 1..20: mol_type = other RNA
ttcgttttatttattgtttt

SEQ ID NO: 1225 - RNA, 20 nt, synthetic construct
  misc_feature 1..20: note = Synthetic: Guide Sequence
  source 1..20: mol_type = other RNA
gtagtctcagtgttcagaca

SEQ ID NO: 1226 - RNA, 20 nt, synthetic construct
  misc_feature 1..20: note = Synthetic: Guide Sequence
  source 1..20: mol_type = other RNA
ttcagacatggccaagtttt

SEQ ID NO: 1227 - RNA, 20 nt, synthetic construct
  misc_feature 1..20: note = Synthetic: Guide Sequence
  source 1..20: mol_type = other RNA
tcagacatggccaagtttta

SEQ ID NO: 1228 - RNA, 20 nt, synthetic construct
  misc_feature 1..20: note = Synthetic: Guide Sequence
  source 1..20: mol_type = other RNA
cagacatggccaagttttag

SEQ ID NO: 1229 - RNA, 20 nt, synthetic construct
  misc_feature 1..20: note = Synthetic: Guide Sequence
  source 1..20: mol_type = other RNA
acatggccaagttttagggg

SEQ ID NO: 1230 - RNA, 20 nt, synthetic construct
  misc_feature 1..20: note = Synthetic: Guide Sequence
  source 1..20: mol_type = other RNA
actaaaccacccctaaaact

SEQ ID NO: 1231 - RNA, 20 nt, synthetic construct
  misc_feature 1..20: note = Synthetic: Guide Sequence
  source 1..20: mol_type = other RNA
tttaggggtggtttagtttt

SEQ ID NO: 1232 - RNA, 20 nt, synthetic construct
  misc_feature 1..20: note = Synthetic: Guide Sequence
  source 1..20: mol_type = other RNA
ttaggggtggtttagtttta

SEQ ID NO: 1233 - RNA, 20 nt, synthetic construct
  misc_feature 1..20: note = Synthetic: Guide Sequence
  source 1..20: mol_type = other RNA
taggggtggtttagttttag

SEQ ID NO: 1234 - RNA, 20 nt, synthetic construct
  misc_feature 1..20: note = Synthetic: Guide Sequence
  source 1..20: mol_type = other RNA
tgtctatttttgctttccac

SEQ ID NO: 1235 - RNA, 20 nt, synthetic construct
  misc_feature 1..20: note = Synthetic: Guide Sequence
  source 1..20: mol_type = other RNA
gtctatttttgctttccact

SEQ ID NO: 1236 - RNA, 20 nt, synthetic construct
  misc_feature 1..20: note = Synthetic: Guide Sequence
  source 1..20: mol_type = other RNA
tctatttttgctttccactg

SEQ ID NO: 1237 - RNA, 20 nt, synthetic construct
  misc_feature 1..20: note = Synthetic: Guide Sequence
  source 1..20: mol_type = other RNA
ataatggaatctcaccccag

SEQ ID NO: 1238 - RNA, 20 nt, synthetic construct
  misc_feature 1..20: note = Synthetic: Guide Sequence
  source 1..20: mol_type = other RNA
tggggtgagattccattatt

SEQ ID NO: 1239 - RNA, 20 nt, synthetic construct
  misc_feature 1..20: note = Synthetic: Guide Sequence
  source 1..20: mol_type = other RNA
ggggtgagattccattattt

SEQ ID NO: 1240 - RNA, 20 nt, synthetic construct
  misc_feature 1..20: note = Synthetic: Guide Sequence
  source 1..20: mol_type = other RNA
gggtgagattccattatttg

SEQ ID NO: 1241 - RNA, 20 nt, synthetic construct
  misc_feature 1..20: note = Synthetic: Guide Sequence
  source 1..20: mol_type = other RNA
ccattatttggggtaatcag

SEQ ID NO: 1242 - RNA, 20 nt, synthetic construct
  misc_feature 1..20: note = Synthetic: Guide Sequence
  source 1..20: mol_type = other RNA
ccactgattaccccaaataa

SEQ ID NO: 1243 - RNA, 20 nt, synthetic construct
  misc_feature 1..20: note = Synthetic: Guide Sequence
  source 1..20: mol_type = other RNA
cattatttggggtaatcagt

SEQ ID NO: 1244 - RNA, 20 nt, synthetic construct
  misc_feature 1..20: note = Synthetic: Guide Sequence
  source 1..20: mol_type = other RNA
atttggggtaatcagtgggt

SEQ ID NO: 1245 - RNA, 20 nt, synthetic construct
  misc_feature 1..20: note = Synthetic: Guide Sequence
  source 1..20: mol_type = other RNA
tttggggtaatcagtgggta

SEQ ID NO: 1246 - RNA, 20 nt, synthetic construct
  misc_feature 1..20: note = Synthetic: Guide Sequence
  source 1..20: mol_type = other RNA
atcagtgggtagggaattga

SEQ ID NO: 1247 - RNA, 20 nt, synthetic construct
  misc_feature 1..20: note = Synthetic: Guide Sequence
  source 1..20: mol_type = other RNA
ttttttttgagttttattac

SEQ ID NO: 1248 - RNA, 20 nt, synthetic construct
  misc_feature 1..20: note = Synthetic: Guide Sequence
  source 1..20: mol_type = other RNA
tgtggtgtgatggaagattc

SEQ ID NO: 1249 - RNA, 20 nt, synthetic construct
  misc_feature 1..20: note = Synthetic: Guide Sequence
  source 1..20: mol_type = other RNA
actataattttgtggtgtga

SEQ ID NO: 1250 - RNA, 20 nt, synthetic construct
  misc_feature 1..20: note = Synthetic: Guide Sequence
  source 1..20: mol_type = other RNA
agtttttaactataattttg

SEQ ID NO: 1251 - RNA, 20 nt, synthetic construct
  misc_feature 1..20: note = Synthetic: Guide Sequence
  source 1..20: mol_type = other RNA
aaagaccttcatatttacca

SEQ ID NO: 1252 - RNA, 20 nt, synthetic construct
  misc_feature 1..20: note = Synthetic: Guide Sequence
  source 1..20: mol_type = other RNA
tgaatccttggtaaatatga

SEQ ID NO: 1253 - RNA, 20 nt, synthetic construct
  misc_feature 1..20: note = Synthetic: Guide Sequence
  source 1..20: mol_type = other RNA
tttttaattggctgaatcct

SEQ ID NO: 1254 - RNA, 20 nt, synthetic construct
  misc_feature 1..20: note = Synthetic: Guide Sequence
  source 1..20: mol_type = other RNA
ggacagtaataatttttaat

SEQ ID NO: 1255 - RNA, 20 nt, synthetic construct
  misc_feature 1..20: note = Synthetic: Guide Sequence
  source 1..20: mol_type = other RNA
actgtcctttagattcctac

SEQ ID NO: 1256 - RNA, 20 nt, synthetic construct
  misc_feature 1..20: note = Synthetic: Guide Sequence
  source 1..20: mol_type = other RNA
agaaaccagtaggaatctaa

SEQ ID NO: 1257 - RNA, 20 nt, synthetic construct
  misc_feature 1..20: note = Synthetic: Guide Sequence
  source 1..20: mol_type = other RNA
cacttcagctagaaaccagt

SEQ ID NO: 1258 - RNA, 20 nt, synthetic construct
  misc_feature 1..20: note = Synthetic: Guide Sequence
  source 1..20: mol_type = other RNA
tggtttctagctgaagtgtt

SEQ ID NO: 1259 - RNA, 20 nt, synthetic construct
  misc_feature 1..20: note = Synthetic: Guide Sequence
  source 1..20: mol_type = other RNA
ggtttctagctgaagtgttt

SEQ ID NO: 1260 - RNA, 20 nt, synthetic construct
  misc_feature 1..20: note = Synthetic: Guide Sequence
  source 1..20: mol_type = other RNA
agtgcggtaagaaagaacgg

SEQ ID NO: 1261 - RNA, 20 nt, synthetic construct
  misc_feature 1..20: note = Synthetic: Guide Sequence
  source 1..20: mol_type = other RNA
ttcagtgcggtaagaaagaa

SEQ ID NO: 1262 - RNA, 20 nt, synthetic construct
  misc_feature 1..20: note = Synthetic: Guide Sequence
  source 1..20: mol_type = other RNA
tgatttactggatttcagtg

SEQ ID NO: 1263 - RNA, 20 nt, synthetic construct
  misc_feature 1..20: note = Synthetic: Guide Sequence
  source 1..20: mol_type = other RNA
caaagagctgagtgatttac

SEQ ID NO: 1264 - RNA, 20 nt, synthetic construct
  misc_feature 1..20: note = Synthetic: Guide Sequence
  source 1..20: mol_type = other RNA
cagctctttgtccgtcccta

SEQ ID NO: 1265 - RNA, 20 nt, synthetic construct
  misc_feature 1..20: note = Synthetic: Guide Sequence
  source 1..20: mol_type = other RNA
gcgaatggctgccttaggga

SEQ ID NO: 1266 - RNA, 20 nt, synthetic construct
  misc_feature 1..20: note = Synthetic: Guide Sequence
  source 1..20: mol_type = other RNA
aacagcgaatggctgcctta

SEQ ID NO: 1267 - RNA, 20 nt, synthetic construct
  misc_feature 1..20: note = Synthetic: Guide Sequence
  source 1..20: mol_type = other RNA
caacagcgaatggctgcctt

SEQ ID NO: 1268 - RNA, 20 nt, synthetic construct
  misc_feature 1..20: note = Synthetic: Guide Sequence
  source 1..20: mol_type = other RNA
ctaaggcagccattcgctgt

SEQ ID NO: 1269 - RNA, 20 nt, synthetic construct
  misc_feature 1..20: note = Synthetic: Guide Sequence
  source 1..20: mol_type = other RNA
aatgcatcaccaacagcgaa

SEQ ID NO: 1270 - RNA, 20 nt, synthetic construct
  misc_feature 1..20: note = Synthetic: Guide Sequence
  source 1..20: mol_type = other RNA
atcacacaaacctagaaaca

SEQ ID NO: 1271 - RNA, 20 nt, synthetic construct
  misc_feature 1..20: note = Synthetic: Guide Sequence
  source 1..20: mol_type = other RNA
gcggttatttccatgtttct

SEQ ID NO: 1272 - RNA, 20 nt, synthetic construct
  misc_feature 1..20: note = Synthetic: Guide Sequence
  source 1..20: mol_type = other RNA
gggactggattttctgattg

SEQ ID NO: 1273 - RNA, 20 nt, synthetic construct
  misc_feature 1..20: note = Synthetic: Guide Sequence
  source 1..20: mol_type = other RNA
gaaaatccagtcccaatcct

SEQ ID NO: 1274 - RNA, 20 nt, synthetic construct
  misc_feature 1..20: note = Synthetic: Guide Sequence
  source 1..20: mol_type = other RNA
ttttctccaaggattgggac

SEQ ID NO: 1275 - RNA, 20 nt, synthetic construct
  misc_feature 1..20: note = Synthetic: Guide Sequence
  source 1..20: mol_type = other RNA
ttgtgttttctccaaggatt

SEQ ID NO: 1276 - RNA, 20 nt, synthetic construct
  misc_feature 1..20: note = Synthetic: Guide Sequence
  source 1..20: mol_type = other RNA
attgtgttttctccaaggat

SEQ ID NO: 1277 - RNA, 20 nt, synthetic construct
  misc_feature 1..20: note = Synthetic: Guide Sequence
  source 1..20: mol_type = other RNA
atccttggagaaaacacaat

SEQ ID NO: 1278 - RNA, 20 nt, synthetic construct
  misc_feature 1..20: note = Synthetic: Guide Sequence
  source 1..20: mol_type = other RNA
atccgattgtgttttctcca

(19) United States
(12) Patent Application Publication
(10) Pub. No.: US 2025/0025580 A1
(43) Pub. Date: Jan. 23, 2025

(54) Compositions and Methods for Treatment of Diseases Associated with Trinucleotide Repeats in Transcription Factor Four

(71) Applicant: Intellia Therapeutics, Inc., Cambridge, MA (US)

(72) Inventors: Sean Michael Burns, Hingham, MA (US); Bradley Andrew Murray, Boston, MA (US); Sarah Beth Hesse, Arlington, MA (US)

(73) Assignee: Intellia Therapeutics, Inc., Cambridge, MA (US)

(21) Appl. No.: 18/415,032

(22) Filed: Jan. 17, 2024

Related U.S. Application Data
(63) Continuation of application No. 16/095,236, filed on Oct. 19, 2018, now abandoned.
(60) Provisional application No. 62/326,700, filed on Apr. 22, 2016.

(51) Int. Cl. A61K 48/00; A61K 9/127; A61K 9/51; A61K 38/46; A61P 25/14; A61P 27/02; C12N 15/10

(52) U.S. Cl. CPC A61K 48/0066; A61K 9/127; A61K 9/5184; A61K 38/465; A61K 48/00; A61K 48/0075; A61P 25/14; A61P 27/02; C12N 15/102; A61K 9/5176; C12N 2310/20

(57) ABSTRACT

This application relates to compositions and methods for excising trinucleotide repeats (TNRs) contained within intron 3 of TCF4, such as is seen in subjects having Fuchs endothelial corneal dystrophy (FECD), PSC, and Schizophrenia. Compositions comprising guide sequences targeting the alpha 2 subunit of collagen VIII are also disclosed for treatment of mutations therein that may contribute to FECD.

This application is a continuation of U.S. application Ser. No. 16/095,236 filed Oct. 19, 2018, which is a national stage entry pursuant to 35 U.S.C. § 371 of International Application No. PCT/US2017/028981 filed Apr. 21, 2017, which claim the benefit of U.S. Provisional Application No. 62/326,700 filed Apr. 22, 2016, the contents of each of which are incorporated herein by reference in their entirety for any purpose.

### REFERENCE TO ELECTRONIC SEQUENCE LISTING

The application contains a Sequence Listing which has been submitted electronically in .XML format and is hereby incorporated by reference in its entirety. Said .XML copy, created on Jul. 18, 2024, is named “01155-0007-01US.xml” and is 1,660,724 bytes in size. The sequence listing contained in this .XML file is part of the specification and is hereby incorporated by reference herein in its entirety.

This application relates to compositions and methods for treatment of diseases associated with trinucleotide repeats in the transcription factor four (TNF4) gene, including Fuchs endothelial corneal dystrophy (FECD), posterior polymorphous corneal dystrophy (PPCD), primary sclerosing cholangitis (PSC), and Schizophrenia.

Fuchs endothelial corneal dystrophy (FECD), also known as Fuchs' dystrophy, is a degenerative disease affecting the internal endothelial cell monolayer of the cornea. The role of the corneal endothelium is to ensure corneal clarity by maintaining an endothelial barrier and performing pump functions. In FECD, there is accumulation of focal outgrowths (termed guttae) and abnormal collagen in the corneal endothelium. The presence of guttae interspersed among the corneal endothelial and stromal cells is considered a clinical hallmark of the disease. Advanced FECD is characterized by extensive guttae, endothelial cell loss, and stromal edema.

FECD can result in vision loss, and advanced FECD is only treatable with corneal transplantation. It is estimated that approximately 5% of middle-aged Caucasians in the United States are affected by FECD. Additionally, it is estimated that FECD accounts for more than 14,000 corneal transplantations each year. Risks associated with corneal transplants include acute rejection, chronic rejection, failure of the graft to adhere to host bed, infection, and injury to the host eye. Most transplants leave the recipient with less than 20/20 vision, involve up to a six month recovery period, and require patients to use immunosuppressant drops for two years or more post-operatively. Extended use of immunosuppressant eye drops can increase the risk for cataracts or glaucoma.

A role for genetic factors in FECD has been reported, including single nucleotide polymorphisms and trinucleotide repeat (TNR) expansions in the transcription factor 4 (TCF4) gene. A TNR in the third intron of the TCF4 gene accounts for most of the inherited predisposition to disease, with repeat length of greater than 50 repeats being associated with clinical diagnosis of FECD (Wieben et al., PLOS One, 7:11, e49083 (2012)). Recent studies have suggested that this TNR expansion causes aggregation of the affected TCF4 RNA and sequestration of key RNA splicing factors (Mootha et al., Invest Ophthalmol Vis Sci. 55 (1): 33-42 (2014); Mootha et al., Invest Ophthalmol Vis Sci. 56 (3): 2003-11 (2015); Vasanth, et al., Invest Ophthalmol Vis Sci. 56 (8): 4531-6 (2015); Soliman et al., JAMA Ophthalmol. 133 (12): 1386-91 (2015)). Such sequestration can lead to global changes in gene expression, inducing profound changes in cellular function which ultimately lead to cell death (Du et al., J Biological Chem. 290:10, 5979-5990 (2015)). TCF4 mutations have also been associated with primary sclerosing cholangitis (PSC) and schizophrenia, see Ellinghas et al., HEPATOLOGY, 58:3, 1074-1083 (2013) and Forrest et al., Trends in Molecular Medicine 20:6 (2014).

In other repeat expansion diseases, RNA toxicity has been proposed. In cases of RNA toxicity, expanded microsatellite DNA sequences can be found in noncoding regions of various genes and the repetitive elements are transcribed into toxic gain-of-function RNAs or toxic protein species (see Mohan et al., Brain Res. 1584, 3-14 (2014)). Recently, RNA toxicity has also been shown in patients with FECD (see Du 2015). Further, it has been proposed that TCF4 TNR transcripts predominantly accumulate in the corneal endothelium and thus lead to the pathogenesis characteristic of FECD. Although the role of RNA toxicity helps to delineate potential disease mechanisms in FECD, treatment is still limited to corneal transplantation.

Other forms of early-onset FECD have been associated with mutations in COL8A2 (see Vedana et al., Clinical Ophthalmology 10 321-330 (2016)). Normally, collagen VIII or COL8 (comprising COL8A1 and COL8A2) is regularly distributed in the Descemet's membrane of the cornea. However, corneas from individuals with mutations in COL8A2 have an irregular mosaic deposition of different amounts of COL8A1 and COL8A2 in a non-coordinated fashion. Three mutations (Gln455Lys, Gln455Val, and Leu450Trp) in COL8A2 result in intracellular accumulation of mutant collagen VIII peptides and can cause early-onset FECD, as well as the related disorder posterior polymorphous corneal dystrophy (PPCD). PPCD is characterized by changes in the Descemet's membrane and endothelial layer of the cornea. The form of PPCD most often associated with mutation in the COL8A2 gene is PPCD2.

Means to directly modulate (CTG), TNRs in TCF4 and point mutations in COL8A2 are needed to treat genetic mutations leading to FECD, PPCD, PSC, and Schizophrenia. A recently investigated gene editing/disruption technique is based on the bacterial CRISPR (clustered regularly interspersed short palindromic repeats) system. CRISPR gene editing relies on a single nuclease, such as that embodied by “CRISPR-associated protein 9” (Cas9) and Cpf1, that can induce site-specific breaks in the DNA. Cas endonucleases are guided to a specific DNA sequence by small RNA molecules, termed trRNA and crRNA, along with a protospacer adjacent motif (PAM) adjacent to the target gene. The trRNA and crRNA together form the guide RNA, also known as gRNA. The trRNA and crRNA can be combined into a single guide RNA (sgRNA) to facilitate targeting of the Cas protein, or can be used at the same time but not combined, as a dual guide (dgRNA) system. Cas endonucleases in combination with trRNA and crRNA is termed the Cas ribonucleoprotein (RNP) complex.

### SUMMARY

We herein describe CRISPR compositions and their methods of use that in some embodiments are designed to excise some or all of the region within TCF4 containing the TNR expansions. In some embodiments these TNR expansions are found in individuals affected with FECD. Doing so prevents the toxicity associated with the expansion. A reduction or elimination in TNRs within TCF4 will reduce downstream effects of the TNRs, such as RNA toxicity, and improve disease course. Thus, guide RNAs complementary to target sequences flanking the TNRs of intron 3 of TCF4 and other modifications of the nuclease (or Cas RNP) may be a means to treat genetic forms of FECD exhibiting TNRs in TCF4, as well as TNRs in PSC and Schizophrenia. Additionally, guide sequences for use in designing guide RNAs that together with a nuclease knock out or edit COL8A2 in forms of FECD and PPCD displaying mutations in the alpha subunit of collagen VIII are also disclosed.

In accordance with the description, in some embodiments compositions of guide RNAs are described that direct CRISPR/Cas endonucleases to regions 5′ and 3′ to TNR expansions in the TCF4 gene. The compositions are useful in excising TNR expansions from the TCF4 gene, as well as in treating FECD, PPCD, PSC, and Schizophrenia. In other embodiments compositions of guide RNAs are also described that target to regions of the COL8A2 gene, including guide RNAs that target to mutant alleles that are associated with FECD. These guide RNAs are to be used together with a CRISPR nuclease to excise TNRs, generate indels, or induce gene correction through homologous recombination (HR) or homology-directed repair (HDR) via double-strand breaks, depending on the design of the guide RNAs and methods used in the treatments.

In one embodiment, the invention comprises a composition comprising at least one guide RNA comprising a guide sequence that directs a nuclease to a target sequence selected from SEQ ID NOs: 1-1084. In some embodiments, the invention comprises a composition comprising at least one guide RNA comprising a guide sequence that is at least 99%, 98%, 97%, 96%, 95%, 94%, 93%, 92%, 91%, or 90% identical to a sequence selected from SEQ ID NOs: 1089-1278.

In some embodiments, a composition comprising at least one guide RNA comprising a guide sequence that is identical to a sequence selected from SEQ ID NOs: 1089-1278 is provided.

In some embodiments, the guide RNA targets a trinucleotide repeat (TNR) in the transcription factor four (TCF4) gene, and directs a nuclease to a target sequence selected from SEQ ID NOs: 1-190. In some embodiments, the invention comprises at least one guide RNA comprising a guide sequence that is at least 99%, 98%, 97%, 96%, 95%, 94%, 93%, 92%, 91%, or 90% identical to a sequence selected from SEQ ID NOs: 1089-1278.

A composition comprising two guide RNAs selected from the following guide RNA pairings is provided:
a. a first guide RNA that directs a nuclease to SEQ ID NO: 83, and a second guide RNA that directs a nuclease to SEQ ID NO: 109;
b. a first guide RNA that directs a nuclease to SEQ ID NO: 85, and a second guide RNA that directs a nuclease to SEQ ID NO: 109;
c. a first guide RNA that directs a nuclease to SEQ ID NO: 86, and a second guide RNA that directs a nuclease to SEQ ID NO: 112;
d. a first guide RNA that directs a nuclease to SEQ ID NO: 85, and a second guide RNA that directs a nuclease to SEQ ID NO: 112;
e. a first guide RNA that directs a nuclease to SEQ ID NO: 86, and a second guide RNA that directs a nuclease to SEQ ID NO: 109;
f. a first guide RNA that directs a nuclease to SEQ ID NO: 85, and a second guide RNA that directs a nuclease to SEQ ID NO: 107;
g. a first guide RNA that directs a nuclease to SEQ ID NO: 83, and a second guide RNA that directs a nuclease to SEQ ID NO: 125;
h. a first guide RNA that directs a nuclease to SEQ ID NO: 86, and a second guide RNA that directs a nuclease to SEQ ID NO: 125;
i. a first guide RNA that directs a nuclease to SEQ ID NO: 86, and a second guide RNA that directs a nuclease to SEQ ID NO: 107;
j. a first guide RNA that directs a nuclease to SEQ ID NO: 64, and a second guide RNA that directs a nuclease to SEQ ID NO: 106;
k. a first guide RNA that directs a nuclease to SEQ ID NO: 85, and a second guide RNA that directs a nuclease to SEQ ID NO: 114;
l. a first guide RNA that directs a nuclease to SEQ ID NO: 86, and a second guide RNA that directs a nuclease to SEQ ID NO: 114;
m. a first guide RNA that directs a nuclease to SEQ ID NO: 83, and a second guide RNA that directs a nuclease to SEQ ID NO: 114;
n. a first guide RNA that directs a nuclease to SEQ ID NO: 53, and a second guide RNA that directs a nuclease to SEQ ID NO: 114;
o. a first guide RNA that directs a nuclease to SEQ ID NO: 83, and a second guide RNA that directs a nuclease to SEQ ID NO: 112; and
p. a first guide RNA that directs a nuclease to SEQ ID NO: 74, and a second guide RNA that directs a nuclease to SEQ ID NO: 114.

In some embodiments comprising two gRNAs, the first guide RNA that directs a nuclease to SEQ ID NO: 83 comprises SEQ ID NO: 1177, and the second guide RNA that directs a nuclease to SEQ ID NO: 109 comprises SEQ ID NO: 1197.

In some embodiments comprising two gRNAs, the first guide RNA that directs a nuclease to SEQ ID NO: 85 comprises SEQ ID NO: 1173, and the second guide RNA that directs a nuclease to SEQ ID NO: 109 comprises SEQ ID NO: 1197.

In some embodiments comprising two gRNAs, the first guide RNA that directs a nuclease to SEQ ID NO: 86 comprises SEQ ID NO: 1174, and the second guide RNA that directs a nuclease to SEQ ID NO: 112 comprises SEQ ID NO: 1200.

In some embodiments comprising two gRNAs, the first guide RNA that directs a nuclease to SEQ ID NO: 85 comprises SEQ ID NO: 1173, and the second guide RNA that directs a nuclease to SEQ ID NO: 112 comprises SEQ ID NO: 1200.

In some embodiments comprising two gRNAs, the first guide RNA that directs a nuclease to SEQ ID NO: 86 comprises SEQ ID NO: 1174, and the second guide RNA that directs a nuclease to SEQ ID NO: 109 comprises SEQ ID NO: 1197.

In some embodiments comprising two gRNAs, the first guide RNA that directs a nuclease to SEQ ID NO: 85 comprises SEQ ID NO: 1173, and the second guide RNA that directs a nuclease to SEQ ID NO: 107 comprises SEQ ID NO: 1195.

In some embodiments comprising two gRNAs, the first guide RNA that directs a nuclease to SEQ ID NO: 83 comprises SEQ ID NO: 1171, and the second guide RNA that directs a nuclease to SEQ ID NO: 125 comprises SEQ ID NO: 1213.

In some embodiments comprising two gRNAs, the first guide RNA that directs a nuclease to SEQ ID NO: 86 comprises SEQ ID NO: 1174, and the second guide RNA that directs a nuclease to SEQ ID NO: 125 comprises SEQ ID NO: 1213.

In some embodiments comprising two gRNAs, the first guide RNA that directs a nuclease to SEQ ID NO: 86 comprises SEQ ID NO: 1174, and the second guide RNA that directs a nuclease to SEQ ID NO: 107 comprises SEQ ID NO: 1195.

In some embodiments comprising two gRNAs, the first guide RNA that directs a nuclease to SEQ ID NO: 64 comprises SEQ ID NO: 1152, and the second guide RNA that directs a nuclease to SEQ ID NO: 106 comprises SEQ ID NO: 1194.

In some embodiments comprising two gRNAs, the first guide RNA that directs a nuclease to SEQ ID NO: 85 comprises SEQ ID NO: 1173, and the second guide RNA that directs a nuclease to SEQ ID NO: 114 comprises SEQ ID NO: 1202.

In some embodiments comprising two gRNAs, the first guide RNA that directs a nuclease to SEQ ID NO: 86 comprises SEQ ID NO: 1174, and the second guide RNA that directs a nuclease to SEQ ID NO: 114 comprises SEQ ID NO: 1202.

In some embodiments comprising two gRNAs, the first guide RNA that directs a nuclease to SEQ ID NO: 83 comprises SEQ ID NO: 1171, and the second guide RNA that directs a nuclease to SEQ ID NO: 114 comprises SEQ ID NO: 1202.

In some embodiments comprising two gRNAs, the first guide RNA that directs a nuclease to SEQ ID NO: 53 comprises SEQ ID NO: 1141, and the second guide RNA that directs a nuclease to SEQ ID NO: 114 comprises SEQ ID NO: 1202.

In some embodiments comprising two gRNAs, the first guide RNA that directs a nuclease to SEQ ID NO: 83 comprises SEQ ID NO: 1171, and the second guide RNA that directs a nuclease to SEQ ID NO: 112 comprises SEQ ID NO: 1200.

In some embodiments comprising two gRNAs, the first guide RNA that directs a nuclease to SEQ ID NO: 74 comprises SEQ ID NO: 1162, and the second guide RNA that directs a nuclease to SEQ ID NO: 114 comprises SEQ ID NO: 1202.

In some embodiments, the guide RNA targets the alpha 2 subunit of collagen type VIII (Col8A2) gene, and directs a nuclease to a target sequence selected from SEQ ID NOs: 191-1063. In some embodiments, the invention comprises at least one guide RNA comprising a guide sequence that is at least 99%, 98%, 97%, 96%, 95%, 94%, 93%, 92%, 91%, or 90% identical to a sequence complementary, or identical, to the first 20 nucleotides of a target sequence selected from SEQ ID NOs: 191-1063 (e.g, the target sequence absent the PAM), wherein the thymines in the first 20 nucleotides of SEQ ID NOs: 191-1063 are replaced with uracil.

In some embodiments, the guide RNA targets the Gln455Lys mutation in the Col8A2 gene product and directs a nuclease to a target sequence selected from SEQ ID NOs: 1064-1069. In some embodiments, the invention comprises at least one guide RNA comprising a guide sequence that is at least 99%, 98%, 97%, 96%, 95%, 94%, 93%, 92%, 91%, or 90% identical to a sequence complementary, or identical, to the first 20 nucleotides of a target sequence selected from SEQ ID NOs: 1064-1069 (e.g, the target sequence absent the PAM), wherein the thymines in the first 20 nucleotides of SEQ ID NOs: 1064-1069 are replaced with uracil.

In some embodiments, the guide RNA targets the Gln455Val mutation in the Col8A2 gene product and directs a nuclease to a target sequence selected from SEQ ID NOs: 1070-1075. In some embodiments, the invention comprises at least one guide RNA comprising a guide sequence that is at least 99%, 98%, 97%, 96%, 95%, 94%, 93%, 92%, 91%, or 90% identical to a sequence complementary, or identical, to the first 20 nucleotides of a target sequence selected from SEQ ID NOs: 1070-1075 (e.g, the target sequence absent the PAM), wherein the thymines in the first 20 nucleotides of SEQ ID NOs: 1070-1075 are replaced with uracil.

In some embodiments, the guide RNA targets the Leu450Trp mutation in the Col8A2 gene product, and directs a nuclease to a target sequence selected from SEQ ID NOs: 1076-1084. In some embodiments, the invention comprises at least one guide RNA comprising a guide sequence that is at least 99%, 98%, 97%, 96%, 95%, 94%, 93%, 92%, 91%, or 90% identical to a sequence complementary, or identical, to the first 20 nucleotides of a target sequence selected from SEQ ID NOs: 1076-1084 (e.g, the target sequence absent the PAM), wherein the thymines in the first 20 nucleotides of SEQ ID NOs: 1070-1075 are replaced with uracil.

In some embodiments, the guide RNA is a dual guide. In some embodiments, the guide RNA is a single guide. In some embodiments, at least one guide RNA comprises a crRNA, a trRNA, or a crRNA and a trRNA.

In some embodiments, at least one guide sequence is encoded on a vector. In some embodiments, a first guide sequence and a second guide sequence are encoded on the same vector. In some embodiments, a first guide sequence and a second guide sequence are encoded on different vectors. In some embodiments, the first guide sequence and the second guide sequence are controlled by the same promotor and/or regulatory sequence.

In some embodiments, the guide sequence is complementary to a target sequence in the positive strand of a target gene. In some embodiments, the guide sequence is complementary to a target sequence in the negative strand of a target gene. In some embodiments, a first guide sequence and second guide sequence are complementary to a first target sequence and a second target sequence in opposite strands of a target gene (i.e., a region of interest such as TNRs in TCF4 in genomic DNA).

In some embodiments, the guide RNA is chemically modified. In some embodiments, the invention further comprises a nuclease. In some embodiments, the nuclease is a Cas protein or other nuclease that cleaves double or single-stranded DNA. In some embodiments, the Cas protein is from the Type-I, Type-II, or Type-III CRISPR/Cas system. In some embodiments, the Cas protein is Cas9 or Cpf1. In some embodiments, the nuclease is a nickase. In some embodiments, the nuclease is modified. In some embodiments, the modified nuclease comprises a nuclear localization signal (NLS).

In some embodiments, the invention comprises a pharmaceutical formulation of a guide RNA and a pharmaceutically acceptable carrier. In some embodiments, the pharmaceutical formulation comprises one or more guide RNA and an mRNA encoding a Cas protein. In some embodiments, the pharmaceutical formulation comprises one or more guide RNA and a Cas protein.

In some embodiments, the invention comprises a method of excising at least a portion of a trinucleotide repeat (TNR) in the transcription factor four (TCF4) gene in a human subject. In some embodiments, two guide RNA are used, wherein the first is complementary to a sequence 5′ of the TNR and the second is complementary to a sequence 3′ of the TNR. When two guide sequences are used, the DNA sequences between the targeted regions of genomic DNA are excised.

In some embodiments, the TNR is equal to or greater than about 40 trinucleotide repeats. In some embodiments, the TNR is equal to or greater than about 50, 45, 40, 35, 30, 25, 20, 15, 10, or 5 trinucleotide repeats. In some embodiments, the TNR is equal to or greater than about 40, 50, 60, 70, 80, 90, 100, 110, 120, 130, 140, 150, 160, 170, 180, 190, or 200 trinucleotide repeats.

In some embodiments, the composition or pharmaceutical formulation comprises at least two guides that excise at least a portion of the TNR. In some embodiments, the entire TNR is excised.

In some embodiments, the composition or pharmaceutical formulation is administered via a viral vector. In some embodiments, the composition or pharmaceutical formulation is administered via lipid nanoparticles. Any lipid nanoparticle known to those of skill in the art is suitable for delivering the one or more guide RNA provided herein, optionally together with an mRNA encoding a Cas protein. In some embodiments, the lipid nanoparticles described in PCT/US2017/024973, filed Mar. 30, 3017, are utilized. In some embodiments, the lipid nanoparticles comprise one or more guide RNA provided herein and an mRNA encoding a Cas protein. In some embodiments, the lipid nanoparticles comprise one or more guide RNA provided herein without an mRNA encoding a Cas protein.

In some embodiments, the invention further comprises co-administration of eye drops or ointments. In some embodiments, the invention further comprising the use of soft contact lenses.

In some embodiments, the human subject has schizophrenia.

In some embodiments, the human subject has primary sclerosing cholangitis (PSC).

In some embodiments, the invention comprises a method of decreasing expression of a mutant allele of the COL8A2 gene, such as Gln455Lys, Gln455Val, or Leu450Trp, or altering the nucleotide sequence to correct said mutant allele in a human subject.

In some embodiments, the human subject has Fuchs endothelial corneal dystrophy (FECD) or posterior polymorphous corneal dystrophy (PPCD). In some embodiments, the human subject has FECD. In some embodiments, the subject has a family history of FECD.

In some embodiment, the subject has an improvement, stabilization, or slowing of decline in visual acuity as a result of administration. In some embodiments, the subject has an improvement, stabilization, or slowing of change as measured by corneal pachymetry as a result of administration. In some embodiments, the subject has an improvement, stabilization, or slowing of change based on specular microscopy as a result of administration. In some embodiments, the subject has a delay in the time until a corneal transplant is needed as a result of administration.

In some embodiments, the invention comprises use of a composition or a pharmaceutical for the preparation of a medicament for treating a human subject having a TNR expansion in the TCF4 gene, or having mutation in the COL8A2 gene leading to a Gln455Lys, Gln455Val, or a Leu450Trp mutation in the gene product.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1 provides a schematic of excision of the TNR expansion region in intron 3 of TCF4 using a pair of gRNAs, with one gRNA having a guide sequence that targets to a region of intron 3 that is 5′ of the TNRs and the other gRNA having a guide sequence that targets to a region of intron 3 that is 3′ of the TNRs. While the drawing shows the excision occurring at the exact boundaries of the TNR, in practice the excision can be larger or smaller, and include upstream and/or downstream regions of the intron.

FIG. 2 provides a schematic showing the predicted sizes of excised fragments for the 93 pairs of gRNAs that were tested for excision. The numbers correspond to the SEQ ID NOs of each target sequence for the guides tested. The pairs are rank ordered by excision percent (the top pair of the list having the highest excision rate). The “0” marks the center of the TNR region.

### DESCRIPTION OF THE SEQUENCES

Table 1 provides a listing of certain sequences referenced herein.

TABLE 1
Description of the Sequences

| | | SEQ ID |
|---|---|---|
| Sequences | Description | NO: |
| Presented in Table 2 | Target | 1-93 |
| | sequences | |
| | 5′ of | |
| | TNRs in | |
| | intron 3 | |
| | of TCF4 | |
| Presented in Table 2 | Target | 94-190 |
| | sequences | |
| | 3′ of | |
| | TNRs in | |
| | intron 3 | |
| | of TCF4 | |
| Presented in Table 3 | Target | 191-1063 |
| | sequences | |
| | for | |
| | wild | |
| | type | |
| | COL8A2 | |
| Presented in Table 4 | Target | 1064-1069 |
| | sequences | |
| | for | |
| | COL8A2 | |
| | Gln455Lys | |
| | Mutation | |
| Presented in Table 5 | Target | 1070-1075 |
| | sequences | |
| | for | |
| | COL8A2 | |
| | Gln455Val | |
| | Mutation | |
| Presented in Table 6 | Target | 1076-1084 |
| | sequences | |
| | for | |
| | COL8A2 | |
| | Leu450Trp | |
| | Mutation | |
| GTTTGTGTGA TTTTGCTAAA ATGCATCACC AACAGCGAAT | TCF4 | 1085 |
| GGCTGCCTTA GGGACGGACA AAGAGCTGAG TGATTTACTG | intron 3 | |
| GATTTCAGTG CGgtaagaaa gaacggtgga aactaacaac | sequence | |
| agctgtgaaa aaaacaaaac aaaaacccaa acacttcagc | with | |
| tagaaaccag taggaatcta aaggacagta ataattttta | flanking | |
| attggctgaa tccttggtaa atatgaaggt ctttttgaca | exons, | |
| agtttttaac tataattttg tggtgtgatg gaagattcag | reverse | |
| gctttttttt ttttttgagt tttattactg gccttcaatt | strand | |
| ccctacccac tgattacccc aaataatgga atctcacccc | (GRCh37/ | |
| agtggaaagc aaaaatagac acccctaaaa ctaaaccacc | hg19). | |
| cctaaaactt ggccatgtct gaacactgag actactaata | While | |
| ctttgcacac tactcttcgt tttatttatt gtttttggaa | commonly | |
| atggaaaata gaaaatagga gacccagttg tctctttaaa | referred | |
| gttttaagct aatgatgctt tggattggta ggacctgttc | to as | |
| cttacatctt acctcctagt tacatctttt cctaggattc | intron | |
| ttaaaactag tatggatatg ctgagcatac attctttaga | 3, many | |
| accttttgga ctgttttggt aaatttcgta gtcgtaggat | alternately | |
| cagcacaaag cggaacttga cacacttgtg gagttttacg | spliced | |
| gctgtacttg gtccttctcc atccctttgc ttccttttcc | isoforms | |
| taaaccaagt cccagacatg tcaggagaat gaattcattt | of the | |
| ttaatgccag atgagtttgg tgtaagatgc atttgtaaag | gene | |
| caaaataaaa agaatccaca aaacacacaa ataaaatcca | exist, | |
| aaccgccttc caagtggggc tctttcatgc tgctgctgct | such | |
| gctgctgctg ctgctgctgc tgctgctgct gctgctgctg | that | |
| ctgctgctgc tgctgctgct gctcctcctc ctcctcctcc | this | |
| ttctcctcct cctcctcctc ttctagacct tcttttggag | intron | |
| aaatggcttt cggaagtttt gccaggaaac gtagccctag | may not | |
| gcaggcagct ttgcagcccc ctttctgctt gttgcacttt | fall | |
| ctccattcgt tcctttgctt tttgcaggct ctgactcagg | between | |
| gaaggtgtgc attatccact agatacgtcg aagaagaggg | the 3^{rd} | |
| aaaccaatta gggtcgaaat aaatgctgga gagagaggga | and 4^{th} | |
| gtgaaagaga gagtgagagt gagagagaga gagagtcttg | exons of | |
| cttcaaattg ctctcctgtt agagacgaaa tgagaattta | every | |
| gtgcaggtgg cacttttatt tttatttggg ttcacatatg | transcript. | |
| acaggcaaat cctatacgag atggaaatgg acattgccac | Bold | |
| gtttatggcc aaggttttca atataaaaca aaacaacttt | font | |
| tttcttctcc ttggtgaaac tagtgttttt ctagagaggc | indicates | |
| tgctggcctc caacctgaat cttgataaca ttatggggac | ctg | |
| tgtgtttgtt ccaaatgtag cagtagtact gcttggccat | repeats | |
| ctaatgaacc tgaggaaaaa gaaagaacag agtgataatg | (TNRs). | |
| ggggctgggg tgggatctgt aatgttgttt ctcttttagt | This | |
| tttaagttgg atggtgatgt attttactaa ataaaccctt | region | |
| agcataaact ctaagctgtt tggtaacagt atgaaagatc | is | |
| tttgaggagc tctgaaggca caagtgtctt cttttcaact | variable | |
| gtaatatttc tttgtttctt ttagATGTTT TCACCTCCTG | in size. | |
| TGAGCAGTGG GAAAAATGGA CCAACTTCTT TGGCAAGTGG | Capital | |
| ACATTTTACT GGCTCAA | letters | |
| | indicate | |
| | sequences | |
| | of | |
| | adjacent | |
| | 5′ and | |
| | 3′ | |
| | exons. | |
| mN*mN*mN*NNNNNNNNNNNNNNNNNGUUUUAG | sgRNA | 1086 |
| AmGmCmUmAmGmAmAmAmUmAmGmCAGUUAA | modified | |
| AUAAGGCUAGUCCGUUAUCAmAmCmUmUmGmAm | sequence | |
| AmAmAmAmGmUmGmGmCmAmCmCmGmAmGmUmC | “N” may | |
| mGmGmUmGmCmU*mU*mU*mU | be any | |
| | natural | |
| | or non- | |
| | natural | |
| | nucleotide. | |
| | * = PS | |
| | linkage; | |
| | ‘m’ = | |
| | 2′-O-Me | |
| | nucleotide | |
| NNNNNNNNNNNNNNNNNNNNGUUUUAGAGCUAUGCUGUUUUG | crRNA | 1087 |
| | sequence | |
| | “N” may | |
| | be any | |
| | natural | |
| | or non- | |
| | natural | |
| | nucleotide. | |
| AACAGCAUAGCAAGUUAAAAUAAGGCUAGUCCGUUAUCAACUUG | trRNA | 1088 |
| AAAAAGUGGCACCGAGUCGGUGCUUUUUUU | sequence | |

### DESCRIPTION OF THE EMBODIMENTS

### Definitions

The term “treatment,” as used herein, covers any administration or application of a therapeutic for disease in a subject, and includes inhibiting the disease, arresting its development, relieving one or more symptoms of the disease, curing the disease, or preventing reoccurrence of one or more symptoms of the disease. For example, treatment of FECD may comprise alleviating symptoms of FECD, as well as reducing the number of TNRs in the TCF4 gene resulting in an amelioration of symptoms of FECD, a slowing of disease progression, or cure/prevention of reoccurrence of symptoms of the disease.

As used herein, “FECD” refers to Fuchs endothelial corneal dystrophy, also known as Fuchs' dystrophy. FECD would also include individuals without symptoms but with a genetic disorder, such as a TNR expansion in intron 3 of TCF4, linked to increased occurrence of FECD. FECD would also include individuals without symptoms, but having a known family history of FECD and a TNR expansion in intron 3 of TCF4.

As used herein, “TNRs” refers to trinucleotide repeats. “Microsatellite repeats” refers to short sequence of DNA consisting of multiple repetitions of a set of two to nine base pairs. The term microsatellite repeats encompasses TNRs. “TNR expansion” refers to a higher than normal number of trinucleotide repeats. For intron 3 of TCF4, for example, a TNR expansion can be characterized by about 50 or more TNRs. The range of TNR expansion associated with disease is usually between 50 and 1000, though some patients with >1000 repeats have been identified. Patients with <50 TNRs in intron 3 of TCF4 are generally not considered to be at increased risk of disease through a TNR expansion mechanism, though they may still benefit from a reduced number of TNRs.

Diseases caused by TNRs and/or characterized by the presence of TNRs may be referred to as “trinucleotide repeat disorders,” “trinucleotide repeat expansion disorders,” “triplet repeat expansion disorders,” or “codon reiteration disorders.”

A “guide RNA” and “gRNA” are used interchangeably herein. The gRNA comprises or consists a CRISPR RNA (crRNA) and a trRNA (also known as tracrRNA). The crRNA and trRNA may be associated on one RNA molecule (single guide RNA (sgRNA)), or may be disassociated on separate RNA molecules (dual guide RNA (dgRNA)).

As used in this application, “the guide sequence” refers to an about 20-base pair sequence within the crRNA or trRNA that is complementary to a target sequence and functions to direct a guide RNA to a target sequence for cleavage by a nuclease. Slightly shorter or longer sequences can also be used as guides, e.g., 15-, 16-, 17-, 18-, 19-, 21-, 22-, 23-, 24-, or 25-base pairs in length. In some embodiments, the length of the guide sequence corresponds to the length of the target sequence, e.g., as described herein.

As used herein, a “target sequence” refers to a sequence of nucleic acid to which the guide RNA directs a nuclease for cleavage. The target sequence is within the genomic DNA of a subject. In some embodiments, a Cas protein may be directed by a guide RNA to a target sequence, where the guide RNA hybridizes with and the nuclease cleaves the target sequence. Target sequences include both the positive and negative strands of genomic DNA (i.e., the sequence given and the sequence's reverse compliment), as a nucleic acid substrate for a Cas protein is a double stranded nucleic acid. Accordingly, where a guide sequence is said to be “complementary to a target sequence”, it is to be understood that the guide sequence may direct a guide RNA (e.g., in a RNP) to bind to the reverse complement of a target sequence provided herein. Thus, in some embodiments, where the guide sequence binds the reverse complement of a target sequence, the guide sequence is identical to the first 20 nucleotides of the target sequence (e.g., the target sequence not including the PAM) except for the substitution of U for T in the guide sequence.

As used herein, a “PAM” or “protospacer adjacent motif” refers to a sequence that must be adjacent to the target sequence. The PAM needed varies depending on the specific CRISPR system. In the CRISPR/Cas system derived from Streptococcus pyogenes, the target DNA must immediately precede a 5′-NGG PAM (where “N” is any nucleobase followed by two guanine nucleobases) for optimal cutting, while other Cas9 orthologs have different PAM requirements. While Streptococcus pyogenes Cas9 can also recognize the 5′-NAG PAM, it appears to cut less efficiently at these PAM sites. The target sequences of Table 2 comprise a PAM.

In some embodiments, the guide RNA and the Cas protein may form a “ribonucleoprotein” (RNP). In some embodiments, the guide RNA guides the nuclease such as Cas9 to a target sequence, and the guide RNA hybridizes with and the nuclease cleaves the target sequence.

As used herein, “indels” refer to insertion/deletion mutations consisting of a number of nucleotides that are either inserted or deleted at the site of double-stranded breaks (DSBs) in the nucleic acid.

As used herein, “excision fragment(s)” refers to deletions of a consecutive number of nucleotides that may occur when two or more guide RNAs are used together with a Cas mRNA or protein.

### Compositions

Compositions useful in the treatment of FECD are described. In some aspects, the compositions comprise a guide RNA that directs a nuclease to a TNR in the TCF4 gene thereby cleaving the TNR thereby treating diseases having TNRs in the TCF4 gene, including FECD, PPCD, PSC, and Schizophrenia. In some embodiments, the composition comprises two guide RNAs that direct nuclease to a first and second location in intron 3 of TCF4, wherein the nuclease cleaves the intron 3 of TCF4 at the first and second locations and excises a fragment of nucleic acid between the first and the second cleavage, thereby excising some or all of the TNRs contained within intron 3 of TCF4 and treating diseases having TNRs in the TCF4 gene, including FECD, PPCD, PSC, and Schizophrenia. In other aspects, the compositions comprise a guide RNA that directs a nuclease to the COL8A2 gene via a target sequence in the DNA thereby mediating NHEJ for the purpose of cleaving the sequence and leading to introduction of indels or mediating HR or HDR wherein a mutation in the DNA can be corrected by use of a template and treating FECD or PPCD. Embodiments of the compositions are described below.

### Guide RNA

In some embodiments, the compositions of the invention comprise guide RNA (gRNA) comprising a guide sequence(s) that directs a nuclease such as Cas9 to a target DNA sequence. The gRNA comprises a crRNA and a trRNA. In each composition and method embodiment described herein, the crRNA and trRNA may be associated on one RNA (sgRNA), or may be disassociated on separate RNAs (dgRNA).

In each of the composition and method embodiments described herein, the guide RNA may comprise two RNA molecules as a “dual guide RNA” or “dgRNA”. The dgRNA comprises a first RNA molecule comprising a crRNA, and a second RNA molecule comprising a trRNA. The first and second RNA molecules are not covalently linked, but may form a RNA duplex via the base pairing between the flagpole regions on the crRNA and the trRNA.

In each of the composition and method embodiments described herein, the guide RNA may comprise a single RNA molecule as a “single guide RNA” or “sgRNA”. The sgRNA comprises a crRNA covalently linked to a trRNA. In some embodiments, the crRNA and the trRNA are covalently linked via a linker. In some embodiments, the sgRNA forms a stem-loop structure via the base pairing between the flagpole regions on the crRNA and the trRNA.

In some embodiments, the trRNA may comprise all or a portion of a wild type trRNA sequence from a naturally-occurring CRISPR/Cas system. In some embodiments, the trRNA comprises a truncated or modified wild type trRNA. The length of the trRNA depends on the CRISPR/Cas system used. In some embodiments, the trRNA comprises or consists of 5, 6, 7, 8, 9, 10, 11, 12, 13, 14, 15, 16, 17, 18, 19, 20, 25, 30, 40, 50, 60, 70, 80, 90, 100, or more than 100 nucleotides. In certain embodiments, the trRNA is at least 26 nucleotides in length. In additional embodiments, the trRNA is at least 40 nucleotides in length. In some embodiments, the trRNA may comprise certain secondary structures, such as, e.g., one or more hairpins or stem-loop structures, or one or more bulge structures.

In some embodiments, the gRNA is chemically modified. A gRNA comprising one or more modified nucleosides or nucleotides is called a “modified” gRNA or “chemically modified” gRNA, to describe the presence of one or more non-naturally and/or naturally occurring components or configurations that are used instead of or in addition to the canonical A, G, C, and U residues. In some embodiments, a modified gRNA is synthesized with a non-canonical nucleoside or nucleotide, is here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

The modifications listed above can be combined to provide modified gRNAs comprising nucleosides and nucleotides (collectively “residues”) that can have two, three, four, or more modifications. For example, a modified residue can have a modified sugar and a modified nucleobase. In some embodiments, every base of a gRNA is modified, e.g., all bases have a modified phosphate group, such as a phosphorothioate group. In certain embodiments, all, or substantially all, of the phosphate groups of an gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%) of the positions in a modified gRNA are modified nucleosides or nucleotides.

Unmodified nucleic acids can be prone to degradation by, e.g., cellular nucleases. For example, nucleases can hydrolyze nucleic acid phosphodiester bonds. Accordingly, in one aspect the gRNAs described herein can contain one or more modified nucleosides or nucleotides, e.g., to introduce stability toward nucleases. In some embodiments, the modified gRNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the 2′ hydroxyl group modification can included “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In embodiments comprising an sgRNA, one or more residues at one or both ends of the sgRNA may be chemically modified, or the entire sgRNA may be chemically modified. Certain embodiments comprise a 5′ end modification. Certain embodiments comprise a 3′ end modification. In certain embodiments, one or more or all of the nucleotides in single stranded overhang of a guide RNA molecule are deoxynucleotides.

In some embodiments, the guide RNAs disclosed herein comprise one of the modification patterns disclosed in U.S. 62/431,756, filed Dec. 8, 2016, titled “Chemically Modified Guide RNAs,” the contents of which are hereby incorporated by reference in their entirety.

In some embodiments, the invention comprises a gRNA comprising one or more modifications. In some embodiments, the modification comprises a 2′-O-methyl (2′-O-Me) modified nucleotide. In some embodiments, the modification comprises a phosphorothioate (PS) bond between nucleotides.

The terms “mA,” “mC,” “mU,” or “mG” may be used to denote a nucleotide that has been modified with 2′-O-Me.

Modification of 2′-O-methyl can be depicted as follows:

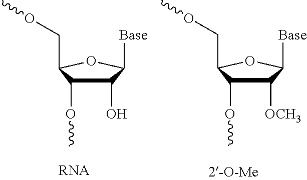

Another chemical modification that has been shown to influence nucleotide sugar rings is halogen substitution. For example, 2′-fluoro (2′-F) substitution on nucleotide sugar rings can increase oligonucleotide binding affinity and nuclease stability.

In this application, the terms “fA,” “fC,” “fU,” or “fG” may be used to denote a nucleotide that has been substituted with 2′-F.

Substitution of 2′-F can be depicted as follows:

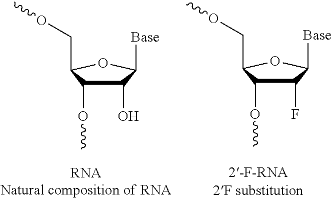

Phosphorothioate (PS) linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos.

A “*” may be used to depict a PS modification. In this application, the terms A*, C*, U*, or G* may be used to denote a nucleotide that is linked to the next (e.g., 3′) nucleotide with a PS bond.

In this application, the terms “mA*,” “mC*,” “mU*,” or “mG*” may be used to denote a nucleotide that has been substituted with 2′-O-Me and that is linked to the next (e.g., 3′) nucleotide with a PS bond.

The diagram below shows the substitution of S-into a nonbridging phosphate oxygen, generating a PS bond in lieu of a phosphodiester bond:

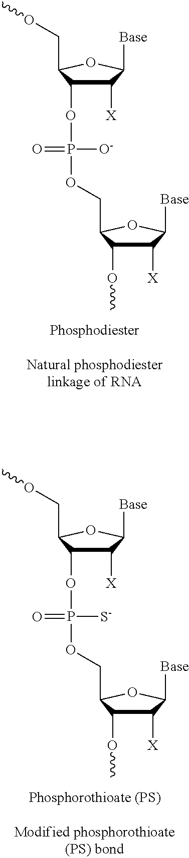

Abasic nucleotides refer to those which lack nitrogenous bases. The figure below depicts an oligonucleotide with an abasic (also known as apurinic) site that lacks a base:

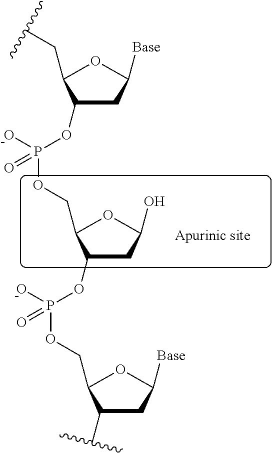

Inverted bases refer to those with linkages that are inverted from the normal 5′ to 3′ linkage (i.e., either a 5′ to 5′ linkage or a 3′ to 3′ linkage). For example:

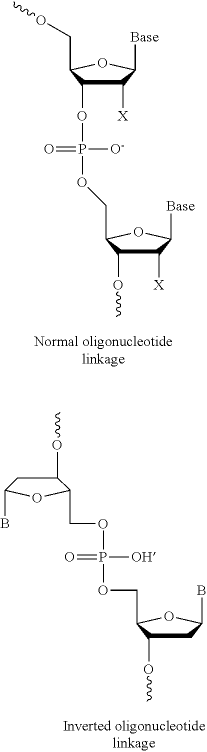

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In some embodiments, one or more of the first three, four, or five nucleotides at the 5′ terminus, and one or more of the last three, four, or five nucleotides at the 3′ terminus of the guide RNA are modified. In some embodiments, the modification is a 2′-O-Me, 2′-F, inverted abasic nucleotide, PS bond, or other nucleotide modification well known in the art to increase stability and/or performance.

In some embodiments, the first four nucleotides at the 5′ terminus, and the last four nucleotides at the 3′ terminus are linked with phosphorothioate (PS) bonds.

In some embodiments, the first three nucleotides at the 5′ terminus, and the last three nucleotides at the 3′ terminus comprise a 2′-O-methyl (2′-O-Me) modified nucleotide. In some embodiments, the first three nucleotides at the 5′ terminus, and the last three nucleotides at the 3′ terminus comprise a 2′-fluoro (2′-F) modified nucleotide. In some embodiments, the first three nucleotides at the 5′ terminus, and the last three nucleotides at the 3′ terminus comprise an inverted abasic nucleotide.

In some embodiments, the guide RNA comprises a modified sgRNA. In some embodiments, the sgRNA comprises the modification pattern shown in SEQ ID NO: 1086, where N is any natural or non-natural nucleotide, and where the totality of the N's comprise a guide sequence as described herein that directs a nuclease to a TC4 target sequence. Guide RNAs for TCF4

In some embodiments, the composition comprises at least one guide RNA (gRNA) comprising or consisting of a guide sequence complementary to any one of the nucleic acids of SEQ ID NOs: 1-190. In some embodiments, the composition comprises at least one guide RNA (gRNA) comprising or consisting of a guide sequence that directs a nuclease to any one of the nucleic acids of SEQ ID NOs: 1-190. In one aspect, the composition comprises at least one gRNA comprising or consisting of a guide sequence complementary to a target sequence that is at least 99%, 98%, 97%, 96%, 95%, 94%, 93%, 92%, 91%, or 90% identical to any of the nucleic acids of SEQ ID NOs: 1-190. In one aspect, the composition comprises at least one gRNA comprising or consisting of a guide sequence that directs a nuclease to a target sequence that is at least 99%, 98%, 97%, 96%, 95%, 94%, 93%, 92%, 91%, or 90% identical to any of the nucleic acids of SEQ ID NOs: 1-190.

In some aspects, the composition comprises at least one gRNA comprising or consisting of a guide sequence that is at least 99%, 98%, 97%, 96%, 95%, 94%, 93%, 92%, 91%, or 90% identical to any of the nucleic acids of SEQ ID NOs: 1089-1278. In some aspects, the composition comprises at least one gRNA comprising or consisting of a guide sequence identical to any of the nucleic acids of SEQ ID NOs: 1089-1278.

In other embodiments, the composition comprises at least two gRNA's comprising or consisting of at least two guide sequences complementary to any one of the target sequences selected from any two or more of the nucleic acids of SEQ ID NOs: 1-190. In some embodiments, the composition comprises at least two gRNA's comprising or consisting of at least two guide sequences complementary to any one of the target sequences selected from any two or more of the nucleic acids that are at least 99%, 98%, 97%, 96%, 95%, 94%, 93%, 92%, 91%, or 90% identical to any of the nucleic acids of SEQ ID NOs: 1-190.

In some embodiments, a gRNA that targets to a sequence 5′ of the TNRs of TCF4 is used together with a gRNA that targets to a sequence 3′ of the TNRs of TCF4 for the purpose of excising the TNRs of TCF4. In some embodiments, a guide sequence complementary to a target sequence of SEQ ID NOs: 1-93 is used together with a guide sequence complementary to a target sequence of SEQ ID NOs: 94-190.

In some embodiments, use of a gRNA that targets to a sequence 5′ of the TNRs of TCF4 together with a gRNA that targets to a sequence 3′ of the TNRs of TCF4 excises the full sequence of TNRs in intron 3 of TCF4 in patients with extended TNR sequences. For example, in some embodiments the combination of gRNAs targeting sequences 5′ and 3′ to the TNR expansion excises a TNR having at least 40, at least 50, at least 60, at least 70, at least 80, at least 90, at least 100, at least 150, at least 200, at least 250, at least 300, at least 400, at least 500, at least 600, at least 700, at least 800, at least 900, or at least 1000 or more repeats. In some embodiments, this approach is used to excise TNR expansions greater than 40 in number. In some embodiments, use of a gRNA that targets to a sequence 5′ of the TNRs of TCF4 together with a gRNA that targets within the TNR repeats, or use of a gRNA that targets within the TNR repeats together with a gRNA that targets to a sequence 3′ of the TNRs of TCF4, excises a portion of the extended TNRs in intron of TCF4 in patients with extended TNR sequences, thereby shortening the length of the TNRs. In some embodiments, the one guide RNA targets a sequence that is 5′ of the TNRs of TCF4, and the other guide RNA targets a sequence that is 3′ of the TNRs of TCF4, thereby excising all of the TNRs.

### Combinations of Two or More Guide RNAs Targeting to TCF4

In certain embodiments, the compositions comprise more than one gRNA. Each gRNA may contain a different guide sequence, such that the associated nuclease cleaves more than one target sequence. In some embodiments, the gRNAs may have the same or differing properties such as activity or stability within the RNP complex. In some embodiments involving vectors, where more than one gRNA is used, each gRNA can be encoded on the same or on different vectors. The promoters used to drive expression of the more than one gRNA may be the same or different. In certain embodiments involving lipid nanoparticles, the two or more gRNAs may be formulated in the same lipid nanoparticle or in separate lipid nanoparticles.

In some embodiments, the guide sequence of each gRNA is complementary to a target sequence in the same strand of the TCF4 gene. In some embodiments, the guide sequence of each gRNA is complementary to a target sequence in the positive strand of the TCF4 gene. In some aspects, the guide sequences of each gRNA is complementary to a target sequence in the negative strand of the TCF4 gene. In some embodiments, the guide sequences of the gRNAs are complementary to target sequences in opposite strands of the TCF4 gene.

In some aspects, the compositions comprise at least two gRNAs, wherein the at least two gRNAs comprise guide sequences that target nucleases to two different locations. In some embodiments, the two gRNAs may flank a TNR of the TCF4 gene (i.e., the two gRNAs are on either side of the TNR; said another way, one gRNA is 5′ to the TNR and another gRNA is 3′ to the TNR). In some embodiments, one gRNA is within a TNR of the TCF4 gene and the other gRNA is outside of the TNR (i.e., flanks the TNR) of the TCF4 gene. In some embodiments, the two gRNAs target nucleases to target sequences that are about 3000, 2500, 2000, 1500, 1000, 500, 400, 300, 200, 150, 100, 50, or 30 nucleotides apart. In some embodiments, the nuclease cleaves each location and a DNA fragment comprising the TNR expansion region of intron 3 of TCF4 is excised.

In some embodiments, only one gRNA is used. In some embodiments, a gRNA that targets to a sequence 5′ of the TNRs of TCF4 is used. In some embodiments the guide sequence is complementary to the target sequence of SEQ ID NO: 1-93. In some embodiments, a gRNA that targets to a sequence 3′ of the TNRs of TCF4 is used. In some embodiments, a guide complementary to the target sequence of SEQ ID NOs: 94-190 is used. In some embodiments, a gRNA that targets a sequence within the TNR repeat expansion in TCF4 is used. In some embodiments, use of a single guide leads to indel formation during NHEJ that reduces or eliminates the TNR sequence. In some embodiments, use of a single guide leads to indel formation during NHEJ that reduces or eliminates a part of the TNR sequence.

### Guide RNAs for COL8A2

In some embodiments, the composition comprises at least one guide RNA (gRNA) comprising or consisting of a guide sequence complementary to any of the nucleic acids of SEQ ID NOs: 191-1084. In one aspect, the composition comprises at least one gRNA comprising or consisting of a guide sequence complementary to a target sequence that is at least 99%, 98%, 97%, 96%, 95%, 94%, 93%, 92%, 91%, or 90% identical to any of the nucleic acids of SEQ ID NOs: 191-1084.

In other embodiments, the composition comprises at least two gRNA's comprising or consisting of at least two guide sequences complementary to any two or more of the nucleic acids of SEQ ID NOs: 191-1084. In some embodiments, the composition comprises at least two gRNA's comprising or consisting of at least two guide sequences complementary to any two or more of the nucleic acids that are at least 99%, 98%, 97%, 96%, 95%, 94%, 93%, 92%, 91%, or 90% identical to a sequence of the nucleic acids of SEQ ID NOs: 191-1084.

In some embodiments, a gRNA that targets to a sequence in wild type COL8A2, without known mutations, is used. In some embodiments, a guide sequence complementary to a target sequence of SEQ ID NOs: 191-1063 is used.

In some embodiments, a gRNA that targets to a sequence corresponding to a mutation in COL8A2 known to produce a Gln455Lys mutation is used. In some embodiments, a guide sequence complementary to a target sequence of SEQ ID NOs: 1064-1069 is used, e.g., to selectively edit the Gln455Lys mutation, caused by the c. 1364C>A nucleotide change.

In some embodiments, a gRNA that targets to a sequence corresponding to a mutation in COL8A2 known to produce a Gln455Val mutation is used. In some embodiments, a guide sequence complementary to a target sequence of SEQ ID NOs: 1070-1075 is used, e.g., to selectively edit the Gln455Val mutation caused by the c. 1363-1364CA>GT nucleotide changes.

In some embodiments, a gRNA that targets to a sequence corresponding to a mutation in COL8A2 known to produce a Leu450Trp mutation is used. In some embodiments, a guide sequence complementary to a target sequence of SEQ ID NOs: 1076-1084 is used, e.g., to selectively edit the Leu450Trp mutation caused by the c. 1349T>G nucleotide change.

### Target Sequences

In some embodiments, the guide RNA targets a nuclease to the COL8A2 gene. In some aspects, the crRNA comprises a guide sequence that is complementary to, and hybridizes with, a target sequence flanking the TNRs in the TCF4 gene. In some embodiments, two gRNAs are utilized. In such embodiments, the two gRNAs may flank a TNR of the TCF4 gene (i.e., the two gRNAs are on either side of the TNR). In some embodiments, one gRNA is within a TNR of the TCF4 gene and the other gRNA is outside of the TNR (i.e., flanks) the TNR of the TCF4 gene. In some embodiments the crRNA further comprises a flagpole region that is complementary to and hybridizes with a portion of a trRNA. In some embodiments, the crRNA may parallel the structure of a naturally occurring crRNA transcribed from a CRISPR locus of a bacteria, where the guide sequence acts as the “spacer” of the CRISPR/Cas9 system, and the flagpole corresponds to a portion of a repeat sequence flanking the spacers on the CRISPR locus.

### Target Sequences for TCF4

The compositions of the present invention may be directed to and cleave a target sequence within or flanking TNRs in the TCF4 gene. For example, the TNR target sequence may be recognized and cleaved by the provided nuclease. In some embodiments, a Cas protein may be directed by a guide RNA to a target sequence flanking TNRs in the TCF4 gene, where the guide sequence of the guide RNA hybridizes with the target sequence or its reverse complement and directs a Cas protein to cleave the target sequence. In some embodiments, a Cas protein may be directed by a guide RNA to a target sequence within TNRs in the TCF4 gene. In some embodiments, a Cas protein may be directed by more than one guide RNA to two target sequences flanking TNRs in the TCF4 gene. In some embodiments, a Cas protein may be directed by more than one guide RNA to two target sequences, wherein one flanks TNRs in the TCF4 gene and another is within the TNRs in the TCF4 gene.

In some embodiments, the selection of the one or more guide RNA is determined based on target sequences near TNRs in the TCF4 gene. For example, in some embodiments, the one or more guide RNA comprises a guide that is complementary to target sequences flanking TNRs in the TCF4 gene. In some embodiments, the crRNA sequence of the one or more guide RNA is complementary to and hybridizes to a target sequence chosen from SEQ ID NOs: 1-190.

In some embodiments, the target sequence may be complementary to the guide sequence of the guide RNA. In some embodiments, the degree of complementarity or identity between a guide sequence of a guide RNA and its corresponding target sequence may be about 50%, 55%, 60%, 65%, 70%, 75%, 80%, 85%, 90%, 95%, 96%, 97%, 98%, 99%, or 100%. In some embodiments, the target sequence and the guide sequence of the gRNA may be 100% complementary or identical. In other embodiments, the target sequence and the guide sequence of the gRNA may contain at least one mismatch. For example, the target sequence and the guide sequence of the gRNA may contain 1, 2, 3, 4, 5, 6, 7, 8, 9, or 10 mismatches, where the total length of the guide sequence is about 20. In some embodiments, the target sequence and the guide sequence of the gRNA may contain 1-6 mismatches where the guide sequence is about 20 nucleic acids. In some embodiments, the target sequence and the guide sequence of the gRNA may contain 1 or 2 mismatches where the guide sequence is about 20 nucleic acids.

The length of the target sequence may depend on the nuclease system used. For example, the target sequence for a CRISPR/Cas system may comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides. In some embodiments, the target sequence may comprise 18-24 nucleotides. In some embodiments, the target sequence may comprise 19-21 nucleotides. In some embodiments, the target sequence may comprise 20 nucleotides. When nickases are used, the target sequence may comprise a pair of target sequences recognized by a pair of nickases on opposite strands of the DNA molecule.

### Target Sequences for COL8A2

The compositions of the present invention may be directed to a target sequence in the COL8A2 gene. For example, the COL8A2 target sequence may be recognized and cleaved by the provided nuclease. In some embodiments, a Cas protein may be directed by a guide RNA to a target sequence of COL8A2, where the guide sequence of the guide RNA hybridizes with and the Cas protein cleaves the target sequence.

In some embodiments, the selection of the one or more guide RNA is determined based on target sequences in the COL8A2 gene. In some embodiments, the crRNA sequence of the one or more guide RNA is complementary to and hybridizes to a target sequence chosen from SEQ ID NOs: 191-1084.

In some embodiments, the selection of the one or more guide RNA is determined based on target sequences in the wild type COL8A2 gene, which does not have known mutations leading to abnormal function of the alpha subunit of collagen VIII (COL8A2). In some embodiments, the crRNA sequence of the one or more guide RNA is complementary to and hybridizes to a target sequence chosen from SEQ ID NOs: 191-1063.

In some embodiments, the selection of the one or more guide RNA is determined based on target sequences in the COL8A2 gene that correspond to Gln455Lys mutations in the COL8A2 protein, caused by the c. 1364C>A nucleotide change. In some embodiments, the crRNA sequence of the one or more guide RNA is complementary to and hybridizes to a target sequence chosen from SEQ ID NOs: 1064-1069.

In some embodiments, the selection of the one or more guide RNA is determined based on target sequences in the COL8A2 gene that correspond to Gln455Val mutations in the COL8A2 protein, caused by the c. 1363-1364CA>GT nucleotide changes. In some embodiments, the crRNA sequence of the one or more guide RNA is complementary to and hybridizes to a target sequence chosen from SEQ ID NOs: 1070-1075.

In some embodiments, the selection of the one or more guide RNA is determined based on target sequences in the COL8A2 gene that correspond to Leu450Trp mutations in the COL8A2 protein, caused by the c. 1349T>G nucleotide change. In some embodiments, the crRNA sequence of the one or more guide RNA is complementary to and hybridizes to a target sequence chosen from SEQ ID NOs: 1076-1084.

In some embodiments, the target sequence may be complementary to the guide sequence of the guide RNA. In some embodiments, the degree of complementarity or identity between a guide sequence of a guide RNA and its corresponding target sequence may be about 50%, 55%, 60%, 65%, 70%, 75%, 80%, 85%, 90%, 95%, 96%, 97%, 98%, 99%, or 100%. In some embodiments, the target sequence and the guide sequence of the gRNA may be 100% complementary or identical. In other embodiments, the target sequence and the guide sequence of the gRNA may contain at least one mismatch. For example, the target sequence and the guide sequence of the gRNA may contain 1, 2, 3, 4, 5, 6, 7, 8, 9, or 10 mismatches, where the total length of the guide sequence is about 20. In some embodiments, the target sequence and the guide sequence of the gRNA may contain 1-6 mismatches where the guide sequence is about 20 nucleic acids. In some embodiments, the target sequence and the guide sequence of the gRNA may contain 1 or 2 mismatches where the guide sequence is about 20 nucleic acids.

The length of the target sequence may depend on the nuclease system used. For example, the target sequence for a CRISPR/Cas system may comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides. In some embodiments, the target sequence may comprise 18-24 nucleotides. In some embodiments, the target sequence may comprise 19-21 nucleotides. In some embodiments, the target sequence may comprise 20 nucleotides. The target sequence may include a PAM. When nickases are used, the target sequence may comprise a pair of target sequences recognized by a pair of nickases on opposite strands of the DNA molecule.

### Vectors

In certain embodiments of the invention, the compositions comprise DNA vectors encoding any of the guide RNAs described herein. In some embodiments, in addition to guide RNA sequences, the vectors further comprise nucleic acids that do not encode guide RNAs. Nucleic acids that do not encode guide RNA include, but are not limited to, promoters, enhancers, regulatory sequences, and nucleic acids encoding a nuclease such as Cas9. In some embodiments, the vector comprises a nucleotide sequence encoding a crRNA, a trRNA, or a crRNA and trRNA. In some embodiments, the nucleotide sequence encoding the crRNA, trRNA, or crRNA and trRNA comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

In some embodiments, the crRNA and the trRNA are encoded by non-contiguous nucleic acids within one vector. In other embodiments, the crRNA and the trRNA may be encoded by a contiguous nucleic acid. In some embodiments, the crRNA and the trRNA are encoded by opposite strands of a single nucleic acid. In other embodiments, the crRNA and the trRNA are encoded by the same strand of a single nucleic acid. In some embodiments, the vector encodes one or more sgRNAs. In other embodiments, the vector encodes two or more sgRNAs.

### Nuclease

In some embodiments, in addition to the at least one gRNA, the composition further comprises a nuclease. In some embodiments, the gRNA together with nuclease is called a ribonucleoprotein complex (RNP). In some embodiments, the nuclease is a Cas protein. In some embodiments, the gRNA together with a Cas protein is called a Cas RNP. In some embodiments, the Cas comprises Type-I, Type-II, or Type-III components. In some embodiments, the Cas protein is from the Type-I CRISPR/Cas system. In some embodiments, the Cas protein is from the Type-II CRISPR/Cas system. In some embodiments, the Cas protein is from the Type-III CRISPR/Cas system. In some embodiments, the Cas protein is Cas9. In some embodiments, the Cas protein is Cpf1. In some embodiments, the Cas protein is the Cas9 protein from the Type-II CRISPR/Cas system. In some embodiment, the gRNA together with Cas9 is called a Cas9 RNP.

In embodiments encompassing a Cas nuclease, the Cas nuclease may be from a Type-IIA, Type-IIB, or Type-IIC system. Non-limiting exemplary species that the Cas nuclease or other RNP components may be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina. In some embodiments, the Cas nuclease is the Cas9 protein from Streptococcus pyogenes. In some embodiments, the Cas nuclease is the Cas9 protein from Streptococcus thermophilus. In some embodiments, the Cas nuclease is the Cas9 protein from Neisseria meningitidis. In some embodiments, the Cas nuclease is the Cas9 protein is from Staphylococcus aureus. In some embodiments, the Cas nuclease is the Cpf1 protein from Francisella novicida. In some embodiments, the Cas nuclease is the Cpf1 protein from Acidaminococcus sp. In some embodiments, the Cas nuclease is the Cpf1 protein from Lachnospiraceae bacterium ND2006.

Wild type Cas9 has two nuclease doacmains: RuvC and HNH. The RuvC domain cleaves the non-target DNA strand, and the HNH domain cleaves the target strand of DNA. In some embodiments, the Cas9 protein comprises more than one RuvC domain and/or more than one HNH domain. In some embodiments, the Cas9 protein is a wild type Cas9. In each of the composition and method embodiments, the Cas induces a double strand break in target DNA.

Modified versions of Cas9 having one catalytic domain, either RuvC or HNH, that is inactive are termed “nickases”. Nickases cut only one strand on the target DNA, thus creating a single-strand break. A single-strand break may also be known as a “nick.” In some embodiments, the compositions and methods comprise nickases. In some embodiments, the compositions and methods comprise a nickase Cas9 that induces a nick rather than a double strand break in the target DNA.

In some embodiments, the Cas protein may be modified to contain only one functional nuclease domain. For example, the Cas protein may be modified such that one of the nuclease domains is mutated or fully or partially deleted to reduce its nucleic acid cleavage activity. In some embodiments, a nickase Cas is used having a RuvC domain with reduced activity. In some embodiments, a nickase Cas is used having an inactive RuvC domain. In some embodiments, a nickase Cas is used having an HNH domain with reduced activity. In some embodiments, a nickase Cas is used having an inactive HNH domain.

In some embodiments, a conserved amino acid within a Cas protein nuclease domain is substituted to reduce or alter nuclease activity. In some embodiments, a Cas protein may comprise an amino acid substitution in the RuvC or RuvC-like nuclease domain. Exemplary amino acid substitutions in the RuvC or RuvC-like nuclease domain include D10A (based on the S. pyogenes Cas9 protein). In some embodiments, the Cas protein may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein).

In some embodiments, the composition comprises a nickase and a pair of guide RNAs. In some embodiments, the pair of guide RNAs are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, use of double nicking may improve specificity and reduce off-target effects. In some embodiments, a nickase Cas is used together with two separate guide RNAs targeting opposite strands of DNA to produce a double nick in the target DNA. In some embodiments, a nickase Cas is used together with two separate guide RNAs that are selected to be in close proximity to produce a double nick in the target DNA.

In some embodiments, chimeric Cas proteins are used, where one domain or region of the protein is replaced by a portion of a different protein. In some embodiments, a Cas nuclease domain may be replaced with a domain from a different nuclease such as Fok1. In some embodiments, a Cas protein may be a modified nuclease.

In some embodiments, a Cas9-deaminase fusion is used, wherein the Cas9 is not capable of cleaving double-stranded DNA (dCas9). The term “deaminase” refers to an enzyme that catalyzes a deamination reaction. In some embodiments, the deaminase is a cytidine deaminase that converts cytidine (C) to uracil (U), which then gets converted by the cell to thymidine (T). In some embodiments, the deaminase is a guanine deaminase that converts guanine (G) to xanthine, which then gets converted by the cell to adenine (A). In some embodiments, the deaminase is an APOBEC 1 family deaminase, an activation-induced cytidine deaminase (AID), and adenosine deaminase such as an ADAT family deaminase, or an adenosine deaminase acting on RNA (ADAR), that converts adenine (A) to hypoxanthine, which then gets converted by the cell to guanine (G).

In other embodiments, the Cas protein may be from a Type-I CRISPR/Cas system. In some embodiments, the Cas protein may be a component of the Cascade complex of a Type-I CRISPR/Cas system. In some embodiments, the Cas protein may be a Cas3 protein. In some embodiments, the Cas protein may be from a Type-III CRISPR/Cas system. In some embodiments, the Cas protein may have an RNA cleavage activity.

### PAM

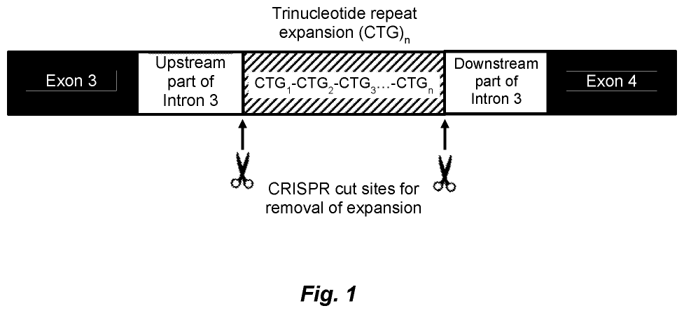

In some embodiments, the target sequence may be adjacent to a PAM. In some embodiments, the PAM may be adjacent to or within 1, 2, 3, or 4, nucleotides of the 3′ end of the target sequence. The length and the sequence of the PAM may depend on the Cas protein used. For example, the PAM may be selected from a consensus or a particular PAM sequence for a specific Cas9 protein or Cas9 ortholog, including those disclosed in FIG. 1 of Ran et al., Nature 520:186-191 (2015), which is incorporated herein by reference. In some embodiments, the PAM may comprise 2, 3, 4, 5, 6, 7, 8, 9, or 10 nucleotides in length. Non-limiting exemplary PAM sequences include NGG, NAG, NGA, NGAG, NGCG, NNGRRT, TTN, NGGNG, NG, NAAAAN, NNAAAAW, NNNNACA, GNNNCNNA, and NNNNGATT (wherein N is defined as any nucleotide, and W is defined as either A or T, and R is defined as either A or G). In some embodiments, the PAM sequence may be NGG. In some embodiments, the PAM sequence may be NGGNG. In some embodiments, the PAM sequence may be NNAAAAW.

### Methods of Excising TNRs

TNRs in TCF4 have been correlated with increased risk of FECD. Additionally, mutations in TCF4 have been associated with schizophrenia and PSC. Delivery of guide RNAs together with a Cas protein (or nucleic acid encoding a Cas protein) may be used as a treatment for these disorders, for example by excising TNRs (or a portion thereof) from the TCF4 gene. Accordingly, certain embodiments provided herein involve methods of excising TNRs from TCF4. In some embodiments, the method of comprises delivering to a cell any one of the CRISPR/Cas compositions provided herein which comprise one or more gRNAs which direct a nuclease to a Target Sequence provided in Table 2 herein. In some embodiments, the method comprises delivering to a cell two gRNAs together with a Cas protein (or nucleic acid encoding a Cas protein), wherein a first gRNA comprises a guide sequence which targets a region 5′ of the TNR and is selected from the group consisting of SEQ ID NOs: 1089-1181 and a second gRNA comprises a guide sequence which targets a region 3′ of the TNR and is selected from the group consisting of SEQ ID NOs: 1182-1278. In some embodiments, the cell is a human cell, for example a human corneal endothelium cell. In some embodiments, the method results in a population of cells wherein some fraction of the population has the TNR excised from a TCF4 gene. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% or more of the cells within the population has the TNR excised from a TCF4 gene. Methods for measuring the percent of exision within a population of cells are known, and include those provided herein, e.g., next generation sequencing (NGS) methods, for example where the excision percentage is defined as the number of sequencing reads containing a deletion of the TNRs divided by the total number of reads overlapping the target region.

Use of the CRISPR/Cas system can lead to double-stranded breaks in the DNA, or single-stranded breaks in the DNA if a nickase enzyme is used.

NHEJ is a process whereby double-stranded breaks (DSBs) in the DNA are repaired via re-ligation of the break ends, which can produce errors in the form of insertion/deletion (indel) mutations. NHEJ can thus be a means to knockout or reduce levels of a specific gene product, as indels occurring within a coding exon can lead to frameshift mutations and premature stop codons.

HR and HDR are alternative major DNA repair pathways that can be leveraged to generate precise, defined modifications at a target locus in the presence of an exogenously introduced repair template. This can be used to correct single base changes, deletions, insertions, inversions, and other mutations. In some cases, a repair template is used that introduces silent (i.e., synonymous) nucleotide changes within the DNA that prevent recognition by the CRISPR nuclease used to initiate the repair process, thereby preventing indel formation within the corrected gene.

In some embodiments, the template may be used in HR, e.g., to modify a target gene such as TCF4 and/or COL8A2. In some embodiments, the HR may result in the integration of the template sequence or a portion of the template sequence into the target nucleic acid molecule. In some embodiments, a single template may be provided. In other embodiments, two or more templates may be provided such that HR may occur at two or more target sites. For example, different templates may be provided to repair a single gene in a cell, or two different genes in a cell. In some embodiments, multiple copies of at least one template are provided to a cell. In some embodiments, the different templates may be provided in independent copy numbers or independent amounts.

In other embodiments, the template may be used in HDR, e.g., to modify a target gene such as TCF4 and/or COL8A2. HDR involves DNA strand invasion at the site of the cleavage in the nucleic acid. In some embodiments, the HDR may result in including the template sequence in the edited target nucleic acid molecule. In some embodiments, a single template may be provided. In other embodiments, two or more templates having different sequences may be used at two or more sites by HDR. For example, different templates may be provided to repair a single gene in a cell, or two different genes in a cell. In some embodiments, multiple copies of at least one template are provided to a cell. In some embodiments, the different templates may be provided in independent copy numbers or independent amounts.

In yet other embodiments, the template may be used in gene editing mediated by NHEJ, e.g., to modify a target gene such as TCF4 and/or COL8A2. In some embodiments, the template sequence has no similarity to the nucleic acid sequence near the cleavage site. In some embodiments, the template or a portion of the template sequence is incorporated. In some embodiments, a single template may be provided. In other embodiments, two or more templates having different sequences may be inserted at two or more sites by NHEJ. For example, different templates may be provided to insert a single template in a cell, or two different templates in a cell. In some embodiments, the different templates may be provided in independent copy numbers. In some embodiments, the template includes flanking inverted terminal repeat (ITR) sequences.

The template may be of any suitable length. In some embodiments, the template may comprise 10, 15, 20, 25, 50, 75, 100, 150, 200, 500, 1000, 1500, 2000, 2500, 3000, 3500, 4000, 4500, 5000, 5500, 6000, or more nucleotides in length. The template may be a single-stranded nucleic acid. The template can be double-stranded or partially double-stranded nucleic acid. In certain embodiments, the single stranded template is 20, 30, 40, 50, 75, 100, 125, 150, 175, or 200 nucleotides in length. In some embodiments, the template may comprise a nucleotide sequence that is complementary to a portion of the target nucleic acid molecule comprising the target sequence (i.e., a “homology arm”). In some embodiments, the template may comprise a homology arm that is complementary to the sequence located upstream or downstream of the cleavage site on the target nucleic acid molecule. In some embodiments, the template may comprise a first homology arm and a second homology arm (also called a first and second nucleotide sequence) that are complementary to sequences located upstream and downstream of the cleavage site, respectively. Where a template contains two homology arms, each arm can be the same length or different lengths, and the sequence between the homology arms can be substantially similar or identical to the target sequence between the homology arms, or it can be entirely unrelated. In some embodiments, the degree of complementarity between the first nucleotide sequence on the template and the sequence upstream of the cleavage site, and between the second nucleotide sequence on the template and the sequence downstream of the cleavage site, may permit homologous recombination, such as, e.g., high-fidelity homologous recombination, between the template and the target nucleic acid molecule. In some embodiments, the degree of complementarity may be about 50%, 55%, 60%, 65%, 70%, 75%, 80%, 85%, 90%, 95%, 97%, 98%, 99%, or 100%. In some embodiments, the degree of complementarity may be about 95%, 97%, 98%, 99%, or 100%. In some embodiments, the degree of complementarity may be at least 98%, 99%, or 100%. In some embodiments, the degree of complementarity may be 100%.

In some embodiments, the template contains ssDNA or dsDNA containing flanking invert-terminal repeat (ITR) sequences. In some embodiments, the template is supplied as a plasmid, minicircle, nanocircle, or PCR product.

### Excision Fragments

Generation of excision fragments is a means to harness the power of CRISPR technology to precisely remove small regions of DNA between two target sequences through use of two guide RNAs complementary to these target sequences. In some embodiments, the two guide RNAs target nucleases to sequences that are about 3000, 2500, 2000, 1500, 1000, 500, 400, 300, 200, 150, 100, 50, or 30 nucleotides apart, leading to excision of a DNA fragment between the target sequences.

Treatment of FECD with CRISPR/Cas Compositions

Any of the compositions described herein may be administered to subjects to treat FECD in individuals with genetic mutations leading to increased risk of FECD.

Any of the compositions described herein may be administered to subjects to treat FECD in individuals with TNR expansion in intron 3 of TCF4. Methods of treating FECD comprising administering any of the compositions described herein are encompassed. In some aspects, the compositions are administered in therapeutically effective amounts. In some embodiments, a method of excising, mutating, reducing copy number of, ameliorating, and/or eradicating TNRs of TCF4 is encompassed, comprising administering one or more of the compositions described herein. In some embodiments, a method of excising, reducing copy number of, ameliorating, and/or eradicating the TNRs of one or both copies of TCF4 per cell in a subject is provided, comprising administering one or more of the compositions described herein. In some embodiments, the cell is a corneal endothelium cell.

In some aspects, a method of reducing, inhibiting, or ameliorating RNA toxicity of TCF4 comprising administering one or more of the compositions described herein is encompassed. In some embodiments, a method of inhibiting RNA toxicity is encompassed comprising administering one or more of the compositions described herein, wherein the level of toxic RNA products of TCF4 does not return to pre-administration levels after treatment, returning normal function to the corneal endothelial cells, and preventing cell death.

In some embodiments, treatment may be with a vector and/or lipid nanoparticle comprising the appropriate guide or guides, delivered into the anterior chamber of the eye. In some embodiments, treatment may be with a vector and/or lipid nanoparticle comprising the appropriate guide or guides, delivered into the posterior chamber of the eye. In some embodiments, treatment may be with a vector and/or lipid nanoparticle comprising the appropriate guide or guides, delivered into the cornea itself. In some embodiments, treatment may be with a vector and/or lipid nanoparticle comprising the appropriate guide or guides, delivered into the corneal stroma. In some embodiments, treatment may be with a vector and/or lipid nanoparticle comprising the appropriate guide or guides, delivered into the corneal limbus. In some embodiments, treatment may be with a vector and/or lipid nanoparticle comprising the appropriate guide or guides, delivered topically onto the epithelial surface of the cornea. In any of the preceding embodiments of this paragraph as well as other embodiments described herein, treatment further comprises delivery of a Cas protein (e.g., Cas9), for example using a lipid nanoparticle, or delivery of a nucleic acid encoding a Cas protein using a vector and/or lipid nanoparticle. In some embodiments, for example those using a lipid nanoparticle, the nucleic acid encoding the Cas protein is mRNA. In some embodiments, a Cas protein or a nucleic acid encoding a Cas protein is delivered via the same vector and/or lipid nanoparticle that is used to deliver the appropriate guide or guides. In some embodiments, a Cas protein or a nucleic acid encoding a Cas protein is delivered via a different vector and/or lipid nanoparticle that is used to deliver the appropriate guide or guides.

In some embodiments, a single administration of the CRISPR compositions of the invention may be sufficient to correct the underlying genetic defect or mutation associated with disease. In other embodiments, more than one administration of the CRISPR therapeutic may be beneficial, to maximize editing across all target cells and all alleles via cumulative effects.

Use of the compositions described herein for the preparation of a medicament for treating FECD are encompassed. In some embodiments, the patient with FECD, possible FECD, and/or a family history suggestive of FECD is screened for TNRs in TCF4 before initiation of treatment with the compositions of the invention. In some embodiments, treatment is initiated in a patient if 50 or more TNR are present in intron 3 of TCF4.

Mutations in COL8A2 have been correlated with an increased risk of FECD and PPCD. Any of the compositions described herein may be administered to subjects to treat FECD in individuals with mutations in COL842 leading to gene products with amino acid mutations. In some embodiments, these amino acid mutations are Gln455Lys, Gln455Val, or Leu450Trp.

Methods of treating FECD comprising administering any of the compositions described herein are encompassed. In some aspects, the compositions are administered in therapeutically effective amounts. In some embodiments, a method of cleaving, mutating, ameliorating, and/or eradicating mutations in COL842 is encompassed, comprising administering one or more of the compositions described herein. In some embodiments, use of CRISPR/Cas compositions is done together with a process of NHEJ, leading to generation of indels and loss of a COL8A2 allele. In some embodiments, use of CRISPR/Cas compositions is done together with either an exogenous template for HR/HDR, or using the endogenous normal allele as template for HR/HDR, for the purpose of correcting a nucleic acid mutation that leads to an amino acid mutation in the alpha 2 subunit of collagen VIII. In some embodiments, the mutation in the COL8A2 gene that is corrected is the Gln455Lys mutation, caused by the c. 1364C>A nucleotide change. In some embodiments, the mutation in the COL8A2 gene that is corrected is the Gln455Val mutation caused by the c. 1363-1364CA>GT nucleotide changes. In some embodiments, the mutation in the ((18A2 gene that is corrected is the Leu450Trp mutation caused by the c. 1349T>G nucleotide change. In some embodiments, use of a template together with a Cas RNP leads to correction of the nucleic acid sequence such that the mutation is no longer present. In some embodiments, the cell is a corneal endothelium cell.

In some aspects, a method of reducing, inhibiting, or ameliorating the abnormal collagen formed by mutant COL8A2, comprising administration of one or more of the compositions described herein is encompassed. In some embodiments, a method of inhibiting production of abnormal alpha subunit of collagen VIII (COL8A2) is encompassed comprising administration of one or more of the compositions described herein, wherein the level of abnormal COL8A2 does not return to pre-administration levels after treatment. In some embodiments, a method of correcting a genetic mutation with HR or HDR, such that only normal collagen is produced, is encompassed comprising administering one or more of the compositions described herein. Reduction or correction of the mutant form of collagen should prevent the abnormal collagen deposition seen in the cornea of FECD patients.

Use of the compositions described herein for the preparation of a medicament for treating FECD are encompassed. In some embodiments, the patient with FECD, possible FECD, and/or a family history suggestive of FECD is screened for mutation in COL8A2 before initiation of treatment with the compositions of the invention. In some embodiments, the patient with PPCD, possible PPCD, and/or a family history suggestive of PPCD is screened for mutation in COL8A2 before initiation of treatment with the compositions of the invention. In some embodiments, treatment is initiated in a patient if a mutation is present, such the Gln455Lys mutation caused by the c. 1364C>A nucleotide change, the Gln455Val mutation caused by the c. 1363-1364CA>GT nucleotide changes, or the Leu450Trp mutation caused by the c. 1349T>G nucleotide change.

In some embodiments, a single administration of the CRISPR compositions of the invention may be sufficient to correct the underlying genetic defect or mutation associated with disease. In other embodiments, more than one administration of the CRISPR therapeutic may be beneficial, to maximize editing across all target cells and all alleles via cumulative effects. In some embodiments, the efficacy of treatment with the compositions of the invention is seen at 1 year, 2 years, 3 years, 4 years, 5 years, or 10 years after delivery.

A number of different types of assessments may be used to determine efficacy of a treatment for FECD, see Eghrari and Gottsch, Expert Rev Ophthalmol. 5 (2): 147-159 (2010). In some embodiments, efficacy of treatment with the compositions is based on assessment by slit-lamp microscopy over time. In some embodiments, efficacy of treatment with the compositions is based on quantitative measurement of disease progression by corneal pachymetry measurements of corneal thickness over time. In some embodiments, efficacy of treatment with the compositions is based on improvement, stabilization, or slowing of change in corneal pachymetry over time.

In some embodiments, efficacy of treatment with the compositions is based on assessment of visual acuity over time. In some embodiments, efficacy of treatment with the compositions is based on improvement, stabilization, or slowing of decline in visual acuity over time.

In some embodiments, efficacy of treatment with the compositions is based on specular microscopy. In some embodiments, this specular microscopy is used to document the presence of guttae. In some embodiments, efficacy of treatment with the compositions is based on a decrease in formation of new guttae. In some embodiments, efficacy of treatment with the compositions is based on a decrease in presence of existing guttae.

In some embodiments, efficacy of treatment with the compositions is based on the patient retaining acceptable visual acuity and avoiding need for a corneal transplant. In some embodiments, efficacy of treatment with the compositions is based on a delay in the time until a corneal transplant is needed. This corneal transplant may be a full corneal transplant or a transplant of the inner layer of the cornea.

In addition to being associated with FECD, genetic variants in the TCF4 gene have been associated with two other conditions, primary sclerosing cholangitis (PSC) and schizophrenia (see Forrest M P et al., Trends Mol Med. 2014 June; 20 (6): 322-31). It remains unclear how noncoding variants in the TCF4 gene increase risk for PSC and schizophrenia. One possibility is that these variants serve as markers for a co-inherited expansion in the same TNR region within intron 3 that has been linked to RNA-mediated toxicity in FECD. While this hypothesis remains unproven, the variants associated with PSC and schizophrenia are located physically and haplotypically close to the TNR-containing region within intron 3, suggesting co-inheritance of variants in these neighboring regions. Moreover, the risk variants associated with PSC and schizophrenia have not been associated with changes in expression of the TCF4 gene, suggesting that another mechanism is involved, such as the RNA toxicity seen in patients with the TNR expansion in intron 3.

### Combination Therapy

In some embodiments, the compositions of the invention are used as a single agent for the treatment of FECD, PPCD, PSC, and/or Schizophrenia.

In some embodiments, the compositions of the invention are used in combination with other therapies for FECD, PPCD, PSC, and/or Schizophrenia. In some embodiments, the combination therapy is soft contact lenses. In some embodiments, these soft contact lenses smooth out microscopic swelling on the surface of the eye. In some embodiments, the compositions of the invention are used in combination with eye drops or ointments that draw fluid out of the cornea. In some embodiments, these eye drops or ointments are Muro 1288 5% (Sodium Chloride Hypertonicity Ophthalmic Solution, 5%, Bausch and Lomb), Muro 128 5% Ointment (Sodium Chloride Hypertonicity Ophthalmic Ointment, 5%) (Bausch and Lomb), or other saline or tear replacements.

In some embodiments, glucocorticoids or corticosteroids are used together with the compositions of the invention to reduce the immune response to the therapeutic.

Combination treatments may be achieved by way of the simultaneous, sequential, or separate dosing of the individual components of the treatment. Where the administration is sequential or separate, the delay in administering the second component should not be such as to lose the beneficial effect of the combination.

### Delivery of CRISPR/Cas Compositions

In some embodiments, the CRISPR/Cas compositions described herein may be administered via a vector and/or lipid nanoparticle comprising the appropriate guide or guides.

### Viral Vectors

CRISPR/Cas composistions can be delivered by a vector system. In some embodiments, the CRISPR/Cas composistions may be provided on one or more vectors. In some embodiments, the vector may be a DNA vector. In other embodiments, the vector may be an RNA vector. In some embodiments, the vector may be circular. In other embodiments, the vector may be linear. In some embodiments, the vector may be enclosed in a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

In some embodiments, the vector may be a viral vector. In some embodiments, the viral vector may be genetically modified from its wild type counterpart. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some embodiments, a portion of the viral genome may be deleted such that the virus is capable of packaging exogenous sequences having a larger size. In some embodiments, the viral vector may have an enhanced transduction efficiency. In some embodiments, the immune response induced by the virus in a host may be reduced. In some embodiments, viral genes (such as, e.g., integrase) that promote integration of the viral sequence into a host genome may be mutated such that the virus becomes non-integrating. In some embodiments, the viral vector may be replication defective. In some embodiments, the viral vector may comprise exogenous transcriptional or translational control sequences to drive expression of coding sequences on the vector. In some embodiments, the virus may be helper-dependent. For example, the virus may need one or more helper virus to supply viral components (such as, e.g., viral proteins) required to amplify and package the vectors into viral particles. In such a case, one or more helper components, including one or more vectors encoding the viral components, may be introduced into a host cell along with the vector system described herein. In other embodiments, the virus may be helper-free. For example, the virus may be capable of amplifying and packaging the vectors without any helper virus. In some embodiments, the vector system described herein may also encode the viral components required for virus amplification and packaging.

Non-limiting exemplary viral vectors include adeno-associated virus (AAV) vector, lentivirus vectors, adenovirus vectors, helper dependent adenoviral vectors (HDAd), herpes simplex virus (HSV-1) vectors, bacteriophage T4, baculovirus vectors, and retrovirus vectors. In some embodiments, the viral vector may be an AAV vector. In some embodiments, the AAV vector has a serotype of 2, 3, 5, 7, 8, 9, or rh. 10. In other embodiments, the viral vector may a lentivirus vector. In some embodiments, the lentivirus may be non-integrating.

In some embodiments, the viral vector may be an adenovirus vector. In some embodiments, the adenovirus may be a high-cloning capacity or “gutless” adenovirus, where all coding viral regions apart from the 5′ and 3′ inverted terminal repeats (ITRs) and the packaging signal (‘I’) are deleted from the virus to increase its packaging capacity. In yet other embodiments, the viral vector may be an HSV-1 vector. In some embodiments, the HSV-1-based vector is helper dependent, and in other embodiments it is helper independent. For example, an amplicon vector that retains only the packaging sequence requires a helper virus with structural components for packaging, while a 30 kb-deleted HSV-1 vector that removes non-essential viral functions does not require helper virus. In additional embodiments, the viral vector may be bacteriophage T4. In some embodiments, the bacteriophage T4 may be able to package any linear or circular DNA or RNA molecules when the head of the virus is emptied. In further embodiments, the viral vector may be a baculovirus vector. In yet further embodiments, the viral vector may be a retrovirus vector. In embodiments using AAV or lentiviral vectors, which have smaller cloning capacity, it may be necessary to use more than one vector to deliver all the components of a vector system as disclosed herein. For example, one AAV vector may contain sequences encoding a Cas protein, while a second AAV vector may contain one or more guide sequences. However, in some embodiments, a single AAV vector may contain sequences encoding a Cas protein and one or more guide sequences. In some embodiments involving use of a single AAV to deliver CRISPR/Cas components described herein, a small Cas9 ortholog is used. In some embodiments, the small Cas9 ortholog is derived from Neisseria meningitidis, Campylobacter jejuni or Staphylococcus aureus.

In some embodiments, the vector may be capable of driving expression of one or more coding sequences in a cell. In some embodiments, the cell may be a prokaryotic cell, such as, e.g., a bacterial cell. In some embodiments, the cell may be a eukaryotic cell, such as, e.g., a yeast, plant, insect, or mammalian cell. In some embodiments, the eukaryotic cell may be a mammalian cell. In some embodiments, the eukaryotic cell may be a rodent cell. In some embodiments, the eukaryotic cell may be a human cell. Suitable promoters to drive expression in different types of cells are known in the art. In some embodiments, the promoter may be wild type. In other embodiments, the promoter may be modified for more efficient or efficacious expression. In yet other embodiments, the promoter may be truncated yet retain its function. For example, the promoter may have a normal size or a reduced size that is suitable for proper packaging of the vector into a virus.

In some embodiments, the vector may comprise a nucleotide sequence encoding the nuclease described herein. In some embodiments, the nuclease encoded by the vector may be a Cas protein. In some embodiments, the vector system may comprise one copy of the nucleotide sequence encoding the nuclease. In other embodiments, the vector system may comprise more than one copy of the nucleotide sequence encoding the nuclease. In some embodiments, the nucleotide sequence encoding the nuclease may be operably linked to at least one transcriptional or translational control sequence. In some embodiments, the nucleotide sequence encoding the nuclease may be operably linked to at least one promoter.

In some embodiments, the promoter may be constitutive, inducible, or tissue-specific. In some embodiments, the promoter may be a constitutive promoter. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EF1a promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On* promoter (Clontech).

In some embodiments, the promoter may be a tissue-specific promoter, e.g., a promoter specific for expression in the corneal endothelium.

The vector may further comprise a nucleotide sequence encoding the guide RNA described herein. In some embodiments, the vector comprises one copy of the guide RNA. In other embodiments, the vector comprises more than one copy of the guide RNA. In embodiments with more than one guide RNA, the guide RNAs may be non-identical such that they target different target sequences, or may be identical in that they target the same target sequence. In some embodiments where the vectors comprise more than one guide RNA, each guide RNA may have other different properties, such as activity or stability within the Cas RNP complex. In some embodiments, the nucleotide sequence encoding the guide RNA may be operably linked to at least one transcriptional or translational control sequence, such as a promoter, a 3′ UTR, or a 5′ UTR. In one embodiment, the promoter may be a tRNA promoter, e.g., tRNALys3, or a tRNA chimera. See Mefferd et al., RNA. 2015 21:1683-9; Scherer et al., Nucleic Acids Res. 2007 35:2620-2628. In some embodiments, the promoter may be recognized by RNA polymerase III (Pol III). Non-limiting examples of Pol III promoters include U6 and H1 promoters. In some embodiments, the nucleotide sequence encoding the guide RNA may be operably linked to a mouse or human U6 promoter. In other embodiments, the nucleotide sequence encoding the guide RNA may be operably linked to a mouse or human H1 promoter. In embodiments with more than one guide RNA, the promoters used to drive expression may be the same or different. In some embodiments, the nucleotide encoding the crRNA of the guide RNA and the nucleotide encoding the trRNA of the guide RNA may be provided on the same vector. In some embodiments, the nucleotide encoding the crRNA and the nucleotide encoding the trRNA may be driven by the same promoter. In some embodiments, the crRNA and trRNA may be transcribed into a single transcript. For example, the crRNA and trRNA may be processed from the single transcript to form a double-molecule guide RNA. Alternatively, the crRNA and trRNA may be transcribed into a single-molecule guide RNA. In other embodiments, the crRNA and the trRNA may be driven by their corresponding promoters on the same vector. In yet other embodiments, the crRNA and the trRNA may be encoded by different vectors.

In some embodiments, the nucleotide sequence encoding the guide RNA may be located on the same vector comprising the nucleotide sequence encoding a Cas protein. In some embodiments, expression of the guide RNA and of the Cas protein may be driven by their own corresponding promoters. In some embodiments, expression of the guide RNA may be driven by the same promoter that drives expression of the Cas9 protein. In some embodiments, the guide RNA and the Cas protein transcript may be contained within a single transcript. For example, the guide RNA may be within an untranslated region (UTR) of the Cas protein transcript. In some embodiments, the guide RNA may be within the 5′ UTR of the Cas protein transcript. In other embodiments, the guide RNA may be within the 3′ UTR of the Cas protein transcript. In some embodiments, the intracellular half-life of the Cas protein transcript may be reduced by containing the guide RNA within its 3′ UTR and thereby shortening the length of its 3′ UTR. In additional embodiments, the guide RNA may be within an intron of the Cas protein transcript. In some embodiments, suitable splice sites may be added at the intron within which the guide RNA is located such that the guide RNA is properly spliced out of the transcript. In some embodiments, expression of the Cas protein and the guide RNA in close proximity on the same vector may facilitate more efficient formation of the CRISPR RNP complex.

In some embodiments, the compositions comprise a vector system, wherein the system comprises more than one vector. In some embodiments, the vector system may comprise one single vector. In other embodiments, the vector system may comprise two vectors. In additional embodiments, the vector system may comprise three vectors. When different guide RNAs are used for multiplexing, or when multiple copies of the guide RNA are used, the vector system may comprise more than three vectors.

In some embodiments, the vector system may comprise inducible promoters to start expression only after it is delivered to a target cell. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In additional embodiments, the vector system may comprise tissue-specific promoters to start expression only after it is delivered into a specific tissue.

The vector may be delivered by liposome, a nanoparticle, an exosome, or a microvesicle. The vector may also be delivered by a lipid nanoparticle; see e.g., PCT/US2017/024973, filed Mar. 30, 2017, claiming priority to U.S. Ser. No. 62/315,602, filed Mar. 30, 2016 and entitled “LIPID NANOPARTICLE FORMULATIONS FOR CRISPR/CAS COMPONENTS,” the contents of which are hereby incorporated by reference in their entirety.

In some embodiments, the vector may be delivered via a solution delivered directly to the cornea. Delivery may be accomplished via topical application, injection into the cornea itself, injection into the anterior chamber, injection into the posterior chamber, injection into the corneal limbus, or other means.

In some embodiments, the vector may be delivered systemically.

### Lipid Nanoparticles (LNPs)

In some embodiments, the guide RNA compositions described herein, alone or encoded on one or more vectors, are administered via a lipid nanoparticle; see e.g., PCT/US2017/024973, filed Mar. 30, 2017, claiming priority to U.S. Ser. No. 62/315,602, filed Mar. 30, 2016 and entitled “LIPID NANOPARTICLE FORMULATIONS FOR CRISPR/CAS COMPONENTS,” the contents of which are hereby incorporated by reference in their entirety. Any lipid nanoparticle known to those of skill in the art to be capable of delivering nucleotides to subjects may be utilized to administer the guide RNAs described herein, as well as either mRNA encoding Cas or Cas-deaminase fusion protein or Cas9 or Cas9-deaminase fusion protein itself.

In some embodiments, the LNP comprises (i) a CCD lipid for encapsulation and for endosomal escape, (ii) a neutral lipid for stabilization, (iii) a helper lipid, also for stabilization, and (iv) a stealth lipid. The LNP carries cargo, which may include any or all of the following: an mRNA encoding a Cas nuclease or Cas-deaminase, such as Cas9 or Cas9-deaminase; one or more guide RNAs or a nucleic acids encoding one or more guide RNA; and one or more viral vectors encoding Cas9 or Cas9-deaminase, one or more guide RNAs, or both Cas9/Cas9-deaminase and guide RNAs. In one embodiment, the LNP comprises a CCD lipid, such as Lipid A, Lipid B, Lipid C, or Lipid D. In some aspects, the CCD lipid is Lipid A. In some aspects, the CCD lipid is Lipid B. In some embodiments, the LNP comprises a CCD lipid, a neutral lipid, a helper lipid, and a stealth lipid. In certain embodiments, the helper lipid is cholesterol. In certain embodiments, the neutral lipid is DSPC. In some embodiments, the stealth lipid is PEG2k-DMG. In additional embodiments, the LNP comprises a CCD lipid selected from Lipid A or Lipid B, cholesterol, DSPC, and PEG2k-DMG.

In some embodiments, suitable LNP formulations include a CCD lipid, along with a helper lipid, a neutral lipid, and a stealth lipid. By “lipid nanoparticle” is meant a particle that comprises a plurality of (i.e. more than one) lipid molecules physically associated with each other by intermolecular forces. The LNPs may be, e.g., microspheres (including unilamellar and multilamellar vesicles, e.g., “liposomes”—lamellar phase lipid bilayers that, in some embodiments, are substantially spherical—and, in more particular embodiments, can comprise an aqueous core, e.g., comprising a substantial portion of RNA molecules), a dispersed phase in an emulsion, micelles, or an internal phase in a suspension. Emulsions, micelles, and suspensions may be suitable compositions for local and/or topical delivery.

In some embodiments, the CCD lipid is Lipid A, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. Lipid A can be depicted as:

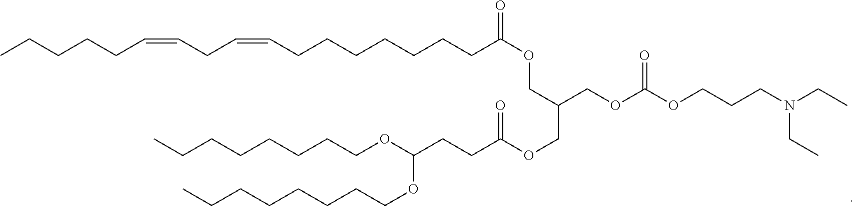 [SMILES: CCCCC/C=C\C/C=C\CCCCCCCC(=O)OCC(COC(=O)CCC(OCCCCCCCC)OCCCCCCCC)COC(=O)OCCCN(CC)CC]

Lipid A may be synthesized according to WO2015/095340 (e.g., pp. 84-86), incorporated by reference in its entirety.

In some embodiments, the CCD lipid is Lipid B, which is ((5-((dimethylamino)methyl)-1,3-phenylene)bis(oxy))bis(octane-8,1-diyl)bis(decanoate), also called ((5-((dimethylamino)methyl)-1,3-phenylene)bis(oxy))bis(octane-8,1-diyl)bis(decanoate). Lipid B can be depicted as:

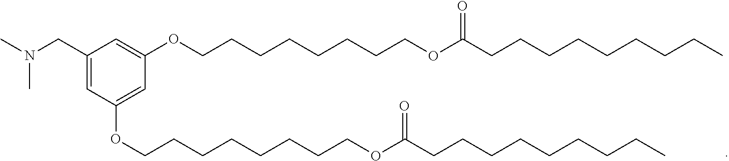 [SMILES: CCCCCCCCCC(=O)OCCCCCCCCOc1cc(CN(C)C)cc(OCCCCCCCCOC(=O)CCCCCCCCC)c1]

Lipid B may be synthesized according to WO2014/136086 (e.g., pp. 107-09), incorporated by reference in its entirety.

In some embodiments, the CCD lipid is Lipid C, which is 2-((4-(((3-(dimethylamino)propoxy)carbonyl)oxy)hexadecanoyl)oxy)propane-1,3-diyl (9Z,9′Z,12Z,12′Z)-bis(octadeca-9,12-dienoate). Lipid C can be depicted as:

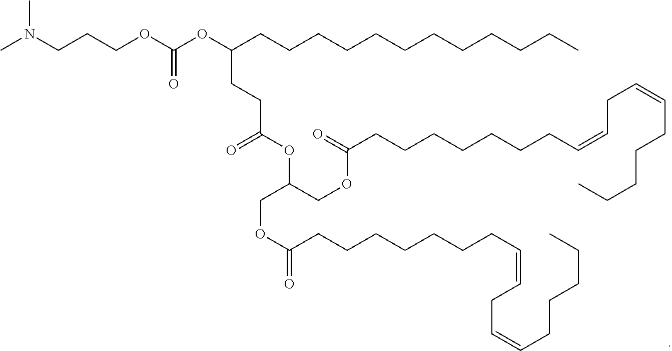 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C]

In some embodiments, the CCD lipid is Lipid D, which is 3-(((3-(dimethylamino)propoxy)carbonyl)oxy)-13-(octanoyloxy)tridecyl 3-octylundecanoate.

Lipid D can be depicted as:

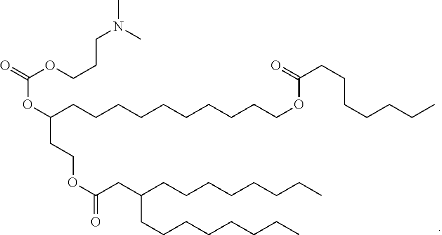 [SMILES: CCCCCCCCC(CCCCCCCC)CC(=O)OCCC(CCCCCCCCCCOC(=O)CCCCCCC)OC(=O)OCCCN(C)C]

Lipid C and Lipid D may be synthesized according to WO2015/095340, incorporated by reference in its entirety.

“Neutral lipids” suitable for use in a lipid composition include, for example, a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine (DSPC), pohsphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the neutral phospholipid may be selected from the group consisting of distearoylphosphatidylcholine (DSPC) and dimyristoyl phosphatidyl ethanolamine (DMPE). In another embodiment, the neutral phospholipid may be distearoylphosphatidylcholine (DSPC). Neutral lipids function to stabilize and improve processing of the LNPs.

“Helper lipids” are lipids that enhance transfection (e.g. transfection of the nanoparticle including the biologically active agent). The mechanism by which the helper lipid enhances transfection includes enhancing particle stability. In certain embodiments, the helper lipid enhances membrane fusogenicity. Helper lipids include steroids, sterols, and alkyl resorcinols. Helper lipids suitable for use in the LNPs include, but are not limited to, cholesterol, 5-heptadecylresorcinol, and cholesterol hemisuccinate. In one embodiment, the helper lipid may be cholesterol. In some embodiments, the helper lipid may be cholesterol hemisuccinate.

“Stealth lipids” are lipids that alter the length of time the nanoparticles can exist in vivo (e.g., in the blood). Stealth lipids may assist in the formulation process by, for example, reducing particle aggregation and controlling particle size. Stealth lipids used herein may modulate pharmacokinetic properties of the LNP. Stealth lipids suitable for use in a lipid composition include, but are not limited to, stealth lipids having a hydrophilic head group linked to a lipid moiety. Stealth lipids suitable for use in a lipid composition of the present disclosure and information about the biochemistry of such lipids can be found in Romberg et al., Pharmaceutical Research, Vol. 25, No. 1, 2008, pg. 55-71 and Hoekstra et al., Biochimica et Biophysica Acta 1660 (2004) 41-52. Additional suitable PEG lipids are disclosed, e.g., in WO 2006/007712.

In one embodiment, the hydrophilic head group of stealth lipid comprises a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids and poly[N-(2-hydroxypropyl) methacrylamide].

Stealth lipids may comprise a lipid moiety. In some embodiments, the lipid moiety of the stealth lipid may be derived from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

Unless otherwise indicated, the term “PEG” as used herein means any polyethylene glycol or other polyalkylene ether polymer. In some embodiments, PEG is an optionally substituted linear or branched polymer of ethylene glycol or ethylene oxide. In some embodiments, PEG is unsubstituted. In some embodiments, the PEG is substituted, e.g., by one or more alkyl, alkoxy, acyl, hydroxy, or aryl groups. In some embodiments, the term includes PEG copolymers such as PEG-polyurethane or PEG-polypropylene (see, e.g., J. Milton Harris, Poly (ethylene glycol) chemistry: biotechnical and biomedical applications (1992)); in another embodiment, the term does not include PEG copolymers. In some embodiments, the PEG has a molecular weight of from about 130 to about 50,000, in a sub-embodiment, about 150 to about 30,000, in a sub-embodiment, about 150 to about 20,000, in a sub-embodiment about 150 to about 15,000, in a sub-embodiment, about 150 to about 10,000, in a sub-embodiment, about 150 to about 6,000, in a sub-embodiment, about 150 to about 5,000, in a sub-embodiment, about 150 to about 4,000, in a sub-embodiment, about 150 to about 3,000, in a sub-embodiment, about 300 to about 3,000, in a sub-embodiment, about 1,000 to about 3,000, and in a sub-embodiment, about 1,500 to about 2,500.

In certain embodiments, the PEG (e.g., conjugated to a lipid, such as a stealth lipid), is a “PEG-2K,” also termed “PEG 2000,” which has an average molecular weight of about 2,000 daltons. PEG-2K is represented herein by the following formula (I), wherein n is 45, meaning that the number averaged degree of polymerization comprises about 45 subunits

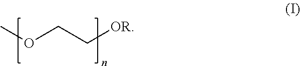

However, other PEG embodiments known in the art may be used, including, e.g., those where the number-averaged degree of polymerization comprises about 23 subunits (n=23), and/or 68 subunits (n=68). In some embodiments, n may range from about 30 to about 60. In some embodiments, n may range from about 35 to about 55. In some embodiments, n may range from about 40 to about 50. In some embodiments, n may range from about 42 to about 48. In some embodiments, n may be 45. In some embodiments, R may be selected from H, substituted alkyl, and unsubstituted alkyl. In some embodiments, R may be unsubstituted alkyl. In some embodiments, R may be methyl.

In any of the embodiments described herein, the stealth lipid may be selected from PEG-dilauroylglycerol, PEG-dimyristoylglycerol (PEG-DMG) (catalog #GM-020 from NOF, Tokyo, Japan), PEG-dipalmitoylglycerol, PEG-distearoylglycerol (PEG-DSPE) (catalog #DSPE-020CN, NOF, Tokyo, Japan), PEG-dilaurylglycamide, PEG-dimyristylglycamide, PEG-dipalmitoylglycamide, and PEG-distearoylglycamide, PEG-cholesterol (1-[8′-(Cholest-5-en-3 [beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy (polyethylene glycol)-2000] (PEG2k-DMG) (cat. #880150P from Avanti Polar Lipids, Alabaster, Alabama, USA), 1,2-distearoyl-sn-glycero-3-phosphoethanolamine-N-[methoxy (polyethylene glycol)-2000] (PEG2k-DSPE) (cat. #880120C from Avanti Polar Lipids, Alabaster, Alabama, USA), 1,2-distearoyl-sn-glycerol, methoxypolyethylene glycol (PEG2k-DSG; GS-020, NOF Tokyo, Japan), poly(ethylene glycol)-2000-dimethacrylate (PEG2k-DMA), and 1,2-distearyloxypropyl-3-amine-N-[methoxy (polyethylene glycol)-2000] (PEG2k-DSA). In one embodiment, the stealth lipid may be PEG2k-DMG. In some embodiments, the stealth lipid may be PEG2k-DSG. In one embodiment, the stealth lipid may be PEG2k-DSPE. In one embodiment, the stealth lipid may be PEG2k-DMA. In one embodiment, the stealth lipid may be PEG2k-DSA. In one embodiment, the stealth lipid may be PEG2k-C11. In some embodiments, the stealth lipid may be PEG2k-C14. In some embodiments, the stealth lipid may be PEG2k-C16. In some embodiments, the stealth lipid may be PEG2k-C18.

Embodiments of the present disclosure also provide lipid compositions described according to the respective molar ratios of the component lipids in the formulation. In one embodiment, the mol-% of the CCD lipid may be from about 30 mol-% to about 60 mol-%. In one embodiment, the mol-% of the CCD lipid may be from about 35 mol-% to about 55 mol-%. In one embodiment, the mol-% of the CCD lipid may be from about 40 mol-% to about 50 mol-%. In one embodiment, the mol-% of the CCD lipid may be from about 42 mol-% to about 47 mol-%. In one embodiment, the mol-% of the CCD lipid may be about 45%. In some embodiments, the CCD lipid mol-% of the LNP batch will be ±30%, ±25%, ±20%, ±15%, ±10%, ±5%, or ±2.5% of the target mol-%. In certain embodiments, LNP inter-lot variability will be less than 15%, less than 10% or less than 5%.

In one embodiment, the mol-% of the helper lipid may be from about 30 mol-% to about 60 mol-%. In one embodiment, the mol-% of the helper lipid may be from about 35 mol-% to about 55 mol-%. In one embodiment, the mol-% of the helper lipid may be from about 40 mol-% to about 50 mol-%. In one embodiment, the mol-% of the helper lipid may be from about 41 mol-% to about 46 mol-%. In one embodiment, the mol-% of the helper lipid may be about 44 mol-%. In some embodiments, the helper mol-% of the LNP batch will be ±30%, ±25%, ±20%, ±15%, ±10%, ±5%, or ±2.5% of the target mol-%. In certain embodiments, LNP inter-lot variability will be less than 15%, less than 10% or less than 5%.

In one embodiment, the mol-% of the neutral lipid may be from about 1 mol-% to about 20 mol-%. In one embodiment, the mol-% of the neutral lipid may be from about 5 mol-% to about 15 mol-%. In one embodiment, the mol-% of the neutral lipid may be from about 7 mol-% to about 12 mol-%. In one embodiment, the mol-% of the neutral lipid may be about 9 mol-%. In some embodiments, the neutral lipid mol-% of the LNP batch will be ±30%, ±25%, ±20%, ±15%, ±10%, ±5%, or ±2.5% of the target mol-%. In certain embodiments, LNP inter-lot variability will be less than 15%, less than 10% or less than 5%.

In one embodiment, the mol-% of the stealth lipid may be from about 1 mol-% to about 10 mol-%. In one embodiment, the mol-% of the stealth lipid may be from about 1 mol-% to about 5 mol-%. In one embodiment, the mol-% of the stealth lipid may be from about 1 mol-% to about 3 mol-%. In one embodiment, the mol-% of the stealth lipid may be about 2 mol-%. In one embodiment, the mol-% of the stealth lipid may be about 1 mol-%. In some embodiments, the stealth lipid mol-% of the LNP batch will be ±30%, ±25%, ±20%, ±15%, ±10%, ±5%, or ±2.5% of the target mol-%. In certain embodiments, LNP inter-lot variability will be less than 15%, less than 10% or less than 5%.

### Location of Administration

In some embodiments, the compositions are delivered into the anterior chamber of the eye. In some embodiments, the compositions are delivered into the posterior chamber of the eye. In some embodiments, the compositions are delivered into the cornea itself. In some embodiments, the compositions are delivered into the corneal stroma. In some embodiments, the compositions are delivered into the corneal limbus. In some embodiments, the compositions are delivered onto the epithelial surface of the cornea. In any of the preceding embodiments of this paragraph as well as other embodiments described herein, treatment further comprises delivery of a Cas protein (e.g., Cas9), for example using a lipid nanoparticle, or delivery of a nucleic acid encoding a Cas protein using a vector and/or lipid nanoparticle. In some embodiments, for example those using a lipid nanoparticle, the nucleic acid encoding the Cas protein is mRNA. In some embodiments, a Cas protein or a nucleic acid encoding a Cas protein is delivered via the same vector and/or lipid nanoparticle that is used to deliver the appropriate guide or guides. In some embodiments, a Cas protein or a nucleic acid encoding a Cas protein is delivered via a different vector and/or lipid nanoparticle that is used to deliver the appropriate guide or guides.

Any of the compositions described herein may be administered to subjects to excise a portion or all of the TNR expansion in intron 3 of TCF4. Methods of treating FECD comprising administering any of the compositions described herein are encompassed. In some aspects, the compositions are administered in therapeutically effective amounts. In some embodiments, a method of excising, mutating, reducing copy number of, ameliorating, and/or eradicating TNRs of TCF4 is encompassed, comprising administering one or more of the compositions described herein. In some embodiments, a method of cleaving, mutating, reducing copy number of, ameliorating, and/or eradicating the TNRs of one or both copies of TCF4 per cell in a subject is provided, comprising administering one or more of the compositions described herein. In some embodiments, the cell is a corneal endothelium cell.

In some embodiments, two gRNAs are used to excise all of the TNRs in TCF4. In some embodiments, a first guide that is 5′ to the TNR is provided with a second guide that is 3′ to the TNR, or vice versa. Where two gRNAs are contemplated, a composition comprising any of the following combinations of guides is provided:
Combination 01: In some embodiments, a composition comprises two gRNAs comprising a first gRNA comprising SEQ ID NO: 1089, and a second gRNA comprising a sequence selected from SEQ ID NOs: 1182-1278.
Combination 02: In some embodiments, the composition comprises two gRNAs comprising a first gRNA comprising SEQ ID NO: 1090, and a second gRNA comprising a sequence selected from SEQ ID NOs: 1182-1278.
Combination 03: In some embodiments, the composition comprises two gRNAs comprising a first gRNA comprising SEQ ID NO: 1091, and a second gRNA comprising a sequence selected from SEQ ID NOs: 1182-1278.
Combination 04: In some embodiments, the composition comprises two gRNAs comprising a first gRNA comprising SEQ ID NO: 1092, and a second gRNA comprising a sequence selected from SEQ ID NOs: 1182-1278.
Combination 05: In some embodiments, the composition comprises two gRNAs comprising a first gRNA comprising SEQ ID NO: 1093, and a second gRNA comprising a sequence selected from SEQ ID NOs: 1182-1278.
Combination 06: In some embodiments, the composition comprises two gRNAs comprising a first gRNA comprising SEQ ID NO: 1094, and a second gRNA comprising a sequence selected from SEQ ID NOs: 1182-1278.
Combination 07: In some embodiments, the composition comprises two gRNAs comprising a first gRNA comprising SEQ ID NO: 1095, and a second gRNA comprising a sequence selected from SEQ ID NOs: 1182-1278.
Combination 08: In some embodiments, the composition comprises two gRNAs comprising a first gRNA comprising SEQ ID NO: 1096, and a second gRNA comprising a sequence selected from SEQ ID NOs: 1182-1278.
Combination 09: In some embodiments, the composition comprises two gRNAs comprising a first gRNA comprising SEQ ID NO: 1097, and a second gRNA comprising a sequence selected from SEQ ID NOs: 1182-1278.
Combination 10: In some embodiments, the composition comprises two gRNAs comprising a first gRNA comprising SEQ ID NO: 1098, and a second gRNA comprising a sequence selected from SEQ ID NOs: 1182-1278.
Combination 11: In some embodiments, the composition comprises two gRNAs comprising a first gRNA comprising SEQ ID NO: 1099, and a second gRNA comprising a sequence selected from SEQ ID NOs: 1182-1278.
Combination 12: In some embodiments, the composition comprises two gRNAs comprising a first gRNA comprising SEQ ID NO: 1100, and a second gRNA comprising a sequence selected from SEQ ID NOs: 1182-1278.
Combination 13: In some embodiments, the composition comprises two gRNAs comprising a first gRNA comprising SEQ ID NO: 1101, and a second gRNA comprising a sequence selected from SEQ ID NOs: 1182-1278.
Combination 14: In some embodiments, the composition comprises two gRNAs comprising a first gRNA comprising SEQ ID NO: 1102, and a second gRNA comprising a sequence selected from SEQ ID NOs: 1182-1278.
Combination 15: In some embodiments, the composition comprises two gRNAs comprising a first gRNA comprising SEQ ID NO: 1103, and a second gRNA comprising a sequence selected from SEQ ID NOs: 1182-1278.
Combination 16: In some embodiments, the composition comprises two gRNAs comprising a first gRNA comprising SEQ ID NO: 1104, and a second gRNA comprising a sequence selected from SEQ ID NOs: 1182-1278.
Combination 17: In some embodiments, the composition comprises two gRNAs comprising a first gRNA comprising SEQ ID NO: 1105, and a second gRNA comprising a sequence selected from SEQ ID NOs: 1182-1278.
Combination 18: In some embodiments, the composition comprises two gRNAs comprising a first gRNA comprising SEQ ID NO: 1106, and a second gRNA comprising a sequence selected from SEQ ID NOs: 1182-1278.
Combination 19: In some embodiments, the composition comprises two gRNAs comprising a first gRNA comprising SEQ ID NO: 1107, and a second gRNA comprising a sequence selected from SEQ ID NOs: 1182-1278.
Combination 20: In some embodiments, the composition comprises two gRNAs comprising a first gRNA comprising SEQ ID NO: 1108, and a second gRNA comprising a sequence selected from SEQ ID NOs: 1182-1278.
Combination 21: In some embodiments, the composition comprises two gRNAs comprising a first gRNA comprising SEQ ID NO: 1109, and a second gRNA comprising a sequence selected from SEQ ID NOs: 1182-1278.
Combination 22: In some embodiments, the composition comprises two gRNAs comprising a first gRNA comprising SEQ ID NO: 1110, and a second gRNA comprising a sequence selected from SEQ ID NOs: 1182-1278.
Combination 23: In some embodiments, the composition comprises two gRNAs comprising a first gRNA comprising SEQ ID NO: 1111, and a second gRNA comprising a sequence selected from SEQ ID NOs: 1182-1278.
Combination 24: In some embodiments, the composition comprises two gRNAs comprising a first gRNA comprising SEQ ID NO: 1112, and a second gRNA comprising a sequence selected from SEQ ID NOs: 1182-1278.
Combination 25: In some embodiments, the composition comprises two gRNAs comprising a first gRNA comprising SEQ ID NO: 1113, and a second gRNA comprising a sequence selected from SEQ ID NOs: 1182-1278.
Combination 26: In some embodiments, the composition comprises two gRNAs comprising a first gRNA comprising SEQ ID NO: 1114, and a second gRNA comprising a sequence selected from SEQ ID NOs: 1182-1278.
Combination 27: In some embodiments, the composition comprises two gRNAs comprising a first gRNA comprising SEQ ID NO: 1115, and a second gRNA comprising a sequence selected from SEQ ID NOs: 1182-1278.
Combination 28: In some embodiments, the composition comprises two gRNAs comprising a first gRNA comprising SEQ ID NO: 1116, and a second gRNA comprising a sequence selected from SEQ ID NOs: 1182-1278.
Combination 29: In some embodiments, the composition comprises two gRNAs comprising a first gRNA comprising SEQ ID NO: 1117, and a second gRNA comprising a sequence selected from SEQ ID NOs: 1182-1278.
Combination 30: In some embodiments, the composition comprises two gRNAs comprising a first gRNA comprising SEQ ID NO: 1118, and a second gRNA comprising a sequence selected from SEQ ID NOs: 1182-1278.
Combination 31: In some embodiments, the composition comprises two gRNAs comprising a first gRNA comprising SEQ ID NO: 1119, and a second gRNA comprising a sequence selected from SEQ ID NOs: 1182-1278.
Combination 32: In some embodiments, the composition comprises two gRNAs comprising a first gRNA comprising SEQ ID NO: 1120, and a second gRNA comprising a sequence selected from SEQ ID NOs: 1182-1278.
Combination 33: In some embodiments, the composition comprises two gRNAs comprising a first gRNA comprising SEQ ID NO: 1121, and a second gRNA comprising a sequence selected from SEQ ID NOs: 1182-1278.
Combination 34: In some embodiments, the composition comprises two gRNAs comprising a first gRNA comprising SEQ ID NO: 1122, and a second gRNA comprising a sequence selected from SEQ ID NOs: 1182-1278.
Combination 35: In some embodiments, the composition comprises two gRNAs comprising a first gRNA comprising SEQ ID NO: 1123, and a second gRNA comprising a sequence selected from SEQ ID NOs: 1182-1278.
Combination 36: In some embodiments, the composition comprises two gRNAs comprising a first gRNA comprising SEQ ID NO: 1124, and a second gRNA comprising a sequence selected from SEQ ID NOs: 1182-1278.
Combination 37: In some embodiments, the composition comprises two gRNAs comprising a first gRNA comprising SEQ ID NO: 1125, and a second gRNA comprising a sequence selected from SEQ ID NOs: 1182-1278.
Combination 38: In some embodiments, the composition comprises two gRNAs comprising a first gRNA comprising SEQ ID NO: 1126, and a second gRNA comprising a sequence selected from SEQ ID NOs: 1182-1278.
Combination 39: In some embodiments, the composition comprises two gRNAs comprising a first gRNA comprising SEQ ID NO: 1127, and a second gRNA comprising a sequence selected from SEQ ID NOs: 1182-1278.
Combination 40: In some embodiments, the composition comprises two gRNAs comprising a first gRNA comprising SEQ ID NO: 1128, and a second gRNA comprising a sequence selected from SEQ ID NOs: 1182-1278.
Combination 41: In some embodiments, the composition comprises two gRNAs comprising a first gRNA comprising SEQ ID NO: 1129, and a second gRNA comprising a sequence selected from SEQ ID NOs: 1182-1278.
Combination 42: In some embodiments, the composition comprises two gRNAs comprising a first gRNA comprising SEQ ID NO: 1130, and a second gRNA comprising a sequence selected from SEQ ID NOs: 1182-1278.
Combination 43: In some embodiments, the composition comprises two gRNAs comprising a first gRNA comprising SEQ ID NO: 1131, and a second gRNA comprising a sequence selected from SEQ ID NOs: 1182-1278.
Combination 44: In some embodiments, the composition comprises two gRNAs comprising a first gRNA comprising SEQ ID NO: 1132, and a second gRNA comprising a sequence selected from SEQ ID NOs: 1182-1278.
Combination 45: In some embodiments, the composition comprises two gRNAs comprising a first gRNA comprising SEQ ID NO: 1133, and a second gRNA comprising a sequence selected from SEQ ID NOs: 1182-1278.
Combination 46: In some embodiments, the composition comprises two gRNAs comprising a first gRNA comprising SEQ ID NO: 1134, and a second gRNA comprising a sequence selected from SEQ ID NOs: 1182-1278.
Combination 47: In some embodiments, the composition comprises two gRNAs comprising a first gRNA comprising SEQ ID NO: 1135, and a second gRNA comprising a sequence selected from SEQ ID NOs: 1182-1278.
Combination 48: In some embodiments, the composition comprises two gRNAs comprising a first gRNA comprising SEQ ID NO: 1136, and a second gRNA comprising a sequence selected from SEQ ID NOs: 1182-1278.
Combination 49: In some embodiments, the composition comprises two gRNAs comprising a first gRNA comprising SEQ ID NO: 1137, and a second gRNA comprising a sequence selected from SEQ ID NOs: 1182-1278.
Combination 50: In some embodiments, the composition comprises two gRNAs comprising a first gRNA comprising SEQ ID NO: 1138, and a second gRNA comprising a sequence selected from SEQ ID NOs: 1182-1278.
Combination 51: In some embodiments, the composition comprises two gRNAs comprising a first gRNA comprising SEQ ID NO: 1139, and a second gRNA comprising a sequence selected from SEQ ID NOs: 1182-1278.
Combination 52: In some embodiments, the composition comprises two gRNAs comprising a first gRNA comprising SEQ ID NO: 1140, and a second gRNA comprising a sequence selected from SEQ ID NOs: 1182-1278.
Combination 53: In some embodiments, the composition comprises two gRNAs comprising a first gRNA comprising SEQ ID NO: 1141, and a second gRNA comprising a sequence selected from SEQ ID NOs: 1182-1278.
Combination 54: In some embodiments, the composition comprises two gRNAs comprising a first gRNA comprising SEQ ID NO: 1142, and a second gRNA comprising a sequence selected from SEQ ID NOs: 1182-1278.
Combination 55: In some embodiments, the composition comprises two gRNAs comprising a first gRNA comprising SEQ ID NO: 1143, and a second gRNA comprising a sequence selected from SEQ ID NOs: 1182-1278.
Combination 56: In some embodiments, the composition comprises two gRNAs comprising a first gRNA comprising SEQ ID NO: 1144, and a second gRNA comprising a sequence selected from SEQ ID NOs: 1182-1278.
Combination 57: In some embodiments, the composition comprises two gRNAs comprising a first gRNA comprising SEQ ID NO: 1145, and a second gRNA comprising a sequence selected from SEQ ID NOs: 1182-1278.
Combination 58: In some embodiments, the composition comprises two gRNAs comprising a first gRNA comprising SEQ ID NO: 1146, and a second gRNA comprising a sequence selected from SEQ ID NOs: 1182-1278.
Combination 59: In some embodiments, the composition comprises two gRNAs comprising a first gRNA comprising SEQ ID NO: 1147, and a second gRNA comprising a sequence selected from SEQ ID NOs: 1182-1278.
Combination 60: In some embodiments, the composition comprises two gRNAs comprising a first gRNA comprising SEQ ID NO: 1148, and a second gRNA comprising a sequence selected from SEQ ID NOs: 1182-1278.
Combination 61: In some embodiments, the composition comprises two gRNAs comprising a first gRNA comprising SEQ ID NO: 1149, and a second gRNA comprising a sequence selected from SEQ ID NOs: 1182-1278.
Combination 62: In some embodiments, the composition comprises two gRNAs comprising a first gRNA comprising SEQ ID NO: 1150, and a second gRNA comprising a sequence selected from SEQ ID NOs: 1182-1278.
Combination 63: In some embodiments, the composition comprises two gRNAs comprising a first gRNA comprising SEQ ID NO: 1151, and a second gRNA comprising a sequence selected from SEQ ID NOs: 1182-1278.
Combination 64: In some embodiments, the composition comprises two gRNAs comprising a first gRNA comprising SEQ ID NO: 1152, and a second gRNA comprising a sequence selected from SEQ ID NOs: 1182-1278.
Combination 65: In some embodiments, the composition comprises two gRNAs comprising a first gRNA comprising SEQ ID NO: 1153, and a second gRNA comprising a sequence selected from SEQ ID NOs: 1182-1278.
Combination 66: In some embodiments, the composition comprises two gRNAs comprising a first gRNA comprising SEQ ID NO: 1154, and a second gRNA comprising a sequence selected from SEQ ID NOs: 1182-1278.
Combination 67: In some embodiments, the composition comprises two gRNAs comprising a first gRNA comprising SEQ ID NO: 1155, and a second gRNA comprising a sequence selected from SEQ ID NOs: 1182-1278.
Combination 68: In some embodiments, the composition comprises two gRNAs comprising a first gRNA comprising SEQ ID NO: 1156, and a second gRNA comprising a sequence selected from SEQ ID NOs: 1182-1278.
Combination 69: In some embodiments, the composition comprises two gRNAs comprising a first gRNA comprising SEQ ID NO: 1157, and a second gRNA comprising a sequence selected from SEQ ID NOs: 1182-1278.
Combination 70: In some embodiments, the composition comprises two gRNAs comprising a first gRNA comprising SEQ ID NO: 1158, and a second gRNA comprising a sequence selected from SEQ ID NOs: 1182-1278.
Combination 71: In some embodiments, the composition comprises two gRNAs comprising a first gRNA comprising SEQ ID NO: 1159, and a second gRNA comprising a sequence selected from SEQ ID NOs: 1182-1278.
Combination 72: In some embodiments, the composition comprises two gRNAs comprising a first gRNA comprising SEQ ID NO: 1160, and a second gRNA comprising a sequence selected from SEQ ID NOs: 1182-1278.
Combination 73: In some embodiments, the composition comprises two gRNAs comprising a first gRNA comprising SEQ ID NO: 1161, and a second gRNA comprising a sequence selected from SEQ ID NOs: 1182-1278.
Combination 74: In some embodiments, the composition comprises two gRNAs comprising a first gRNA comprising SEQ ID NO: 1162, and a second gRNA comprising a sequence selected from SEQ ID NOs: 1182-1278.
Combination 75: In some embodiments, the composition comprises two gRNAs comprising a first gRNA comprising SEQ ID NO: 1163, and a second gRNA comprising a sequence selected from SEQ ID NOs: 1182-1278.
Combination 76: In some embodiments, the composition comprises two gRNAs comprising a first gRNA comprising SEQ ID NO: 1164, and a second gRNA comprising a sequence selected from SEQ ID NOs: 1182-1278.
Combination 77: In some embodiments, the composition comprises two gRNAs comprising a first gRNA comprising SEQ ID NO: 1165, and a second gRNA comprising a sequence selected from SEQ ID NOs: 1182-1278.
Combination 78: In some embodiments, the composition comprises two gRNAs comprising a first gRNA comprising SEQ ID NO: 1166, and a second gRNA comprising a sequence selected from SEQ ID NOs: 1182-1278.
Combination 79: In some embodiments, the composition comprises two gRNAs comprising a first gRNA comprising SEQ ID NO: 1167, and a second gRNA comprising a sequence selected from SEQ ID NOs: 1182-1278.
Combination 80: In some embodiments, the composition comprises two gRNAs comprising a first gRNA comprising SEQ ID NO: 1168, and a second gRNA comprising a sequence selected from SEQ ID NOs: 1182-1278.
Combination 81: In some embodiments, the composition comprises two gRNAs comprising a first gRNA comprising SEQ ID NO: 1169, and a second gRNA comprising a sequence selected from SEQ ID NOs: 1182-1278.
Combination 82: In some embodiments, the composition comprises two gRNAs comprising a first gRNA comprising SEQ ID NO: 1170, and a second gRNA comprising a sequence selected from SEQ ID NOs: 1182-1278.
Combination 83: In some embodiments, the composition comprises two gRNAs comprising a first gRNA comprising SEQ ID NO: 1171, and a second gRNA comprising a sequence selected from SEQ ID NOs: 1182-1278.
Combination 84: In some embodiments, the composition comprises two gRNAs comprising a first gRNA comprising SEQ ID NO: 1172, and a second gRNA comprising a sequence selected from SEQ ID NOs: 1182-1278.
Combination 85: In some embodiments, the composition comprises two gRNAs comprising a first gRNA comprising SEQ ID NO: 1173, and a second gRNA comprising a sequence selected from SEQ ID NOs: 1182-1278.
Combination 86: In some embodiments, the composition comprises two gRNAs comprising a first gRNA comprising SEQ ID NO: 1174, and a second gRNA comprising a sequence selected from SEQ ID NOs: 1182-1278.
Combination 87: In some embodiments, the composition comprises two gRNAs comprising a first gRNA comprising SEQ ID NO: 1175, and a second gRNA comprising a sequence selected from SEQ ID NOs: 1182-1278.
Combination 88: In some embodiments, the composition comprises two gRNAs comprising a first gRNA comprising SEQ ID NO: 1176, and a second gRNA comprising a sequence selected from SEQ ID NOs: 1182-1278.
Combination 89: In some embodiments, the composition comprises two gRNAs comprising a first gRNA comprising SEQ ID NO: 1177, and a second gRNA comprising a sequence selected from SEQ ID NOs: 1182-1278.
Combination 90: In some embodiments, the composition comprises two gRNAs comprising a first gRNA comprising SEQ ID NO: 1178, and a second gRNA comprising a sequence selected from SEQ ID NOs: 1182-1278.
Combination 91: In some embodiments, the composition comprises two gRNAs comprising a first gRNA comprising SEQ ID NO: 1179, and a second gRNA comprising a sequence selected from SEQ ID NOs: 1182-1278.
Combination 92: In some embodiments, the composition comprises two gRNAs comprising a first gRNA comprising SEQ ID NO: 1180, and a second gRNA comprising a sequence selected from SEQ ID NOs: 1182-1278.
Combination 93: In some embodiments, the composition comprises two gRNAs comprising a first gRNA comprising SEQ ID NO: 1181, and a second gRNA comprising a sequence selected from SEQ ID NOs: 1182-1278.
Combination 94: In some embodiments, the composition comprises two gRNAs comprising a first gRNA comprising SEQ ID NO: 1182 and a second gRNA comprising a sequence selected from SEQ ID NOs: 1089-1181.
Combination 95: In some embodiments, the composition comprises two gRNAs comprising a first gRNA comprising SEQ ID NO: 1183 and a second gRNA comprising a sequence selected from SEQ ID NOs: 1089-1181.
Combination 96: In some embodiments, the composition comprises two gRNAs comprising a first gRNA comprising SEQ ID NO: 1184 and a second gRNA comprising a sequence selected from SEQ ID NOs: 1089-1181.
Combination 97: In some embodiments, the composition comprises two gRNAs comprising a first gRNA comprising SEQ ID NO: 1185 and a second gRNA comprising a sequence selected from SEQ ID NOs: 1089-1181.
Combination 98: In some embodiments, the composition comprises two gRNAs comprising a first gRNA comprising SEQ ID NO: 1186 and a second gRNA comprising a sequence selected from SEQ ID NOs: 1089-1181.
Combination 99: In some embodiments, the composition comprises two gRNAs comprising a first gRNA comprising SEQ ID NO: 1187 and a second gRNA comprising a sequence selected from SEQ ID NOs: 1089-1181.
Combination 100: In some embodiments, the composition comprises two gRNAs comprising a first gRNA comprising SEQ ID NO: 1188 and a second gRNA comprising a sequence selected from SEQ ID NOs: 1089-1181.
Combination 101: In some embodiments, the composition comprises two gRNAs comprising a first gRNA comprising SEQ ID NO: 1189 and a second gRNA comprising a sequence selected from SEQ ID NOs: 1089-1181.
Combination 102: In some embodiments, the composition comprises two gRNAs comprising a first gRNA comprising SEQ ID NO: 1190 and a second gRNA comprising a sequence selected from SEQ ID NOs: 1089-1181.
Combination 103: In some embodiments, the composition comprises two gRNAs comprising a first gRNA comprising SEQ ID NO: 1191 and a second gRNA comprising a sequence selected from SEQ ID NOs: 1089-1181.
Combination 104: In some embodiments, the composition comprises two gRNAs comprising a first gRNA comprising SEQ ID NO: 1192 and a second gRNA comprising a sequence selected from SEQ ID NOs: 1089-1181.
Combination 105: In some embodiments, the composition comprises two gRNAs comprising a first gRNA comprising SEQ ID NO: 1193 and a second gRNA comprising a sequence selected from SEQ ID NOs: 1089-1181.
Combination 106: In some embodiments, the composition comprises two gRNAs comprising a first gRNA comprising SEQ ID NO: 1194 and a second gRNA comprising a sequence selected from SEQ ID NOs: 1089-1181.
Combination 107: In some embodiments, the composition comprises two gRNAs comprising a first gRNA comprising SEQ ID NO: 1195 and a second gRNA comprising a sequence selected from SEQ ID NOs: 1089-1181.
Combination 108: In some embodiments, the composition comprises two gRNAs comprising a first gRNA comprising SEQ ID NO: 1196 and a second gRNA comprising a sequence selected from SEQ ID NOs: 1089-1181.
Combination 109: In some embodiments, the composition comprises two gRNAs comprising a first gRNA comprising SEQ ID NO: 1197 and a second gRNA comprising a sequence selected from SEQ ID NOs: 1089-1181.
Combination 110: In some embodiments, the composition comprises two gRNAs comprising a first gRNA comprising SEQ ID NO: 1198 and a second gRNA comprising a sequence selected from SEQ ID NOs: 1089-1181.
Combination 111: In some embodiments, the composition comprises two gRNAs comprising a first gRNA comprising SEQ ID NO: 1199 and a second gRNA comprising a sequence selected from SEQ ID NOs: 1089-1181.
Combination 112: In some embodiments, the composition comprises two gRNAs comprising a first gRNA comprising SEQ ID NO: 1200 and a second gRNA comprising a sequence selected from SEQ ID NOs: 1089-1181.
Combination 113: In some embodiments, the composition comprises two gRNAs comprising a first gRNA comprising SEQ ID NO: 1201 and a second gRNA comprising a sequence selected from SEQ ID NOs: 1089-1181.
Combination 114: In some embodiments, the composition comprises two gRNAs comprising a first gRNA comprising SEQ ID NO: 1202 and a second gRNA comprising a sequence selected from SEQ ID NOs: 1089-1181.
Combination 115: In some embodiments, the composition comprises two gRNAs comprising a first gRNA comprising SEQ ID NO: 1203 and a second gRNA comprising a sequence selected from SEQ ID NOs: 1089-1181.
Combination 116: In some embodiments, the composition comprises two gRNAs comprising a first gRNA comprising SEQ ID NO: 1204 and a second gRNA comprising a sequence selected from SEQ ID NOs: 1089-1181.
Combination 117: In some embodiments, the composition comprises two gRNAs comprising a first gRNA comprising SEQ ID NO: 1205 and a second gRNA comprising a sequence selected from SEQ ID NOs: 1089-1181.
Combination 118: In some embodiments, the composition comprises two gRNAs comprising a first gRNA comprising SEQ ID NO: 1206 and a second gRNA comprising a sequence selected from SEQ ID NOs: 1089-1181.
Combination 119: In some embodiments, the composition comprises two gRNAs comprising a first gRNA comprising SEQ ID NO: 1207 and a second gRNA comprising a sequence selected from SEQ ID NOs: 1089-1181.
Combination 120: In some embodiments, the composition comprises two gRNAs comprising a first gRNA comprising SEQ ID NO: 1208 and a second gRNA comprising a sequence selected from SEQ ID NOs: 1089-1181.
Combination 121: In some embodiments, the composition comprises two gRNAs comprising a first gRNA comprising SEQ ID NO: 1209 and a second gRNA comprising a sequence selected from SEQ ID NOs: 1089-1181.
Combination 122: In some embodiments, the composition comprises two gRNAs comprising a first gRNA comprising SEQ ID NO: 1210 and a second gRNA comprising a sequence selected from SEQ ID NOs: 1089-1181.
Combination 123: In some embodiments, the composition comprises two gRNAs comprising a first gRNA comprising SEQ ID NO: 1211 and a second gRNA comprising a sequence selected from SEQ ID NOs: 1089-1181.
Combination 124: In some embodiments, the composition comprises two gRNAs comprising a first gRNA comprising SEQ ID NO: 1212 and a second gRNA comprising a sequence selected from SEQ ID NOs: 1089-1181.
Combination 125: In some embodiments, the composition comprises two gRNAs comprising a first gRNA comprising SEQ ID NO: 1213 and a second gRNA comprising a sequence selected from SEQ ID NOs: 1089-1181.
Combination 126: In some embodiments, the composition comprises two gRNAs comprising a first gRNA comprising SEQ ID NO: 1214 and a second gRNA comprising a sequence selected from SEQ ID NOs: 1089-1181.
Combination 127: In some embodiments, the composition comprises two gRNAs comprising a first gRNA comprising SEQ ID NO: 1215 and a second gRNA comprising a sequence selected from SEQ ID NOs: 1089-1181.
Combination 128: In some embodiments, the composition comprises two gRNAs comprising a first gRNA comprising SEQ ID NO: 1216 and a second gRNA comprising a sequence selected from SEQ ID NOs: 1089-1181.
Combination 129: In some embodiments, the composition comprises two gRNAs comprising a first gRNA comprising SEQ ID NO: 1217 and a second gRNA comprising a sequence selected from SEQ ID NOs: 1089-1181.
Combination 130: In some embodiments, the composition comprises two gRNAs comprising a first gRNA comprising SEQ ID NO: 1218 and a second gRNA comprising a sequence selected from SEQ ID NOs: 1089-1181.
Combination 131: In some embodiments, the composition comprises two gRNAs comprising a first gRNA comprising SEQ ID NO: 1219 and a second gRNA comprising a sequence selected from SEQ ID NOs: 1089-1181.
Combination 132: In some embodiments, the composition comprises two gRNAs comprising a first gRNA comprising SEQ ID NO: 1220 and a second gRNA comprising a sequence selected from SEQ ID NOs: 1089-1181.
Combination 133: In some embodiments, the composition comprises two gRNAs comprising a first gRNA comprising SEQ ID NO: 1221 and a second gRNA comprising a sequence selected from SEQ ID NOs: 1089-1181.
Combination 134: In some embodiments, the composition comprises two gRNAs comprising a first gRNA comprising SEQ ID NO: 1222 and a second gRNA comprising a sequence selected from SEQ ID NOs: 1089-1181.
Combination 135: In some embodiments, the composition comprises two gRNAs comprising a first gRNA comprising SEQ ID NO: 1223 and a second gRNA comprising a sequence selected from SEQ ID NOs: 1089-1181.
Combination 136: In some embodiments, the composition comprises two gRNAs comprising a first gRNA comprising SEQ ID NO: 1224 and a second gRNA comprising a sequence selected from SEQ ID NOs: 1089-1181.
Combination 137: In some embodiments, the composition comprises two gRNAs comprising a first gRNA comprising SEQ ID NO: 1225 and a second gRNA comprising a sequence selected from SEQ ID NOs: 1089-1181.
Combination 138: In some embodiments, the composition comprises two gRNAs comprising a first gRNA comprising SEQ ID NO: 1226 and a second gRNA comprising a sequence selected from SEQ ID NOs: 1089-1181.
Combination 139: In some embodiments, the composition comprises two gRNAs comprising a first gRNA comprising SEQ ID NO: 1227 and a second gRNA comprising a sequence selected from SEQ ID NOs: 1089-1181.
Combination 140: In some embodiments, the composition comprises two gRNAs comprising a first gRNA comprising SEQ ID NO: 1228 and a second gRNA comprising a sequence selected from SEQ ID NOs: 1089-1181.
Combination 141: In some embodiments, the composition comprises two gRNAs comprising a first gRNA comprising SEQ ID NO: 1229 and a second gRNA comprising a sequence selected from SEQ ID NOs: 1089-1181.
Combination 142: In some embodiments, the composition comprises two gRNAs comprising a first gRNA comprising SEQ ID NO: 1230 and a second gRNA comprising a sequence selected from SEQ ID NOs: 1089-1181.
Combination 143: In some embodiments, the composition comprises two gRNAs comprising a first gRNA comprising SEQ ID NO: 1231 and a second gRNA comprising a sequence selected from SEQ ID NOs: 1089-1181.
Combination 144: In some embodiments, the composition comprises two gRNAs comprising a first gRNA comprising SEQ ID NO: 1232 and a second gRNA comprising a sequence selected from SEQ ID NOs: 1089-1181.
Combination 145: In some embodiments, the composition comprises two gRNAs comprising a first gRNA comprising SEQ ID NO: 1233 and a second gRNA comprising a sequence selected from SEQ ID NOs: 1089-1181.
Combination 146: In some embodiments, the composition comprises two gRNAs comprising a first gRNA comprising SEQ ID NO: 1234 and a second gRNA comprising a sequence selected from SEQ ID NOs: 1089-1181.
Combination 147: In some embodiments, the composition comprises two gRNAs comprising a first gRNA comprising SEQ ID NO: 1235 and a second gRNA comprising a sequence selected from SEQ ID NOs: 1089-1181.
Combination 148: In some embodiments, the composition comprises two gRNAs comprising a first gRNA comprising SEQ ID NO: 1236 and a second gRNA comprising a sequence selected from SEQ ID Nos: 1089-1181.
Combination 149: In some embodiments, the composition comprises two gRNAs comprising a first gRNA comprising SEQ ID NO: 1237 and a second gRNA comprising a sequence selected from SEQ ID Nos: 1089-1181.
Combination 150: In some embodiments, the composition comprises two gRNAs comprising a first gRNA comprising SEQ ID NO: 1238 and a second gRNA comprising a sequence selected from SEQ ID Nos: 1089-1181.
Combination 151: In some embodiments, the composition comprises two gRNAs comprising a first gRNA comprising SEQ ID NO: 1239 and a second gRNA comprising a sequence selected from SEQ ID Nos: 1089-1181.
Combination 152: In some embodiments, the composition comprises two gRNAs comprising a first gRNA comprising SEQ ID NO: 1240 and a second gRNA comprising a sequence selected from SEQ ID Nos: 1089-1181.
Combination 153: In some embodiments, the composition comprises two gRNAs comprising a first gRNA comprising SEQ ID NO: 1241 and a second gRNA comprising a sequence selected from SEQ ID Nos: 1089-1181.
Combination 154: In some embodiments, the composition comprises two gRNAs comprising a first gRNA comprising SEQ ID NO: 1242 and a second gRNA comprising a sequence selected from SEQ ID Nos: 1089-1181.
Combination 155: In some embodiments, the composition comprises two gRNAs comprising a first gRNA comprising SEQ ID NO: 1243 and a second gRNA comprising a sequence selected from SEQ ID Nos: 1089-1181.
Combination 156: In some embodiments, the composition comprises two gRNAs comprising a first gRNA comprising SEQ ID NO: 1244 and a second gRNA comprising a sequence selected from SEQ ID Nos: 1089-1181.
Combination 157: In some embodiments, the composition comprises two gRNAs comprising a first gRNA comprising SEQ ID NO: 1245 and a second gRNA comprising a sequence selected from SEQ ID Nos: 1089-1181.
Combination 158: In some embodiments, the composition comprises two gRNAs comprising a first gRNA comprising SEQ ID NO: 1246 and a second gRNA comprising a sequence selected from SEQ ID Nos: 1089-1181.
Combination 159: In some embodiments, the composition comprises two gRNAs comprising a first gRNA comprising SEQ ID NO: 1247 and a second gRNA comprising a sequence selected from SEQ ID Nos: 1089-1181.
Combination 160: In some embodiments, the composition comprises two gRNAs comprising a first gRNA comprising SEQ ID NO: 1248 and a second gRNA comprising a sequence selected from SEQ ID Nos: 1089-1181.
Combination 161: In some embodiments, the composition comprises two gRNAs comprising a first gRNA comprising SEQ ID NO: 1249 and a second gRNA comprising a sequence selected from SEQ ID Nos: 1089-1181.
Combination 162: In some embodiments, the composition comprises two gRNAs comprising a first gRNA comprising SEQ ID NO: 1250 and a second gRNA comprising a sequence selected from SEQ ID Nos: 1089-1181.
Combination 163: In some embodiments, the composition comprises two gRNAs comprising a first gRNA comprising SEQ ID NO: 1251 and a second gRNA comprising a sequence selected from SEQ ID Nos: 1089-1181.
Combination 164: In some embodiments, the composition comprises two gRNAs comprising a first gRNA comprising SEQ ID NO: 1252 and a second gRNA comprising a sequence selected from SEQ ID Nos: 1089-1181.
Combination 165: In some embodiments, the composition comprises two gRNAs comprising a first gRNA comprising SEQ ID NO: 1253 and a second gRNA comprising a sequence selected from SEQ ID Nos: 1089-1181.
Combination 166: In some embodiments, the composition comprises two gRNAs comprising a first gRNA comprising SEQ ID NO: 1254 and a second gRNA comprising a sequence selected from SEQ ID Nos: 1089-1181.
Combination 167: In some embodiments, the composition comprises two gRNAs comprising a first gRNA comprising SEQ ID NO: 1255 and a second gRNA comprising a sequence selected from SEQ ID Nos: 1089-1181.
Combination 168: In some embodiments, the composition comprises two gRNAs comprising a first gRNA comprising SEQ ID NO: 1256 and a second gRNA comprising a sequence selected from SEQ ID Nos: 1089-1181.
Combination 169: In some embodiments, the composition comprises two gRNAs comprising a first gRNA comprising SEQ ID NO: 1257 and a second gRNA comprising a sequence selected from SEQ ID Nos: 1089-1181.
Combination 170: In some embodiments, the composition comprises two gRNAs comprising a first gRNA comprising SEQ ID NO: 1258 and a second gRNA comprising a sequence selected from SEQ ID Nos: 1089-1181.
Combination 171: In some embodiments, the composition comprises two gRNAs comprising a first gRNA comprising SEQ ID NO: 1259 and a second gRNA comprising a sequence selected from SEQ ID Nos: 1089-1181.
Combination 172: In some embodiments, the composition comprises two gRNAs comprising a first gRNA comprising SEQ ID NO: 1260 and a second gRNA comprising a sequence selected from SEQ ID Nos: 1089-1181.
Combination 173: In some embodiments, the composition comprises two gRNAs comprising a first gRNA comprising SEQ ID NO: 1261 and a second gRNA comprising a sequence selected from SEQ ID Nos: 1089-1181.
Combination 174: In some embodiments, the composition comprises two gRNAs comprising a first gRNA comprising SEQ ID NO: 1262 and a second gRNA comprising a sequence selected from SEQ ID Nos: 1089-1181.
Combination 175: In some embodiments, the composition comprises two gRNAs comprising a first gRNA comprising SEQ ID NO: 1263 and a second gRNA comprising a sequence selected from SEQ ID Nos: 1089-1181.
Combination 176: In some embodiments, the composition comprises two gRNAs comprising a first gRNA comprising SEQ ID NO: 1264 and a second gRNA comprising a sequence selected from SEQ ID Nos: 1089-1181.
Combination 177: In some embodiments, the composition comprises two gRNAs comprising a first gRNA comprising SEQ ID NO: 1265 and a second gRNA comprising a sequence selected from SEQ ID Nos: 1089-1181.
Combination 178: In some embodiments, the composition comprises two gRNAs comprising a first gRNA comprising SEQ ID NO: 1266 and a second gRNA comprising a sequence selected from SEQ ID Nos: 1089-1181.
Combination 179: In some embodiments, the composition comprises two gRNAs comprising a first gRNA comprising SEQ ID NO: 1267 and a second gRNA comprising a sequence selected from SEQ ID Nos: 1089-1181.
Combination 180: In some embodiments, the composition comprises two gRNAs comprising a first gRNA comprising SEQ ID NO: 1268 and a second gRNA comprising a sequence selected from SEQ ID Nos: 1089-1181.
Combination 181: In some embodiments, the composition comprises two gRNAs comprising a first gRNA comprising SEQ ID NO: 1269 and a second gRNA comprising a sequence selected from SEQ ID Nos: 1089-1181.
Combination 182: In some embodiments, the composition comprises two gRNAs comprising a first gRNA comprising SEQ ID NO: 1270 and a second gRNA comprising a sequence selected from SEQ ID Nos: 1089-1181.
Combination 183: In some embodiments, the composition comprises two gRNAs comprising a first gRNA comprising SEQ ID NO: 1271 and a second gRNA comprising a sequence selected from SEQ ID Nos: 1089-1181.
Combination 184: In some embodiments, the composition comprises two gRNAs comprising a first gRNA comprising SEQ ID NO: 1272 and a second gRNA comprising a sequence selected from SEQ ID Nos: 1089-1181.
Combination 185: In some embodiments, the composition comprises two gRNAs comprising a first gRNA comprising SEQ ID NO: 1273 and a second gRNA comprising a sequence selected from SEQ ID Nos: 1089-1181.
Combination 186: In some embodiments, the composition comprises two gRNAs comprising a first gRNA comprising SEQ ID NO: 1274 and a second gRNA comprising a sequence selected from SEQ ID Nos: 1089-1181.
Combination 187: In some embodiments, the composition comprises two gRNAs comprising a first gRNA comprising SEQ ID NO: 1275 and a second gRNA comprising a sequence selected from SEQ ID Nos: 1089-1181.
Combination 188: In some embodiments, the composition comprises two gRNAs comprising a first gRNA comprising SEQ ID NO: 1276 and a second gRNA comprising a sequence selected from SEQ ID Nos: 1089-1181.
Combination 189: In some embodiments, the composition comprises two gRNAs comprising a first gRNA comprising SEQ ID NO: 1277 and a second gRNA comprising a sequence selected from SEQ ID Nos: 1089-1181.
Combination 190: In some embodiments, the composition comprises two gRNAs comprising a first gRNA comprising SEQ ID NO: 1278 and a second gRNA comprising a sequence selected from SEQ ID Nos: 1089-1181.

### EXAMPLES

### Example 1. Use of Pairs of gRNAs to Excise TNR Expansions from TCF4

To remove the TNRs from TCF4 and limit the production of toxic RNAs, CRISPR guides have been designed to simultaneously cut on either side of the expansion using specific target sequences. These gRNAs have been designed to work with wild type S. pyogenes Cas9 (“Spy Cas9”). Other gRNAs, suitable for use with other CRISPR nucleases, could be designed in a similar manner.

Target sequences were selected using the sequence of the TCF4 intron 3 sequence with flanking exons (SEQ ID NO: 1085). This sequence is based on UCSC Genome browser, Human, February 2009 (GRCh37/hg19) assembly. This sequence contains a set of 24 CTG repeats (TNRs) at range 53253387-53253458 within the intron position chr18: 53252584-53254275. The exact range of CTG repeats in this intron will vary based on the number of repeats, where a number of repeats >40 is associated with increased risk for developing disease. In the hg38 build, the repeats are located at chr18: 55,586,156-55,586,228, within the intron spanning chr18: 55,585,280-55,587,136. Target sequences and corresponding guide sequences are listed in Table 2 (SEQ ID NOs: 1-190 (target sequences) and SEQ ID NOs: 1089-1278 (guide sequences)). The particular forms of the crRNAs and trRNAs used in this Example 1 are provided in Table 1 as SEQ ID NO: 1087 and SEQ ID NO: 1088, respectively. The target sequence for the 5′ guide sequences (SEQ ID NOs: 1089-1181) is located between Chr18: 55,585,285-55,586,153 and is upstream of the location of the TNRs. The target sequence for the 3′ guide sequences (SEQ ID NOs: 94-190) is located between Chr18: 55586225-55587203 and is downstream of the location of the TNRs. Table 2 lists SEQ ID NOs: 1-190 (target sequences) and SEQ ID NOs: 1089-1278 (guide sequences that direct a nuclease to a corresponding target sequence and bind to the reverse compliment of the target sequences). Cutting Frequency Determination (CFD) scores were generated for each guide sequence in silico, according to the methodology reported by Doench et al., Nat Biotechnol. 2016 February; 34 (2): 184-191. These scores (which have been multiplied by a factor of 100 to convert to decimals as compared to how Doench et al report scores) provide a measure of the off-target potential for a given gRNA.

| | | | | | Dis- | | | | |
|---|---|---|---|---|---|---|---|---|---|
| | | | | | tance | | | | |
| | Target | | | | to | | | | |
| SEQ | sequence | | | | start | SEQ | | | Average |
| ID | (including | | | | of | ID | Guide | CFD | Editing |
| NO | PAM) | Chromosomal location | Strand | Orientation | TNR | No | Sequence | Score | Percent |
| 1 | TTGGCAAGTGGA | Chr18: 55585285-55585307 | − | 5′ of TNRs of | −871 | 1089 | UUGGCAAGUGG | 422.48 | NA |
| | CATTTTACTGG | | | TCF4 | | | ACAUUUUAC | | |
| 2 | TGTCCACTTGCCA | Chr18: 55585294-55585316 | + | 5′ of TNRs of | −862 | 1090 | UGUCCACUUGC | 619.25 | NA |
| | AAGAAGTTGG | | | TCF4 | | | CAAAGAAGU | | |
| 3 | GGACCAACTTCTT | Chr18: 55585297-55585319 | − | 5′ of TNRs of | −859 | 1091 | GGACCAACUUC | 402.71 | NA |
| | TGGCAAGTGG | | | TCF4 | | | UUUGGCAAG | | |
| 4 | GAAAAATGGACC | Chr18: 55585304-55585326 | − | 5′ of TNRs of | −852 | 1092 | GAAAAAUGGAC | 1569.22 | NA |
| | AACTTCTTTGG | | | TCF4 | | | CAACUUCUU | | |
| 5 | CCATTTTTCCCAC | Chr18: 55585318-55585340 | + | 5′ of TNRs of | −838 | 1093 | CCAUUUUUCCC | 809.81 | NA |
| | TGCTCACAGG | | | TCF4 | | | ACUGCUCAC | | |
| 6 | CCTGTGAGCAGT | Chr18: 55585318-55585340 | − | 5′ of TNRs of | −838 | 1094 | CCUGUGAGCAG | 773.35 | NA |
| | GGGAAAAATGG | | | TCF4 | | | UGGGAAAAA | | |
| 7 | TTTTTCCCACTGC | Chr18: 55585321-55585343 | + | 5′ of TNRs of | −835 | 1095 | UUUUUCCCACU | 1673.79 | NA |
| | TCACAGGAGG | | | TCF4 | | | GCUCACAGG | | |
| 8 | TTTCACCTCCTGT | Chr18: 55585326-55585348 | − | 5′ of TNRs of | −830 | 1096 | UUUCACCUCCU | 1250.27 | NA |
| | GAGCAGTGGG | | | TCF4 | | | GUGAGCAGU | | |
| 9 | TTTTCACCTCCTG | Chr18: 55585327-55585349 | − | 5′ of TNRs of | −829 | 1097 | UUUUCACCUCC | 1372.08 | NA |
| | TGAGCAGTGG | | | TCF4 | | | UGUGAGCAG | | |
| 10 | AGATCTTTGAGG | Chr18: 55585399-55585421 | − | 5′ of TNRs of | −757 | 1098 | AGAUCUUUGAG | 147.38 | 27.9 |
| | AGCTCTGAAGG | | | TCF4 | | | GAGCUCUGA | | |
| 11 | AACAGTATGAAA | Chr18: 55585410-55585432 | − | 5′ of TNRs of | −746 | 1099 | AACAGUAUGAA | 369.96 | 32.87 |
| | GATCTTTGAGG | | | TCF4 | | | AGAUCUUUG | | |
| 12 | AGCATAAACTCTA | Chr18: 55585434-55585456 | − | 5′ of TNRs of | −722 | 1100 | AGCAUAAACUC | 37.08 | 1.83 |
| | AGCTGTTTGG | | | TCF4 | | | UAAGCUGUU | | |
| 13 | ACAGCTTAGAGT | Chr18: 55585438-55585460 | + | 5′ of TNRs of | −718 | 1101 | ACAGCUUAGAG | 197.78 | 7.6 |
| | TTATGCTAAGG | | | TCF4 | | | UUUAUGCUA | | |
| 14 | CAGCTTAGAGTTT | Chr18: 55585439-55585461 | + | 5′ of TNRs of | −717 | 1102 | CAGCUUAGAGU | 178.67 | 1.93 |
| | ATGCTAAGGG | | | TCF4 | | | UUAUGCUAA | | |
| 15 | TCTTTTAGTTTTA | Chr18: 55585483-55585505 | − | 5′ of TNRs of | −673 | 1103 | UCUUUUAGUU | 232.52 | 10.57 |
| | AGTTGGATGG | | | TCF4 | | | UUAAGUUGGA | | |
| 16 | TTTCTCTTTTAGTT | Chr18: 55585487-55585509 | − | 5′ of TNRs of | −669 | 1104 | UUUCUCUUUUA | 619.21 | 2.07 |
| | TTAAGTTGG | | | TCF4 | | | GUUUUAAGU | | |
| 17 | GTGATAATGGGG | Chr18: 55585523-55585545 | − | 5′ of TNRs of | −633 | 1105 | GUGAUAAUGG | 635.78 | 15.53 |
| | GCTGGGGTGGG | | | TCF4 | | | GGGCUGGGGU | | |
| 18 | AGTGATAATGGG | Chr18: 55585524-55585546 | − | 5′ of TNRs of | −632 | 1106 | AGUGAUAAUGG | 633.13 | 11.3 |
| | GGCTGGGGTGG | | | TCF4 | | | GGGCUGGGG | | |
| 19 | CAGAGTGATAAT | Chr18: 55585527-55585549 | − | 5′ of TNRs of | −629 | 1107 | CAGAGUGAUAA | 350.31 | 17.2 |
| | GGGGGCTGGGG | | | TCF4 | | | UGGGGGCUG | | |
| 20 | ACAGAGTGATAA | Chr18: 55585528-55585550 | − | 5′ of TNRs of | −628 | 1108 | ACAGAGUGAUA | 331.09 | 10.3 |
| | TGGGGGCTGGG | | | TCF4 | | | AUGGGGGCU | | |
| 21 | AACAGAGTGATA | Chr18: 55585529-55585551 | − | 5′ of TNRs of | −627 | 1109 | AACAGAGUGAU | 3776.91 | 12.53 |
| | ATGGGGGCTGG | | | TCF4 | | | AAUGGGGGC | | |
| 22 | AAAGAACAGAGT | Chr18: 55585533-55585555 | − | 5′ of TNRs of | −623 | 1110 | AAAGAACAGAG | 372.71 | 34 |
| | GATAATGGGGG | | | TCF4 | | | UGAUAAUGG | | |
| 23 | GAAAGAACAGAG | Chr18: 55585534-55585556 | − | 5′ of TNRs of | −622 | 1111 | GAAAGAACAGA | 5837.99 | 17.57 |
| | TGATAATGGGG | | | TCF4 | | | GUGAUAAUG | | |
| 24 | AGAAAGAACAGA | Chr18: 55585535-55585557 | − | 5′ of TNRs of | −621 | 1112 | AGAAAGAACAG | 1439.12 | 17.37 |
| | GTGATAATGGG | | | TCF4 | | | AGUGAUAAU | | |
| 25 | AAGAAAGAACAG | Chr18: 55585536-55585558 | − | 5′ of TNRs of | −620 | 1113 | AAGAAAGAACA | 418.32 | 4 |
| | AGTGATAATGG | | | TCF4 | | | GAGUGAUAA | | |
| 26 | TCTGTTCTTTCTTT | Chr18: 55585546-55585568 | + | 5′ of TNRs of | −610 | 1114 | UCUGUUCUUUC | 722.67 | 4.1 |
| | TTCCTCAGG | | | TCF4 | | | UUUUUCCUC | | |
| 27 | TTTTCCTCAGGTT | Chr18: 55585558-55585580 | + | 5′ of TNRs of | −598 | 1115 | UUUUCCUCAGG | 740.15 | 14.7 |
| | CATTAGATGG | | | TCF4 | | | UUCAUUAGA | | |
| 28 | TTGGCCATCTAAT | Chr18: 55585562-55585584 | − | 5′ of TNRs of | −594 | 1116 | UUGGCCAUCUA | 201.82 | 28.2 |
| | GAACCTGAGG | | | TCF4 | | | AUGAACCUG | | |
| 29 | AATGTAGCAGTA | Chr18: 55585581-55585603 | − | 5′ of TNRs of | −575 | 1117 | AAUGUAGCAGU | 932.03 | 23 |
| | GTACTGCTTGG | | | TCF4 | | | AGUACUGCU | | |
| 30 | AGCAGTACTACT | Chr18: 55585584-55585606 | + | 5′ of TNRs of | −572 | 1118 | AGCAGUACUAC | 975.76 | 4.43 |
| | GCTACATTTGG | | | TCF4 | | | UGCUACAUU | | |
| 31 | TGAATCTTGATAA | Chr18: 55585619-55585641 | − | 5′ of TNRs of | −537 | 1119 | UGAAUCUUGAU | 430.8 | 22.13 |
| | CATTATGGGG | | | TCF4 | | | AACAUUAUG | | |
| 32 | CTGAATCTTGATA | Chr18: 55585620-55585642 | − | 5′ of TNRs of | −536 | 1120 | CUGAAUCUUGA | 603.7 | 32.73 |
| | ACATTATGGG | | | TCF4 | | | UAACAUUAU | | |
| 33 | CCATAATGTTATC | Chr18: 55585621-55585643 | + | 5′ of TNRs of | −535 | 1121 | CCAUAAUGUUA | 473.28 | 15.53 |
| | AAGATTCAGG | | | TCF4 | | | UCAAGAUUC | | |
| 34 | CCTGAATCTTGAT | Chr18: 55585621-55585643 | − | 5′ of TNRs of | −535 | 1122 | CCUGAAUCUUG | 342.57 | 36.07 |
| | AACATTATGG | | | TCF4 | | | AUAACAUUA | | |
| 35 | AATGTTATCAAG | Chr18: 55585625-55585647 | + | 5′ of TNRs of | −531 | 1123 | AAUGUUAUCAA | 405.03 | 15.6 |
| | ATTCAGGTTGG | | | TCF4 | | | GAUUCAGGU | | |
| 36 | GTTATCAAGATTC | Chr18: 55585628-55585650 | + | 5′ of TNRs of | −528 | 1124 | GUUAUCAAGAU | 355.48 | 21.3 |
| | AGGTTGGAGG | | | TCF4 | | | UCAGGUUGG | | |
| 37 | TGTTTTTCTAGAG | Chr18: 55585651-55585673 | − | 5′ of TNRs of | −505 | 1125 | UGUUUUUCUA | 267.41 | 3.53 |
| | AGGCTGCTGG | | | TCF4 | | | GAGAGGCUGC | | |
| 38 | AAACTAGTGTTTT | Chr18: 55585658-55585680 | − | 5′ of TNRs of | −498 | 1126 | AAACUAGUGUU | 609.65 | 7.43 |
| | TCTAGAGAGG | | | TCF4 | | | UUUCUAGAG | | |
| 39 | GAAAAACACTAG | Chr18: 55585666-55585688 | + | 5′ of TNRs of | −490 | 1127 | GAAAAACACUA | 1273.03 | 22.27 |
| | TTTCACCAAGG | | | TCF4 | | | GUUUCACCA | | |
| 40 | AACAACTTTTTTC | Chr18: 55585683-55585705 | − | 5′ of TNRs of | −473 | 1128 | AACAACUUUUU | 187.55 | 3.37 |
| | TTCTCCTTGG | | | TCF4 | | | UCUUCUCCU | | |
| 41 | TTGTTTTATATTG | Chr18: 55585706-55585728 | + | 5′ of TNRs of | −450 | 1129 | UUGUUUUAUA | 330.57 | 5.57 |
| | AAAACCTTGG | | | TCF4 | | | UUGAAAACCU | | |
| 42 | GAAAACCTTGGC | Chr18: 55585718-55585740 | + | 5′ of TNRs of | −438 | 1130 | GAAAACCUUGG | 242.99 | 24.23 |
| | CATAAACGTGG | | | TCF4 | | | CCAUAAACG | | |
| 43 | CATTGCCACGTTT | Chr18: 55585723-55585745 | − | 5′ of TNRs of | −433 | 1131 | CAUUGCCACGU | 374.68 | 2.3 |
| | ATGGCCAAGG | | | TCF4 | | | UUAUGGCCA | | |
| 44 | AATGGACATTGC | Chr18: 55585729-55585751 | − | 5′ of TNRs of | −427 | 1132 | AAUGGACAUUG | 221.28 | 19.5 |
| | CACGTTTATGG | | | TCF4 | | | CCACGUUUA | | |
| 45 | TGTCCATTTCCAT | Chr18: 55585744-55585766 | + | 5′ of TNRs of | −412 | 1133 | UGUCCAUUUCC | 7973.48 | 12.53 |
| | CTCGTATAGG | | | TCF4 | | | AUCUCGUAU | | |
| 46 | AATCCTATACGA | Chr18: 55585747-55585769 | − | 5′ of TNRs of | −409 | 1134 | AAUCCUAUACG | 24066.2 | 6.87 |
| | GATGGAAATGG | | | TCF4 | | | AGAUGGAAA | | |
| 47 | CAGGCAAATCCT | Chr18: 55585753-55585775 | − | 5′ of TNRs of | −403 | 1135 | CAGGCAAAUCC | 1112.86 | 7.3 |
| | ATACGAGATGG | | | TCF4 | | | UAUACGAGA | | |
| 48 | TATTTGGGTTCAC | Chr18: 55585772-55585794 | − | 5′ of TNRs of | −384 | 1136 | UAUUUGGGUU | 1223.1 | 11.3 |
| | ATATGACAGG | | | TCF4 | | | CACAUAUGAC | | |
| 49 | TGGCACTTTTATT | Chr18: 55585787-55585809 | − | 5′ of TNRs of | −369 | 1137 | UGGCACUUUUA | 1409 | 1.37 |
| | TTTATTTGGG | | | TCF4 | | | UUUUUAUUU | | |
| 50 | GTGGCACTTTTAT | Chr18: 55585788-55585810 | − | 5′ of TNRs of | −368 | 1138 | GUGGCACUUUU | 8296.18 | 1.17 |
| | TTTTATTTGG | | | TCF4 | | | AUUUUUAUU | | |
| 51 | AAATGAGAATTT | Chr18: 55585807-55585829 | − | 5′ of TNRs of | −349 | 1139 | AAAUGAGAAUU | 780.66 | 4.73 |
| | AGTGCAGGTGG | | | TCF4 | | | UAGUGCAGG | | |
| 52 | ACGAAATGAGAA | Chr18: 55585810-55585832 | − | 5′ of TNRs of | −346 | 1140 | ACGAAAUGAGA | 372.43 | 8.9 |
| | TTTAGTGCAGG | | | TCF4 | | | AUUUAGUGC | | |
| 53 | ATTCTCATTTCGT | Chr18: 55585820-55585842 | + | 5′ of TNRs of | −336 | 1141 | AUUCUCAUUUC | 182.73 | 19.17 |
| | CTCTAACAGG | | | TCF4 | | | GUCUCUAAC | | |
| 54 | AAATAAATGCTG | Chr18: 55585898-55585920 | − | 5′ of TNRs of | −258 | 1142 | AAAUAAAUGCU | 283.11 | 32.93 |
| | GAGAGAGAGGG | | | TCF4 | | | GGAGAGAGA | | |
| 55 | GAAATAAATGCT | Chr18: 55585899-55585921 | − | 5′ of TNRs of | −257 | 1143 | GAAAUAAAUGC | 516.92 | 20.5 |
| | GGAGAGAGAGG | | | TCF4 | | | UGGAGAGAG | | |
| 56 | ATTAGGGTCGAA | Chr18: 55585908-55585930 | − | 5′ of TNRs of | −248 | 1144 | AUUAGGGUCGA | 2074.54 | 31.6 |
| | ATAAATGCTGG | | | TCF4 | | | AAUAAAUGC | | |
| 57 | GCATTTATTTCGA | Chr18: 55585911-55585933 | + | 5′ of TNRs of | −245 | 1145 | GCAUUUAUUUC | 430.39 | 12.77 |
| | CCCTAATTGG | | | TCF4 | | | GACCCUAAU | | |
| 58 | AAGAAGAGGGA | Chr18: 55585924-55585946 | − | 5′ of TNRs of | −232 | 1146 | AAGAAGAGGGA | 1894.27 | 47.23 |
| | AACCAATTAGGG | | | TCF4 | | | AACCAAUUA | | |
| 59 | GAAGAAGAGGG | Chr18: 55585925-55585947 | − | 5′ of TNRs of | −231 | 1147 | GAAGAAGAGGG | 632.04 | 24 |
| | AAACCAATTAGG | | | TCF4 | | | AAACCAAUU | | |
| 60 | ACTAGATACGTC | Chr18: 55585937-55585959 | − | 5′ of TNRs of | −219 | 1148 | ACUAGAUACGU | 554.05 | 18.97 |
| | GAAGAAGAGGG | | | TCF4 | | | CGAAGAAGA | | |
| 61 | CACTAGATACGTC | Chr18: 55585938-55585960 | − | 5′ of TNRs of | −218 | 1149 | CACUAGAUACG | 355.06 | 11.53 |
| | GAAGAAGAGG | | | TCF4 | | | UCGAAGAAG | | |
| 62 | CTCTTCTTCGACG | Chr18: 55585939-55585961 | + | 5′ of TNRs of | −217 | 1150 | CUCUUCUUCGA | 397.65 | 18.03 |
| | TATCTAGTGG | | | TCF4 | | | CGUAUCUAG | | |
| 63 | TGCAGGCTCTGA | Chr18: 55585972-55585994 | − | 5′ of TNRs of | −184 | 1151 | UGCAGGCUCUG | 611.76 | 5.97 |
| | CTCAGGGAAGG | | | TCF4 | | | ACUCAGGGA | | |
| 64 | TTTTTGCAGGCTC | Chr18: 55585976-55585998 | − | 5′ of TNRs of | −180 | 1152 | UUUUUGCAGGC | 471.42 | 4.37 |
| | TGACTCAGGG | | | TCF4 | | | UCUGACUCA | | |
| 65 | CTTTTTGCAGGCT | Chr18: 55585977-55585999 | − | 5′ of TNRs of | −179 | 1153 | CUUUUUGCAGG | 588.04 | 2.13 |
| | CTGACTCAGG | | | TCF4 | | | CUCUGACUC | | |
| 66 | TCAGAGCCTGCA | Chr18: 55585983-55586005 | + | 5′ of TNRs of | −173 | 1154 | UCAGAGCCUGC | 523.08 | 13.97 |
| | AAAAGCAAAGG | | | TCF4 | | | AAAAAGCAA | | |
| 67 | TTCGTTCCTTTGC | Chr18: 55585989-55586011 | − | 5′ of TNRs of | −167 | 1155 | UUCGUUCCUUU | 638.97 | 3.03 |
| | TTTTTGCAGG | | | TCF4 | | | GCUUUUUGC | | |
| 68 | GCAAAAAGCAAA | Chr18: 55585992-55586014 | + | 5′ of TNRs of | −164 | 1156 | GCAAAAAGCAA | 287.37 | 9.73 |
| | GGAACGAATGG | | | TCF4 | | | AGGAACGAA | | |
| 69 | AGAAAGTGCAAC | Chr18: 55586015-55586037 | + | 5′ of TNRs of | −141 | 1157 | AGAAAGUGCAA | 563.9 | 9.17 |
| | AAGCAGAAAGG | | | TCF4 | | | CAAGCAGAA | | |
| 70 | GAAAGTGCAACA | Chr18: 55586016-55586038 | + | 5′ of TNRs of | −140 | 1158 | GAAAGUGCAAC | 820.22 | 7.43 |
| | AGCAGAAAGGG | | | TCF4 | | | AAGCAGAAA | | |
| 71 | AAAGTGCAACAA | Chr18: 55586017-55586039 | + | 5′ of TNRs of | −139 | 1159 | AAAGUGCAACA | 677.96 | 30.07 |
| | GCAGAAAGGGG | | | TCF4 | | | AGCAGAAAG | | |
| 72 | AAGTGCAACAAG | Chr18: 55586018-55586040 | + | 5′ of TNRs of | −138 | 1160 | AAGUGCAACAA | 423.94 | 16.47 |
| | CAGAAAGGGGG | | | TCF4 | | | GCAGAAAGG | | |
| 73 | GGCTGCAAAGCT | Chr18: 55586039-55586061 | + | 5′ of TNRs of | −117 | 1161 | GGCUGCAAAGC | 295.09 | 1.43 |
| | GCCTGCCTAGG | | | TCF4 | | | UGCCUGCCU | | |
| 74 | GCTGCAAAGCTG | Chr18: 55586040-55586062 | + | 5′ of TNRs of | −116 | 1162 | GCUGCAAAGCU | 1404649 | 37.6 |
| | CCTGCCTAGGG | | | TCF4 | | | GCCUGCCUA | | |
| 75 | CAGGAAACGTAG | Chr18: 55586052-55586074 | − | 5′ of TNRs of | −104 | 1163 | CAGGAAACGUA | 189.68 | 8.43 |
| | CCCTAGGCAGG | | | TCF4 | | | GCCCUAGGC | | |
| 76 | CTGCCTAGGGCT | Chr18: 55586053-55586075 | + | 5′ of TNRs of | −103 | 1164 | CUGCCUAGGGC | 139.26 | 15 |
| | ACGTTTCCTGG | | | TCF4 | | | UACGUUUCC | | |
| 77 | TTGCCAGGAAAC | Chr18: 55586056-55586078 | − | 5′ of TNRs of | −100 | 1165 | UUGCCAGGAAA | 68.07 | 31.3 |
| | GTAGCCCTAGG | | | TCF4 | | | CGUAGCCCU | | |
| 78 | TGGCTTTCGGAA | Chr18: 55586071-55586093 | − | 5′ of TNRs of | −85 | 1166 | UGGCUUUCGGA | 1223977 | 17.97 |
| | GTTTTGCCAGG | | | TCF4 | | | AGUUUUGCC | | |
| 79 | TCTTTTGGAGAAA | Chr18: 55586084-55586106 | − | 5′ of TNRs of | −72 | 1167 | UCUUUUGGAG | 48.33 | 18.67 |
| | TGGCTTTCGG | | | TCF4 | | | AAAUGGCUUU | | |
| 80 | AAAGCCATTTCTC | Chr18: 55586087-55586109 | + | 5′ of TNRs of | −69 | 1168 | AAAGCCAUUUC | 12428.9 | 22.93 |
| | CAAAAGAAGG | | | TCF4 | | | UCCAAAAGA | | |
| 81 | TAGACCTTCTTTT | Chr18: 55586091-55586113 | − | 5′ of TNRs of | −65 | 1169 | UAGACCUUCUU | 581837 | 13 |
| | GGAGAAATGG | | | TCF4 | | | UUGGAGAAA | | |
| 82 | TCCAAAAGAAGG | Chr18: 55586098-55586120 | + | 5′ of TNRs of | −58 | 1170 | UCCAAAAGAAG | 1467679 | 21.4 |
| | TCTAGAAGAGG | | | TCF4 | | | GUCUAGAAG | | |
| 83 | TCCTCTTCTAGAC | Chr18: 55586099-55586121 | − | 5′ of TNRs of | −57 | 1171 | UCCUCUUCUAG | 5256.53 | 29.4 |
| | CTTCTTTTGG | | | TCF4 | | | ACCUUCUUU | | |
| 84 | AAAAGAAGGTCT | Chr18: 55586101-55586123 | + | 5′ of TNRs of | −55 | 1172 | AAAAGAAGGUC | 1030102 | 23.23 |
| | AGAAGAGGAGG | | | TCF4 | | | UAGAAGAGG | | |
| 85 | AGAAGGTCTAGA | Chr18: 55586104-55586126 | + | 5′ of TNRs of | −52 | 1173 | AGAAGGUCUAG | 1040794 | 31.1 |
| | AGAGGAGGAGG | | | TCF4 | | | AAGAGGAGG | | |
| 86 | AGGTCTAGAAGA | Chr18: 55586107-55586129 | + | 5′ of TNRs of | −49 | 1174 | AGGUCUAGAAG | 2449.47 | 39.2 |
| | GGAGGAGGAGG | | | TCF4 | | | AGGAGGAGG | | |
| 87 | TCTAGAAGAGGA | Chr18: 55586110-55586132 | + | 5′ of TNRs of | −46 | 1175 | UCUAGAAGAGG | 1657.42 | 8.33 |
| | GGAGGAGGAGG | | | TCF4 | | | AGGAGGAGG | | |
| 88 | AGAGGAGGAGG | Chr18: 55586116-55586138 | + | 5′ of TNRs of | −40 | 1176 | AGAGGAGGAGG | 773.69 | 15.67 |
| | AGGAGGAGAAGG | | | TCF4 | | | AGGAGGAGA | | |
| 89 | GGAGGAGGAGG | Chr18: 55586119-55586141 | + | 5′ of TNRs of | −37 | 1177 | GGAGGAGGAGG | 420.41 | 17.23 |
| | AGGAGAAGGAGG | | | TCF4 | | | AGGAGAAGG | | |
| 90 | GGAGGAGGAGG | Chr18: 55586122-55586144 | + | 5′ of TNRs of | −34 | 1178 | GGAGGAGGAGG | 394.07 | 8.03 |
| | AGAAGGAGGAGG | | | TCF4 | | | AGAAGGAGG | | |
| 91 | GGAGGAGGAGA | Chr18: 55586125-55586147 | + | 5′ of TNRs of | −31 | 1179 | GGAGGAGGAGA | 947.52 | 5.03 |
| | AGGAGGAGGAGG | | | TCF4 | | | AGGAGGAGG | | |
| 92 | GGAGGAGAAGG | Chr18: 55586128-55586150 | + | 5′ of TNRs of | −28 | 1180 | GGAGGAGAAGG | 448.19 | 5.73 |
| | AGGAGGAGGAGG | | | TCF4 | | | AGGAGGAGG | | |
| 93 | GGAGAAGGAGG | Chr18: 55586131-55586153 | + | 5′ of TNRs of | −25 | 1181 | GGAGAAGGAGG | 598.33 | 6 |
| | AGGAGGAGGAGG | | | TCF4 | | | AGGAGGAGG | | |
| 94 | CAGCATGAAAGA | Chr18: 55586225-55586247 | + | 3′ of TNRs of | 69 | 1182 | CAGCAUGAAAG | 6355.32 | 18.63 |
| | GCCCCACTTGG | | | TCF4 | | | AGCCCCACU | | |
| 95 | ATGAAAGAGCCC | Chr18: 55586229-55586251 | + | 3′ of TNRs of | 73 | 1183 | AUGAAAGAGCC | 697.17 | 26.83 |
| | CACTTGGAAGG | | | TCF4 | | | CCACUUGGA | | |
| 96 | AAAGAGCCCCAC | Chr18: 55586232-55586254 | + | 3′ of TNRs of | 76 | 1184 | AAAGAGCCCCA | 130.15 | 22.7 |
| | TTGGAAGGCGG | | | TCF4 | | | CUUGGAAGG | | |
| 97 | GCCCCACTTGGA | Chr18: 55586237-55586259 | + | 3′ of TNRs of | 81 | 1185 | GCCCCACUUGG | 203.63 | 6.7 |
| | AGGCGGTTTGG | | | TCF4 | | | AAGGCGGUU | | |
| 98 | TCCAAACCGCCTT | Chr18: 55586238-55586260 | − | 3′ of TNRs of | 82 | 1186 | UCCAAACCGCC | 203.16 | 8.07 |
| | CCAAGTGGGG | | | TCF4 | | | UUCCAAGUG | | |
| 99 | ATCCAAACCGCCT | Chr18: 55586239-55586261 | − | 3′ of TNRs of | 83 | 1187 | AUCCAAACCGCC | 105.14 | 11.4 |
| | TCCAAGTGGG | | | TCF4 | | | UUCCAAGU | | |
| 100 | AATCCAAACCGC | Chr18: 55586240-55586262 | − | 3′ of TNRs of | 84 | 1188 | AAUCCAAACCG | 160.67 | 18.07 |
| | CTTCCAAGTGG | | | TCF4 | | | CCUUCCAAG | | |
| 101 | GATTTTATTTGTG | Chr18: 55586259-55586281 | + | 3′ of TNRs of | 103 | 1189 | GAUUUUAUUU | 329.17 | 0.23 |
| | TGTTTTGTGG | | | TCF4 | | | GUGUGUUUUG | | |
| 102 | CATCTTACACCAA | Chr18: 55586308-55586330 | + | 3′ of TNRs of | 152 | 1190 | CAUCUUACACC | 405.23 | 12.2 |
| | ACTCATCTGG | | | TCF4 | | | AAACUCAUC | | |
| 103 | TTTTTAATGCCAG | Chr18: 55586317-55586339 | − | 3′ of TNRs of | 161 | 1191 | UUUUUAAUGCC | 282.35 | 8.63 |
| | ATGAGTTTGG | | | TCF4 | | | AGAUGAGUU | | |
| 104 | ATTCATTCTCCTG | Chr18: 55586343-55586365 | + | 3′ of TNRs of | 187 | 1192 | AUUCAUUCUCC | 2000.64 | 8.23 |
| | ACATGTCTGG | | | TCF4 | | | UGACAUGUC | | |
| 105 | TTCATTCTCCTGA | Chr18: 55586344-55586366 | + | 3′ of TNRs of | 188 | 1193 | UUCAUUCUCCU | 35953.9 | 12.3 |
| | CATGTCTGGG | | | TCF4 | | | GACAUGUCU | | |
| 106 | CTCCTGACATGTC | Chr18: 55586350-55586372 | + | 3′ of TNRs of | 194 | 1194 | CUCCUGACAUG | 683.98 | 7.03 |
| | TGGGACTTGG | | | TCF4 | | | UCUGGGACU | | |
| 107 | AACCAAGTCCCA | Chr18: 55586352-55586374 | − | 3′ of TNRs of | 196 | 1195 | AACCAAGUCCC | 5020.06 | 22.2 |
| | GACATGTCAGG | | | TCF4 | | | AGACAUGUC | | |
| 108 | ACATGTCTGGGA | Chr18: 55586356-55586378 | + | 3′ of TNRs of | 200 | 1196 | ACAUGUCUGGG | 1201.43 | 21.03 |
| | CTTGGTTTAGG | | | TCF4 | | | ACUUGGUUU | | |
| 109 | CTGGGACTTGGT | Chr18: 55586362-55586384 | + | 3′ of TNRs of | 206 | 1197 | CUGGGACUUGG | 1784.35 | 32 |
| | TTAGGAAAAGG | | | TCF4 | | | UUUAGGAAA | | |
| 110 | GGTTTAGGAAAA | Chr18: 55586371-55586393 | + | 3′ of TNRs of | 215 | 1198 | GGUUUAGGAAA | 1362.04 | 11.57 |
| | GGAAGCAAAGG | | | TCF4 | | | AGGAAGCAA | | |
| 111 | GTTTAGGAAAAG | Chr18: 55586372-55586394 | + | 3′ of TNRs of | 216 | 1199 | GUUUAGGAAAA | 4810.53 | 12.17 |
| | GAAGCAAAGGG | | | TCF4 | | | GGAAGCAAA | | |
| 112 | AGGAAAAGGAA | Chr18: 55586376-55586398 | + | 3′ of TNRs of | 220 | 1200 | AGGAAAAGGAA | 814.55 | 20.47 |
| | GCAAAGGGATGG | | | TCF4 | | | GCAAAGGGA | | |
| 113 | AGGAAGCAAAGG | Chr18: 55586382-55586404 | + | 3′ of TNRs of | 226 | 1201 | AGGAAGCAAAG | 878.55 | 16.2 |
| | GATGGAGAAGG | | | TCF4 | | | GGAUGGAGA | | |
| 114 | TGGAGTTTTACG | Chr18: 55586406-55586428 | − | 3′ of TNRs of | 250 | 1202 | UGGAGUUUUA | 315.87 | 25.63 |
| | GCTGTACTTGG | | | TCF4 | | | CGGCUGUACU | | |
| 115 | GACACACTTGTG | Chr18: 55586416-55586438 | − | 3′ of TNRs of | 260 | 1203 | GACACACUUGU | 177.25 | 20.47 |
| | GAGTTTTACGG | | | TCF4 | | | GGAGUUUUA | | |
| 116 | AGCGGAACTTGA | Chr18: 55586426-55586448 | − | 3′ of TNRs of | 270 | 1204 | AGCGGAACUUG | 135.84 | 17.3 |
| | CACACTTGTGG | | | TCF4 | | | ACACACUUG | | |
| 117 | GTCGTAGGATCA | Chr18: 55586444-55586466 | − | 3′ of TNRs of | 288 | 1205 | GUCGUAGGAUC | 797.01 | 20.3 |
| | GCACAAAGCGG | | | TCF4 | | | AGCACAAAG | | |
| 118 | TTGGTAAATTTCG | Chr18: 55586459-55586481 | − | 3′ of TNRs of | 303 | 1206 | UUGGUAAAUU | 200.12 | 9.3 |
| | TAGTCGTAGG | | | TCF4 | | | UCGUAGUCGU | | |
| 119 | ATTTACCAAAACA | Chr18: 55586473-55586495 | + | 3′ of TNRs of | 317 | 1207 | AUUUACCAAAA | 1602.25 | NA |
| | GTCCAAAAGG | | | TCF4 | | | CAGUCCAAA | | |
| 120 | TAGAACCTTTTGG | Chr18: 55586478-55586500 | − | 3′ of TNRs of | 322 | 1208 | UAGAACCUUUU | 5716.11 | 5 |
| | ACTGTTTTGG | | | TCF4 | | | GGACUGUUU | | |
| 121 | ATACATTCTTTAG | Chr18: 55586488-55586510 | − | 3′ of TNRs of | 332 | 1209 | AUACAUUCUUU | 345.52 | 7.5 |
| | AACCTTTTGG | | | TCF4 | | | AGAACCUUU | | |
| 122 | TAGGATTCTTAAA | Chr18: 55586522-55586544 | − | 3′ of TNRs of | 366 | 1210 | UAGGAUUCUUA | 1052.11 | 1.83 |
| | ACTAGTATGG | | | TCF4 | | | AAACUAGUA | | |
| 123 | ATACTAGTTTTAA | Chr18: 55586524-55586546 | + | 3′ of TNRs of | 368 | 1211 | AUACUAGUUUU | 1437.37 | 10.03 |
| | GAATCCTAGG | | | TCF4 | | | AAGAAUCCU | | |
| 124 | TCCTAGGAAAAG | Chr18: 55586540-55586562 | + | 3′ of TNRs of | 384 | 1212 | UCCUAGGAAAA | 2172.51 | 20.9 |
| | ATGTAACTAGG | | | TCF4 | | | GAUGUAACU | | |
| 125 | TCCTAGTTACATC | Chr18: 55586541-55586563 | − | 3′ of TNRs of | 385 | 1213 | UCCUAGUUACA | 1136.69 | 15.03 |
| | TTTTCCTAGG | | | TCF4 | | | UCUUUUCCU | | |
| 126 | TAGGAAAAGATG | Chr18: 55586543-55586565 | + | 3′ of TNRs of | 387 | 1214 | UAGGAAAAGAU | 1044.91 | 23.3 |
| | TAACTAGGAGG | | | TCF4 | | | GUAACUAGG | | |
| 127 | TAACTAGGAGGT | Chr18: 55586555-55586577 | + | 3′ of TNRs of | 399 | 1215 | UAACUAGGAGG | 707.33 | 22.5 |
| | AAGATGTAAGG | | | TCF4 | | | UAAGAUGUA | | |
| 128 | GGAGGTAAGATG | Chr18: 55586561-55586583 | + | 3′ of TNRs of | 405 | 1216 | GGAGGUAAGAU | 473.79 | 16.03 |
| | TAAGGAACAGG | | | TCF4 | | | GUAAGGAAC | | |
| 129 | TAATGATGCTTTG | Chr18: 55586585-55586607 | − | 3′ of TNRs of | 429 | 1217 | UAAUGAUGCUU | 7.55 | 19.93 |
| | GATTGGTAGG | | | TCF4 | | | UGGAUUGGU | | |
| 130 | AAGCTAATGATG | Chr18: 55586589-55586611 | − | 3′ of TNRs of | 433 | 1218 | AAGCUAAUGAU | 48.63 | 15.27 |
| | CTTTGGATTGG | | | TCF4 | | | GCUUUGGAU | | |
| 131 | GTTTTAAGCTAAT | Chr18: 55586594-55586616 | − | 3′ of TNRs of | 438 | 1219 | GUUUUAAGCUA | 1051.28 | 3.67 |
| | ATGCTTTGG | | | TCF4 | | | AUGAUGCUU | | |
| 132 | TAAAACTTTAAAG | Chr18: 55586611-55586633 | + | 3′ of TNRs of | 455 | 1220 | UAAAACUUUAA | 83.63 | 12.03 |
| | AGACAACTGG | | | TCF4 | | | AGAGACAAC | | |
| 133 | AAAACTTTAAAG | Chr18: 55586612-55586634 | + | 3′ of TNRs of | 456 | 1221 | AAAACUUUAAA | 841.09 | 32.53 |
| | AGACAACTGGG | | | TCF4 | | | GAGACAACU | | |
| 134 | GGAAATGGAAAA | Chr18: 55586638-55586660 | − | 3′ of TNRs of | 482 | 1222 | GGAAAUGGAAA | 22.4 | 13.73 |
| | TAGAAAATAGG | | | TCF4 | | | AUAGAAAAU | | |
| 135 | TTATTTATTGTTTT | Chr18: 55586653-55586675 | − | 3′ of TNRs of | 497 | 1223 | UUAUUUAUUG | 2366.77 | 0.13 |
| | TGGAAATGG | | | TCF4 | | | UUUUUGGAAA | | |
| 136 | TTCGTTTTATTTAT | Chr18: 55586659-55586681 | − | 3′ of TNRs of | 503 | 1224 | UUCGUUUUAU | 1039.95 | 0.07 |
| | TGTTTTTGG | | | TCF4 | | | UUAUUGUUUU | | |
| 137 | GTAGTCTCAGTGT | Chr18: 55586702-55586724 | + | 3′ of TNRs of | 546 | 1225 | GUAGUCUCAGU | 1965.79 | 5.37 |
| | TCAGACATGG | | | TCF4 | | | GUUCAGACA | | |
| 138 | TTCAGACATGGC | Chr18: 55586714-55586736 | + | 3′ of TNRs of | 558 | 1226 | UUCAGACAUGG | 3320.5 | 2.33 |
| | CAAGTTTTAGG | | | TCF4 | | | CCAAGUUUU | | |
| 139 | TCAGACATGGCC | Chr18: 55586715-55586737 | + | 3′ of TNRs of | 559 | 1227 | UCAGACAUGGC | 717.05 | 5.9 |
| | AAGTTTTAGGG | | | TCF4 | | | CAAGUUUUA | | |
| 140 | CAGACATGGCCA | Chr18: 55586716-55586738 | + | 3′ of TNRs of | 560 | 1228 | CAGACAUGGCC | 300.9 | 6.37 |
| | AGTTTTAGGGG | | | TCF4 | | | AAGUUUUAG | | |
| 141 | ACATGGCCAAGT | Chr18: 55586719-55586741 | + | 3′ of TNRs of | 563 | 1229 | ACAUGGCCAAG | 301.24 | 12.73 |
| | TTTAGGGGTGG | | | TCF4 | | | UUUUAGGGG | | |
| 142 | ACTAAACCACCCC | Chr18: 55586725-55586747 | − | 3′ of TNRs of | 569 | 1230 | ACUAAACCACCC | 333.64 | 1.57 |
| | TAAAACTTGG | | | TCF4 | | | CUAAAACU | | |
| 143 | TTTAGGGGTGGT | Chr18: 55586731-55586753 | + | 3′ of TNRs of | 575 | 1231 | UUUAGGGGUG | 171.1 | 3.2 |
| | TTAGTTTTAGG | | | TCF4 | | | GUUUAGUUUU | | |
| 144 | TTAGGGGTGGTT | Chr18: 55586732-55586754 | + | 3′ of TNRs of | 576 | 1232 | UUAGGGGUGG | 214.26 | 6.8 |
| | TAGTTTTAGGG | | | TCF4 | | | UUUAGUUUUA | | |
| 145 | TAGGGGTGGTTT | Chr18: 55586733-55586755 | + | 3′ of TNRs of | 577 | 1233 | UAGGGGUGGU | 147.48 | 10.37 |
| | AGTTTTAGGGG | | | TCF4 | | | UUAGUUUUAG | | |
| 146 | TGTCTATTTTTGC | Chr18: 55586756-55586778 | + | 3′ of TNRs of | 600 | 1234 | UGUCUAUUUU | 995.21 | 4.33 |
| | TTTCCACTGG | | | TCF4 | | | UGCUUUCCAC | | |
| 147 | GTCTATTTTTGCT | Chr18: 55586757-55586779 | + | 3′ of TNRs of | 601 | 1235 | GUCUAUUUUU | 174.31 | 1.7 |
| | TTCCACTGGG | | | TCF4 | | | GCUUUCCACU | | |
| 148 | TCTATTTTTGCTTT | Chr18: 55586758-55586780 | + | 3′ of TNRs of | 602 | 1236 | UCUAUUUUUGC | 84.57 | 5.7 |
| | CCACTGGGG | | | TCF4 | | | UUUCCACUG | | |
| 149 | ATAATGGAATCTC | Chr18: 55586772-55586794 | − | 3′ of TNRs of | 616 | 1237 | AUAAUGGAAUC | 298.73 | 14.83 |
| | ACCCCAGTGG | | | TCF4 | | | UCACCCCAG | | |
| 150 | TGGGGTGAGATT | Chr18: 55586776-55586798 | + | 3′ of TNRs of | 620 | 1238 | UGGGGUGAGA | 2434.89 | 4.53 |
| | CCATTATTTGG | | | TCF4 | | | UUCCAUUAUU | | |
| 151 | GGGGTGAGATTC | Chr18: 55586777-55586799 | + | 3′ of TNRs of | 621 | 1239 | GGGGUGAGAU | 1205.02 | 4.8 |
| | CATTATTTGGG | | | TCF4 | | | UCCAUUAUUU | | |
| 152 | GGGTGAGATTCC | Chr18: 55586778-55586800 | + | 3′ of TNRs of | 622 | 1240 | GGGUGAGAUUC | 2784.14 | 4.63 |
| | ATTATTTGGGG | | | TCF4 | | | CAUUAUUUG | | |
| 153 | CCATTATTTGGGG | Chr18: 55586788-55586810 | + | 3′ of TNRs of | 632 | 1241 | CCAUUAUUUGG | 978.57 | 17.53 |
| | TAATCAGTGG | | | TCF4 | | | GGUAAUCAG | | |
| 154 | CCACTGATTACCC | Chr18: 55586788-55586810 | − | 3′ of TNRs of | 632 | 1242 | CCACUGAUUAC | 42.74 | 12.17 |
| | CAAATAATGG | | | TCF4 | | | CCCAAAUAA | | |
| 155 | CATTATTTGGGGT | Chr18: 55586789-55586811 | + | 3′ of TNRs of | 633 | 1243 | CAUUAUUUGG | 1266.08 | 19.47 |
| | AATCAGTGGG | | | TCF4 | | | GGUAAUCAGU | | |
| 156 | ATTTGGGGTAAT | Chr18: 55586793-55586815 | + | 3′ of TNRs of | 637 | 1244 | AUUUGGGGUA | 251.48 | 6.2 |
| | CAGTGGGTAGG | | | TCF4 | | | AUCAGUGGGU | | |
| 157 | TTTGGGGTAATC | Chr18: 55586794-55586816 | + | 3′ of TNRs of | 638 | 1245 | UUUGGGGUAA | 443.03 | 8.7 |
| | AGTGGGTAGGG | | | TCF4 | | | UCAGUGGGUA | | |
| 158 | ATCAGTGGGTAG | Chr18: 55586803-55586825 | + | 3′ of TNRs of | 647 | 1246 | AUCAGUGGGUA | 616.38 | 7.2 |
| | GGAATTGAAGG | | | TCF4 | | | GGGAAUUGA | | |
| 159 | TTTTTTTTGAGTT | Chr18: 55586826-55586848 | − | 3′ of TNRs of | 670 | 1247 | UUUUUUUUGA | 843.87 | 1.1 |
| | TTATTACTGG | | | TCF4 | | | GUUUUAUUAC | | |
| 160 | TGTGGTGTGATG | Chr18: 55586856-55586878 | − | 3′ of TNRs of | 700 | 1248 | UGUGGUGUGA | 565.01 | 6.47 |
| | GAAGATTCAGG | | | TCF4 | | | UGGAAGAUUC | | |
| 161 | ACTATAATTTTGT | Chr18: 55586866-55586888 | − | 3′ of TNRs of | 710 | 1249 | ACUAUAAUUUU | 4828.97 | 0.5 |
| | GGTGTGATGG | | | TCF4 | | | GUGGUGUGA | | |
| 162 | AGTTTTTAACTAT | Chr18: 55586874-55586896 | − | 3′ of TNRs of | 718 | 1250 | AGUUUUUAACU | 339.02 | 1.1 |
| | AATTTTGTGG | | | TCF4 | | | AUAAUUUUG | | |
| 163 | AAAGACCTTCATA | Chr18: 55586903-55586925 | + | 3′ of TNRs of | 747 | 1251 | AAAGACCUUCA | 142.27 | 5.87 |
| | TTTACCAAGG | | | TCF4 | | | UAUUUACCA | | |
| 164 | TGAATCCTTGGTA | Chr18: 55586908-55586930 | − | 3′ of TNRs of | 752 | 1252 | UGAAUCCUUGG | 789.33 | 3.17 |
| | AATATGAAGG | | | TCF4 | | | UAAAUAUGA | | |
| 165 | TTTTTAATTGGCT | Chr18: 55586920-55586942 | − | 3′ of TNRs of | 764 | 1253 | UUUUUAAUUG | 3433.08 | 8.07 |
| | GAATCCTTGG | | | TCF4 | | | GCUGAAUCCU | | |
| 166 | GGACAGTAATAA | Chr18: 55586932-55586954 | − | 3′ of TNRs of | 776 | 1254 | GGACAGUAAUA | 187.99 | 0.83 |
| | TTTTTAATTGG | | | TCF4 | | | AUUUUUAAU | | |
| 167 | ACTGTCCTTTAGA | Chr18: 55586948-55586970 | + | 3′ of TNRs of | 792 | 1255 | ACUGUCCUUUA | 3697.81 | 8.13 |
| | TTCCTACTGG | | | TCF4 | | | GAUUCCUAC | | |
| 168 | AGAAACCAGTAG | Chr18: 55586953-55586975 | − | 3′ of TNRs of | 797 | 1256 | AGAAACCAGUA | 1485.36 | 5.8 |
| | GAATCTAAAGG | | | TCF4 | | | GGAAUCUAA | | |
| 169 | CACTTCAGCTAGA | Chr18: 55586963-55586985 | − | 3′ of TNRs of | 807 | 1257 | CACUUCAGCUA | 1419.43 | 7.7 |
| | AACCAGTAGG | | | TCF4 | | | GAAACCAGU | | |
| 170 | TGGTTTCTAGCTG | Chr18: 55586968-55586990 | + | 3′ of TNRs of | 812 | 1258 | UGGUUUCUAGC | 1064.11 | 6.83 |
| | AAGTGTTTGG | | | TCF4 | | | UGAAGUGUU | | |
| 171 | GGTTTCTAGCTGA | Chr18: 55586969-55586991 | + | 3′ of TNRs of | 813 | 1259 | GGUUUCUAGCU | 742.1 | 8.47 |
| | AGTGTTTGGG | | | TCF4 | | | GAAGUGUUU | | |
| 172 | AGTGCGGTAAGA | Chr18: 55587028-55587050 | − | 3′ of TNRs of | 872 | 1260 | AGUGCGGUAAG | 1308.2 | 23.43 |
| | AAGAACGGTGG | | | TCF4 | | | AAAGAACGG | | |
| 173 | TTCAGTGCGGTA | Chr18: 55587031-55587053 | − | 3′ of TNRs of | 875 | 1261 | UUCAGUGCGGU | 833.82 | 23.33 |
| | AGAAAGAACGG | | | TCF4 | | | AAGAAAGAA | | |
| 174 | TGATTTACTGGAT | Chr18: 55587044-55587066 | − | 3′ of TNRs of | 888 | 1262 | UGAUUUACUG | 1281.47 | NA |
| | TTCAGTGCGG | | | TCF4 | | | GAUUUCAGUG | | |
| 175 | CAAAGAGCTGAG | Chr18: 55587056-55587078 | − | 3′ of TNRs of | 900 | 1263 | CAAAGAGCUGA | 1093.05 | NA |
| | TGATTTACTGG | | | TCF4 | | | GUGAUUUAC | | |
| 176 | CAGCTCTTTGTCC | Chr18: 55587069-55587091 | + | 3′ of TNRs of | 913 | 1264 | CAGCUCUUUGU | 2384.95 | NA |
| | GTCCCTAAGG | | | TCF4 | | | CCGUCCCUA | | |
| 177 | GCGAATGGCTGC | Chr18: 55587080-55587102 | − | 3′ of TNRs of | 924 | 1265 | GCGAAUGGCUG | 136.05 | NA |
| | CTTAGGGACGG | | | TCF4 | | | CCUUAGGGA | | |
| 178 | AACAGCGAATGG | Chr18: 55587084-55587106 | − | 3′ of TNRs of | 928 | 1266 | AACAGCGAAUG | 1946.76 | NA |
| | CTGCCTTAGGG | | | TCF4 | | | GCUGCCUUA | | |
| 179 | CAACAGCGAATG | Chr18: 55587085-55587107 | − | 3′ of TNRs of | 929 | 1267 | CAACAGCGAAU | 922.31 | NA |
| | CTGCCTTAGG | | | TCF4 | | | GGCUGCCUU | | |
| 180 | CTAAGGCAGCCA | Chr18: 55587086-55587108 | + | 3′ of TNRs of | 930 | 1268 | CUAAGGCAGCC | 1288.59 | NA |
| | TTCGCTGTTGG | | | TCF4 | | | AUUCGCUGU | | |
| 181 | AATGCATCACCA | Chr18: 55587095-55587117 | − | 3′ of TNRs of | 939 | 1269 | AAUGCAUCACC | 221.14 | NA |
| | ACAGCGAATGG | | | TCF4 | | | AACAGCGAA | | |
| 182 | ATCACACAAACCT | Chr18: 55587126-55587148 | + | 3′ of TNRs of | 970 | 1270 | AUCACACAAACC | 1315.96 | NA |
| | AGAAACATGG | | | TCF4 | | | UAGAAACA | | |
| 183 | GCGGTTATTTCCA | Chr18: 55587136-55587158 | − | 3′ of TNRs of | 980 | 1271 | GCGGUUAUUUC | 1600 | NA |
| | TGTTTCTAGG | | | TCF4 | | | CAUGUUUCU | | |
| 184 | GGGACTGGATTT | Chr18: 55587155-55587177 | − | 3′ of TNRs of | 999 | 1272 | GGGACUGGAUU | 1287.34 | NA |
| | TCTGATTGCGG | | | TCF4 | | | UUCUGAUUG | | |
| 185 | GAAAATCCAGTC | Chr18: 55587164-55587186 | + | 3′ of TNRs of | 1008 | 1273 | GAAAAUCCAGU | 1557.06 | NA |
| | CCAATCCTTGG | | | TCF4 | | | CCCAAUCCU | | |
| 186 | TTTTCTCCAAGGA | Chr18: 55587170-55587192 | − | 3′ of TNRs of | 1014 | 1274 | UUUUCUCCAAG | 1644.63 | NA |
| | TTGGGACTGG | | | TCF4 | | | GAUUGGGAC | | |
| 187 | TTGTGTTTTCTCC | Chr18: 55587175-55587197 | − | 3′ of TNRs of | 1019 | 1275 | UUGUGUUUUC | 495.78 | NA |
| | AAGGATTGGG | | | TCF4 | | | UCCAAGGAUU | | |
| 188 | ATTGTGTTTTCTC | Chr18: 55587176-55587198 | − | 3′ of TNRs of | 1020 | 1276 | AUUGUGUUUU | 2305.18 | NA |
| | CAAGGATTGG | | | TCF4 | | | CUCCAAGGAU | | |
| 189 | ATCCTTGGAGAA | Chr18: 55587179-55587201 | + | 3′ of TNRs of | 1023 | 1277 | AUCCUUGGAGA | 527.93 | NA |
| | AACACAATCGG | | | TCF4 | | | AAACACAAU | | |
| 190 | ATCCGATTGTGTT | Chr18: 55587181-55587203 | − | 3′ of TNRs of | 1025 | 1278 | AUCCGAUUGUG | 125.71 | NA |
| | TTCTCCAAGG | | | TCF4 | | | UUUUCUCCA | | |

gRNAs having guide sequences provided in Table 2 were screened in a 96-well format to determine their editing (e.g., indel forming) efficiency. To this end, a HEK293 cell line constitutively expressing Spy Cas9 (“HEK293_Cas9”) was cultured in DMEM media supplemented with 10% fetal bovine serum and 500 μg/ml G418. Cells were plated at a density of 10,000 cells/well in a 96-well plate 20 hours prior to transfection. Cells were transfected with Lipofectamine RNAiMAX (ThermoFisher, Cat. 13778150) according to the manufacturer's protocol. Cells were transfected with a lipoplex containing individual crRNA (25 nM), trRNA (25 nM), Lipofectamine RNAiMAX (0.3 μL/well) and OptiMem. Genomic DNA was extracted from each well using 50 μL/well BuccalAmp DNA Extraction solution (Epicentre, Cat. QE09050) according to manufacturer's protocol.

To quantitatively determine the efficiency of editing at the target location in the genome, deep sequencing was utilized to identify the presence of insertions and deletions (“indels”) introduced by gene editing. PCR primers were designed around the target sites and the genomic area of interest was amplified. Additional PCR was performed according to the manufacturer's protocols (Illumina) to add the necessary chemistry for sequencing. The amplicons were sequenced on an Illumina MiSeq instrument. The reads were aligned to the human reference genome after eliminating those having low quality scores. The resulting files containing the reads were mapped to the reference genome (BAM files), where reads that overlapped the target region of interest were selected and the number of wild type reads versus the number of reads which contain an insertion, substitution, or deletion was calculated. The editing percentage (e.g., the “editing efficiency” or “percent editing”) is defined as the total number of sequence reads with insertions or deletions over the total number of sequence reads, including wild type. The editing efficiency numbers for each gRNA used are reported in Table 2.

After completing the initial evaluation above to identify those with optimal editing efficiency, pairs of gRNAs were screened to determine pairs capable of removing the intervening section of DNA containing the TNR, as shown in FIG. 1. Following excision of the intervening section, the break will then be repaired by the cell through the non-homologous end joining (NHEJ) DNA repair pathway, which is highly efficient even in non-dividing cells such as those in the corneal endothelium. This process follows excision of the DNA fragment between the two guide sequences, which can occur at high frequency even when the guide sequences are >3000 nucleotides apart. No additional homologous template DNA is required for this editing approach, greatly simplifying the process. As the deleted range is contained within an intron, no effect on the gene product of TCF4 would be expected as the intron does not affect the final mRNA product or the protein product.

After removal of the TNR repeat, the TCF4 RNA transcript should no longer aggregate within the cell, nor sequester the splicing factors that are required for normal cellular function. Removal of the relevant region within intron 3 is unlikely to have any detrimental effects on RNA stability or the expression of the TCF4 gene itself, because this intron would normally be removed by RNA splicing during maturation of the final RNA product. Thus, the region of DNA within intron 3 is not be contained within the final RNA product used for translation of the TCF4 protein. Without the TNR, the mRNA and gene product of TCF4 should function normally, much the same as a normal allele with minimal TNR expansion. Moreover, as corneal endothelial cells are essentially non-dividing, correction of the cells once should result in a permanent amelioration of the disease. Treatment should halt the abnormal deposition of collagen (i.e., guttae) characteristic of the disease, and may over time lead to resorption of existing guttae. It is also proposed that treatment of individuals with a known predisposition to FECD, such as those with family histories of the disease and who are confirmed to have TNR expansion of intron 3 of TCF4, using this technology may prevent development of disease.

To demonstrate excision of the TNR, pairs of RNPs were formed, each having a gRNA targeting one side of the TNR. Brifely, a 50 μM solution of pre-annealed gRNA (e.g., a dgRNA having a crRNA and trRNA) was prepared by heating crRNA and trRNA at eqimolar amounts in water at 95° C. for 2 minutes, and allowing them to cool at room temperature. The pre-annleaed gRNA was added to Spy Cas9 protein (at 50 μM concentration) and was incubated at room temperature for 10 minutes, giving a final RNP solution having gRNA at 3.33 μM and Cas9 protein at 1.66 μM. HEK293 cells which do not constitutively express Cas9 were plated in SF electroporation buffer (Lonza) in 96-well format at ˜50,000 cells/well in a volume of 20 μL. 5 μl of each RNP solution (e.g., for each pair being tested) was added to the wells and the cells were electroporated using a Lonza Amaxa instrument. After electroporation, 80 μL of cell culture media was added to the wells and the cells were transferred to a 96-well flat bottom tissue culture plate and incubated at 37° C. for 24 hours. The cells were then lysed and genomic DNA was extracted as described above.

To determine efficiciences of TNR excision, a similar NGS analysis was performed as described above for editing efficiency. Brifely, deep sequencing was performed to identify deletions caused by gene editing of two locations flanking the TNRs. PCR primers were designed around the target site (the TNR in intron 3 of TCF4), and the genomic area of interest was amplified. Additional PCR was performed according to the manufacturer's protocols (Illumina) to add the necessary chemistry for sequencing. The resulting amplicons were sequenced on an Illumina MiSeq instrument. Reads were filtered to eliminate those with low quality scores, and the resulting reads were mapped to the reference genome. Reads overlapping the target region were further filtered and locally realigned to identify large deletions. The number of reads containing deletions spanning the two targeted regions was calculated. The excision percentage is defined as the number of sequencing reads containing a deletion of the TNRs divided by the total number of reads overlapping the target region. The excision percentages for each pair tested are reported in Table 7.

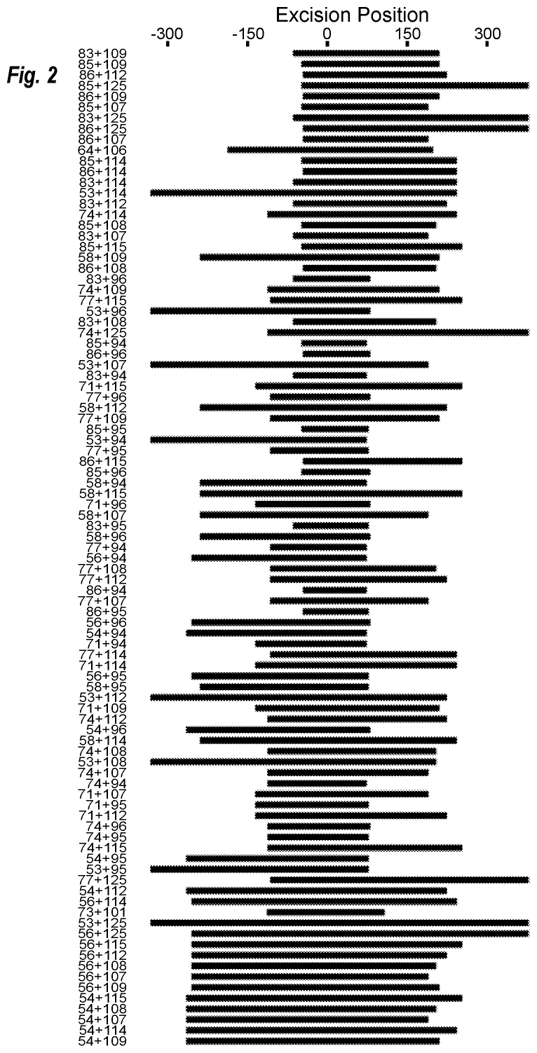

As shown in Table 7 and FIG. 2, 93 pairs of gRNAs were tested, with some pairs achieving greater than 80% excision, with one pair in particular achieving over 88% excision (e.g., using gRNAs having guide sequences directing a nuclease to a target sequence comprising SEQ ID NO:83 and SEQ ID NO: 109; corresponding to guide RNAs comprising SEQ ID NO: 1177 and SEQ ID NO: 1197, respectively).

TABLE 7

| SEQ ID NOs (5′ | SEQ ID NOs (3′ | Excision |
|---|---|---|
| Target Sequence) | Target Sequence) | Percent |
| 83 | 109 | 88.71 |
| 85 | 109 | 85.56 |
| 86 | 112 | 81.58 |
| 85 | 125 | 81.08 |
| 86 | 109 | 79.99 |
| 85 | 107 | 78.44 |
| 83 | 125 | 76.78 |
| 86 | 125 | 76.67 |
| 86 | 107 | 71.68 |
| 64 | 106 | 66.1 |
| 85 | 114 | 65.86 |
| 86 | 114 | 61.58 |
| 83 | 114 | 59.88 |
| 53 | 114 | 43.8 |
| 83 | 112 | 27.6 |
| 74 | 114 | 20.7 |
| 85 | 108 | 7.35 |
| 83 | 107 | 6.69 |
| 85 | 115 | 6.44 |
| 58 | 109 | 5.69 |
| 86 | 108 | 5.57 |
| 83 | 96 | 5.17 |
| 74 | 109 | 4.46 |
| 77 | 115 | 4.45 |
| 53 | 96 | 4.44 |
| 83 | 108 | 4.4 |
| 74 | 125 | 4.3 |
| 85 | 94 | 4.17 |
| 86 | 96 | 3.53 |
| 53 | 107 | 3.42 |
| 83 | 94 | 3.21 |
| 71 | 115 | 3.21 |
| 77 | 96 | 3.12 |
| 58 | 112 | 3.11 |
| 77 | 109 | 3.08 |
| 85 | 95 | 3 |
| 53 | 94 | 2.9 |
| 77 | 95 | 2.82 |
| 86 | 115 | 2.75 |
| 85 | 96 | 2.65 |
| 58 | 94 | 2.61 |
| 58 | 115 | 2.61 |
| 71 | 96 | 2.56 |
| 58 | 107 | 2.53 |
| 83 | 95 | 2.43 |
| 58 | 96 | 2.36 |
| 77 | 94 | 2.24 |
| 56 | 94 | 2.21 |
| 77 | 108 | 2.17 |
| 77 | 112 | 2.16 |
| 86 | 94 | 2.08 |
| 77 | 107 | 1.9 |
| 86 | 95 | 1.87 |
| 56 | 96 | 1.87 |
| 54 | 94 | 1.72 |
| 71 | 94 | 1.69 |
| 77 | 114 | 1.65 |
| 71 | 114 | 1.64 |
| 56 | 95 | 1.63 |
| 58 | 95 | 1.5 |
| 53 | 112 | 1.32 |
| 71 | 109 | 1.3 |
| 74 | 112 | 1.28 |
| 54 | 96 | 1.17 |
| 58 | 114 | 1.15 |
| 74 | 108 | 1.09 |
| 53 | 108 | 0.79 |
| 74 | 107 | 0.62 |
| 74 | 94 | 0.61 |
| 71 | 107 | 0.56 |
| 71 | 95 | 0.55 |
| 71 | 112 | 0.55 |
| 74 | 96 | 0.47 |
| 74 | 95 | 0.46 |
| 74 | 115 | 0.41 |
| 54 | 95 | 0.37 |
| 53 | 95 | 0.35 |
| 77 | 125 | 0.33 |
| 54 | 112 | 0.09 |
| 56 | 114 | 0.01 |
| 73 | 101 | 0.01 |
| 54 | 109 | 0 |
| 54 | 114 | 0 |
| 54 | 107 | 0 |
| 54 | 108 | 0 |
| 54 | 115 | 0 |
| 56 | 109 | 0 |
| 56 | 107 | 0 |
| 56 | 108 | 0 |
| 56 | 112 | 0 |
| 56 | 115 | 0 |
| 56 | 125 | 0 |
| 53 | 125 | 0 |

### Example 2. Use of gRNAs to Treat Mutations in COL8A2

Three mutations in COL8A2, Gln455Lys, Gln455Val, and Leu450Trp, have been associated with early-onset FECD and posterior polymorphous corneal dystrophy (PPDC), and knock-in animal studies have shown a pathology consistent with human early-onset FECD. These models are associated with abnormal intracellular accumulation of mutant collagen VIII peptides with altered stability of the triple helical structure. Therefore, decreasing mutant collagen VIII in patients with diagnosis or family history of mutations in COL8A2 may improve disease course. Alternatively, selectively reducing levels of COL8A2 with mutation at either Gln455Lys, Gln455Val, or Leu450Trp may reduce levels of mutant collagen VIII peptides and improve disease course. Another approach would be to correct mutations in the DNA leading to amino acid mutations in the alpha subunit 2 of collagen VIII (COL8A2) and thereby remove the abnormal gene product.

Target sequences were selected for developing Cas RNP therapies using NCBI Reference Sequence NM_005202.3 of transcript variant 1 of the COL8A2 gene. This sequence does not contain mutations known to occur at positions 455 and 450 in the amino acid sequence of the collagen VIII gene product and may be termed the “wild type COL8A2 sequence.” Target sequences were selected between Chrl: 36097532-36100270 (hg38 version), as listed in Table 3 (SEQ ID NOs: 191-1063). Guide sequences complementary to the target sequences can be used to generate gRNAs for use with RNPs to target COL8A2.

TABLE 3
Target sequences for wild type COL8A2

| SEQ ID | Chromosomal | | |
|---|---|---|---|
| No | location | Strand | Target sequence |
| 191 | Chr1: 36097532-36097554 | + | GGGGAGGAGGCCAGGGCAGCAGG |
| 192 | Chr1: 36097545-36097567 | + | GGGCAGCAGGACCCCCCCCGCGG |
| 193 | Chr1: 36097546-36097568 | + | GGCAGCAGGACCCCCCCCGCGGG |
| 194 | Chr1: 36097554-36097576 | + | GACCCCCCCCGCGGGTTATGTGG |
| 195 | Chr1: 36097555-36097577 | + | ACCCCCCCCGCGGGTTATGTGGG |
| 196 | Chr1: 36097556-36097578 | + | CCCCCCCCGCGGGTTATGTGGGG |
| 197 | Chr1: 36097556-36097578 | − | CCCCACATAACCCGCGGGGGGGG |
| 198 | Chr1: 36097557-36097579 | − | GCCCCACATAACCCGCGGGGGGG |
| 199 | Chr1: 36097558-36097580 | − | TGCCCCACATAACCCGCGGGGGG |
| 200 | Chr1: 36097559-36097581 | − | CTGCCCCACATAACCCGCGGGGG |
| 201 | Chr1: 36097560-36097582 | − | TCTGCCCCACATAACCCGCGGGG |
| 202 | Chr1: 36097561-36097583 | − | CTCTGCCCCACATAACCCGCGGG |
| 203 | Chr1: 36097562-36097584 | − | GCTCTGCCCCACATAACCCGCGG |
| 204 | Chr1: 36097578-36097600 | + | GCAGAGCAAGAATCCTGAAAAGG |
| 205 | Chr1: 36097581-36097603 | + | GAGCAAGAATCCTGAAAAGGAGG |
| 206 | Chr1: 36097586-36097608 | + | AGAATCCTGAAAAGGAGGAGTGG |
| 207 | Chr1: 36097591-36097613 | − | TACATCCACTCCTCCTTTTCAGG |
| 208 | Chr1: 36097599-36097621 | + | GGAGGAGTGGATGTACTCCGTGG |
| 209 | Chr1: 36097607-36097629 | + | GGATGTACTCCGTGGAGTAGAGG |
| 210 | Chr1: 36097614-36097636 | + | CTCCGTGGAGTAGAGGCCGTTGG |
| 211 | Chr1: 36097616-36097638 | − | GGCCAACGGCCTCTACTCCACGG |
| 212 | Chr1: 36097619-36097641 | + | TGGAGTAGAGGCCGTTGGCCTGG |
| 213 | Chr1: 36097627-36097649 | + | AGGCCGTTGGCCTGGTCCGACGG |
| 214 | Chr1: 36097630-36097652 | − | ATGCCGTCGGACCAGGCCAACGG |
| 215 | Chr1: 36097637-36097659 | − | GGTGCAGATGCCGTCGGACCAGG |
| 216 | Chr1: 36097643-36097665 | − | GGTCTGGGTGCAGATGCCGTCGG |
| 217 | Chr1: 36097646-36097668 | + | ACGGCATCTGCACCCAGACCTGG |
| 218 | Chr1: 36097653-36097675 | + | CTGCACCCAGACCTGGTCGTTGG |
| 219 | Chr1: 36097654-36097676 | + | TGCACCCAGACCTGGTCGTTGGG |
| 220 | Chr1: 36097658-36097680 | − | GCGGCCCAACGACCAGGTCTGGG |
| 221 | Chr1: 36097659-36097681 | − | TGCGGCCCAACGACCAGGTCTGG |
| 222 | Chr1: 36097664-36097686 | + | CCTGGTCGTTGGGCCGCAGCTGG |
| 223 | Chr1: 36097664-36097686 | − | CCAGCTGCGGCCCAACGACCAGG |
| 224 | Chr1: 36097671-36097693 | + | GTTGGGCCGCAGCTGGAGCACGG |
| 225 | Chr1: 36097677-36097699 | − | GTGGGGCCGTGCTCCAGCTGCGG |
| 226 | Chr1: 36097688-36097710 | + | GCACGGCCCCACCAGATGCCTGG |
| 227 | Chr1: 36097694-36097716 | + | CCCCACCAGATGCCTGGTCCAGG |
| 228 | Chr1: 36097694-36097716 | − | CCTGGACCAGGCATCTGGTGGGG |
| 229 | Chr1: 36097695-36097717 | − | ACCTGGACCAGGCATCTGGTGGG |
| 230 | Chr1: 36097696-36097718 | − | TACCTGGACCAGGCATCTGGTGG |
| 231 | Chr1: 36097699-36097721 | − | GGCTACCTGGACCAGGCATCTGG |
| 232 | Chr1: 36097706-36097728 | − | CAAGAAGGGCTACCTGGACCAGG |
| 233 | Chr1: 36097712-36097734 | − | TGAGTACAAGAAGGGCTACCTGG |
| 234 | Chr1: 36097719-36097741 | + | GCCCTTCTTGTACTCATCGTAGG |
| 235 | Chr1: 36097720-36097742 | − | ACCTACGATGAGTACAAGAAGGG |
| 236 | Chr1: 36097721-36097743 | − | TACCTACGATGAGTACAAGAAGG |
| 237 | Chr1: 36097725-36097747 | + | CTTGTACTCATCGTAGGTATAGG |
| 238 | Chr1: 36097728-36097750 | + | GTACTCATCGTAGGTATAGGTGG |
| 239 | Chr1: 36097732-36097754 | + | TCATCGTAGGTATAGGTGGCCGG |
| 240 | Chr1: 36097751-36097773 | + | CCGGCACGTTGTTCTTGTACAGG |
| 241 | Chr1: 36097751-36097773 | − | CCTGTACAAGAACAACGTGCCGG |
| 242 | Chr1: 36097752-36097774 | + | CGGCACGTTGTTCTTGTACAGGG |
| 243 | Chr1: 36097767-36097789 | + | GTACAGGGCCACCCACACGTTGG |
| 244 | Chr1: 36097775-36097797 | − | CAAGGGCACCAACGTGTGGGTGG |
| 245 | Chr1: 36097778-36097800 | − | CGTCAAGGGCACCAACGTGTGGG |
| 246 | Chr1: 36097779-36097801 | − | ACGTCAAGGGCACCAACGTGTGG |
| 247 | Chr1: 36097787-36097809 | + | TGGTGCCCTTGACGTGCACATGG |
| 248 | Chr1: 36097792-36097814 | − | GCTTACCATGTGCACGTCAAGGG |
| 249 | Chr1: 36097793-36097815 | − | TGCTTACCATGTGCACGTCAAGG |
| 250 | Chr1: 36097816-36097838 | + | AAGTAGTAGACGCCGCCCACAGG |
| 251 | Chr1: 36097817-36097839 | + | AGTAGTAGACGCCGCCCACAGGG |
| 252 | Chr1: 36097821-36097843 | + | GTAGACGCCGCCCACAGGGCAGG |
| 253 | Chr1: 36097828-36097850 | − | ATCTTCACCTGCCCTGTGGGCGG |
| 254 | Chr1: 36097831-36097853 | − | GGCATCTTCACCTGCCCTGTGGG |
| 255 | Chr1: 36097832-36097854 | − | TGGCATCTTCACCTGCCCTGTGG |
| 256 | Chr1: 36097836-36097858 | + | AGGGCAGGTGAAGATGCCAGTGG |
| 257 | Chr1: 36097840-36097862 | + | CAGGTGAAGATGCCAGTGGCTGG |
| 258 | Chr1: 36097841-36097863 | + | AGGTGAAGATGCCAGTGGCTGGG |
| 259 | Chr1: 36097852-36097874 | − | AGCGGCTACAACCCAGCCACTGG |
| 260 | Chr1: 36097856-36097878 | + | TGGCTGGGTTGTAGCCGCTGTGG |
| 261 | Chr1: 36097870-36097892 | − | ACTCTCTACAATGGCCACAGCGG |
| 262 | Chr1: 36097874-36097896 | + | TGTGGCCATTGTAGAGAGTCCGG |
| 263 | Chr1: 36097879-36097901 | − | TTTGACCGGACTCTCTACAATGG |
| 264 | Chr1: 36097887-36097909 | + | GAGAGTCCGGTCAAATTTCACGG |
| 265 | Chr1: 36097888-36097910 | + | AGAGTCCGGTCAAATTTCACGGG |
| 266 | Chr1: 36097893-36097915 | − | GCATGCCCGTGAAATTTGACCGG |
| 267 | Chr1: 36097899-36097921 | + | AAATTTCACGGGCATGCCCGAGG |
| 268 | Chr1: 36097902-36097924 | + | TTTCACGGGCATGCCCGAGGCGG |
| 269 | Chr1: 36097903-36097925 | + | TTCACGGGCATGCCCGAGGCGGG |
| 270 | Chr1: 36097904-36097926 | + | TCACGGGCATGCCCGAGGCGGGG |
| 271 | Chr1: 36097908-36097930 | + | GGGCATGCCCGAGGCGGGGAAGG |
| 272 | Chr1: 36097909-36097931 | + | GGCATGCCCGAGGCGGGGAAGGG |
| 273 | Chr1: 36097914-36097936 | + | GCCCGAGGCGGGGAAGGGCGAGG |
| 274 | Chr1: 36097915-36097937 | − | ACCTCGCCCTTCCCCGCCTCGGG |
| 275 | Chr1: 36097916-36097938 | − | CACCTCGCCCTTCCCCGCCTCGG |
| 276 | Chr1: 36097932-36097954 | + | CGAGGTGAGCACCGCAGTGAAGG |
| 277 | Chr1: 36097936-36097958 | + | GTGAGCACCGCAGTGAAGGCCGG |
| 278 | Chr1: 36097941-36097963 | + | CACCGCAGTGAAGGCCGGTGTGG |
| 279 | Chr1: 36097943-36097965 | − | TGCCACACCGGCCTTCACTGCGG |
| 280 | Chr1: 36097946-36097968 | + | CAGTGAAGGCCGGTGTGGCATGG |
| 281 | Chr1: 36097947-36097969 | + | AGTGAAGGCCGGTGTGGCATGGG |
| 282 | Chr1: 36097955-36097977 | − | GCTGTCTGCCCATGCCACACCGG |
| 283 | Chr1: 36097975-36097997 | + | AGCTCGCCCAGCCCAAACTGTGG |
| 284 | Chr1: 36097981-36098003 | − | GGCAAGCCACAGTTTGGGCTGGG |
| 285 | Chr1: 36097982-36098004 | − | GGGCAAGCCACAGTTTGGGCTGG |
| 286 | Chr1: 36097986-36098008 | − | AGGGGGGCAAGCCACAGTTTGGG |
| 287 | Chr1: 36097987-36098009 | − | AAGGGGGGCAAGCCACAGTTTGG |
| 288 | Chr1: 36097998-36098020 | + | CTTGCCCCCCTTGCCCAGCACGG |
| 289 | Chr1: 36098002-36098024 | − | GGTGCCGTGCTGGGCAAGGGGGG |
| 290 | Chr1: 36098003-36098025 | − | GGGTGCCGTGCTGGGCAAGGGGG |
| 291 | Chr1: 36098004-36098026 | − | AGGGTGCCGTGCTGGGCAAGGGG |
| 292 | Chr1: 36098005-36098027 | − | GAGGGTGCCGTGCTGGGCAAGGG |
| 293 | Chr1: 36098006-36098028 | − | GGAGGGTGCCGTGCTGGGCAAGG |
| 294 | Chr1: 36098011-36098033 | − | GGTGTGGAGGGTGCCGTGCTGGG |
| 295 | Chr1: 36098012-36098034 | − | CGGTGTGGAGGGTGCCGTGCTGG |
| 296 | Chr1: 36098019-36098041 | + | GGCACCCTCCACACCGCCGTTGG |
| 297 | Chr1: 36098020-36098042 | + | GCACCCTCCACACCGCCGTTGGG |
| 298 | Chr1: 36098023-36098045 | − | CTGCCCAACGGCGGTGTGGAGGG |
| 299 | Chr1: 36098024-36098046 | + | CCTCCACACCGCCGTTGGGCAGG |
| 300 | Chr1: 36098024-36098046 | − | CCTGCCCAACGGCGGTGTGGAGG |
| 301 | Chr1: 36098027-36098049 | − | GCACCTGCCCAACGGCGGTGTGG |
| 302 | Chr1: 36098032-36098054 | − | GGCTTGCACCTGCCCAACGGCGG |
| 303 | Chr1: 36098035-36098057 | − | GCAGGCTTGCACCTGCCCAACGG |
| 304 | Chr1: 36098053-36098075 | − | TTCGATGAGACTGGCATCGCAGG |
| 305 | Chr1: 36098055-36098077 | + | TGCGATGCCAGTCTCATCGAAGG |
| 306 | Chr1: 36098062-36098084 | + | CCAGTCTCATCGAAGGCCCCAGG |
| 307 | Chr1: 36098062-36098084 | − | CCTGGGGCCTTCGATGAGACTGG |
| 308 | Chr1: 36098063-36098085 | + | CAGTCTCATCGAAGGCCCCAGGG |
| 309 | Chr1: 36098064-36098086 | + | AGTCTCATCGAAGGCCCCAGGGG |
| 310 | Chr1: 36098071-36098093 | + | TCGAAGGCCCCAGGGGCACCAGG |
| 311 | Chr1: 36098072-36098094 | + | CGAAGGCCCCAGGGGCACCAGGG |
| 312 | Chr1: 36098073-36098095 | + | GAAGGCCCCAGGGGCACCAGGGG |
| 313 | Chr1: 36098074-36098096 | + | AAGGCCCCAGGGGCACCAGGGGG |
| 314 | Chr1: 36098078-36098100 | − | GGGACCCCCTGGTGCCCCTGGGG |
| 315 | Chr1: 36098079-36098101 | − | CGGGACCCCCTGGTGCCCCTGGG |
| 316 | Chr1: 36098080-36098102 | + | CCAGGGGCACCAGGGGGTCCCGG |
| 317 | Chr1: 36098080-36098102 | − | CCGGGACCCCCTGGTGCCCCTGG |
| 318 | Chr1: 36098081-36098103 | + | CAGGGGCACCAGGGGGTCCCGGG |
| 319 | Chr1: 36098082-36098104 | + | AGGGGCACCAGGGGGTCCCGGGG |
| 320 | Chr1: 36098083-36098105 | + | GGGGCACCAGGGGGTCCCGGGGG |
| 321 | Chr1: 36098088-36098110 | + | ACCAGGGGGTCCCGGGGGCCCGG |
| 322 | Chr1: 36098089-36098111 | + | CCAGGGGGTCCCGGGGGCCCGGG |
| 323 | Chr1: 36098089-36098111 | − | CCCGGGCCCCCGGGACCCCCTGG |
| 324 | Chr1: 36098092-36098114 | + | GGGGGTCCCGGGGGCCCGGGAGG |
| 325 | Chr1: 36098098-36098120 | + | CCCGGGGGCCCGGGAGGCCCCGG |
| 326 | Chr1: 36098098-36098120 | − | CCGGGGCCTCCCGGGCCCCCGGG |
| 327 | Chr1: 36098099-36098121 | − | TCCGGGGCCTCCCGGGCCCCCGG |
| 328 | Chr1: 36098101-36098123 | + | GGGGGCCCGGGAGGCCCCGGAGG |
| 329 | Chr1: 36098102-36098124 | + | GGGGCCCGGGAGGCCCCGGAGGG |
| 330 | Chr1: 36098106-36098128 | − | CGGGCCCTCCGGGGCCTCCCGGG |
| 331 | Chr1: 36098107-36098129 | − | ACGGGCCCTCCGGGGCCTCCCGG |
| 332 | Chr1: 36098115-36098137 | − | CTGGAATCACGGGCCCTCCGGGG |
| 333 | Chr1: 36098116-36098138 | + | CCCGGAGGGCCCGTGATTCCAGG |
| 334 | Chr1: 36098116-36098138 | − | CCTGGAATCACGGGCCCTCCGGG |
| 335 | Chr1: 36098117-36098139 | + | CCGGAGGGCCCGTGATTCCAGGG |
| 336 | Chr1: 36098117-36098139 | − | CCCTGGAATCACGGGCCCTCCGG |
| 337 | Chr1: 36098118-36098140 | + | CGGAGGGCCCGTGATTCCAGGGG |
| 338 | Chr1: 36098125-36098147 | + | CCCGTGATTCCAGGGGAGCCAGG |
| 339 | Chr1: 36098125-36098147 | − | CCTGGCTCCCCTGGAATCACGGG |
| 340 | Chr1: 36098126-36098148 | + | CCGTGATTCCAGGGGAGCCAGGG |
| 341 | Chr1: 36098126-36098148 | − | CCCTGGCTCCCCTGGAATCACGG |
| 342 | Chr1: 36098134-36098156 | + | CCAGGGGAGCCAGGGACCCCTGG |
| 343 | Chr1: 36098134-36098156 | − | CCAGGGGTCCCTGGCTCCCCTGG |
| 344 | Chr1: 36098135-36098157 | + | CAGGGGAGCCAGGGACCCCTGGG |
| 345 | Chr1: 36098136-36098158 | + | AGGGGAGCCAGGGACCCCTGGGG |
| 346 | Chr1: 36098137-36098159 | + | GGGGAGCCAGGGACCCCTGGGGG |
| 347 | Chr1: 36098143-36098165 | − | ACGGGGCCCCCAGGGGTCCCTGG |
| 348 | Chr1: 36098145-36098167 | + | AGGGACCCCTGGGGGCCCCGTGG |
| 349 | Chr1: 36098146-36098168 | + | GGGACCCCTGGGGGCCCCGTGGG |
| 350 | Chr1: 36098150-36098172 | − | TGGGCCCACGGGGCCCCCAGGGG |
| 351 | Chr1: 36098151-36098173 | − | CTGGGCCCACGGGGCCCCCAGGG |
| 352 | Chr1: 36098152-36098174 | − | GCTGGGCCCACGGGGCCCCCAGG |
| 353 | Chr1: 36098160-36098182 | − | CTGGCACGGCTGGGCCCACGGGG |
| 354 | Chr1: 36098161-36098183 | + | CCCGTGGGCCCAGCCGTGCCAGG |
| 355 | Chr1: 36098161-36098183 | − | CCTGGCACGGCTGGGCCCACGGG |
| 356 | Chr1: 36098162-36098184 | − | ACCTGGCACGGCTGGGCCCACGG |
| 357 | Chr1: 36098169-36098191 | − | CAGGGGAACCTGGCACGGCTGGG |
| 358 | Chr1: 36098170-36098192 | − | GCAGGGGAACCTGGCACGGCTGG |
| 359 | Chr1: 36098174-36098196 | − | GAGAGCAGGGGAACCTGGCACGG |
| 360 | Chr1: 36098179-36098201 | − | GAGGGGAGAGCAGGGGAACCTGG |
| 361 | Chr1: 36098185-36098207 | + | TCCCCTGCTCTCCCCTCTCCAGG |
| 362 | Chr1: 36098186-36098208 | + | CCCCTGCTCTCCCCTCTCCAGGG |
| 363 | Chr1: 36098186-36098208 | − | CCCTGGAGAGGGGAGAGCAGGGG |
| 364 | Chr1: 36098187-36098209 | + | CCCTGCTCTCCCCTCTCCAGGGG |
| 365 | Chr1: 36098187-36098209 | − | CCCCTGGAGAGGGGAGAGCAGGG |
| 366 | Chr1: 36098188-36098210 | + | CCTGCTCTCCCCTCTCCAGGGGG |
| 367 | Chr1: 36098188-36098210 | − | CCCCCTGGAGAGGGGAGAGCAGG |
| 368 | Chr1: 36098194-36098216 | + | CTCCCCTCTCCAGGGGGCCCTGG |
| 369 | Chr1: 36098196-36098218 | − | TGCCAGGGCCCCCTGGAGAGGGG |
| 370 | Chr1: 36098197-36098219 | − | CTGCCAGGGCCCCCTGGAGAGGG |
| 371 | Chr1: 36098198-36098220 | + | CCTCTCCAGGGGGCCCTGGCAGG |
| 372 | Chr1: 36098198-36098220 | − | CCTGCCAGGGCCCCCTGGAGAGG |
| 373 | Chr1: 36098203-36098225 | + | CCAGGGGGCCCTGGCAGGCCTGG |
| 374 | Chr1: 36098203-36098225 | − | CCAGGCCTGCCAGGGCCCCCTGG |
| 375 | Chr1: 36098211-36098233 | − | AGGGGGAACCAGGCCTGCCAGGG |
| 376 | Chr1: 36098212-36098234 | − | AAGGGGGAACCAGGCCTGCCAGG |
| 377 | Chr1: 36098216-36098238 | + | GCAGGCCTGGTTCCCCCTTCAGG |
| 378 | Chr1: 36098221-36098243 | + | CCTGGTTCCCCCTTCAGGCCCGG |
| 379 | Chr1: 36098221-36098243 | − | CCGGGCCTGAAGGGGGAACCAGG |
| 380 | Chr1: 36098225-36098247 | + | GTTCCCCCTTCAGGCCCGGCAGG |
| 381 | Chr1: 36098228-36098250 | − | AGGCCTGCCGGGCCTGAAGGGGG |
| 382 | Chr1: 36098229-36098251 | − | AAGGCCTGCCGGGCCTGAAGGGG |
| 383 | Chr1: 36098230-36098252 | − | CAAGGCCTGCCGGGCCTGAAGGG |
| 384 | Chr1: 36098231-36098253 | + | CCTTCAGGCCCGGCAGGCCTTGG |
| 385 | Chr1: 36098231-36098253 | − | CCAAGGCCTGCCGGGCCTGAAGG |
| 386 | Chr1: 36098232-36098254 | + | CTTCAGGCCCGGCAGGCCTTGGG |
| 387 | Chr1: 36098233-36098255 | + | TTCAGGCCCGGCAGGCCTTGGGG |
| 388 | Chr1: 36098239-36098261 | − | ATTGGGCCCCAAGGCCTGCCGGG |
| 389 | Chr1: 36098240-36098262 | − | TATTGGGCCCCAAGGCCTGCCGG |
| 390 | Chr1: 36098242-36098264 | + | GGCAGGCCTTGGGGCCCAATAGG |
| 391 | Chr1: 36098243-36098265 | + | GCAGGCCTTGGGGCCCAATAGGG |
| 392 | Chr1: 36098248-36098270 | − | GCTGGCCCTATTGGGCCCCAAGG |
| 393 | Chr1: 36098251-36098273 | + | TGGGGCCCAATAGGGCCAGCTGG |
| 394 | Chr1: 36098256-36098278 | − | AGGGTCCAGCTGGCCCTATTGGG |
| 395 | Chr1: 36098257-36098279 | − | CAGGGTCCAGCTGGCCCTATTGG |
| 396 | Chr1: 36098258-36098280 | + | CAATAGGGCCAGCTGGACCCTGG |
| 397 | Chr1: 36098266-36098288 | + | CCAGCTGGACCCTGGAGTCCTGG |
| 398 | Chr1: 36098266-36098288 | − | CCAGGACTCCAGGGTCCAGCTGG |
| 399 | Chr1: 36098267-36098289 | + | CAGCTGGACCCTGGAGTCCTGGG |
| 400 | Chr1: 36098275-36098297 | − | TCAGGAATCCCAGGACTCCAGGG |
| 401 | Chr1: 36098276-36098298 | − | CTCAGGAATCCCAGGACTCCAGG |
| 402 | Chr1: 36098277-36098299 | + | CTGGAGTCCTGGGATTCCTGAGG |
| 403 | Chr1: 36098278-36098300 | + | TGGAGTCCTGGGATTCCTGAGGG |
| 404 | Chr1: 36098284-36098306 | − | AGGGGTCCCTCAGGAATCCCAGG |
| 405 | Chr1: 36098288-36098310 | + | GGATTCCTGAGGGACCCCTCAGG |
| 406 | Chr1: 36098293-36098315 | + | CCTGAGGGACCCCTCAGGCCAGG |
| 407 | Chr1: 36098293-36098315 | − | CCTGGCCTGAGGGGTCCCTCAGG |
| 408 | Chr1: 36098302-36098324 | + | CCCCTCAGGCCAGGCTGCCCAGG |
| 409 | Chr1: 36098302-36098324 | − | CCTGGGCAGCCTGGCCTGAGGGG |
| 410 | Chr1: 36098303-36098325 | + | CCCTCAGGCCAGGCTGCCCAGGG |
| 411 | Chr1: 36098303-36098325 | − | CCCTGGGCAGCCTGGCCTGAGGG |
| 412 | Chr1: 36098304-36098326 | − | TCCCTGGGCAGCCTGGCCTGAGG |
| 413 | Chr1: 36098311-36098333 | − | TTGGGGCTCCCTGGGCAGCCTGG |
| 414 | Chr1: 36098319-36098341 | − | AAGGTGACTTGGGGCTCCCTGGG |
| 415 | Chr1: 36098320-36098342 | − | AAAGGTGACTTGGGGCTCCCTGG |
| 416 | Chr1: 36098328-36098350 | − | TGGGGCAGAAAGGTGACTTGGGG |
| 417 | Chr1: 36098329-36098351 | − | CTGGGGCAGAAAGGTGACTTGGG |
| 418 | Chr1: 36098330-36098352 | + | CCAAGTCACCTTTCTGCCCCAGG |
| 419 | Chr1: 36098330-36098352 | − | CCTGGGGCAGAAAGGTGACTTGG |
| 420 | Chr1: 36098331-36098353 | + | CAAGTCACCTTTCTGCCCCAGGG |
| 421 | Chr1: 36098338-36098360 | − | GCAGGAGCCCTGGGGCAGAAAGG |
| 422 | Chr1: 36098346-36098368 | − | CAGGGGTGGCAGGAGCCCTGGGG |
| 423 | Chr1: 36098347-36098369 | + | CCCAGGGCTCCTGCCACCCCTGG |
| 424 | Chr1: 36098347-36098369 | − | CCAGGGGTGGCAGGAGCCCTGGG |
| 425 | Chr1: 36098348-36098370 | − | ACCAGGGGTGGCAGGAGCCCTGG |
| 426 | Chr1: 36098356-36098378 | + | CCTGCCACCCCTGGTCCTCCAGG |
| 427 | Chr1: 36098356-36098378 | − | CCTGGAGGACCAGGGGTGGCAGG |
| 428 | Chr1: 36098357-36098379 | + | CTGCCACCCCTGGTCCTCCAGGG |
| 429 | Chr1: 36098360-36098382 | − | TCGCCCTGGAGGACCAGGGGTGG |
| 430 | Chr1: 36098363-36098385 | − | GGGTCGCCCTGGAGGACCAGGGG |
| 431 | Chr1: 36098364-36098386 | − | CGGGTCGCCCTGGAGGACCAGGG |
| 432 | Chr1: 36098365-36098387 | − | ACGGGTCGCCCTGGAGGACCAGG |
| 433 | Chr1: 36098371-36098393 | − | GGTTTCACGGGTCGCCCTGGAGG |
| 434 | Chr1: 36098374-36098396 | + | CCAGGGCGACCCGTGAAACCCGG |
| 435 | Chr1: 36098374-36098396 | − | CCGGGTTTCACGGGTCGCCCTGG |
| 436 | Chr1: 36098383-36098405 | − | AAGGGTGAGCCGGGTTTCACGGG |
| 437 | Chr1: 36098384-36098406 | − | CAAGGGTGAGCCGGGTTTCACGG |
| 438 | Chr1: 36098385-36098407 | + | CGTGAAACCCGGCTCACCCTTGG |
| 439 | Chr1: 36098386-36098408 | + | GTGAAACCCGGCTCACCCTTGGG |
| 440 | Chr1: 36098392-36098414 | − | ACTGGGCCCAAGGGTGAGCCGGG |
| 441 | Chr1: 36098393-36098415 | − | AACTGGGCCCAAGGGTGAGCCGG |
| 442 | Chr1: 36098395-36098417 | + | GGCTCACCCTTGGGCCCAGTTGG |
| 443 | Chr1: 36098401-36098423 | + | CCCTTGGGCCCAGTTGGTCCAGG |
| 444 | Chr1: 36098401-36098423 | − | CCTGGACCAACTGGGCCCAAGGG |
| 445 | Chr1: 36098402-36098424 | + | CCTTGGGCCCAGTTGGTCCAGGG |
| 446 | Chr1: 36098402-36098424 | − | CCCTGGACCAACTGGGCCCAAGG |
| 447 | Chr1: 36098403-36098425 | + | CTTGGGCCCAGTTGGTCCAGGGG |
| 448 | Chr1: 36098404-36098426 | + | TTGGGCCCAGTTGGTCCAGGGGG |
| 449 | Chr1: 36098409-36098431 | − | ATGGACCCCCTGGACCAACTGGG |
| 450 | Chr1: 36098410-36098432 | − | CATGGACCCCCTGGACCAACTGG |
| 451 | Chr1: 36098411-36098433 | + | CAGTTGGTCCAGGGGGTCCATGG |
| 452 | Chr1: 36098412-36098434 | + | AGTTGGTCCAGGGGGTCCATGGG |
| 453 | Chr1: 36098419-36098441 | + | CCAGGGGGTCCATGGGCCCCAGG |
| 454 | Chr1: 36098419-36098441 | − | CCTGGGGCCCATGGACCCCCTGG |
| 455 | Chr1: 36098428-36098450 | − | AGGGGACTTCCTGGGGCCCATGG |
| 456 | Chr1: 36098435-36098457 | − | AGGTGAGAGGGGACTTCCTGGGG |
| 457 | Chr1: 36098436-36098458 | − | CAGGTGAGAGGGGACTTCCTGGG |
| 458 | Chr1: 36098437-36098459 | + | CCAGGAAGTCCCCTCTCACCTGG |
| 459 | Chr1: 36098437-36098459 | − | CCAGGTGAGAGGGGACTTCCTGG |
| 460 | Chr1: 36098438-36098460 | + | CAGGAAGTCCCCTCTCACCTGGG |
| 461 | Chr1: 36098446-36098468 | + | CCCCTCTCACCTGGGACCCCTGG |
| 462 | Chr1: 36098446-36098468 | − | CCAGGGGTCCCAGGTGAGAGGGG |
| 463 | Chr1: 36098447-36098469 | − | ACCAGGGGTCCCAGGTGAGAGGG |
| 464 | Chr1: 36098448-36098470 | − | AACCAGGGGTCCCAGGTGAGAGG |
| 465 | Chr1: 36098455-36098477 | − | GCTGGGAAACCAGGGGTCCCAGG |
| 466 | Chr1: 36098459-36098481 | + | GGACCCCTGGTTTCCCAGCCAGG |
| 467 | Chr1: 36098462-36098484 | − | TGGCCTGGCTGGGAAACCAGGGG |
| 468 | Chr1: 36098463-36098485 | − | GTGGCCTGGCTGGGAAACCAGGG |
| 469 | Chr1: 36098464-36098486 | − | AGTGGCCTGGCTGGGAAACCAGG |
| 470 | Chr1: 36098467-36098489 | + | GGTTTCCCAGCCAGGCCACTAGG |
| 471 | Chr1: 36098472-36098494 | − | AGGGGCCTAGTGGCCTGGCTGGG |
| 472 | Chr1: 36098473-36098495 | − | CAGGGGCCTAGTGGCCTGGCTGG |
| 473 | Chr1: 36098474-36098496 | + | CAGCCAGGCCACTAGGCCCCTGG |
| 474 | Chr1: 36098477-36098499 | − | TGACCAGGGGCCTAGTGGCCTGG |
| 475 | Chr1: 36098482-36098504 | − | CGAGGTGACCAGGGGCCTAGTGG |
| 476 | Chr1: 36098490-36098512 | − | CTGGCATTCGAGGTGACCAGGGG |
| 477 | Chr1: 36098491-36098513 | + | CCCTGGTCACCTCGAATGCCAGG |
| 478 | Chr1: 36098491-36098513 | − | CCTGGCATTCGAGGTGACCAGGG |
| 479 | Chr1: 36098492-36098514 | − | GCCTGGCATTCGAGGTGACCAGG |
| 480 | Chr1: 36098500-36098522 | + | CCTCGAATGCCAGGCACTCCTGG |
| 481 | Chr1: 36098500-36098522 | − | CCAGGAGTGCCTGGCATTCGAGG |
| 482 | Chr1: 36098501-36098523 | + | CTCGAATGCCAGGCACTCCTGGG |
| 483 | Chr1: 36098502-36098524 | + | TCGAATGCCAGGCACTCCTGGGG |
| 484 | Chr1: 36098503-36098525 | + | CGAATGCCAGGCACTCCTGGGGG |
| 485 | Chr1: 36098509-36098531 | − | GGAGGACCCCCAGGAGTGCCTGG |
| 486 | Chr1: 36098512-36098534 | + | GGCACTCCTGGGGGTCCTCCAGG |
| 487 | Chr1: 36098518-36098540 | − | GCAGGGCCTGGAGGACCCCCAGG |
| 488 | Chr1: 36098527-36098549 | − | AAGGGTGAGGCAGGGCCTGGAGG |
| 489 | Chr1: 36098530-36098552 | + | CCAGGCCCTGCCTCACCCTTAGG |
| 490 | Chr1: 36098530-36098552 | − | CCTAAGGGTGAGGCAGGGCCTGG |
| 491 | Chr1: 36098535-36098557 | − | CTGGGCCTAAGGGTGAGGCAGGG |
| 492 | Chr1: 36098536-36098558 | + | CCTGCCTCACCCTTAGGCCCAGG |
| 493 | Chr1: 36098536-36098558 | − | CCTGGGCCTAAGGGTGAGGCAGG |
| 494 | Chr1: 36098537-36098559 | + | CTGCCTCACCCTTAGGCCCAGGG |
| 495 | Chr1: 36098538-36098560 | + | TGCCTCACCCTTAGGCCCAGGGG |
| 496 | Chr1: 36098539-36098561 | + | GCCTCACCCTTAGGCCCAGGGGG |
| 497 | Chr1: 36098540-36098562 | − | GCCCCCTGGGCCTAAGGGTGAGG |
| 498 | Chr1: 36098545-36098567 | − | CGTGGGCCCCCTGGGCCTAAGGG |
| 499 | Chr1: 36098546-36098568 | − | ACGTGGGCCCCCTGGGCCTAAGG |
| 500 | Chr1: 36098553-36098575 | − | CTGGCAGACGTGGGCCCCCTGGG |
| 501 | Chr1: 36098554-36098576 | + | CCAGGGGGCCCACGTCTGCCAGG |
| 502 | Chr1: 36098554-36098576 | − | CCTGGCAGACGTGGGCCCCCTGG |
| 503 | Chr1: 36098562-36098584 | − | CAGGGCTTCCTGGCAGACGTGGG |
| 504 | Chr1: 36098563-36098585 | − | GCAGGGCTTCCTGGCAGACGTGG |
| 505 | Chr1: 36098572-36098594 | + | CCAGGAAGCCCTGCAGACCCAGG |
| 506 | Chr1: 36098572-36098594 | − | CCTGGGTCTGCAGGGCTTCCTGG |
| 507 | Chr1: 36098580-36098602 | − | CTGGACTTCCTGGGTCTGCAGGG |
| 508 | Chr1: 36098581-36098603 | + | CCTGCAGACCCAGGAAGTCCAGG |
| 509 | Chr1: 36098581-36098603 | − | CCTGGACTTCCTGGGTCTGCAGG |
| 510 | Chr1: 36098582-36098604 | + | CTGCAGACCCAGGAAGTCCAGGG |
| 511 | Chr1: 36098583-36098605 | + | TGCAGACCCAGGAAGTCCAGGGG |
| 512 | Chr1: 36098584-36098606 | + | GCAGACCCAGGAAGTCCAGGGGG |
| 513 | Chr1: 36098589-36098611 | − | GGGGTCCCCCTGGACTTCCTGGG |
| 514 | Chr1: 36098590-36098612 | − | GGGGGTCCCCCTGGACTTCCTGG |
| 515 | Chr1: 36098599-36098621 | − | CAGGGTCTTGGGGGTCCCCCTGG |
| 516 | Chr1: 36098602-36098624 | + | GGGGGACCCCCAAGACCCTGTGG |
| 517 | Chr1: 36098603-36098625 | + | GGGGACCCCCAAGACCCTGTGGG |
| 518 | Chr1: 36098608-36098630 | − | CAGGGCCCACAGGGTCTTGGGGG |
| 519 | Chr1: 36098609-36098631 | − | GCAGGGCCCACAGGGTCTTGGGG |
| 520 | Chr1: 36098610-36098632 | − | AGCAGGGCCCACAGGGTCTTGGG |
| 521 | Chr1: 36098611-36098633 | − | GAGCAGGGCCCACAGGGTCTTGG |
| 522 | Chr1: 36098617-36098639 | + | CCCTGTGGGCCCTGCTCCCCTGG |
| 523 | Chr1: 36098617-36098639 | − | CCAGGGGAGCAGGGCCCACAGGG |
| 524 | Chr1: 36098618-36098640 | − | GCCAGGGGAGCAGGGCCCACAGG |
| 525 | Chr1: 36098626-36098648 | − | GATGGGGAGCCAGGGGAGCAGGG |
| 526 | Chr1: 36098627-36098649 | − | GGATGGGGAGCCAGGGGAGCAGG |
| 527 | Chr1: 36098633-36098655 | − | AGGGGAGGATGGGGAGCCAGGGG |
| 528 | Chr1: 36098634-36098656 | − | CAGGGGAGGATGGGGAGCCAGGG |
| 529 | Chr1: 36098635-36098657 | + | CCTGGCTCCCCATCCTCCCCTGG |
| 530 | Chr1: 36098635-36098657 | − | CCAGGGGAGGATGGGGAGCCAGG |
| 531 | Chr1: 36098642-36098664 | − | GGGTGAGCCAGGGGAGGATGGGG |
| 532 | Chr1: 36098643-36098665 | − | GGGGTGAGCCAGGGGAGGATGGG |
| 533 | Chr1: 36098644-36098666 | − | AGGGGTGAGCCAGGGGAGGATGG |
| 534 | Chr1: 36098648-36098670 | − | GGACAGGGGTGAGCCAGGGGAGG |
| 535 | Chr1: 36098651-36098673 | − | GGGGGACAGGGGTGAGCCAGGGG |
| 536 | Chr1: 36098652-36098674 | − | TGGGGGACAGGGGTGAGCCAGGG |
| 537 | Chr1: 36098653-36098675 | − | TTGGGGGACAGGGGTGAGCCAGG |
| 538 | Chr1: 36098662-36098684 | + | CCCCTGTCCCCCAAGAGTCCTGG |
| 539 | Chr1: 36098662-36098684 | − | CCAGGACTCTTGGGGGACAGGGG |
| 540 | Chr1: 36098663-36098685 | + | CCCTGTCCCCCAAGAGTCCTGGG |
| 541 | Chr1: 36098663-36098685 | − | CCCAGGACTCTTGGGGGACAGGG |
| 542 | Chr1: 36098664-36098686 | − | TCCCAGGACTCTTGGGGGACAGG |
| 543 | Chr1: 36098669-36098691 | − | TGGGGTCCCAGGACTCTTGGGGG |
| 544 | Chr1: 36098670-36098692 | − | CTGGGGTCCCAGGACTCTTGGGG |
| 545 | Chr1: 36098671-36098693 | − | GCTGGGGTCCCAGGACTCTTGGG |
| 546 | Chr1: 36098672-36098694 | − | AGCTGGGGTCCCAGGACTCTTGG |
| 547 | Chr1: 36098674-36098696 | + | AAGAGTCCTGGGACCCCAGCTGG |
| 548 | Chr1: 36098675-36098697 | + | AGAGTCCTGGGACCCCAGCTGGG |
| 549 | Chr1: 36098680-36098702 | − | AGGGGCCCAGCTGGGGTCCCAGG |
| 550 | Chr1: 36098687-36098709 | − | GGGGGACAGGGGCCCAGCTGGGG |
| 551 | Chr1: 36098688-36098710 | − | AGGGGGACAGGGGCCCAGCTGGG |
| 552 | Chr1: 36098689-36098711 | − | AAGGGGGACAGGGGCCCAGCTGG |
| 553 | Chr1: 36098691-36098713 | + | AGCTGGGCCCCTGTCCCCCTTGG |
| 554 | Chr1: 36098692-36098714 | + | GCTGGGCCCCTGTCCCCCTTGGG |
| 555 | Chr1: 36098693-36098715 | + | CTGGGCCCCTGTCCCCCTTGGGG |
| 556 | Chr1: 36098698-36098720 | + | CCCCTGTCCCCCTTGGGGCCTGG |
| 557 | Chr1: 36098698-36098720 | − | CCAGGCCCCAAGGGGGACAGGGG |
| 558 | Chr1: 36098699-36098721 | − | GCCAGGCCCCAAGGGGGACAGGG |
| 559 | Chr1: 36098700-36098722 | − | TGCCAGGCCCCAAGGGGGACAGG |
| 560 | Chr1: 36098705-36098727 | − | AGGACTGCCAGGCCCCAAGGGGG |
| 561 | Chr1: 36098706-36098728 | − | CAGGACTGCCAGGCCCCAAGGGG |
| 562 | Chr1: 36098707-36098729 | + | CCCTTGGGGCCTGGCAGTCCTGG |
| 563 | Chr1: 36098707-36098729 | − | CCAGGACTGCCAGGCCCCAAGGG |
| 564 | Chr1: 36098708-36098730 | − | GCCAGGACTGCCAGGCCCCAAGG |
| 565 | Chr1: 36098716-36098738 | − | TATGGGATGCCAGGACTGCCAGG |
| 566 | Chr1: 36098724-36098746 | + | TCCTGGCATCCCATAGCCAGTGG |
| 567 | Chr1: 36098725-36098747 | + | CCTGGCATCCCATAGCCAGTGGG |
| 568 | Chr1: 36098725-36098747 | − | CCCACTGGCTATGGGATGCCAGG |
| 569 | Chr1: 36098726-36098748 | + | CTGGCATCCCATAGCCAGTGGGG |
| 570 | Chr1: 36098733-36098755 | − | TGATAGGCCCCACTGGCTATGGG |
| 571 | Chr1: 36098734-36098756 | − | CTGATAGGCCCCACTGGCTATGG |
| 572 | Chr1: 36098740-36098762 | + | CCAGTGGGGCCTATCAGCCCAGG |
| 573 | Chr1: 36098740-36098762 | − | CCTGGGCTGATAGGCCCCACTGG |
| 574 | Chr1: 36098741-36098763 | + | CAGTGGGGCCTATCAGCCCAGGG |
| 575 | Chr1: 36098742-36098764 | + | AGTGGGGCCTATCAGCCCAGGGG |
| 576 | Chr1: 36098743-36098765 | + | GTGGGGCCTATCAGCCCAGGGGG |
| 577 | Chr1: 36098744-36098766 | + | TGGGGCCTATCAGCCCAGGGGGG |
| 578 | Chr1: 36098749-36098771 | − | CGGGGCCCCCCTGGGCTGATAGG |
| 579 | Chr1: 36098750-36098772 | + | CTATCAGCCCAGGGGGGCCCCGG |
| 580 | Chr1: 36098751-36098773 | + | TATCAGCCCAGGGGGGCCCCGGG |
| 581 | Chr1: 36098757-36098779 | − | CAGGGACCCGGGGCCCCCCTGGG |
| 582 | Chr1: 36098758-36098780 | + | CCAGGGGGGCCCCGGGTCCCTGG |
| 583 | Chr1: 36098758-36098780 | − | CCAGGGACCCGGGGCCCCCCTGG |
| 584 | Chr1: 36098767-36098789 | − | AAAGGGGAGCCAGGGACCCGGGG |
| 585 | Chr1: 36098768-36098790 | − | CAAAGGGGAGCCAGGGACCCGGG |
| 586 | Chr1: 36098769-36098791 | + | CCGGGTCCCTGGCTCCCCTTTGG |
| 587 | Chr1: 36098769-36098791 | − | CCAAAGGGGAGCCAGGGACCCGG |
| 588 | Chr1: 36098775-36098797 | − | CAGGGGCCAAAGGGGAGCCAGGG |
| 589 | Chr1: 36098776-36098798 | − | TCAGGGGCCAAAGGGGAGCCAGG |
| 590 | Chr1: 36098779-36098801 | + | GGCTCCCCTTTGGCCCCTGATGG |
| 591 | Chr1: 36098780-36098802 | + | GCTCCCCTTTGGCCCCTGATGGG |
| 592 | Chr1: 36098783-36098805 | − | GGGCCCATCAGGGGCCAAAGGGG |
| 593 | Chr1: 36098784-36098806 | − | AGGGCCCATCAGGGGCCAAAGGG |
| 594 | Chr1: 36098785-36098807 | − | CAGGGCCCATCAGGGGCCAAAGG |
| 595 | Chr1: 36098788-36098810 | + | TTGGCCCCTGATGGGCCCTGTGG |
| 596 | Chr1: 36098792-36098814 | − | AGGACCACAGGGCCCATCAGGGG |
| 597 | Chr1: 36098793-36098815 | − | CAGGACCACAGGGCCCATCAGGG |
| 598 | Chr1: 36098794-36098816 | + | CCTGATGGGCCCTGTGGTCCTGG |
| 599 | Chr1: 36098794-36098816 | − | CCAGGACCACAGGGCCCATCAGG |
| 600 | Chr1: 36098803-36098825 | − | GCAGGGTTGCCAGGACCACAGGG |
| 601 | Chr1: 36098804-36098826 | − | AGCAGGGTTGCCAGGACCACAGG |
| 602 | Chr1: 36098812-36098834 | + | CCTGGCAACCCTGCTGCCCCTGG |
| 603 | Chr1: 36098812-36098834 | − | CCAGGGGCAGCAGGGTTGCCAGG |
| 604 | Chr1: 36098813-36098835 | + | CTGGCAACCCTGCTGCCCCTGGG |
| 605 | Chr1: 36098820-36098842 | − | TGGGAGTCCCAGGGGCAGCAGGG |
| 606 | Chr1: 36098821-36098843 | − | GTGGGAGTCCCAGGGGCAGCAGG |
| 607 | Chr1: 36098828-36098850 | − | AGACGGTGTGGGAGTCCCAGGGG |
| 608 | Chr1: 36098829-36098851 | − | TAGACGGTGTGGGAGTCCCAGGG |
| 609 | Chr1: 36098830-36098852 | − | GTAGACGGTGTGGGAGTCCCAGG |
| 610 | Chr1: 36098836-36098858 | + | ACTCCCACACCGTCTACTCCAGG |
| 611 | Chr1: 36098839-36098861 | + | CCCACACCGTCTACTCCAGGAGG |
| 612 | Chr1: 36098839-36098861 | − | CCTCCTGGAGTAGACGGTGTGGG |
| 613 | Chr1: 36098840-36098862 | − | ACCTCCTGGAGTAGACGGTGTGG |
| 614 | Chr1: 36098845-36098867 | − | AAAGGACCTCCTGGAGTAGACGG |
| 615 | Chr1: 36098848-36098870 | + | TCTACTCCAGGAGGTCCTTTTGG |
| 616 | Chr1: 36098849-36098871 | + | CTACTCCAGGAGGTCCTTTTGGG |
| 617 | Chr1: 36098854-36098876 | − | GTGGGCCCAAAAGGACCTCCTGG |
| 618 | Chr1: 36098863-36098885 | + | CCTTTTGGGCCCACAGCTCCTGG |
| 619 | Chr1: 36098863-36098885 | − | CCAGGAGCTGTGGGCCCAAAAGG |
| 620 | Chr1: 36098872-36098894 | − | AGGGGGGAGCCAGGAGCTGTGGG |
| 621 | Chr1: 36098873-36098895 | − | CAGGGGGGAGCCAGGAGCTGTGG |
| 622 | Chr1: 36098874-36098896 | + | CACAGCTCCTGGCTCCCCCCTGG |
| 623 | Chr1: 36098875-36098897 | + | ACAGCTCCTGGCTCCCCCCTGGG |
| 624 | Chr1: 36098876-36098898 | + | CAGCTCCTGGCTCCCCCCTGGGG |
| 625 | Chr1: 36098881-36098903 | + | CCTGGCTCCCCCCTGGGGCCTGG |
| 626 | Chr1: 36098881-36098903 | − | CCAGGCCCCAGGGGGGAGCCAGG |
| 627 | Chr1: 36098888-36098910 | − | TGGAGTTCCAGGCCCCAGGGGGG |
| 628 | Chr1: 36098889-36098911 | − | CTGGAGTTCCAGGCCCCAGGGGG |
| 629 | Chr1: 36098890-36098912 | + | CCCCTGGGGCCTGGAACTCCAGG |
| 630 | Chr1: 36098890-36098912 | − | CCTGGAGTTCCAGGCCCCAGGGG |
| 631 | Chr1: 36098891-36098913 | − | TCCTGGAGTTCCAGGCCCCAGGG |
| 632 | Chr1: 36098892-36098914 | − | CTCCTGGAGTTCCAGGCCCCAGG |
| 633 | Chr1: 36098893-36098915 | + | CTGGGGCCTGGAACTCCAGGAGG |
| 634 | Chr1: 36098899-36098921 | − | TCTGGGCCTCCTGGAGTTCCAGG |
| 635 | Chr1: 36098908-36098930 | − | AAGGGTGAGTCTGGGCCTCCTGG |
| 636 | Chr1: 36098916-36098938 | − | CAGGAGACAAGGGTGAGTCTGGG |
| 637 | Chr1: 36098917-36098939 | + | CCAGACTCACCCTTGTCTCCTGG |
| 638 | Chr1: 36098917-36098939 | − | CCAGGAGACAAGGGTGAGTCTGG |
| 639 | Chr1: 36098918-36098940 | + | CAGACTCACCCTTGTCTCCTGGG |
| 640 | Chr1: 36098919-36098941 | + | AGACTCACCCTTGTCTCCTGGGG |
| 641 | Chr1: 36098926-36098948 | + | CCCTTGTCTCCTGGGGCCCCAGG |
| 642 | Chr1: 36098926-36098948 | − | CCTGGGGCCCCAGGAGACAAGGG |
| 643 | Chr1: 36098927-36098949 | − | TCCTGGGGCCCCAGGAGACAAGG |
| 644 | Chr1: 36098935-36098957 | − | GATGGGCTTCCTGGGGCCCCAGG |
| 645 | Chr1: 36098942-36098964 | − | TGGTTTGGATGGGCTTCCTGGGG |
| 646 | Chr1: 36098943-36098965 | − | CTGGTTTGGATGGGCTTCCTGGG |
| 647 | Chr1: 36098944-36098966 | + | CCAGGAAGCCCATCCAAACCAGG |
| 648 | Chr1: 36098944-36098966 | − | CCTGGTTTGGATGGGCTTCCTGG |
| 649 | Chr1: 36098952-36098974 | − | TAGGCAAACCTGGTTTGGATGGG |
| 650 | Chr1: 36098953-36098975 | − | TTAGGCAAACCTGGTTTGGATGG |
| 651 | Chr1: 36098957-36098979 | − | TGGCTTAGGCAAACCTGGTTTGG |
| 652 | Chr1: 36098962-36098984 | + | CCAGGTTTGCCTAAGCCAGCTGG |
| 653 | Chr1: 36098962-36098984 | − | CCAGCTGGCTTAGGCAAACCTGG |
| 654 | Chr1: 36098968-36098990 | + | TTGCCTAAGCCAGCTGGACCAGG |
| 655 | Chr1: 36098969-36098991 | + | TGCCTAAGCCAGCTGGACCAGGG |
| 656 | Chr1: 36098971-36098993 | − | CTCCCTGGTCCAGCTGGCTTAGG |
| 657 | Chr1: 36098972-36098994 | + | CTAAGCCAGCTGGACCAGGGAGG |
| 658 | Chr1: 36098976-36098998 | + | GCCAGCTGGACCAGGGAGGCCGG |
| 659 | Chr1: 36098977-36098999 | + | CCAGCTGGACCAGGGAGGCCGGG |
| 660 | Chr1: 36098977-36098999 | − | CCCGGCCTCCCTGGTCCAGCTGG |
| 661 | Chr1: 36098978-36099000 | + | CAGCTGGACCAGGGAGGCCGGGG |
| 662 | Chr1: 36098979-36099001 | + | AGCTGGACCAGGGAGGCCGGGGG |
| 663 | Chr1: 36098980-36099002 | + | GCTGGACCAGGGAGGCCGGGGGG |
| 664 | Chr1: 36098981-36099003 | + | CTGGACCAGGGAGGCCGGGGGGG |
| 665 | Chr1: 36098985-36099007 | + | ACCAGGGAGGCCGGGGGGGCCGG |
| 666 | Chr1: 36098986-36099008 | + | CCAGGGAGGCCGGGGGGGCCGGG |
| 667 | Chr1: 36098986-36099008 | − | CCCGGCCCCCCCGGCCTCCCTGG |
| 668 | Chr1: 36098987-36099009 | + | CAGGGAGGCCGGGGGGGCCGGGG |
| 669 | Chr1: 36098988-36099010 | + | AGGGAGGCCGGGGGGGCCGGGGG |
| 670 | Chr1: 36098995-36099017 | − | GGGGGTGCCCCCGGCCCCCCCGG |
| 671 | Chr1: 36099004-36099026 | + | CCGGGGGCACCCCCCTGCCCTGG |
| 672 | Chr1: 36099004-36099026 | − | CCAGGGCAGGGGGGTGCCCCCGG |
| 673 | Chr1: 36099005-36099027 | + | CGGGGGCACCCCCCTGCCCTGGG |
| 674 | Chr1: 36099006-36099028 | + | GGGGGCACCCCCCTGCCCTGGGG |
| 675 | Chr1: 36099013-36099035 | + | CCCCCCTGCCCTGGGGCCCCAGG |
| 676 | Chr1: 36099013-36099035 | − | CCTGGGGCCCCAGGGCAGGGGGG |
| 677 | Chr1: 36099014-36099036 | − | GCCTGGGGCCCCAGGGCAGGGGG |
| 678 | Chr1: 36099015-36099037 | − | TGCCTGGGGCCCCAGGGCAGGGG |
| 679 | Chr1: 36099016-36099038 | − | CTGCCTGGGGCCCCAGGGCAGGG |
| 680 | Chr1: 36099017-36099039 | − | GCTGCCTGGGGCCCCAGGGCAGG |
| 681 | Chr1: 36099021-36099043 | + | CCCTGGGGCCCCAGGCAGCCCGG |
| 682 | Chr1: 36099021-36099043 | − | CCGGGCTGCCTGGGGCCCCAGGG |
| 683 | Chr1: 36099022-36099044 | + | CCTGGGGCCCCAGGCAGCCCGGG |
| 684 | Chr1: 36099022-36099044 | − | CCCGGGCTGCCTGGGGCCCCAGG |
| 685 | Chr1: 36099026-36099048 | + | GGGCCCCAGGCAGCCCGGGCTGG |
| 686 | Chr1: 36099029-36099051 | − | GGGCCAGCCCGGGCTGCCTGGGG |
| 687 | Chr1: 36099030-36099052 | − | TGGGCCAGCCCGGGCTGCCTGGG |
| 688 | Chr1: 36099031-36099053 | − | GTGGGCCAGCCCGGGCTGCCTGG |
| 689 | Chr1: 36099039-36099061 | − | ATAATGGAGTGGGCCAGCCCGGG |
| 690 | Chr1: 36099040-36099062 | − | GATAATGGAGTGGGCCAGCCCGG |
| 691 | Chr1: 36099049-36099071 | − | CTCAAGGGGGATAATGGAGTGGG |
| 692 | Chr1: 36099050-36099072 | + | CCACTCCATTATCCCCCTTGAGG |
| 693 | Chr1: 36099050-36099072 | − | CCTCAAGGGGGATAATGGAGTGG |
| 694 | Chr1: 36099055-36099077 | − | CGAGGCCTCAAGGGGGATAATGG |
| 695 | Chr1: 36099062-36099084 | − | AGGTGATCGAGGCCTCAAGGGGG |
| 696 | Chr1: 36099063-36099085 | − | CAGGTGATCGAGGCCTCAAGGGG |
| 697 | Chr1: 36099064-36099086 | + | CCCTTGAGGCCTCGATCACCTGG |
| 698 | Chr1: 36099064-36099086 | − | CCAGGTGATCGAGGCCTCAAGGG |
| 699 | Chr1: 36099065-36099087 | + | CCTTGAGGCCTCGATCACCTGGG |
| 700 | Chr1: 36099065-36099087 | − | CCCAGGTGATCGAGGCCTCAAGG |
| 701 | Chr1: 36099066-36099088 | + | CTTGAGGCCTCGATCACCTGGGG |
| 702 | Chr1: 36099067-36099089 | + | TTGAGGCCTCGATCACCTGGGGG |
| 703 | Chr1: 36099073-36099095 | + | CCTCGATCACCTGGGGGCCCAGG |
| 704 | Chr1: 36099073-36099095 | − | CCTGGGCCCCCAGGTGATCGAGG |
| 705 | Chr1: 36099082-36099104 | − | CAGGGGGAGCCTGGGCCCCCAGG |
| 706 | Chr1: 36099083-36099105 | + | CTGGGGGCCCAGGCTCCCCCTGG |
| 707 | Chr1: 36099084-36099106 | + | TGGGGGCCCAGGCTCCCCCTGGG |
| 708 | Chr1: 36099085-36099107 | + | GGGGGCCCAGGCTCCCCCTGGGG |
| 709 | Chr1: 36099090-36099112 | − | CAGGGCCCCAGGGGGAGCCTGGG |
| 710 | Chr1: 36099091-36099113 | + | CCAGGCTCCCCCTGGGGCCCTGG |
| 711 | Chr1: 36099091-36099113 | − | CCAGGGCCCCAGGGGGAGCCTGG |
| 712 | Chr1: 36099098-36099120 | − | GGGGGAACCAGGGCCCCAGGGGG |
| 713 | Chr1: 36099099-36099121 | − | AGGGGGAACCAGGGCCCCAGGGG |
| 714 | Chr1: 36099100-36099122 | − | CAGGGGGAACCAGGGCCCCAGGG |
| 715 | Chr1: 36099101-36099123 | + | CCTGGGGCCCTGGTTCCCCCTGG |
| 716 | Chr1: 36099101-36099123 | − | CCAGGGGGAACCAGGGCCCCAGG |
| 717 | Chr1: 36099108-36099130 | − | CAGGATTCCAGGGGGAACCAGGG |
| 718 | Chr1: 36099109-36099131 | + | CCTGGTTCCCCCTGGAATCCTGG |
| 719 | Chr1: 36099109-36099131 | − | CCAGGATTCCAGGGGGAACCAGG |
| 720 | Chr1: 36099110-36099132 | + | CTGGTTCCCCCTGGAATCCTGGG |
| 721 | Chr1: 36099111-36099133 | + | TGGTTCCCCCTGGAATCCTGGGG |
| 722 | Chr1: 36099112-36099134 | + | GGTTCCCCCTGGAATCCTGGGGG |
| 723 | Chr1: 36099116-36099138 | − | AGGGCCCCCAGGATTCCAGGGGG |
| 724 | Chr1: 36099117-36099139 | − | CAGGGCCCCCAGGATTCCAGGGG |
| 725 | Chr1: 36099118-36099140 | + | CCCTGGAATCCTGGGGGCCCTGG |
| 726 | Chr1: 36099118-36099140 | − | CCAGGGCCCCCAGGATTCCAGGG |
| 727 | Chr1: 36099119-36099141 | − | GCCAGGGCCCCCAGGATTCCAGG |
| 728 | Chr1: 36099127-36099149 | − | CAAGGGGTGCCAGGGCCCCCAGG |
| 729 | Chr1: 36099128-36099150 | + | CTGGGGGCCCTGGCACCCCTTGG |
| 730 | Chr1: 36099129-36099151 | + | TGGGGGCCCTGGCACCCCTTGGG |
| 731 | Chr1: 36099135-36099157 | − | CAGGTGCCCAAGGGGTGCCAGGG |
| 732 | Chr1: 36099136-36099158 | + | CCTGGCACCCCTTGGGCACCTGG |
| 733 | Chr1: 36099136-36099158 | − | CCAGGTGCCCAAGGGGTGCCAGG |
| 734 | Chr1: 36099143-36099165 | − | TGGAAAACCAGGTGCCCAAGGGG |
| 735 | Chr1: 36099144-36099166 | − | CTGGAAAACCAGGTGCCCAAGGG |
| 736 | Chr1: 36099145-36099167 | + | CCTTGGGCACCTGGTTTTCCAGG |
| 737 | Chr1: 36099145-36099167 | − | CCTGGAAAACCAGGTGCCCAAGG |
| 738 | Chr1: 36099146-36099168 | + | CTTGGGCACCTGGTTTTCCAGGG |
| 739 | Chr1: 36099154-36099176 | − | ATTACTATCCCTGGAAAACCAGG |
| 740 | Chr1: 36099162-36099184 | + | TCCAGGGATAGTAATGCCTGAGG |
| 741 | Chr1: 36099163-36099185 | + | CCAGGGATAGTAATGCCTGAGGG |
| 742 | Chr1: 36099163-36099185 | − | CCCTCAGGCATTACTATCCCTGG |
| 743 | Chr1: 36099164-36099186 | + | CAGGGATAGTAATGCCTGAGGGG |
| 744 | Chr1: 36099169-36099191 | + | ATAGTAATGCCTGAGGGGCCCGG |
| 745 | Chr1: 36099170-36099192 | + | TAGTAATGCCTGAGGGGCCCGGG |
| 746 | Chr1: 36099173-36099195 | + | TAATGCCTGAGGGGCCCGGGAGG |
| 747 | Chr1: 36099178-36099200 | + | CCTGAGGGGCCCGGGAGGCCAGG |
| 748 | Chr1: 36099178-36099200 | − | CCTGGCCTCCCGGGCCCCTCAGG |
| 749 | Chr1: 36099179-36099201 | + | CTGAGGGGCCCGGGAGGCCAGGG |
| 750 | Chr1: 36099180-36099202 | + | TGAGGGGCCCGGGAGGCCAGGGG |
| 751 | Chr1: 36099181-36099203 | + | GAGGGGCCCGGGAGGCCAGGGGG |
| 752 | Chr1: 36099187-36099209 | + | CCCGGGAGGCCAGGGGGTCCTGG |
| 753 | Chr1: 36099187-36099209 | − | CCAGGACCCCCTGGCCTCCCGGG |
| 754 | Chr1: 36099188-36099210 | + | CCGGGAGGCCAGGGGGTCCTGGG |
| 755 | Chr1: 36099188-36099210 | − | CCCAGGACCCCCTGGCCTCCCGG |
| 756 | Chr1: 36099189-36099211 | + | CGGGAGGCCAGGGGGTCCTGGGG |
| 757 | Chr1: 36099190-36099212 | + | GGGAGGCCAGGGGGTCCTGGGGG |
| 758 | Chr1: 36099196-36099218 | − | CGGGGACCCCCAGGACCCCCTGG |
| 759 | Chr1: 36099197-36099219 | + | CAGGGGGTCCTGGGGGTCCCCGG |
| 760 | Chr1: 36099200-36099222 | + | GGGGTCCTGGGGGTCCCCGGAGG |
| 761 | Chr1: 36099205-36099227 | − | CAGGGCCTCCGGGGACCCCCAGG |
| 762 | Chr1: 36099206-36099228 | + | CTGGGGGTCCCCGGAGGCCCTGG |
| 763 | Chr1: 36099214-36099236 | − | CGAGGGGACCAGGGCCTCCGGGG |
| 764 | Chr1: 36099215-36099237 | − | ACGAGGGGACCAGGGCCTCCGGG |
| 765 | Chr1: 36099216-36099238 | − | TACGAGGGGACCAGGGCCTCCGG |
| 766 | Chr1: 36099223-36099245 | + | CCCTGGTCCCCTCGTATTCCTGG |
| 767 | Chr1: 36099223-36099245 | − | CCAGGAATACGAGGGGACCAGGG |
| 768 | Chr1: 36099224-36099246 | − | GCCAGGAATACGAGGGGACCAGG |
| 769 | Chr1: 36099230-36099252 | − | GGGGGAGCCAGGAATACGAGGGG |
| 770 | Chr1: 36099231-36099253 | − | GGGGGGAGCCAGGAATACGAGGG |
| 771 | Chr1: 36099232-36099254 | − | CGGGGGGAGCCAGGAATACGAGG |
| 772 | Chr1: 36099241-36099263 | + | CCTGGCTCCCCCCGAAGCCCCGG |
| 773 | Chr1: 36099241-36099263 | − | CCGGGGCTTCGGGGGGAGCCAGG |
| 774 | Chr1: 36099248-36099270 | − | AGGGCAGCCGGGGCTTCGGGGGG |
| 775 | Chr1: 36099249-36099271 | − | CAGGGCAGCCGGGGCTTCGGGGG |
| 776 | Chr1: 36099250-36099272 | + | CCCCGAAGCCCCGGCTGCCCTGG |
| 777 | Chr1: 36099250-36099272 | − | CCAGGGCAGCCGGGGCTTCGGGG |
| 778 | Chr1: 36099251-36099273 | − | ACCAGGGCAGCCGGGGCTTCGGG |
| 779 | Chr1: 36099252-36099274 | − | CACCAGGGCAGCCGGGGCTTCGG |
| 780 | Chr1: 36099253-36099275 | + | CGAAGCCCCGGCTGCCCTGGTGG |
| 781 | Chr1: 36099258-36099280 | − | TCGGGCCACCAGGGCAGCCGGGG |
| 782 | Chr1: 36099259-36099281 | − | GTCGGGCCACCAGGGCAGCCGGG |
| 783 | Chr1: 36099260-36099282 | − | GGTCGGGCCACCAGGGCAGCCGG |
| 784 | Chr1: 36099267-36099289 | − | CTGGCAAGGTCGGGCCACCAGGG |
| 785 | Chr1: 36099268-36099290 | + | CCTGGTGGCCCGACCTTGCCAGG |
| 786 | Chr1: 36099268-36099290 | − | CCTGGCAAGGTCGGGCCACCAGG |
| 787 | Chr1: 36099269-36099291 | + | CTGGTGGCCCGACCTTGCCAGGG |
| 788 | Chr1: 36099276-36099298 | − | CAGGGCTCCCTGGCAAGGTCGGG |
| 789 | Chr1: 36099277-36099299 | + | CCGACCTTGCCAGGGAGCCCTGG |
| 790 | Chr1: 36099277-36099299 | − | CCAGGGCTCCCTGGCAAGGTCGG |
| 791 | Chr1: 36099278-36099300 | + | CGACCTTGCCAGGGAGCCCTGGG |
| 792 | Chr1: 36099279-36099301 | + | GACCTTGCCAGGGAGCCCTGGGG |
| 793 | Chr1: 36099280-36099302 | + | ACCTTGCCAGGGAGCCCTGGGGG |
| 794 | Chr1: 36099281-36099303 | − | TCCCCCAGGGCTCCCTGGCAAGG |
| 795 | Chr1: 36099286-36099308 | − | GCTGGTCCCCCAGGGCTCCCTGG |
| 796 | Chr1: 36099294-36099316 | − | TGGGCAAGGCTGGTCCCCCAGGG |
| 797 | Chr1: 36099295-36099317 | − | ATGGGCAAGGCTGGTCCCCCAGG |
| 798 | Chr1: 36099299-36099321 | + | GGGGACCAGCCTTGCCCATCCGG |
| 799 | Chr1: 36099300-36099322 | + | GGGACCAGCCTTGCCCATCCGGG |
| 800 | Chr1: 36099304-36099326 | − | TTCTCCCGGATGGGCAAGGCTGG |
| 801 | Chr1: 36099308-36099330 | − | TGGCTTCTCCCGGATGGGCAAGG |
| 802 | Chr1: 36099310-36099332 | + | TTGCCCATCCGGGAGAAGCCAGG |
| 803 | Chr1: 36099311-36099333 | + | TGCCCATCCGGGAGAAGCCAGGG |
| 804 | Chr1: 36099312-36099334 | + | GCCCATCCGGGAGAAGCCAGGGG |
| 805 | Chr1: 36099313-36099335 | + | CCCATCCGGGAGAAGCCAGGGGG |
| 806 | Chr1: 36099313-36099335 | − | CCCCCTGGCTTCTCCCGGATGGG |
| 807 | Chr1: 36099314-36099336 | − | GCCCCCTGGCTTCTCCCGGATGG |
| 808 | Chr1: 36099318-36099340 | − | CTGGGCCCCCTGGCTTCTCCCGG |
| 809 | Chr1: 36099322-36099344 | + | GAGAAGCCAGGGGGCCCAGCAGG |
| 810 | Chr1: 36099323-36099345 | + | AGAAGCCAGGGGGCCCAGCAGGG |
| 811 | Chr1: 36099328-36099350 | + | CCAGGGGGCCCAGCAGGGCCAGG |
| 812 | Chr1: 36099328-36099350 | − | CCTGGCCCTGCTGGGCCCCCTGG |
| 813 | Chr1: 36099336-36099358 | − | ATGGGCAGCCTGGCCCTGCTGGG |
| 814 | Chr1: 36099337-36099359 | − | CATGGGCAGCCTGGCCCTGCTGG |
| 815 | Chr1: 36099338-36099360 | + | CAGCAGGGCCAGGCTGCCCATGG |
| 816 | Chr1: 36099346-36099368 | + | CCAGGCTGCCCATGGAGTCCTGG |
| 817 | Chr1: 36099346-36099368 | − | CCAGGACTCCATGGGCAGCCTGG |
| 818 | Chr1: 36099354-36099376 | − | TGGGAAAGCCAGGACTCCATGGG |
| 819 | Chr1: 36099355-36099377 | − | ATGGGAAAGCCAGGACTCCATGG |
| 820 | Chr1: 36099361-36099383 | + | AGTCCTGGCTTTCCCATGCCTGG |
| 821 | Chr1: 36099364-36099386 | − | AAACCAGGCATGGGAAAGCCAGG |
| 822 | Chr1: 36099370-36099392 | + | TTTCCCATGCCTGGTTTTCCTGG |
| 823 | Chr1: 36099371-36099393 | + | TTCCCATGCCTGGTTTTCCTGGG |
| 824 | Chr1: 36099373-36099395 | − | TTCCCAGGAAAACCAGGCATGGG |
| 825 | Chr1: 36099374-36099396 | − | CTTCCCAGGAAAACCAGGCATGG |
| 826 | Chr1: 36099379-36099401 | + | CCTGGTTTTCCTGGGAAGCCAGG |
| 827 | Chr1: 36099379-36099401 | − | CCTGGCTTCCCAGGAAAACCAGG |
| 828 | Chr1: 36099380-36099402 | + | CTGGTTTTCCTGGGAAGCCAGGG |
| 829 | Chr1: 36099381-36099403 | + | TGGTTTTCCTGGGAAGCCAGGGG |
| 830 | Chr1: 36099382-36099404 | + | GGTTTTCCTGGGAAGCCAGGGGG |
| 831 | Chr1: 36099383-36099405 | + | GTTTTCCTGGGAAGCCAGGGGGG |
| 832 | Chr1: 36099388-36099410 | + | CCTGGGAAGCCAGGGGGGCCAGG |
| 833 | Chr1: 36099388-36099410 | − | CCTGGCCCCCCTGGCTTCCCAGG |
| 834 | Chr1: 36099389-36099411 | + | CTGGGAAGCCAGGGGGGCCAGGG |
| 835 | Chr1: 36099390-36099412 | + | TGGGAAGCCAGGGGGGCCAGGGG |
| 836 | Chr1: 36099391-36099413 | + | GGGAAGCCAGGGGGGCCAGGGGG |
| 837 | Chr1: 36099397-36099419 | − | CGGGGTCCCCCTGGCCCCCCTGG |
| 838 | Chr1: 36099400-36099422 | + | GGGGGGCCAGGGGGACCCCGAGG |
| 839 | Chr1: 36099405-36099427 | + | GCCAGGGGGACCCCGAGGCCCGG |
| 840 | Chr1: 36099406-36099428 | + | CCAGGGGGACCCCGAGGCCCGGG |
| 841 | Chr1: 36099406-36099428 | − | CCCGGGCCTCGGGGTCCCCCTGG |
| 842 | Chr1: 36099415-36099437 | + | CCCCGAGGCCCGGGCTTCCCAGG |
| 843 | Chr1: 36099415-36099437 | − | CCTGGGAAGCCCGGGCCTCGGGG |
| 844 | Chr1: 36099416-36099438 | + | CCCGAGGCCCGGGCTTCCCAGGG |
| 845 | Chr1: 36099416-36099438 | − | CCCTGGGAAGCCCGGGCCTCGGG |
| 846 | Chr1: 36099417-36099439 | + | CCGAGGCCCGGGCTTCCCAGGGG |
| 847 | Chr1: 36099417-36099439 | − | CCCCTGGGAAGCCCGGGCCTCGG |
| 848 | Chr1: 36099418-36099440 | + | CGAGGCCCGGGCTTCCCAGGGGG |
| 849 | Chr1: 36099419-36099441 | + | GAGGCCCGGGCTTCCCAGGGGGG |
| 850 | Chr1: 36099423-36099445 | + | CCCGGGCTTCCCAGGGGGGCCGG |
| 851 | Chr1: 36099423-36099445 | − | CCGGCCCCCCTGGGAAGCCCGGG |
| 852 | Chr1: 36099424-36099446 | + | CCGGGCTTCCCAGGGGGGCCGGG |
| 853 | Chr1: 36099424-36099446 | − | CCCGGCCCCCCTGGGAAGCCCGG |
| 854 | Chr1: 36099432-36099454 | − | AGGGAGAGCCCGGCCCCCCTGGG |
| 855 | Chr1: 36099433-36099455 | − | AAGGGAGAGCCCGGCCCCCCTGG |
| 856 | Chr1: 36099437-36099459 | + | GGGGGCCGGGCTCTCCCTTCAGG |
| 857 | Chr1: 36099442-36099464 | − | ATGGACCTGAAGGGAGAGCCCGG |
| 858 | Chr1: 36099445-36099467 | + | GGCTCTCCCTTCAGGTCCATCGG |
| 859 | Chr1: 36099451-36099473 | − | CTGCTGCCGATGGACCTGAAGGG |
| 860 | Chr1: 36099452-36099474 | − | GCTGCTGCCGATGGACCTGAAGG |
| 861 | Chr1: 36099454-36099476 | + | TTCAGGTCCATCGGCAGCAGCGG |
| 862 | Chr1: 36099460-36099482 | + | TCCATCGGCAGCAGCGGTAGAGG |
| 863 | Chr1: 36099461-36099483 | − | GCCTCTACCGCTGCTGCCGATGG |
| 864 | Chr1: 36099485-36099507 | + | TTTCTGAGAAAGAAAGAGAAAGG |
| 865 | Chr1: 36099486-36099508 | + | TTCTGAGAAAGAAAGAGAAAGGG |
| 866 | Chr1: 36099487-36099509 | + | TCTGAGAAAGAAAGAGAAAGGGG |
| 867 | Chr1: 36099495-36099517 | + | AGAAAGAGAAAGGGGCAGTCAGG |
| 868 | Chr1: 36099496-36099518 | + | GAAAGAGAAAGGGGCAGTCAGGG |
| 869 | Chr1: 36099497-36099519 | + | AAAGAGAAAGGGGCAGTCAGGGG |
| 870 | Chr1: 36099509-36099531 | + | GCAGTCAGGGGCCTGAACTGTGG |
| 871 | Chr1: 36099510-36099532 | + | CAGTCAGGGGCCTGAACTGTGGG |
| 872 | Chr1: 36099511-36099533 | + | AGTCAGGGGCCTGAACTGTGGGG |
| 873 | Chr1: 36099516-36099538 | + | GGGGCCTGAACTGTGGGGACAGG |
| 874 | Chr1: 36099517-36099539 | + | GGGCCTGAACTGTGGGGACAGGG |
| 875 | Chr1: 36099518-36099540 | + | GGCCTGAACTGTGGGGACAGGGG |
| 876 | Chr1: 36099520-36099542 | − | GTCCCCTGTCCCCACAGTTCAGG |
| 877 | Chr1: 36099542-36099564 | − | AATGGGGGAATGGGTAGATGGGG |
| 878 | Chr1: 36099543-36099565 | − | GAATGGGGGAATGGGTAGATGGG |
| 879 | Chr1: 36099544-36099566 | − | GGAATGGGGGAATGGGTAGATGG |
| 880 | Chr1: 36099551-36099573 | − | TCATACTGGAATGGGGGAATGGG |
| 881 | Chr1: 36099552-36099574 | − | CTCATACTGGAATGGGGGAATGG |
| 882 | Chr1: 36099553-36099575 | + | CATTCCCCCATTCCAGTATGAGG |
| 883 | Chr1: 36099557-36099579 | − | TGTACCTCATACTGGAATGGGGG |
| 884 | Chr1: 36099558-36099580 | − | GTGTACCTCATACTGGAATGGGG |
| 885 | Chr1: 36099559-36099581 | − | CGTGTACCTCATACTGGAATGGG |
| 886 | Chr1: 36099560-36099582 | + | CCATTCCAGTATGAGGTACACGG |
| 887 | Chr1: 36099560-36099582 | − | CCGTGTACCTCATACTGGAATGG |
| 888 | Chr1: 36099561-36099583 | + | CATTCCAGTATGAGGTACACGGG |
| 889 | Chr1: 36099565-36099587 | − | CTCTCCCGTGTACCTCATACTGG |
| 890 | Chr1: 36099566-36099588 | + | CAGTATGAGGTACACGGGAGAGG |
| 891 | Chr1: 36099574-36099596 | + | GGTACACGGGAGAGGAAGAATGG |
| 892 | Chr1: 36099575-36099597 | + | GTACACGGGAGAGGAAGAATGGG |
| 893 | Chr1: 36099576-36099598 | + | TACACGGGAGAGGAAGAATGGGG |
| 894 | Chr1: 36099598-36099620 | + | GCTGCCCCTTCCTGCTCTCATGG |
| 895 | Chr1: 36099602-36099624 | − | TCTTCCATGAGAGCAGGAAGGGG |
| 896 | Chr1: 36099603-36099625 | − | ATCTTCCATGAGAGCAGGAAGGG |
| 897 | Chr1: 36099604-36099626 | − | CATCTTCCATGAGAGCAGGAAGG |
| 898 | Chr1: 36099605-36099627 | + | CTTCCTGCTCTCATGGAAGATGG |
| 899 | Chr1: 36099606-36099628 | + | TTCCTGCTCTCATGGAAGATGGG |
| 900 | Chr1: 36099607-36099629 | + | TCCTGCTCTCATGGAAGATGGGG |
| 901 | Chr1: 36099608-36099630 | − | ACCCCATCTTCCATGAGAGCAGG |
| 902 | Chr1: 36099612-36099634 | + | CTCTCATGGAAGATGGGGTTTGG |
| 903 | Chr1: 36099613-36099635 | + | TCTCATGGAAGATGGGGTTTGGG |
| 904 | Chr1: 36099614-36099636 | + | CTCATGGAAGATGGGGTTTGGGG |
| 905 | Chr1: 36099615-36099637 | + | TCATGGAAGATGGGGTTTGGGGG |
| 906 | Chr1: 36099618-36099640 | + | TGGAAGATGGGGTTTGGGGGTGG |
| 907 | Chr1: 36099624-36099646 | + | ATGGGGTTTGGGGGTGGCCCAGG |
| 908 | Chr1: 36099625-36099647 | + | TGGGGTTTGGGGGTGGCCCAGGG |
| 909 | Chr1: 36099626-36099648 | + | GGGGTTTGGGGGTGGCCCAGGGG |
| 910 | Chr1: 36099635-36099657 | + | GGGTGGCCCAGGGGACATCTTGG |
| 911 | Chr1: 36099636-36099658 | + | GGTGGCCCAGGGGACATCTTGGG |
| 912 | Chr1: 36099637-36099659 | + | GTGGCCCAGGGGACATCTTGGGG |
| 913 | Chr1: 36099638-36099660 | + | TGGCCCAGGGGACATCTTGGGGG |
| 914 | Chr1: 36099641-36099663 | − | TTGCCCCCAAGATGTCCCCTGGG |
| 915 | Chr1: 36099642-36099664 | − | GTTGCCCCCAAGATGTCCCCTGG |
| 916 | Chr1: 36099645-36099667 | + | GGGGACATCTTGGGGGCAACAGG |
| 917 | Chr1: 36099646-36099668 | + | GGGACATCTTGGGGGCAACAGGG |
| 918 | Chr1: 36099660-36099682 | + | GCAACAGGGTGTCCTCCTTAAGG |
| 919 | Chr1: 36099661-36099683 | + | CAACAGGGTGTCCTCCTTAAGGG |
| 920 | Chr1: 36099672-36099694 | − | GGTGTTAGGAGCCCTTAAGGAGG |
| 921 | Chr1: 36099675-36099697 | − | TTGGGTGTTAGGAGCCCTTAAGG |
| 922 | Chr1: 36099685-36099707 | + | TCCTAACACCCAACCTACCTAGG |
| 923 | Chr1: 36099686-36099708 | − | GCCTAGGTAGGTTGGGTGTTAGG |
| 924 | Chr1: 36099689-36099711 | + | AACACCCAACCTACCTAGGCTGG |
| 925 | Chr1: 36099690-36099712 | + | ACACCCAACCTACCTAGGCTGGG |
| 926 | Chr1: 36099693-36099715 | − | AGGCCCAGCCTAGGTAGGTTGGG |
| 927 | Chr1: 36099694-36099716 | − | GAGGCCCAGCCTAGGTAGGTTGG |
| 928 | Chr1: 36099698-36099720 | − | GGAGGAGGCCCAGCCTAGGTAGG |
| 929 | Chr1: 36099702-36099724 | − | TCATGGAGGAGGCCCAGCCTAGG |
| 930 | Chr1: 36099708-36099730 | + | CTGGGCCTCCTCCATGAGCCTGG |
| 931 | Chr1: 36099713-36099735 | − | ATCAGCCAGGCTCATGGAGGAGG |
| 932 | Chr1: 36099716-36099738 | − | AGAATCAGCCAGGCTCATGGAGG |
| 933 | Chr1: 36099719-36099741 | − | GTGAGAATCAGCCAGGCTCATGG |
| 934 | Chr1: 36099726-36099748 | − | ATGAGAGGTGAGAATCAGCCAGG |
| 935 | Chr1: 36099741-36099763 | − | TCAGGTCATGCAGGGATGAGAGG |
| 936 | Chr1: 36099744-36099766 | + | CTCATCCCTGCATGACCTGAAGG |
| 937 | Chr1: 36099747-36099769 | + | ATCCCTGCATGACCTGAAGGTGG |
| 938 | Chr1: 36099749-36099771 | − | CTCCACCTTCAGGTCATGCAGGG |
| 939 | Chr1: 36099750-36099772 | − | ACTCCACCTTCAGGTCATGCAGG |
| 940 | Chr1: 36099752-36099774 | + | TGCATGACCTGAAGGTGGAGTGG |
| 941 | Chr1: 36099759-36099781 | − | CTGGTGGCCACTCCACCTTCAGG |
| 942 | Chr1: 36099760-36099782 | + | CTGAAGGTGGAGTGGCCACCAGG |
| 943 | Chr1: 36099763-36099785 | + | AAGGTGGAGTGGCCACCAGGTGG |
| 944 | Chr1: 36099775-36099797 | − | GGGCTGCTGGTGCCACCTGGTGG |
| 945 | Chr1: 36099778-36099800 | − | GGTGGGCTGCTGGTGCCACCTGG |
| 946 | Chr1: 36099788-36099810 | − | CGGGCTCTAAGGTGGGCTGCTGG |
| 947 | Chr1: 36099791-36099813 | + | GCAGCCCACCTTAGAGCCCGTGG |
| 948 | Chr1: 36099792-36099814 | + | CAGCCCACCTTAGAGCCCGTGGG |
| 949 | Chr1: 36099795-36099817 | − | GCTCCCACGGGCTCTAAGGTGGG |
| 950 | Chr1: 36099796-36099818 | − | TGCTCCCACGGGCTCTAAGGTGG |
| 951 | Chr1: 36099799-36099821 | − | CTCTGCTCCCACGGGCTCTAAGG |
| 952 | Chr1: 36099807-36099829 | − | AGGTGGGGCTCTGCTCCCACGGG |
| 953 | Chr1: 36099808-36099830 | − | GAGGTGGGGCTCTGCTCCCACGG |
| 954 | Chr1: 36099822-36099844 | − | AACTGGGAAGTTGGGAGGTGGGG |
| 955 | Chr1: 36099823-36099845 | − | GAACTGGGAAGTTGGGAGGTGGG |
| 956 | Chr1: 36099824-36099846 | − | TGAACTGGGAAGTTGGGAGGTGG |
| 957 | Chr1: 36099827-36099849 | − | AGATGAACTGGGAAGTTGGGAGG |
| 958 | Chr1: 36099830-36099852 | − | GGGAGATGAACTGGGAAGTTGGG |
| 959 | Chr1: 36099831-36099853 | − | GGGGAGATGAACTGGGAAGTTGG |
| 960 | Chr1: 36099836-36099858 | + | TTCCCAGTTCATCTCCCCCTTGG |
| 961 | Chr1: 36099838-36099860 | − | TTCCAAGGGGGAGATGAACTGGG |
| 962 | Chr1: 36099839-36099861 | − | CTTCCAAGGGGGAGATGAACTGG |
| 963 | Chr1: 36099850-36099872 | − | GCACAGGTGGTCTTCCAAGGGGG |
| 964 | Chr1: 36099851-36099873 | − | GGCACAGGTGGTCTTCCAAGGGG |
| 965 | Chr1: 36099852-36099874 | − | TGGCACAGGTGGTCTTCCAAGGG |
| 966 | Chr1: 36099853-36099875 | − | CTGGCACAGGTGGTCTTCCAAGG |
| 967 | Chr1: 36099863-36099885 | − | GTGCAGTTAGCTGGCACAGGTGG |
| 968 | Chr1: 36099866-36099888 | − | ACGGTGCAGTTAGCTGGCACAGG |
| 969 | Chr1: 36099872-36099894 | − | CTGGAAACGGTGCAGTTAGCTGG |
| 970 | Chr1: 36099873-36099895 | + | CAGCTAACTGCACCGTTTCCAGG |
| 971 | Chr1: 36099881-36099903 | + | TGCACCGTTTCCAGGCCCTCTGG |
| 972 | Chr1: 36099882-36099904 | + | GCACCGTTTCCAGGCCCTCTGGG |
| 973 | Chr1: 36099883-36099905 | + | CACCGTTTCCAGGCCCTCTGGGG |
| 974 | Chr1: 36099885-36099907 | − | TACCCCAGAGGGCCTGGAAACGG |
| 975 | Chr1: 36099890-36099912 | + | TCCAGGCCCTCTGGGGTATTAGG |
| 976 | Chr1: 36099891-36099913 | − | TCCTAATACCCCAGAGGGCCTGG |
| 977 | Chr1: 36099896-36099918 | − | GTTTTTCCTAATACCCCAGAGGG |
| 978 | Chr1: 36099897-36099919 | − | TGTTTTTCCTAATACCCCAGAGG |
| 979 | Chr1: 36099904-36099926 | + | GGTATTAGGAAAAACACTGAAGG |
| 980 | Chr1: 36099908-36099930 | + | TTAGGAAAAACACTGAAGGTAGG |
| 981 | Chr1: 36099916-36099938 | + | AACACTGAAGGTAGGAAAATTGG |
| 982 | Chr1: 36099919-36099941 | + | ACTGAAGGTAGGAAAATTGGTGG |
| 983 | Chr1: 36099920-36099942 | + | CTGAAGGTAGGAAAATTGGTGGG |
| 984 | Chr1: 36099921-36099943 | + | TGAAGGTAGGAAAATTGGTGGGG |
| 985 | Chr1: 36099928-36099950 | + | AGGAAAATTGGTGGGGAATGAGG |
| 986 | Chr1: 36099936-36099958 | + | TGGTGGGGAATGAGGAGCTGTGG |
| 987 | Chr1: 36099939-36099961 | + | TGGGGAATGAGGAGCTGTGGAGG |
| 988 | Chr1: 36099940-36099962 | + | GGGGAATGAGGAGCTGTGGAGGG |
| 989 | Chr1: 36099949-36099971 | + | GGAGCTGTGGAGGGCGCCTGAGG |
| 990 | Chr1: 36099958-36099980 | + | GAGGGCGCCTGAGGATCTGATGG |
| 991 | Chr1: 36099965-36099987 | − | CTGAGAGCCATCAGATCCTCAGG |
| 992 | Chr1: 36099966-36099988 | + | CTGAGGATCTGATGGCTCTCAGG |
| 993 | Chr1: 36099967-36099989 | + | TGAGGATCTGATGGCTCTCAGGG |
| 994 | Chr1: 36099970-36099992 | + | GGATCTGATGGCTCTCAGGGAGG |
| 995 | Chr1: 36099974-36099996 | + | CTGATGGCTCTCAGGGAGGCAGG |
| 996 | Chr1: 36099975-36099997 | + | TGATGGCTCTCAGGGAGGCAGGG |
| 997 | Chr1: 36099976-36099998 | + | GATGGCTCTCAGGGAGGCAGGGG |
| 998 | Chr1: 36099982-36100004 | + | TCTCAGGGAGGCAGGGGATTTGG |
| 999 | Chr1: 36099983-36100005 | + | CTCAGGGAGGCAGGGGATTTGGG |
| 1000 | Chr1: 36099984-36100006 | + | TCAGGGAGGCAGGGGATTTGGGG |
| 1001 | Chr1: 36099985-36100007 | + | CAGGGAGGCAGGGGATTTGGGGG |
| 1002 | Chr1: 36099989-36100011 | + | GAGGCAGGGGATTTGGGGGCTGG |
| 1003 | Chr1: 36099990-36100012 | + | AGGCAGGGGATTTGGGGGCTGGG |
| 1004 | Chr1: 36100002-36100024 | + | TGGGGGCTGGGAGCGATTTGAGG |
| 1005 | Chr1: 36100010-36100032 | + | GGGAGCGATTTGAGGCACTGTGG |
| 1006 | Chr1: 36100011-36100033 | + | GGAGCGATTTGAGGCACTGTGGG |
| 1007 | Chr1: 36100012-36100034 | + | GAGCGATTTGAGGCACTGTGGGG |
| 1008 | Chr1: 36100017-36100039 | + | ATTTGAGGCACTGTGGGGTGAGG |
| 1009 | Chr1: 36100020-36100042 | + | TGAGGCACTGTGGGGTGAGGAGG |
| 1010 | Chr1: 36100032-36100054 | + | GGGTGAGGAGGCTCTCACCCAGG |
| 1011 | Chr1: 36100038-36100060 | + | GGAGGCTCTCACCCAGGTACTGG |
| 1012 | Chr1: 36100049-36100071 | − | GAGGGCAAAGGCCAGTACCTGGG |
| 1013 | Chr1: 36100050-36100072 | − | TGAGGGCAAAGGCCAGTACCTGG |
| 1014 | Chr1: 36100053-36100075 | + | GGTACTGGCCTTTGCCCTCACGG |
| 1015 | Chr1: 36100057-36100079 | + | CTGGCCTTTGCCCTCACGGAAGG |
| 1016 | Chr1: 36100058-36100080 | + | TGGCCTTTGCCCTCACGGAAGGG |
| 1017 | Chr1: 36100061-36100083 | + | CCTTTGCCCTCACGGAAGGGCGG |
| 1018 | Chr1: 36100061-36100083 | − | CCGCCCTTCCGTGAGGGCAAAGG |
| 1019 | Chr1: 36100067-36100089 | − | GTGGGACCGCCCTTCCGTGAGGG |
| 1020 | Chr1: 36100068-36100090 | − | TGTGGGACCGCCCTTCCGTGAGG |
| 1021 | Chr1: 36100070-36100092 | + | TCACGGAAGGGCGGTCCCACAGG |
| 1022 | Chr1: 36100084-36100106 | + | TCCCACAGGTCCTTTCTGCATGG |
| 1023 | Chr1: 36100085-36100107 | + | CCCACAGGTCCTTTCTGCATGGG |
| 1024 | Chr1: 36100085-36100107 | − | CCCATGCAGAAAGGACCTGTGGG |
| 1025 | Chr1: 36100086-36100108 | − | GCCCATGCAGAAAGGACCTGTGG |
| 1026 | Chr1: 36100089-36100111 | + | CAGGTCCTTTCTGCATGGGCTGG |
| 1027 | Chr1: 36100094-36100116 | − | TACATCCAGCCCATGCAGAAAGG |
| 1028 | Chr1: 36100103-36100125 | + | ATGGGCTGGATGTACTTCACTGG |
| 1029 | Chr1: 36100104-36100126 | + | TGGGCTGGATGTACTTCACTGGG |
| 1030 | Chr1: 36100105-36100127 | + | GGGCTGGATGTACTTCACTGGGG |
| 1031 | Chr1: 36100126-36100148 | + | GGCATAGCCCGCCGCCCCACCGG |
| 1032 | Chr1: 36100133-36100155 | − | GGCGGGGCCGGTGGGGCGGCGGG |
| 1033 | Chr1: 36100134-36100156 | − | TGGCGGGGCCGGTGGGGCGGCGG |
| 1034 | Chr1: 36100137-36100159 | − | TGGTGGCGGGGCCGGTGGGGCGG |
| 1035 | Chr1: 36100140-36100162 | − | CTCTGGTGGCGGGGCCGGTGGGG |
| 1036 | Chr1: 36100141-36100163 | + | CCCACCGGCCCCGCCACCAGAGG |
| 1037 | Chr1: 36100141-36100163 | − | CCTCTGGTGGCGGGGCCGGTGGG |
| 1038 | Chr1: 36100142-36100164 | − | TCCTCTGGTGGCGGGGCCGGTGG |
| 1039 | Chr1: 36100145-36100167 | − | GCGTCCTCTGGTGGCGGGGCCGG |
| 1040 | Chr1: 36100149-36100171 | − | GCGGGCGTCCTCTGGTGGCGGGG |
| 1041 | Chr1: 36100150-36100172 | − | CGCGGGCGTCCTCTGGTGGCGGG |
| 1042 | Chr1: 36100151-36100173 | + | CCGCCACCAGAGGACGCCCGCGG |
| 1043 | Chr1: 36100151-36100173 | − | CCGCGGGCGTCCTCTGGTGGCGG |
| 1044 | Chr1: 36100154-36100176 | − | GGGCCGCGGGCGTCCTCTGGTGG |
| 1045 | Chr1: 36100157-36100179 | − | TGTGGGCCGCGGGCGTCCTCTGG |
| 1046 | Chr1: 36100167-36100189 | − | GGTGCTGGGGTGTGGGCCGCGGG |
| 1047 | Chr1: 36100168-36100190 | − | TGGTGCTGGGGTGTGGGCCGCGG |
| 1048 | Chr1: 36100174-36100196 | − | TGGTGCTGGTGCTGGGGTGTGGG |
| 1049 | Chr1: 36100175-36100197 | − | CTGGTGCTGGTGCTGGGGTGTGG |
| 1050 | Chr1: 36100180-36100202 | − | TGCTACTGGTGCTGGTGCTGGGG |
| 1051 | Chr1: 36100181-36100203 | − | CTGCTACTGGTGCTGGTGCTGGG |
| 1052 | Chr1: 36100182-36100204 | − | GCTGCTACTGGTGCTGGTGCTGG |
| 1053 | Chr1: 36100188-36100210 | − | GCTGCTGCTGCTACTGGTGCTGG |
| 1054 | Chr1: 36100194-36100216 | − | TTCGCTGCTGCTGCTGCTACTGG |
| 1055 | Chr1: 36100200-36100222 | + | GCAGCAGCAGCAGCGAAGACAGG |
| 1056 | Chr1: 36100201-36100223 | + | CAGCAGCAGCAGCGAAGACAGGG |
| 1057 | Chr1: 36100202-36100224 | + | AGCAGCAGCAGCGAAGACAGGGG |
| 1058 | Chr1: 36100222-36100244 | + | GGGTGTCAGAGTCCCCAGCATGG |
| 1059 | Chr1: 36100231-36100253 | + | AGTCCCCAGCATGGCGTCCGTGG |
| 1060 | Chr1: 36100234-36100256 | − | CGTCCACGGACGCCATGCTGGGG |
| 1061 | Chr1: 36100235-36100257 | − | ACGTCCACGGACGCCATGCTGGG |
| 1062 | Chr1: 36100236-36100258 | − | CACGTCCACGGACGCCATGCTGG |
| 1063 | Chr1: 36100248-36100270 | − | TCTTCTTTGCAGCACGTCCACGG |

Use of gRNAs comprising guide sequences complementary to SEQ ID NOs: 191-1063, or that bind the reverse compliment of SEQ ID NOs: 191-1063 would be expected to target an nuclease (e.g., Cas9 or Cas9 RNP) to sequences of COL8A2. As heterozygous mutants of COL8A2 have been characterized in early-onset FECD, targeting a Cas RNP with a gRNA comprising a guide sequence complementary to a target sequence of SEQ ID NOs: 191-1063 could lead to the creation of indels via NHEJ. The generation of indels could decrease the expression of COL8A2, thereby decreasing the resulting toxic alpha-2 subunit of the collagen-8 protein. A decrease in the toxic COL8A2 product may improve the disease course of early-onset FECD, as other forms of collagen may take the place of the alpha-2 subunit. Certain guides may also be useful for excising the region of the COL8A2 gene that contains known disease-associated mutations, or changing the splicing pattern to favor isoforms that do not contain such mutations. Knockout of the COL8A2 gene using certain guides could also be used in conjunction with a wild type COL8A2 replacement strategy. For example the wild type COL8A2 coding sequence could be expressed via transgenic means, after removing expression of the endogenous, dominant-negative mutant form.

Based on the differences in nucleotide sequences for the mutant alleles, target sequences specific to the mutant alleles were also identified.

Table 4 lists target sequences specific for mutations leading to Gln455Lys, caused by the c. 1364C>A nucleotide change. Use of gRNA comprising guide sequences complementary to SEQ ID NOs: 1064-1069 would target to the mutant allele, while not targeting or targeting less efficiently to the wild type allele. As individuals with the Gln455Lys mutation usually have only one affected allele, selective generation of indels due to NHEJ mediated by a Cas RNP targeted to the mutant allele of COL8A2 would be expected to only cause loss of this allele while preserving the other wild type COL8A2 allele. Alternatively, a gRNA comprising guide sequences complementary to SEQ ID NOs: 1064-1069, or guide sequences that bind to the reverse compliment of SEQ ID NOs: 1064-1069 also could be used together with a template to mediate correction of the mutation.

TABLE 4
Target sequences for COL8A2 with Gln455Lys Mutation

| | Target | Target | |
|---|---|---|---|
| SEQ ID No | Location | Strand | Target Sequence |
| 1064 | Chr1: 36098302-36098324 | + | CCCCTCAGGCCAGGCTTCCCAGG |
| 1065 | Chr1: 36098302-36098324 | − | CCTGGGAAGCCTGGCCTGAGGGG |
| 1066 | Chr1: 36098303-36098325 | + | CCCTCAGGCCAGGTTGCCCAGGG |
| 1067 | Chr1: 36098303-36098325 | − | CCCTGGGAAGCCTGGCCTGAGGG |
| 1068 | Chr1: 36098304-36098326 | − | TCCCTGGGAAGCCTGGCCTGAGG |
| 1069 | Chr1: 36098311-36098333 | − | TTGGGGCTCCCTGGGAAGCCTGG |

Table 5 lists target sequences specific for a point mutation leading to Gln455Val, caused by the c. 1363-1364CA>GT nucleotide changes. Use of gRNA comprising guide sequences that directs a nuclease to SEQ ID NOs: 1070-1075 would target to the mutant allele, while not targeting or targeting less efficiently to the wild type allele. As individuals with the Gln455Val mutation usually have only one affected allele, selective generation of indels due to NHEJ mediated by a nuclease (e.g., Cas RNP) targeted to the mutant allele of COL8A2 would be expected to only cause loss of this allele while preserving the other wild type COL8A2 allele. Alternatively, a gRNA comprising guide sequences complementary to SEQ ID NOs: 1070-1075 also could be used together with a template to mediate correction of the mutation.

TABLE 5
Target sequences for COL8A2 with Gln455Val Mutation

| SEQ ID | Target | Target | |
|---|---|---|---|
| No | Location | Strand | Target Sequence |
| 1070 | Chr1: 36098302-36098324 | + | CCCCTCAGGCCAGGCACCCCAGG |
| 1071 | Chr1: 36098302-36098324 | − | CCTGGGGTGCCTGGCCTGAGGGG |
| 1072 | Chr1: 36098303-36098325 | + | CCCTCAGGCCAGGCACCCCAGGG |
| 1073 | Chr1: 36098303-36098325 | − | CCCTGGGGTGCCTGGCCTGAGGG |
| 1074 | Chr1: 36098304-36098326 | − | TCCCTGGGGTGCCTGGCCTGAGG |
| 1075 | Chr1: 36098311-36098333 | − | TTGGGGCTCCCTGGGGTGCCTGG |

Table 6 lists target sequences specific for a point mutation leading to Leu450Trp, caused by the c. 1349T>G nucleotide change. Use of gRNA comprising guide sequences complementary to SEQ ID NOs: 1076-1084 would target to the mutant allele, while not targeting or targeting less efficiently to the wild type allele. As individuals with the Leu450Trp mutation usually have only one affected allele, selective generation of indels due to NHEJ mediated by a Cas RNP targeted to the mutant allele of COL8A2 would be expected to only cause loss of this allele while preserving the other wild type COL8A2 allele. Alternatively, a gRNA comprising guide sequences complementary to SEQ ID NOs: 1076-1084 also could be used together with a template to mediate correction of the mutation.

TABLE 6
Target sequences for COL8A2 with Leu450Trp Mutation

| SEQ ID | | Target | |
|---|---|---|---|
| No | Target Location | Strand | Target Sequence |
| 1076 | Chr1: 36098311-36098333 | − | TGGGGGCTCCCTGGGCAGCCTGG |
| 1077 | Chr1: 36098319-36098341 | − | AAGGTGACTGGGGGCTCCCTGGG |
| 1078 | Chr1: 36098320-36098342 | − | AAAGGTGACTGGGGGCTCCCTGG |
| 1079 | Chr1: 36098328-36098350 | − | TGGGGCAGAAAGGTGACTGGGGG |
| 1080 | Chr1: 36098329-36098351 | − | CTGGGGCAGAAAGGTGACTGGGG |
| 1081 | Chr1: 36098330-36098352 | + | CCCAGTCACCTTTCTGCCCCAGG |
| 1082 | Chr1: 36098330-36098352 | − | CCTGGGGCAGAAAGGTGACTGGG |
| 1083 | Chr1: 36098331-36098353 | + | CCAGTCACCTTTCTGCCCCAGGG |
| 1084 | Chr1: 36098331-36098353 | − | CCCTGGGGCAGAAAGGTGACTGG |

A template could be used together with a Cas RNP to correct a nucleotide mutation that leads to generation of collagen VIII with either a Gln455Lys, Gln455Val, or Leu450Trp mutation. In this way, the Cas RNP could target to the mutation, initiate NHEJ, and then mediate correction of the mutation based on an exogenous template. Targeting of a Cas RNP to correct mutations leading to expression of a Gln455Lys product could be done using a gRNA comprising a guide sequence complementary to a target sequence of SEQ ID NOs: 1064-1069 together with a template. Targeting of a Cas RNP to correct mutations leading to expression of a Gln455Val product could be done using a gRNA comprising a guide sequence complementary to a target sequence of SEQ ID NOs: 1070-1075 together with a template. Targeting of a Cas RNP to correct mutations leading to expression of a Leu450Trp gene product could be done using a gRNA comprising a guide sequence complementary to a target sequence of SEQ ID NOs: 1076-1084 together with a template. In this manner, selective editing of the mutant allele could be performed to correct defective collagen VIII caused by either Gln455Lys, Gln455Val, or Leu450Trp.

Thus, use of Cas RNP comprising gRNAs comprising guide sequences complementary to target sequences of COL842 may be novel means to treat FECD or PPCD. Target sequences include those to wild type COL8A2 as well as target sequences specific to mutations that can cause a mutant allele of COL8A2 and lead to gene products with Gln455Lys, Gln455Val, or Leu450Trp mutations. Mutation-specific target sequences listed in Tables 4, 5, and 6 can be used to develop guide RNAs for use with Cas (e.g., in Cas RNPs) with specificity for introducing further mutations in the mutant allele to eliminate its function or, alternatively, to use together with a template to correct the causative nucleotide mutation in COL8A2.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

1. A composition comprising at least one guide RNA comprising a guide sequence that directs a nuclease to a target sequence selected from SEQ ID NOs: 1-1084.

2. A composition comprising at least one guide RNA comprising a guide sequence that is identical or at least 99%, 98%, 97%, 96%, 95%, 94%, 93%, 92%, 91%, or 90% identical to a sequence selected from SEQ ID NOs: 1089-1278.

3. (canceled)

4. The composition of claim 1, wherein the guide RNA targets a sequence at or near a tri-nucleotide repeat (TNR) in the transcription factor four (TCF4) gene, and directs a nuclease to a target sequence selected from SEQ ID NOs: 1-190.

5. The composition of claim 4 comprising at least one guide RNA comprising a guide sequence that is at least 99%, 98%, 97%, 96%, 95%, 94%, 93%, 92%, 91%, or 90% identical to a sequence selected from SEQ ID NOs: 1089-1278.

6. A composition comprising two guide RNAs selected from the following guide RNA pairings:
a. a first guide RNA that directs a nuclease to SEQ ID NO: 83, and a second guide RNA that directs a nuclease to SEQ ID NO: 109;
b. a first guide RNA that directs a nuclease to SEQ ID NO: 85, and a second guide RNA that directs a nuclease to SEQ ID NO: 109;
c. a first guide RNA that directs a nuclease to SEQ ID NO: 86, and a second guide RNA that directs a nuclease to SEQ ID NO: 112;
d. a first guide RNA that directs a nuclease to SEQ ID NO: 85, and a second guide RNA that directs a nuclease to SEQ ID NO: 125;
e. a first guide RNA that directs a nuclease to SEQ ID NO: 86, and a second guide RNA that directs a nuclease to SEQ ID NO: 109;
f. a first guide RNA that directs a nuclease to SEQ ID NO: 85, and a second guide RNA that directs a nuclease to SEQ ID NO: 107;
g. a first guide RNA that directs a nuclease to SEQ ID NO: 83, and a second guide RNA that directs a nuclease to SEQ ID NO: 125;
h. a first guide RNA that directs a nuclease to SEQ ID NO: 86, and a second guide RNA that directs a nuclease to SEQ ID NO: 125;
i. a first guide RNA that directs a nuclease to SEQ ID NO: 86, and a second guide RNA that directs a nuclease to SEQ ID NO: 107;
j. a first guide RNA that directs a nuclease to SEQ ID NO: 64, and a second guide RNA that directs a nuclease to SEQ ID NO: 106;
k. a first guide RNA that directs a nuclease to SEQ ID NO: 85, and a second guide RNA that directs a nuclease to SEQ ID NO: 114;
l. a first guide RNA that directs a nuclease to SEQ ID NO: 86, and a second guide RNA that directs a nuclease to SEQ ID NO: 114;
m. a first guide RNA that directs a nuclease to SEQ ID NO: 83, and a second guide RNA that directs a nuclease to SEQ ID NO: 114;
n. a first guide RNA that directs a nuclease to SEQ ID NO: 53, and a second guide RNA that directs a nuclease to SEQ ID NO: 114;
o. a first guide RNA that directs a nuclease to SEQ ID NO: 83, and a second guide RNA that directs a nuclease to SEQ ID NO: 112; and
p. a first guide RNA that directs a nuclease to SEQ ID NO: 74, and a second guide RNA that directs a nuclease to SEQ ID NO: 114.

7. The composition of claim 6,
wherein the first guide RNA that directs a nuclease to SEQ ID NO: 83 comprises SEQ ID NO: 1171, and the second guide RNA that directs a nuclease to SEQ ID NO: 109 comprises SEQ ID NO: 1197; or
wherein the first guide RNA that directs a nuclease to SEQ ID NO: 85 comprises SEQ ID NO: 1173, and the second guide RNA that directs a nuclease to SEQ ID NO: 109 comprises SEQ ID NO: 1197; or
wherein the first guide RNA that directs a nuclease to SEQ ID NO: 86 comprises SEQ ID NO: 1174, and the second guide RNA that directs a nuclease to SEQ ID NO: 112 comprises SEQ ID NO: 1200; or
wherein the first guide RNA that directs a nuclease to SEQ ID NO: 85 comprises SEQ ID NO: 1173, and the second guide RNA that directs a nuclease to SEQ ID NO: 125 comprises SEQ ID NO: 1213; or
wherein the first guide RNA that directs a nuclease to SEQ ID NO: 86 comprises SEQ ID NO: 1174, and the second guide RNA that directs a nuclease to SEQ ID NO: 109 comprises SEQ ID NO: 1197; or
wherein the first guide RNA that directs a nuclease to SEQ ID NO: 85 comprises SEQ ID NO: 1173, and the second guide RNA that directs a nuclease to SEQ ID NO: 107 comprises SEQ ID NO: 1195; or
wherein the first guide RNA that directs a nuclease to SEQ ID NO: 83 comprises SEQ ID NO: 1171, and the second guide RNA that directs a nuclease to SEQ ID NO: 125 comprises SEQ ID NO: 1213; or
wherein the first guide RNA that directs a nuclease to SEQ ID NO: 86 comprises SEQ ID NO: 1174, and the second guide RNA that directs a nuclease to SEQ ID NO: 125 comprises SEQ ID NO: 1213; or
wherein the first guide RNA that directs a nuclease to SEQ ID NO: 86 comprises SEQ ID NO: 1174, and the second guide RNA that directs a nuclease to SEQ ID NO: 107 comprises SEQ ID NO: 1195; or
wherein the first guide RNA that directs a nuclease to SEQ ID NO: 64 comprises SEQ ID NO: 1152, and the second guide RNA that directs a nuclease to SEQ ID NO: 106 comprises SEQ ID NO: 1194; or
wherein the first guide RNA that directs a nuclease to SEQ ID NO: 85 comprises SEQ ID NO: 1173, and the second guide RNA that directs a nuclease to SEQ ID NO: 114 comprises SEQ ID NO: 1202; or,
wherein the first guide RNA that directs a nuclease to SEQ ID NO: 86 comprises SEQ ID NO: 1174, and the second guide RNA that directs a nuclease to SEQ ID NO: 114 comprises SEQ ID NO: 1202; or
wherein the first guide RNA that directs a nuclease to SEQ ID NO: 83 comprises SEQ ID NO: 1171, and the second guide RNA that directs a nuclease to SEQ ID NO: 114 comprises SEQ ID NO: 1202; or
wherein the first guide RNA that directs a nuclease to SEQ ID NO: 53 comprises SEQ ID NO: 1141, and the second guide RNA that directs a nuclease to SEQ ID NO: 114 comprises SEQ ID NO: 1202; or
wherein the first guide RNA that directs a nuclease to SEQ ID NO: 83 comprises SEQ ID NO: 1171, and the second guide RNA that directs a nuclease to SEQ ID NO: 112 comprises SEQ ID NO: 1200; or
wherein the first guide RNA that directs a nuclease to SEQ ID NO: 74 comprises SEQ ID NO: 1162, and the second guide RNA that directs a nuclease to SEQ ID NO: 114 comprises SEQ ID NO: 1202.

8-22. (canceled)

23. The composition of claim 1,
wherein the guide RNA targets the alpha 2 subunit of collagen type VIII (Col8A2) gene, and directs a nuclease to a target sequence selected from SEQ ID NOs: 191-1063, or wherein the composition comprises a guide sequence that is at least 99%, 98%, 97%, 96%, 95%, 94%, 93%, 92%, 91%, or 90% identical to a sequence complementary, or identical, to the first 20 nucleotides of a target sequence selected from SEQ ID NOs: 191-1063, wherein the thymines in the first 20 nucleotides of SEQ ID NOs: 191-1063 are replaced with uracil.

24. (canceled)

25. The composition of claim 1, wherein the guide RNA targets the Gln455Lys mutation in the Col8A2 gene product, and directs a nuclease to a target sequence selected from SEQ ID NOs: 1064-1069, or wherein the composition comprises at least one guide RNA comprising a guide sequence that is at least 99%, 98%, 97%, 96%, 95%, 94%, 93%, 92%, 91%, or 90% identical to a sequence complementary, or identical, to the first 20 nucleotides of a target sequence selected from SEQ ID NOs: 1064-1069, wherein the thymines in the first 20 nucleotides of SEQ ID NOs: 1064-1069 are replaced with uracil.

26. (canceled)

27. The composition of claim 1, wherein the guide RNA targets the Gln455Val mutation in the Col8A2 gene product, and directs a nuclease to a target sequence selected from SEQ ID NOs: 1070-1075, or wherein the composition comprises at least one guide RNA comprising a guide sequence that is at least 99%, 98%, 97%, 96%, 95%, 94%, 93%, 92%, 91%, or 90% identical to a sequence complementary, or identical, to the first 20 nucleotides of a target sequence selected from SEQ ID NOs: 1070-1075, wherein the thymines in the first 20 nucleotides of SEQ ID NOs: 1070-1075 are replaced with uracil.

28. (canceled)

29. The composition of claim 1, wherein the guide RNA targets the Leu450Trp mutation in the Col842 gene product, and directs a nuclease to a target sequence selected from SEQ ID NOs: 1076-1084, or wherein the composition comprises at least one guide RNA comprising a guide sequence that is at least 99%, 98%, 97%, 96%, 95%, 94%, 93%, 92%, 91%, or 90% identical to a sequence complementary, or identical, to the first 20 nucleotides of a target sequence selected from SEQ ID NOs: 1076-1084, wherein the thymines in the first 20 nucleotides of SEQ ID NOs: 1076-1084 are replaced with uracil.

30. (canceled)

31. The composition of claim 1, wherein the guide RNA is a dual guide or a single guide.

32. (canceled)

33. The composition of claim 1, wherein at least one guide RNA comprises a crRNA, a trRNA, or a crRNA and a trRNA.

34. The composition of claim 1, wherein at least one guide sequence is encoded on a vector.

35-39. (canceled)

40. The composition of claim 1, wherein a first guide sequence and second guide sequence are complementary to a first target sequence and a second target sequence in opposite strands of a target gene.

41. (canceled)

42. The composition of claim 1, further comprising a nuclease.

43. The composition of claim 42, wherein the nuclease is a Cas protein.

44-49. (canceled)

50. A pharmaceutical formulation comprising the composition of claim 1, and a pharmaceutically acceptable carrier.

51. A method of excising at least a portion of a trinucleotide repeat (TNR) in the transcription factor four (TCF4) gene in a human subject, comprising administering the composition of claim 6.

52. The method of claim 51, wherein two guide RNA are used, wherein the first directs a nuclease to a sequence 5′ of the TNR and the second directs a nuclease to a sequence 3′ of the TNR.

53. The method of claim 51, wherein the human subject has Fuchs endothelial corneal dystrophy (FECD).

54. (canceled)

55. The method of claim 51, wherein the subject has an improvement, stabilization, or slowing of decline in visual acuity as a result of administration.

56-66. (canceled)

67. A method of decreasing expression of a mutant allele of the COL8A2 gene, such as Gln455Lys, Gln455Val, or Leu450Trp, or altering the nucleotide sequence to correct said mutant allele in a human subject, comprising administering the composition of claim 1.

68-73. (canceled)

74. The method of claim 67, wherein the mutation leading to expression of a Gln455Lys, Gln455Val or a Leu450Trp gene product is c.1364C>A, c. 1363-1364CA>GT, or c. 1349T>G, respectively.

75-79. (canceled)
